(19) United States
(12) Patent Application Publication
(10) Pub. No.: US 2011/0152345 A1
(43) Pub. Date: Jun. 23, 2011

(54) EBI3, DLX5, NPTX1 AND CDKN3 FOR TARGET GENES OF LUNG CANCER THERAPY AND DIAGNOSIS

(75) Inventors: Yusuke Nakamura, Tokyo (JP); Yataro Daigo, Tokyo (JP); Shuichi Nakatsuru, Kanagawa (JP)

(73) Assignee: Oncotherapy Science, Inc., Kanagawa (JP)

(21) Appl. No.: 12/674,759

(22) PCT Filed: Aug. 21, 2008
(86) PCT No.: PCT/JP2008/065352

§ 371 Date: Sep. 17, 2010

Related U.S. Application Data
(60) Provisional application No. 60/957,956, filed on Aug. 24, 2007.
(60) Provisional application No. 60/977,360, filed on Oct. 3, 2007.

(51) Int. Cl. A61K 31/713; C07H 21/00; C12N 15/63; C12Q 1/68; C12Q 1/02; G01N 33/68; G01N 33/53; C12Q 1/42; A61P 35/00

(52) U.S. Cl. 514/44 A; 536/24.5; 435/320.1; 435/6.1; 435/6.19; 435/29; 436/86; 435/7.92; 436/501; 435/21; 435/6.13

(57) ABSTRACT

The present invention relates to methods for treating or preventing lung cancer by administering a double-stranded molecule against one or more of EBI3, DLX5, NPTX1, CDKN3 or EF-I delta genes or compositions, vectors or cells containing such a double-stranded molecule. The present invention also features methods for diagnosing lung cancer, especially NSCLC or SCLC, using one or more over-expressed genes selected from among EBI3, DLX5, NPTX1, CDKN3 and/or EF-I delta. Also disclosed are methods of identifying compounds for treating and preventing lung cancer, using as an index their effect on the over-expression of one or more of EBI3, DLX5, CDKN3 and/or EF-I delta in the lung cancer, the cell proliferation function of one or more of EBI3, DLX5, NPTX1, CDKN3 and/or EF-I delta or the interaction between CDKN3 and VRS, EF-I beta, EF-I gamma and/or EF-I delta.

### CROSS-REFERENCE TO RELATED APPLICATIONS

This application claims the benefit of U.S. Provisional Application Ser. No. 60/957,956 filed Aug. 24, 2007, and Ser. No. 60/977,360 filed Oct. 3, 2007, the contents of which are hereby incorporated by reference in their entirety.

### TECHNICAL FIELD

The present invention relates to the field of biological science, more specifically to the field of cancer research, cancer diagnosis and cancer therapy. In particular, the present invention relates to methods for detecting and diagnosing lung cancer as well as methods for treating and preventing lung cancer. Moreover, the present invention relates to methods for screening an agent for treating and/or preventing lung cancer.

### BACKGROUND ART

Lung cancer is one of the most common causes of cancer death worldwide, and non-small cell lung cancer (NSCLC) accounts for nearly 80% of those cases (Greenlee, R. T., et al., CA. Cancer J. Clin. 51: 15-36 (2001)). Because the majority of NSCLCs are not diagnosed until advanced stages, it tends to be a fatal diagnosis, with an overall ten-year survival rate hovering around 10% despite recent advances in multi-modality therapy. Even the most innovative therapeutic regimens have only minor effect on outcome, increasing the overall 5-year survival rates for NSCLC to only 10-15%. Although many genetic alterations associated with development and progression of lung cancer have been reported, the precise molecular mechanisms remain unclear (Sozzi, G. Eur. J. Cancer 37: 63-73 (2001)). Therefore, a better understanding of the molecular pathogenesis of lung cancer is an urgent issue in order to develop effective diagnostic approaches and molecular-targeted therapies.

Over the last decade, newly developed cytotoxic agents such as paclitaxel, docetaxel, gemcitabine, and vinorelbine have emerged to offer multiple therapeutic choices for patients with advanced NSCLC; however, each of the new regimens can provide only modest survival benefits compared with cisplatin-based therapies (Kelly, K., et al., J. Clin. Oncol. 19: 3210-3218 (2001)). Recently, molecular-targeted agents, including anti-EGFR or anti-VEGF monoclonal antibody, cetuximab (Erbitux) or Bevacizumab (Avastin), and small molecule inhibitors of EGFR tyrosine kinase, such as gefitinib (Iressa) and erlotinib (Tarceva), have been examined and/or approved for clinical use (Giaccone, G. J Clin Oncol. 23: 3235-3242 (2005); Sridhar, S. S., Lancet Oncol. 4: 397-406 (2003); Pal, S. K. and Pegram, M. Anticancer Drugs 16: 483-494 (2005)). While these agents display to a certain extent activity against recurrent NSCLC, the number of patients who could receive a survival benefit is still limited. As for diagnosis, several tumor markers for lung cancer, including NSE, CEA, CYFRA21-1, and ProGRP, are presently used in clinical setting (M. Seike, G. A. Chen, B. K. Shin); however, their usefulness in early detection of cancers and prediction of clinical outcome is still very limited, mainly due to the low sensitivity and/or specificity. Therefore, the discovery of highly sensitive and specific cancer biomarkers that can assist clinicians in diagnosis and monitoring of the disease is urgently required. Hence, new therapeutic strategies, such as development of more selective and effective molecular-targeted agents and markers, are eagerly awaited.

Some evidence suggests that tumor cells express cell-surface and/or secretory markers unique to each histological type at particular stages of differentiation. Since cell-surface and secretory proteins are considered more accessible to immune mechanisms and drug-delivery systems, identification of these types of proteins is an important initial step in the development of novel diagnostic and therapeutic strategies. Furthermore, the systematic analysis of expression levels of thousands of genes on cDNA microarrays is an effective approach to identify unknown molecules involved in pathways of carcinogenesis, and can therefore reveal candidate targets for development of novel anti-cancer drugs and tumor biomarkers (Kikuchi, T., et al., Oncogene 22: 2192-2205 (2003); Kikuchi, T., et al., Int J Oncol. 28: 799-805 (2006); Kakiuchi, S., et al., Mol Cancer Res. 1: 485-499 (2003); Kakiuchi, S., et al., Hum Mol Genet. 13: 3029-3043 (2004); Taniwaki M., et al., Int J Oncol. 29: 567-575 (2006); Yamabuki. T., et al., Int J Oncol. 28: 1375-1384 (2006)). The present inventors have been attempting to isolate novel molecular targets for diagnosis, treatment and prevention of lung cancer by analyzing genome-wide expression profiles of various types of lung cancer cells on a cDNA microarray containing 27,648 genes, using pure populations of tumor cells prepared from 101 lung cancer tissues by laser microdissection (Kikuchi, T., et al., Oncogene 22: 2192-2205 (2003); Kikuchi, T., et al., Int J Oncol. 28: 799-805 (2006); Kakiuchi, S., et al., Hum Mol Genet. 13: 3029-3043 (2004); Taniwaki M., et al., Int J Oncol. 29: 567-575 (2006)). To verify the biological and clinicopathological significance of the respective gene products, the present inventors have been performing a combination assay of the tumor-tissue microarray analysis of clinical lung-cancer materials with RNA interference (RNAi) technique (Ishikawa, N., et al., Clin Cancer Res. 10: 8363-8370 (2004); Ishikawa, N., et al., Cancer Res. 65: 9176-9184 (2005); Ishikawa, N., et al., Cancer Sci. 97: 737-745 (2006); Kato, T., et al., Cancer Res. 65: 5638-5646 (2005); Kato T, et al., Clin. Cancer Res. 13: 434-442. (2007); Furukawa, C., et al., Cancer Res. 65: 7102-7110 (2005); Suzuki, C., Cancer Res. 63: 7038-7041 (2003); Suzuki, C., Cancer Res. 65: 11314-11325 (2005); Suzuki, C., et al., Mol Cancer Ther. 6: 542-551 (2007); Takahashi K, et al., Cancer Res. 66: 9408-9419 (2006); Hayama, S., et al., Cancer Res. 66: 10339-10348 (2006); Hayama S, et al., Cancer Res. 67: 4113-4122 (2007); Yamabuki T, et al., Cancer Res. 67: 2517-2525 (2007)).

From this systematic approach, a number of genes have been identified as overexpressed in certain cancers. See, for example, WO 2004/31413, WO 2004/31409, WO 2007/13665 and WO/2007/13671, the contents of which are incorporated by reference herein. Herein, the present inventors focused on four genes for further investigation; an Epstein-Barr virus induced gene 3 (EBI3) (SEQ ID NO 1; GenBank accession number: NM_{—}005755); a secreted glycoprotein, distal-less homeobox 5 (DLX5) (SEQ ID NO 3; GenBank accession number: BC006226); cyclin-dependent kinase inhibitor 3 (CDKN3; alias KAP1) (SEQ ID NO 5; GenBank accession number: L27711); and Neuronal pentraxin I (NPTX1) (SEQ ID NO 78; GenBank accession number: NM_{—}002522.2 or GenBank accession number: NM_{—}002522).

The expression of the EBI3 gene was first noted in B cell lines transformed in vitro by EBV (Devergne O, et al., J Virol 70: 1143-1153 (1996)). EBI3 is a component of IL-27, formed by heterodimerizing with p28, an IL-12 p35-related subunit (Pflanz S, et al., Immunity 16: 779-90 (2002)). IL-27 is believed to play an important role in the Th1 immunoresponse initiation that is necessary for the immune response induced by IFN-gamma. On the other hand, a recent report has suggested that EBI3 expression is found in extravillous cytotrophoblasts of placenta during human pregnancy (Devergne O, et al., Am J Pathol 159: 1763-76 (2001)) and that EBI3 may modulate maternal-placental immune relationship, such as maternal immunotolerance. While the overexpression of EBI3 in human hematologic malignancy was recently reported (Larousserie, F., et al., Am J Pathol. 166: 1217-1228 (2005), Niedobitek G, et al., J Pathol 198: 310-316 (2002)), its functional role in these tumors and the involvement of EBI3 in human solid tumorigenesis has not yet reported.

Homeobox genes are transcription factors of fundamental importance associated with development throughout evolutionarily diverse species. The redundant function of the Dlx genes is presumed to result from their nearly identical homeodomains, whereas their individual unique functions are presumed to arise from the divergence of their amino acid sequences in other domains (Liu J K, et al., Dev Dyn 210: 498-512 (1997)). Inactivation of homeobox genes has been implicated in many congenital malformations as well as the development of cancers (Downing J R, et al., Cancer Cell 2: 437-45 (2002)). DLX5 is considered to be a master regulatory protein essential in initiation of the cascade involved in osteoblast differentiation and to play a critical role in regulation of mammalian limb development, as demonstrated by the evidence that the targeted disruption or ablation of Dlx5 and Dlx6 results in developmental abnormalities of bone and inner ear, and craniofacial defects (Robledo R F, et al., Genes Dev 16: 1089-101 (2002)). However, the role of DLX5 activation in carcinogenesis has not been elucidated.

NPTX1 is a member of a newly recognized subfamily of “long pentraxin” (Goodman). The NPTX1 gene encodes a secretory protein of 430 amino acids with a N-terminal signal peptide and C-terminal pentraxin domain. NPTX1 was identified as a rat protein that may mediate the uptake of synaptic material and the presynaptic snake venom toxin, taipoxin. The “long pentraxins”, a newly recognized subfamily of proteins, have several structural and functional characteristics that may play a role in promoting exciatory synapse formation and synaptic remodeling (Schlimgen; Kirkpatrick). Members of this subfamily include NPTX1 and NPTX2, both of which interact with neuronal pentraxin receptor (NPTXR) (Schlimgen; Kirkpatrick; Goodman; Dodds), and have superadditive synaptogenic activity. Further, the present inventor has revealed that NPTX1 can be used for serological marker or prognostic marker for lung cancer (WO2008/23840). However, the role of “long pentraxins” during carcinogenesis and its function in mammalian cells have not been elucidated.

CDKN3 was first identified as a G1 and S phase dual-specificity protein phosphatase that associates with cdk2 and/or cdc2 and is involved in cell cycle regulation (Gyuris, J., et al., Cell 75: 791-803 (1993); Hannon, G. J., et al., Proc Natl Acad Sci USA. 91: 1731-1735 (1994)). Full activation of cdk2 requires phosphorylation of Thr160 and dephosphorylation of Thr14 and Tyr15. The binding of cyclin A to cdk2 inhibited the dephosphorylation of Thr160, but CDKN3 can only dephosphorylate cdk2 when cyclin A is degraded or dissociated (Poon R Y and Hunter T., Science 270: 90-93 (1995)). Although previous reports suggest a functional role of CDKN3 in cell cycle control, its contribution to cell proliferation has not yet been reported. While CDKN3 overexpression has previously been reported in breast and prostate cancer (Lee, S. W., et al., Mol Cell Biol. 20: 1723-1732 (2000)), the mechanism by which CDKN3 overexpression promotes the lung cancer progression remains unclear.

On the other hand, eukaryotic translation elongation factor 1 delta (EF-1delta) (SEQ ID NO 7; GenBank accession number: BC009907) is a component of the elongation factor-1 complex that constitutes a group of nucleotide exchange proteins that could bind guanosine 5′-triphosphate (GTP) and aminoacyl-tRNA and result in codon-dependent placement of aminoacyl-tRNA on 80S ribosomes, inducing peptide chain elongation of protein synthesis (Riis, B., et al., Trends Biochem Sci. 15: 420-424 (1990); Proud, C. G. Mol Biol Rep. 19: 161-170 (1994)). EF-1delta has also been identified and characterized as a cadmium-responsive proto-oncogene (Joseph P., et al., J Biol Chem. 277: 6131-6136 (2002)). Recent reports indicate that EF-1delta mRNA is overexpressed in esophageal carcinoma tissues, and is correlated with lymph node metastasis, advanced disease stages, and poor prognosis (Ogawa, K., et al., Br J Cancer 91: 282-286 (2004)). Accordingly, a more complete understanding of the role of the activation of EF-1 pathway in cancer may lead to the development of new types of potent inhibitors for cancer treatment.

The present invention addresses the need in the art for improved compositions and methods for lung cancer diagnosis and therapy through the discovery of molecules involved in pathways of carcinogenesis that can serve as or reveal candidate targets for development of novel anti-cancer drugs and tumor biomarkers.

### SUMMARY OF THE INVENTION

As noted above, the present invention relates four genes, EBI3, DLX5, CDKN3 and NPTX1, and the roles they play in lung cancer carcinogenesis. As such, the present invention relates to novel composition and methods for detecting, diagnosing, treating and/or preventing lung cancer as well as methods for screening for useful agents therefor.

In particular, the present invention arises from the discovery that double-stranded molecules composed of specific sequences (in particular, SEQ ID NOs: 18, 20, 49, 51, 84 and 85) are effective for inhibiting cellular growth of lung cancer cells. Specifically, small interfering RNAs (siRNAs) targeting EBI3, NPTXR, CDKN3 or EF-1delta genes are provided by the present invention. These double-stranded molecules may be utilized in an isolated state or encoded in vectors and expressed from the vectors. Accordingly, it is an object of the present invention to provide such double stranded molecules as well as vectors and host cells expressing them.

In one aspect, the present invention provides methods for inhibiting cell growth and treating lung cancer by administering the double-stranded molecules or vectors of the present invention to a subject in need thereof. Such methods encompass administering to a subject a composition composed of one or more of the double-stranded molecules or vectors.

In another aspect, the present invention provides compositions for treating a cancer containing at least one of the double-stranded molecules or vectors of the present invention.

In yet another aspect, the present invention provides a method of diagnosing or determining a predisposition to lung cancer in a subject by determining an expression level of EBI3, DLX5, and/or CDKN3 in a patient derived biological sample. An increase in the expression level of one or more of the genes as compared to a normal control level of the genes indicates that the subject suffers from or is at risk of developing lung cancer.

Moreover, the present invention relates to the discovery that a high expression level of EBI3, DLX5, CDKN3 and/or EF-1delta correlates to poor survival rate. Therefore, the present invention provides a method for assessing or determining the prognosis of a patient with lung cancer, which method includes the steps of detecting the expression level of one or more gene selected from among EBI3, DLX5, CDKN3 and EF-1delta, comparing it to a pre-determined reference expression level and determining the prognosis of the patient from the difference therebetween.

The level of EBI3 expression has been shown to decrease after the removal of the initial tumor. Accordingly, the present invention provides a method for monitoring treatment or assessing the efficacy of a therapy for an individual diagnosed with lung cancer, such a method including the steps of determining the level of EBI3 expression before and after therapy. A decrease in the level of EBI3 expression after therapy correlates to efficacious therapy.

The discovery of elevated levels of EBI3 in the blood of lung cancer patients is novel to the present invention. Therefore, the present invention provides a method for diagnosing lung cancer in a subject, such a method including the steps of determining the level of EBI3 expression in a subject-derived blood samples and comparing this level to that found in a reference sample, typically a normal control. A high level of EBI3 expression in a sample indicates that the subject either suffers from or is at risk for developing lung cancer.

In a further aspect, the present invention provides a method of screening for a compound for treating and/or preventing lung cancer. Such a compound would bind with EBI3, DLX5, and/or CDKN3 deltagene or reduce the biological activity of EBI3, DLX5, and/or CDKN3, gene or reduce the expression of EBI3, DLX5, and/or CDKN3 gene or reporter gene surrogating the EBI3, DLX5, and/or CDKN3 gene. Moreover, compounds that inhibit the binding between CDKN3 and VRS, EF-1alfa, EF-1beta, EF-1gamma or EF-1delta, or between NPTX1 and NPTXR are expected to reduce a symptom of lung cancer. In particular, a compound which inhibits the binding between a fragment containing amino acid residues 72 to 160 of EF-1gamma and CDKN3 can be identified by the methods of the present invention.

In yet a further aspect, the present invention provides methods for treating and/or preventing lung cancer in a subject by administering to a subject in need thereof an EF-1delta mutant having a dominant negative effect, or a polynucleotide encoding such a mutant. Such an EF-1delta mutant preferably includes an amino acid sequence that includes a CDKN3 binding region, e.g. the part of an EF-1delta protein that includes all or part of the leucine zipper of EF-1delta (see FIG. 20A). In a preferred embodiment, the EF-1delta mutant has the amino acid sequence of SEQ ID NO: 61. The EF-1delta mutant may alternatively have the following general formula: [R]-[D], wherein [R] is a membrane transducing agent, and [D] is a polypeptide having the amino acid sequence of SEQ ID NO: 61. The membrane transducing agent can be selected from among:

poly-arginine;
SEQ ID NO: 63
Tat/RKKRRQRRR/;
SEQ ID NO: 64
Penetratin/RQIKIWFQNRRMKWKK/;
SEQ ID NO: 65
Buforin II/TRSSRAGLQFPVGRVHRLLRK/;
SEQ ID NO: 66
Transportan/GWTLNSAGYLLGKINLKALAALAKKIL/
SEQ ID NO: 67
MAP (model amphipathic peptide)/
KLALKLALKALKAALKLA/;
SEQ ID NO: 68
K-FGF/AAVALLPAVLLALLAP/;
SEQ ID NO: 69
Ku70/VPMLK/;
SEQ ID NO: 70
Ku70/PMLKE/;
SEQ ID NO: 71
Prion/MANLGYWLLALFVTMWTDVGLCKKRPKP/;
SEQ ID NO: 72
pVEC/LLIILRRRIRKQAHAHSK/;
SEQ ID NO: 73
Pep-1/KETWWETWWTEWSQPKKKRKV/;
SEQ ID NO: 74
SynB1/RGGRLSYSRRRFSTSTGR/;
SEQ ID NO: 75
Pep-7/SDLWEMMMVSLACQY/;
and
SEQ ID NO: 76
HN-1/TSPLNIHNGQKL/.

In a further aspect, the present invention provides an antibody binding to the NPTX1 fragment. This antibody has a neutralizing activity. In one aspect, present invention provides a method of treating or preventing lung cancer by administering this antibody.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF THE DRAWINGS

Various aspects and applications of the present invention will become apparent to the skilled artisan upon consideration of the brief description of the figures and the detailed description of the present invention and its preferred embodiments that follows:

FIG. 1: Analysis of EBI3 expression in tumor tissues, cell lines and normal tissue. Part A, Expression of EBI3 in 15 pairs of clinical lung cancer and surrounding normal lung tissue samples (upper panels) [lung adenocarcinoma (ADC), lung squamous cell carcinoma (SCC) and small cell lung carcinoma (SCLC); top] and 23 lung cancer cell lines (lower panels) detected by semiquantitative RT-PCR analysis. Part B depicts the expression and subcellular localization of endogenous EBI3 protein in cancer cell lines and bronchial epithelial cells. EBI3 was stained at the cytoplasm of the cell with granular appearance in NCI-H1373 and LC319 cell lines, whereas no staining in NCI-H2170 and bronchial epithelia derived BEAS-2B cell lines. Part C, depicts detection of secreted EBI3 by ELISA from lung cancer cell lines in culture medium. Secreted EBI3 was detected in the culture medium of EBI3 expressing cell lines.

Panel D depicts the results of Northern blot analysis of the EBI3 transcript in 16 normal adult human tissues. A strong signal was observed in placenta. Panel E depicts the comparison of EBI3 protein expression between normal and tumor tissues by immunohistochemistry.

FIG. 2 depicts the association of EBI3 overexpression with poor prognosis of NSCLC patients. Part A presents examples of strong, weak, and absent EBI3 expression in lung cancer tissues and a normal tissue. Original magnification, ×100 (upper lane), ×200 (lower lane). Panel B depicts the results of Kaplan-Meier analysis of survival of patients with NSCLC (P=0.0011 by log-rank test) according to expression of EBI3.

FIG. 3: Serologic concentration of EBI3 determined by ELISA in patients with lung cancer and in healthy controls or nonneoplastic lung disease patients with COPD. Part A, Distribution of EBI3 in sera from patients with lung ADC, lung SCC, or SCLC. Black lines, average serum levels. Differences were significant between ADC patients (P<0.001, respectively, Mann-Whitney U test), and healthy individuals/COPD patients, between SCC patients and healthy individuals/COPD patients (P<0.001), and between SCLC patients and healthy/COPD individuals (P<0.001), whereas the difference between healthy individuals and COPD patients was not significant (P=0.160). Part B, Distribution of EBI3 in sera from patients at various clinical stages of lung ADC, lung SCC, or SCLC. LD indicates limited disease; ED, extensive disease.

FIG. 4 depicts the serologic concentration of EBI3 in patient with lung cancer or patient post-operation, the comparison of ROC curve analysis of EBI with that of CEA (in NSCLC) or pro-GRP (SCLC), and the inhibition of growth of lung cancer cells by siRNAs against EBI3. Part A, left panel presents the ROC curve analysis of EBI3 as a serum marker for lung cancer. X axis, 1-specificity; Y axis, sensitivity. The cutoff level was set to provide optimal diagnostic accuracy and likelihood ratios (minimal false-negative and false-positive results) for EBI3 [i.e., 11.8 units/mL]. Part A, right panel presents the serum levels of EBI3 before and after primary NSCLC resection. Post operation serums were obtained two months after the surgery. Part B, Serum EBI3 levels (U/mL) and the expression levels of EBI3 in primary tumor tissues in the same NSCLC patients. Part C, top panels: ROC curve analysis of EBI3 (blue) and other conventional tumor markers (CEA as red, CYFRA as green, and ProGRP as yellow) as serum markers for each histological types of lung cancer. X axis, 1-specificity; Y axis, sensitivity. Bottom panels, combination analysis of EBI3 and other tumor markers. Right bars in the both of sensitivity and false positive indicate the sensitivity or false positivity of combination assay using EBI3 and either of three tumor markers (CEA, CYFRA, and ProGRP) in each histological types of lung cancer.

Part D depicts inhibition of growth of lung cancer cells by siRNAs against EBI3. top panels, Gene knockdown effect on EBI3 expression in A549 cells and LC319 cells by si-EBI3s (#1 and #2) and control siRNAs (si-CNT/On-target, si-LUC/Luciferase), analyzed by semiquantitative RT-PCR. Bottom panels, Colony formation and MTT assays of A549 cells and LC319 cells transfected with si-EBI3s or control siRNAs. Columns, relative absorbance of triplicate assays; bars, SD. Part E, two independent transfectants expressing high levels of EBI3 (COS-7-EBI3-#1 and -#2, top panels) and controls (COS-7-M1 and -M2) were each cultured in triplicate; after 120 hours the cell viability was evaluated by the MTT assay and colony formation assay (bottom panels).

FIG. 5: Presents the expression of DLX5 in lung tumors and normal tissues. Part A depicts the expression of distal-less homeobox 5 (DLX5) in clinical samples of NSCLC (adenocarcinoma and squamous-cell carcinoma) and normal lung tissues, examined by semiquantitative RT-PCR. Part B depicts the expression of DLX5 in lung-cancer cell lines, as revealed by semiquantitative RT-PCR. Expression of beta-actin (ACTB) served as a quantity control. Part C depicts the subcellulardistribution of the DLX5 proteins examined by confocal microscopy. Part D depicts the expression of DLX5 in normal human tissues, detected by northern-blot analysis.

FIG. 6: Presents the immunohistochemicalevaluation of DLX5 protein expression and the association of its overexpression with poor prognosis for NSCLC patients and Inhibition of growth by siRNA against DLX5 in SBC-5 cancer cells. Part A depicts the expression of DLX5 in five normal human tissues as well as lung SCC, detected by immunohistochemical staining using the rabbit polyclonal anti-DLX5 antibody; counterstaining with hematoxylin (×200). Positive staining appeared in the cytoplasm and/or nucleus of syncytiotrophoblasts in the placenta (arrows) and lung-cancer cells. Part B depicts a representative example of the expression of DLX5 in lung cancer (SCCs, ×100) and normal lung (×100), and magnified view of SCC positive case (×200). Part C presents the results of Kaplan-Meier analysis of tumor specific survival in NSCLC patients according to DLX5 expression level. Part D presents the level of DLX5 expression detected by semiquantitative RT-PCR in SBC-5 cells. The effect of treatment with either control siRNAs (si-EGFP or si-Scramble/SCR) or si-DLX5 is shown in the upper panels. The effect of siRNA against DLX5 on cell viability, detected by MTT assays is shown in lower panels.

FIG. 7: Presents the expression of NPTX1 in lung tumors. Part A, Upper panels, depicts the expression of NPTX1 in 15 clinical samples of lung cancer (10 NSCLC and 5 SCLC) (7) and their corresponding normal lung tissues (N), examined by semiquantitative RT-PCR. Appropriate dilutions of each single-stranded cDNA were prepared from mRNAs of clinical lung cancer samples, taking the level of β-actin (ACTB) expression as a quantitative control. Part A, Lower panels, depicts the expression of NPTX1 in 23 lung cancer cell lines, examined by semiquantitative RT-PCR. Part B depicts the expression of NPTX1 protein in 4 lung cancer cell lines, examined by Western blot analysis. Part C depicts the subcellular localization of endogenous NPTX1 protein in the 4 lung cancer cell lines. NPTX1 was stained at the cytoplasm of the cell with granular appearance in NCI-H226, NCI-H520, and SBC-5 cells, but not in NCI-H2170 cells. Part D depicts the detection of secreted NPTX1 protein with ELISA in conditioned medium from NPTX1-expressing NCI-H226, NCI-H520, and SBC-5 cells as well as NPTX1-non-expressing NCI-H2170 cells. Part E Expressions of NPTX1 and NPTXR in nine clinical lung cancers (lower panel) and 23 lung cancer cell lines (upper panel), examined by semiquantitative RT-PCR.

FIG. 8: Presents the expression of NPTX1 in normal tissues and lung cancer tissues. Part A depicts the expression of NPTX1 in normal human tissues detected by Northern blot analysis. Part B presents the results of immunohistochemical evaluation of NPTX1 protein in representative lung adenocarcimona (ADC) tissue and five normal tissues; heart, liver, kidney, adrenal gland. Part C presents the results of immunohistochemical staining of NPTX1 in representative lung adenocarcimona ADC, lung squamous cell carcinoma (SCC), and small cell lung cancer (SCLC), using anti-NPTX1 antibody on tissue microarrays (original magnification ×200), Part D, upper panels, presents examples of strong, weak, and absent NPTX1 expression in lung ADCs. Part D, Lower panel, Kaplan-Meier analysis of tumor-specific survival in patients with NSCLC according to NPTX1 expression (P<0.0001; Log-rank test).

FIG. 9: Presents the serologic concentration of NPTX1 determined by ELISA in patients with lung cancers and in healthy donors or non-neoplastic lung disease patients with COPD. Part A depicts the distribution of NPTX1 in sera from patients with lung ADC, lung SCC, or SCLC. Differences were significant between ADC patients and healthy/COPD individuals (P<0.001, Mann-Whitney U test), between SCC patients and healthy/COPD individuals (P=0.005) and between SCLC patients and healthy/COPD individuals (P=0.0051). The difference between healthy individuals and COPD was not significant. Part B depicts the distribution of NPTX1 in sera from patients at various clinical stages of lung cancers. LD indicates limited disease; ED, extensive disease. Part C, Serologic concentration of NPTX1 before and after surgery (postoperative days at 2 months) in patients with NSCLC. Part D, Serum NPTX1 levels and the expression levels of NPTX1 in primary tumor tissues in the same NSCLC patients (original magnification ×100).

FIG. 10. Presents the autocrine cellular growth effect of NPTX1. Part A depicts the inhibition of growth of lung cancer cells by siRNA against NPTX1. The upper panels of Part A depict the expression of NPTX1 in response to si-NPTX1s (si-1, -2) or control siRNAs (LUC or SCR) in A549 and SBC-5 cells, analyzed by RT-PCR analysis. The middle panels of Part A present images of colonies examined by colony-formation assays of the A549 and SBC-5 cells transfected with specific siRNAs for NPTX1 or control plasmids. The bottom panels of Part A present the viability of the A549 or SBC-5 cells evaluated by MTT assay in response to si-NPTX1s, -LUC, or -SCR. All assays were performed three times, and in triplicate wells. Part B presents growth-promoting effect of NPTX1 transiently overexpressed in COS-7 cells. Top panel, Transient expression of NPTX1 in COS-7 cells, detected by Western blot analysis. The bottom panels, Viability of the COS-7 cells evaluated by MTT (left) and colony formation assays (right). C, Left panel, Autocrine/paracrine effect of NPTX1 on the growth of mammalian cells. Cell viability counted by MTT assays (COS-7 cells treated with NPTX1 in final concentrations of 0, 0.1, or 1 nM) (right lanes indicated by PBS). MTT assay evaluating the competitive-neutralizing effect of anti-NPTX1 monoclonal antibody (mAb-75-1; 50 nM) and control IgG (normal mice; 50 nM) on the activity of NPTX1 protein (0, 0.1, or 1 nM) in the culture medium of COS-7 cells (left and middle lanes indicated by Anti-NPTX1 mAb and IgG). Right panel, Inhibition of in vitro growth of lung cancer A549 cells that overexpressed NPTX1 by anti-NPTX1 monoclonal antibody (25 nM or 50 nM) in a dose dependent manner. Each experiment was done in triplicate. Part D, Inhibition of in vitro growth of various lung cancer cells by anti-NPTX1 antibody. MTT assay evaluating the effect of anti-NPTX1 monoclonal antibody (mAb-75-1; 50 nM) on the growth of a NPTX1-overexpressing lung cancer cell line SBC-5 (P=0.012; each paired t-test) and NPTX1-non-expressing lung cancer cell lines, SBC-3 and NCI-H2170. Each experiment was done in triplicate.

FIG. 11: Presents the enhanced invasiveness of mammalian cells transfected with NPTX1-expressing plasmids. Assays demonstrating the invasive nature of NIH-3T3 cells in Matrigel matrix after transfection with expression plasmids for human NPTX1. Left upper panels, Transient expression of NPTX1 in the NIH-3T3 cells, detected by western-blot analysis. Lower panels, Giemsa staining (×200) and the number of cells migrating through the Matrigel-coated filters. Assays were performed three times, and each in triplicate wells.

FIG. 12. Effect of anti-NPTX1 monoclonal antibody against A549 cells transplanted to nude mice. Top panel, Average tumor volumes of three mice treated twice a week with anti-NPTX1 monoclonal antibody (mAb-75-1; 300 micro g/body) or normal mice IgG (control-1; 300 micro g/body) and those without treatment (control-2) were plotted. Values are expressed as mean±s.e. tumor volume. Animals were administered twice a week by intraperitoneal injections with each of the antibodies for 30 days. The bottom panels, Histopathological examination of HE-stained tumors (A549) treated with anti-NPTX1 antibody. At day 30 after treatment with NPTX1 antibody, a fibromatic change and more significant decrease of viable cancer cells were observed in tumor tissues treated with anti-NPTX1 antibody, compared with those with control IgG or without treatment.

FIG. 13. presents interaction of NPTX1 and NPTXR in a growth-promoting pathway.

Part A, Confocal microscopy was carried out with COS-7 cells expressing NPTX1 or NPTXR. Green: NPTX1 (myc); Red: NPTXR. Left panel, COS-7 cells were permialized by Triton X-100 and stained by anti-myc antibodies detecting NPTX1. Right panels, COS-7 cells were stained for extracellular surface staining with antibodies to NPTX1 (myc-tag) and NPTXR antibodies. Part B, C, Confocal microscopy was carried out using COS-7 cells (B) and SBC-5 cells (C) expressing NPTX1 or NPTXR. The left panels, COS-7 cells and SBC-5 cells were stained for extracellular surface staining with NPTX1 (myc) and NPTXR antibodies. The right panels, Glycine treatment were performed to remove NPTX1 on the cell surface. Part D, Inhibition of growth of lung cancer cells by siRNA against NPTXR. The top panels, Expression of NPTX1 in response to si-NPTX1s (si-1 and si-2) or control siRNAs (si-LUC and si-SCR) in A549 and SBC-5 cells, analyzed by RT-PCR analysis. The bottom panels, Image of colonies examined by colony-formation assays of the A549 and SBC-5 cells transfected with specific siRNAs for NPTXR or control siRNAs. The middle panels, Viability of the A549 or SBC-5 cells evaluated by MTT assay in response to si-NPTXRs, -LUC, or -SCR. All assays were performed three times, and in triplicate wells.

FIG. 14: Presents internalization of NPTX1 after binding with NPTXR Part A, B, Recipient COS-7 cells (A) or SBC-5 cells (B) were incubated with conditioned medium from NPTX1-transfected (+) donor COS-7 cells or SBC-5 cells, respectively. c-myc-tagged NPTX1 was detected 3 hours after treatment of recipient cells with donor's conditioned medium. Green: NPTX1. Nuclei were visualized by DAPI. (a) Cells were stained for extracellular surface staining with anti-myc antibody for detecting NPTX1. (b) Cells were permialized by Triton X-100 and stained for NPTX1 (myc). (c) 3 hours treatment with PBS. Part C, Recipient COS-7 cells appeared to uptake in a time-dependent manner the secreted NPTX1 in conditioned medium from donor NPTX1 transfected (+) COS-7 cells. 1 or 3 hours after treatment of recipient COS-7 cells with conditioned medium from donor NPTX1-transfected (+) COS-7 cells, internalized NPTX1 was detected by western blotting using anti-myc antibodies.

FIG. 15. Part A Detection of secreted exogenous NPTX1 protein with Western blot analysis in conditioned medium from NPTX1-expressing COS-7 cells. Part B Binding of NPTX1 to NPTXR proteins in COS-7 cells expressing exogenous NPTX1 were detected by immunoprecipitation analysis.

FIG. 16. Presents the expression of CDKN3 in lung cancers and brain metastasis. Part A depicts the expression of CDKN3 in clinical samples of NSCLC (T) and corresponding normal lung tissues (N), examined by semiquantitative RT-PCR. Part B depicts the expression of CDKN3 in clinical samples of early primary NSCLC (stage I-IIIa), advanced primary NSCLC (stage IIIb-IV), and metastatic brain tumor from ADC (T) and normal lung tissues (N), examined by semiquantitative RT-PCR (upper panel). Densitometric intensity of PCR product was quantified by image analysis software (lower panel). Part C depicts the expression of CDKN3 in normal human tissues, detected by northern-blot analysis.

FIG. 17: Presents the expression of CDKN3 in lung cancers and its association with poor clinical outcome for NSCLC patients. Part A depicts the expression of CDKN3 in six normal human tissues as well as a case of NSCLC, detected by immunohistochemical staining using the mouse monoclonal anti-CDKN3 antibody; counterstaining with hematoxylin (×200). Part B depicts the results of immunohistochemical staining of representative surgically-resected NSCLC (lung-SCC) and normal lung, using anti-CDKN3 antibody on tissue microarrays (×100). C, Kaplan-Meier analysis of tumor-specific survival in patients with NSCLC according to CDKN3 expression (P<0.0001 by the Log-rank test).

FIG. 18: Presents the identification of EF-1beta, gamma, delta/ValRS as the novel molecules interacting with CDKN3. Part A depicts the screening of proteins that interact with CDKN3. The 140-, 50-, 31-, and 25-kDa bands shown by silver staining, which were seen in cell lysates from LC319 cells immunoprecipitated with anti-CDKN3 monoclonal antibody, but not seen in those with normal mouse IgG, were extracted. Their peptide sequences by MALDI-TOF mass spectrometric sequencing defined the individual bands to be VARS, EF-1gamma, EF-1delta, EF-1beta, respectively. The CDKN3 protein band is marked by asterisk. Positions of molecular weight markers (in kDa) are indicated on the left side. Part B depicts the expression of CDKN3, ValRS, EF-1gamma, EF-1delta, EF-1beta, and their related molecule, CDK1 in NSCLC cell lines, detected by semiquantitative RT-PCR analysis.

FIG. 19: Presents the expression of EF-1delta in lung cancers and its association with poor clinical outcome for NSCLC patients. Part A depicts the expression of CDKN3 and EF-1delta proteins in lung-cancer cell lines, detected by western-blot analysis. Part B depicts the results of immunohistochemical staining of representative surgically-resected samples including NSCLC (lung-SCC) as well as normal lung, using anti-EF-1delta antibody on tissue microarrays (×100). Part C depicts the association of EF-1delta expression with poor clinical outcomes among NSCLC patients. Kaplan-Meier analysis of tumor-specific survival in patients with NSCLC according to EF-1delta expression was shown (P=0.0006, by the Log-rank test).

FIG. 20: Presents the dephosphorylation of EF-1delta by CDKN3. Part A depicts the association of CDKN3 with EF-1delta in lung cancer cells, confirmed by immunoprecipitation of endogenous CDKN3 and EF-1delta from extracts of LC319 cells. IP; immunoprecipitation, IB; immunoblot. Part B depicts the co-localization of endogenous CDKN3 (green), and endogenous EF-1delta (red) in LC319 cells at various cell cycle phases. Part C depicts the phosphorylation of exogenous and endogenous EF-1delta. The cell extracts from COS-7 cells that overexpressed exogenous EF-1delta (left panel) and those from LC319 cells (right panel) were treated with Lambda Protein Phosphatase (lambda-PPase). The shifted band was detected in lambda-PPase-treated extracts of the cells. The open and closed arrows indicate phosphorylated EF-1delta and dephosphorylated EF-1delta, respectively. Part D depicts dephosphorylation of endogenous EF-1delta by exogenously overexpressed CDKN3 in LC319 cells. CDKN3-expression vectors were transfected to LC319 cells.

FIG. 21: Identifies the CDKN3-binding region in EF-1delta. Part A depicts the dephosphorylation of exogenous EF-1delta in COS-7 cells that were transiently overexpressed CDKN3. COS-7 cells that weakly expressed endogenous CDKN3 and EF-1delta, were transfected with the Flag-HA-tagged CDKN3-expression vector, the Flag-HA-tagged EF-1delta-expression vector, or both two expression vectors. Whole cell extracts from these cells were used for western-blot analysis with anti-HA antibody (left panel). The oblique lined, open, and closed arrows indicate CDKN3, phosphorylated EF-1delta, and dephosphorylated EF-1delta, respectively. These cell extracts immunoprecipitated with anti-Flag antibody were immunoblotted using anti-phospho-serine antibody (right panel). The open arrow indicates phosphorylated EF-1delta. IP; immunoprecipitation, IB; immunoblot. Part B depicts the sequence scheme of EF-1delta. One full-length and four deletion constructs of EF-1delta are shown. Part C depicts the identification of the region in EF-1delta that binds to CDKN3 by immunoprecipitation experiments. The EF-1delta 161-281 construct, which lacked N-terminal 160 amino-acid polypeptides in EF-1delta, did not retain any ability to interact with endogenous CDKN3 in LC319 cells, suggesting that the 89 amino-acid polypeptide (codons 72-160) containing leucine zipper motif in EF-1delta should play an important role in the interaction with CDKN3. IP; immunoprecipitation, IB; immunoblot.

FIG. 22: Depicts the effect of CDKN3 or EF-1delta on growth of lung cancer cells. A left upper panel, Expression of CDKN3 in response to si-CDKN3 (si-A and -B) or control siRNAs (EGFP, luciferase (LUC), or scramble (SCR)) in LC319 cells, analyzed by semiquantitative RT-PCR. Part A, right upper panel, depicts the viability of LC319 cells evaluated by MTT assay in response to si-CDKN3s, -EGFR, -LUC, or -SCR. Part A, lower panel, Colony-formation assays of LC319 cells transfected with specific siRNAs or control plasmids. Part B, left upper panel, depicts the expression of EF-1delta in response to si-EF-1delta (si-1 and -2) or control siRNAs (EGFP, luciferase (LUC), or scramble (SCR)) in LC319 cells, analyzed by semiquantitative RT-PCR. Part B, right upper panel, depicts the viability of LC319 cells evaluated by MTT assay in response to si-EF-1delta or control siRNAs. Part B, lower panel, depicts the results of colony-formation assays of LC319 cells transfected with si-EF-1delta or control siRNAs.

FIG. 23: Demonstrates the ability of CDKN3 to increase cellular invasive activity and activate Akt. Part A presents the results of Matrigel invasion assays demonstrating the increased invasive ability of NIH-3T3 cells transfected with mock-vector or CDKN3-expression vector. The number of invading cells through Matrigel-coated filters are shown. Part B depicts the expression of EF-1alpha1 and EF-1alpha2 in NSCLC cell lines, detected by semiquantitative RT-PCR analysis. Part C depicts the association of CDKN3 with EF-1alpha in lung cancer cells, confirmed by immunoprecipitation using extracts of LC319 cells. IP; immunoprecipitation, IB; immunoblot (left panel). Part D depicts the Akt-phosphorylation in LC319 cells transfected with CDKN3-expression vector. Total protein extracts from CDKN3-expressing cells were detected by western-blot analysis using anti-Akt, anti-phospho-Akt (Ser473), anti-Flag antibodies or anti-c-Myc antibodies. The protein extracts from cells transfected mock-vector were used as controls and beta-actin used as a loading control. Part E, NIH-3T3 cells transfected with mock-vector or CDKN3-expression vector were pre-incubated with LY294002 or DMSO (vehicle) and subjected to the matrigel invasion assay demonstrating the increased invasive ability. The number of invading cells through Matrigel-coated filters was shown.

FIG. 24: Identifies the CDKN3-binding region in EF-1delta. Part A presents a schematic drawing of five cell permeable peptides linked covalently at its NH₂-terminus to a membrane transducing 11 poly-arginine sequence. The sequence of leucine zipper motif in EF-1delta and five cell permeable peptides derived from EF-1delta are shown. Part B presents the viability of LC319 cells evaluated by MTT assay in response to five cell permeable peptides (upper panel). Reduction of the complex formation detected by immunoprecipitation between endogenous CDKN3 and EF-1delta proteins in LC319 cells that were treated with the 11R-EF-1delta₉₀₋₁₀₈ peptides (lower panel).

### THE DISCLOSURE OF THE INVENTION

Although methods and materials similar or equivalent to those described herein can be used in the practice or testing of embodiments of the present invention, the preferred methods and materials are now described. However, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the present invention which will be limited only by the appended claims.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the present invention, the following definitions apply:

### DEFINITIONS

The words “a”, “an”, and “the” as used herein mean “at least one” unless otherwise specifically indicated.

As used herein, the term “biological sample” refers to a whole organism or a subset of its tissues, cells or component parts (e.g., body fluids, including but not limited to blood, mucus, lymphatic fluid, synovial fluid, cerebrospinal fluid, saliva, amniotic fluid, amniotic cord blood, urine, vaginal fluid and semen). “Biological sample” further refers to a homogenate, lysate, extract, cell culture or tissue culture prepared from a whole organism or a subset of its cells, tissues or component parts, or a fraction or portion thereof. Lastly, “biological sample” refers to a medium, such as a nutrient broth or gel in which an organism has been propagated, which contains cellular components, such as proteins or polynucleotides.

The term “polynucleotide”, “oligonucleotide” “nucleotide”, “nucleic acid”, and “nucleic acid molecule” are used interchangeably herein to refer to a polymer of nucleic acid residues and, unless otherwise specifically indicated are referred to by their commonly accepted single-letter codes. The terms apply to nucleic acid (nucleotide) polymers in which one or more nucleic acids are linked by ester bonding. The nucleic acid polymers may be composed of DNA, RNA or a combination thereof and encompass both naturally-occurring and non-naturally occurring nucleic acid polymers.

The terms “polypeptide”, “peptide”, and “protein” are used interchangeably herein to refer to a polymer of amino acid residues. The terms apply to amino acid polymers in which one or more amino acid residue is a modified residue, or a non-naturally occurring residue, such as an artificial chemical mimetic of a corresponding naturally occurring amino acid, as well as to naturally occurring amino acid polymers.

### Genes or Proteins

The nucleic acid and polypeptide sequences of genes in present invention are shown in the following numbers, but not limited to those;

EBI3: SEQ ID NO: 1 and 2;

DLX5: SEQ ID NO: 3 and 4;

CDKN3: SEQ ID NO: 5 and 6;

EF-1delta: SEQ ID NO: 7 and 8;

ValRS: SEQ ID NO: 26 or 28, and 27 or 29;

EF-1beta: SEQ ID NO: 30 and 31;

EF-1gamma: SEQ ID NO: 32 and 33;

EF-1 alfa: SEQ ID NO: 57 or 90 and 58 or 91;

Akt: SEQ ID NO: 59 and 60;

NPTX1: SEQ ID NO: 78 and 79; and

NPTXR: SEQ ID NO: 86 and 87.

Furthermore, the sequence data are also available via following accession numbers.

EBI3: NM_{—}005755;

DLX5: BC006226;

CDKN3: L27711;

EF-1delta: BC009907;

ValRS: NM_{—}006295 or BC012808;

EF-1beta: NM_{—}001959;

EF-1gamma: BC009865;

EF-1 alfa: NM_{—}001402 or NM_{—}001958;

NPTX1: SEQ ID NO: NM_{—}002522 or NM_{—}002522.2; and

NPTXR: SEQ ID NO: NM_{—}014293.

According to an aspect of the present invention, functional equivalents are also considered to be above “polypeptides”. Herein, a “functional equivalent” of a protein is a polypeptide that has a biological activity equivalent to the protein. Namely, any polypeptide that retains the biological ability may be used as such a functional equivalent in the present invention. Such functional equivalents include those wherein one or more amino acids are substituted, deleted, added, or inserted to the natural occurring amino acid sequence of the protein. Alternatively, the polypeptide may be composed an amino acid sequence having at least about 80% homology (also referred to as sequence identity) to the sequence of the respective protein, more preferably at least about 90% to 95% homology. In other embodiments, the polypeptide can be encoded by a polynucleotide that hybridizes under stringent conditions to the natural occurring nucleotide sequence of the gene.

A polypeptide of the present invention may have variations in amino acid sequence, molecular weight, isoelectric point, the presence or absence of sugar chains, or form, depending on the cell or host used to produce it or the purification method utilized. Nevertheless, so long as it has a function equivalent to that of the human protein of the present invention, it is within the scope of the present invention.

The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10 degrees C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may also be achieved with the addition of destabilizing agents such as formamide. For selective or specific hybridization, a positive signal is at least two times of background, preferably 10 times of background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 50° C.

In the context of the present invention, a condition of hybridization for isolating a DNA encoding a polypeptide functionally equivalent to the avobe human protein can be routinely selected by a person skilled in the art. For example, hybridization may be performed by conducting pre-hybridization at 68 degrees C. for 30 min or longer using “Rapid-hyb buffer” (Amersham LIFE SCIENCE), adding a labeled probe, and warming at 68 degrees C. for 1 hour or longer. The following washing step can be conducted, for example, in a low stringent condition. An exemplary low stringent condition may include 42° C., 2×SSC, 0.1% SDS, preferably 50° C., 2×SSC, 0.1% SDS. High stringency conditions are often preferably used. An exemplary high stringency condition may include washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors, such as temperature and salt concentration, can influence the stringency of hybridization and one skilled in the art can suitably select the factors to achieve the requisite stringency.

Generally, it is known that modifications of one or more amino acid in a protein do not influence the function of the protein. In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids or those considered to be a “conservative modifications”, wherein the alteration of a protein results in a protein with similar functions, are acceptable in the context of the instant invention.

So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, preferably 20 amino acids or less, more preferably 10 amino acids or less, more preferably 6 amino acids or less, and even more preferably 3 amino acids or less.

An amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Conservative substitution tables providing functionally similar amino acids are well known in the art. For example, the following eight groups each contain amino acids that are conservative substitutions for one another:

1) Alanine (A), Glycine (G);

2) Aspartic acid (D), Glutamic acid (E);

3) Aspargine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cystein (C), Methionine (M) (see, e.g., Creighton, Proteins 1984).

Such conservatively modified polypeptides are included in the present protein. However, the present invention is not restricted thereto and the protein includes non-conservative modifications, so long as at least one biological activity of the protein is retained. Furthermore, the modified proteins do not exclude polymorphic variants, interspecies homologues, and those encoded by alleles of these proteins.

Moreover, the gene of the present invention encompasses polynucleotides that encode such functional equivalents of the protein. In addition to hybridization, a gene amplification method, for example, the polymerase chain reaction (PCR) method, can be utilized to isolate a polynucleotide encoding a polypeptide functionally equivalent to the protein, using a primer synthesized based on the sequence above information. Polynucleotides and polypeptides that are functionally equivalent to the human gene and protein, respectively, normally have a high homology to the originating nucleotide or amino acid sequence of. “High homology” typically refers to a homology of 40% or higher, preferably 60% or higher, more preferably 80% or higher, even more preferably 90% to 95% or higher. The homology of a particular polynucleotide or polypeptide can be determined by following the algorithm in “Wilbur and Lipman, Proc Natl Acad Sci USA 80: 726-30 (1983)”.

### Antibodies:

The terms “antibody” as used herein is intended to include immunoglobulins and fragments thereof which are specifically reactive to the designated protein or peptide thereof. An antibody can include human antibodies, primatized antibodies, chimeric antibodies, bispecific antibodies, humanized antibodies, antibodies fused to other proteins or radiolabels, and antibody fragments. Furthermore, an antibody herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. An “antibody” indicates all classes (e.g. IgA, IgD, IgE, IgG and IgM).

The subject invention utilizes antibodies against a CDKN3 binding region (at the position of 72-160aa) of EF-1delta for interrupting a binding or interaction between CDKN3 and EF-1delta. Because both of two genes are up-regulated in lung cancer (FIGS. 16, 17, 18B and 19) and the interaction is determined in lung cancer cell (FIGS. 18 and 20). Furthermore, antibody against NPTX1 was useful for the neutralizing secreted NPTX1 protein and inhibiting cancer cell proliferation (FIGS. 10B and C). Therefore the antibodies of the present invention can be useful for treating lung cancer. These antibodies will be provided by known methods. Exemplary techniques for the production of the antibodies used in accordance with the present invention are described.

### (i) Polyclonal Antibodies:

Polyclonal antibodies are preferably raised in animals by multiple subcutaneous (sc) or intraperitoneal (ip) injections of the relevant antigen and an adjuvant. In the present invention that antigens are, but are not limited to, polypeptide comprising SEQ ID NO: 88 or 89 or the CDKN3 binding region of EF-1delta, such as SEQ ID NO: 61. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R′N═C═NR, where R′ and R are different alkyl groups.

Animals are immunized against the antigen, immunogenic conjugates, or derivatives by combining, e.g. 100 mcg or 5 mcg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent.

Conjugates also can be made in recombinant cell culture as protein fusions. Also, aggregating agents such as alum are suitably used to enhance the immune response.

### (ii) Monoclonal Antibodies:

Monoclonal antibodies are obtained from a population of substantially homogeneous antibodies, i.e., the individual antibodies comprising the population are identical except for possible naturally occurring mutations that may be present in minor amounts. Thus, the modifier “monoclonal” indicates the character of the antibody as not being a mixture of discrete antibodies.

For example, the monoclonal antibodies may be made using the hybridoma method first described by Kohler G & Milstein C. Nature. 1975 Aug. 7; 256(5517):495-7, or may be made by recombinant DNA methods (U.S. Pat. No. 4,816,567).

In the hybridoma method, a mouse or other appropriate host animal, such as a hamster, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Manassas, Va., USA. Human myeloma and mouse-human heteromyeloma cell lines also have been described for the production of human monoclonal antibodies (Kozbor D, et al., J Immunol. 1984 December; 133(6):3001-5; Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

The binding affinity of the monoclonal antibody can, for example, be determined by the 30 Scatchard analysis of Munson P J & Rodbard D. Anal Biochem. 1980 Sep. 1; 107(1):220-39.

After hybridoma cells are identified that produce antibodies of the desired specificity, affinity, and/or activity, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce immunoglobulin protein, to obtain the synthesis of monoclonal antibodies in the recombinant host cells. Review articles on recombinant expression in bacteria of DNA encoding the antibody include Skerra A. Curr Opin Immunol. 1993 April; 5(2):256-62 and Plückthun A. Immunol Rev. 1992 December; 130:151-88.

Another method of generating specific antibodies, or antibody fragments, reactive against a CDKN3 binding region (at the position of 72-160aa) of EF-1delta is to screen expression libraries encoding immunoglobulin genes, or portions thereof, expressed in bacteria with a CDKN3 binding region (at the position of 72-160aa) of EF-1delta. For example, complete Fab fragments, VH regions and Fv regions can be expressed in bacteria using phage expression libraries. See for example, Ward E S, et al., Nature. 1989 Oct. 12; 341(6242):544-6; Huse W D, et al., Science. 1989 Dec. 8; 246(4935):1275-81; and McCafferty J, et al., Nature. 1990 Dec. 6; 348(6301):552-4. Screening such libraries with, a CDKN3 binding region (at the position of 72-160aa) of EF-1delta, can identify immunoglobulin fragments reactive with the a CDKN3 binding region (at the position of 72-160aa) of EF-1delta. Alternatively, the SCID-hu-mouse (available from Genpharm) can be used to produce antibodies or fragments thereof.

In a further embodiment, antibodies or antibody fragments can be isolated from antibody phage libraries generated using the techniques described in McCafferty J, et al., Nature. 1990 Dec. 6; 348(6301):552-4; Clarkson T, et al., Nature. 1991 Aug. 15; 352(6336):624-8; and Marks J D, et al., J MoL BioL., 222: 581-597 (1991) J Mol Biol. 1991 Dec. 5; 222(3):581-97 describe the isolation of murine and human antibodies, respectively, using phage libraries. Subsequent publications describe the production of high affinity (nM range) human antibodies by chain shuffling (Marks J D, et al., Biotechnology (N Y). 1992 July; 10(7):779-83), as well as combinatorial infection and in vivo recombination as a strategy for constructing very large phage libraries (Waterhouse P, et al., Nucleic Acids Res. 1993 May 11; 21(9):2265-6). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison S L, et al., Proc Natl Acad Sci USA. 1984 November; 81(21):6851-5), or by covalently joining to the immunoglobulin coding sequence all or part of the coding sequence for a non-immunoglobulin polypeptide.

Typically, such non-immunoglobulin polypeptides are substituted for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

(iii) Humanized Antibodies:

Methods for humanizing non-human antibodies have been described in the art. Preferably, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones P T, et al., Nature. 1986 May 29-Jun. 4; 321(6069):522-5; Riechmann L, et al., Nature. 1988 Mar. 24; 332(6162):323-7; Verhoeyen M, et al., Science. 1988 Mar. 25; 239(4847):1534-6), by substituting hypervariable region sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some hypervariable region residues and possibly some FR residues are substituted by residues from analogous sites in rodent antibodies.

The choice of human variable domains, both light and heavy, to be used in making the humanized antibodies is very important to reduce antigenicity. According to the so called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims M J, et al., J Immunol. 1993 Aug. 15; 151(4):2296-308; Chothia C & Lesk A M. J Mol Biol. 1987 Aug. 20; 196(4):901-17). Another method uses a particular framework region derived from the consensus sequence of all human antibodies of a particular subgroup of light or heavy chains. The same framework may be used for several different humanized antibodies (Carter P, et al., Proc Natl Acad Sci USA. 1992 May 15; 89(10):4285-9; Presta L G, et al., J Immunol. 1993 Sep. 1; 151(5):2623-32).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen, is achieved. In general, the hypervariable region residues are directly and most substantially involved in influencing antigen binding.

### (iv) Human Antibodies:

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ line mutant mice will result in the production of human antibodies upon antigen challenge. See, e.g., Jakobovits A, et al., Proc Natl Acad Sci USA. 1993 Mar. 15; 90(6):2551-5; Nature. 1993 Mar. 18; 362(6417):255-8; Brüggemann M, et al., Year Immunol. 1993; 7:33-40; and U.S. Pat. Nos. 5,591,669; 5,589,369 and 5,545,807.

Alternatively, phage display technology (McCafferty J, et al., Nature. 1990 Dec. 6; 348(6301):552-4) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson K S & Chiswell D J. Curr Opin Struct Biol. 1993; 3:564-71. Several sources of V-gene segments can be used for phage display.

Clackson T, et al., Nature. 1991 Aug. 15; 352(6336):624-8 isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self antigens) can be isolated essentially following the techniques described by Marks J D, et al., J Mol Biol. 1991 Dec. 5; 222(3):581-97, or Griffiths A D, et al., EMBO J. 1993 February; 12(2):725-34. See, also, U.S. Pat. Nos. 5,565,332 and 5,573,905.

Human antibodies may also be generated by in vitro activated B cells (see U.S. Pat. Nos. 5,567,610 and 5,229,275). A preferred means of generating human antibodies using SCID mice is disclosed in commonly-owned, co-pending applications.

### (v) Antibody Fragments:

Various techniques have been developed for the production of antibody fragments. Traditionally, these fragments were derived via proteolytic digestion of intact antibodies (see, e.g., Morimoto K & Inouye K. J Biochem Biophys Methods. 1992 March; 24(1-2):107-17; Brennan M, et al., Science. 1985 Jul. 5; 229(4708):81-3). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F (ab′) 2 fragments (Carter P, et al., Biotechnology (N Y). 1992 February; 10(2):163-7). According to another approach, F (ab′) 2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. Nos. 5,571,894 and 5,587,458. The antibody fragment may also be a “linear antibody”, e.g., as described in U.S. Pat. No. 5,641,870 for example. Such linear antibody fragments may be monospecific or bispecific.

### (vi) Non-Antibody Binding Protein:

The terms “non-antibody binding protein” or “non-antibody ligand” or “antigen binding protein” interchangeably refer to antibody mimics that use non-immunoglobulin protein scaffolds, including adnectins, avimers, single chain polypeptide binding molecules, and antibody-like binding peptidomimetics, as discussed in more detail below.

Other compounds have been developed that target and bind to targets in a manner similar to antibodies. Certain of these “antibody mimics” use non-immunoglobulin protein scaffolds as alternative protein frameworks for the variable regions of antibodies.

For example, Ladner et al. (U.S. Pat. No. 5,260,203) describe single polypeptide chain binding molecules with binding specificity similar to that of the aggregated, but molecularly separate, light and heavy chain variable region of antibodies. The single-chain binding molecule contains the antigen binding sites of both the heavy and light chain variable regions of an antibody connected by a peptide linker and will fold into a structure similar to that of the two peptide antibody. The single-chain binding molecule displays several advantages over conventional antibodies, including, smaller size, greater stability and are more easily modified.

Ku et al. (Proc Natl Acad Sci USA 92(14):6552-6556 (1995)) describe an alternative to antibodies based on cytochrome b562. Ku et al. (1995) generated a library in which two of the loops of cytochrome b562 were randomized and selected for binding against bovine serum albumin. The individual mutants were found to bind selectively with BSA similarly with anti-BSA antibodies.

Lipovsek et al. (U.S. Pat. Nos. 6,818,418 and 7,115,396) describe an antibody mimic featuring a fibronectin or fibronectin-like protein scaffold and at least one variable loop. Known as Adnectins, these fibronectin-based antibody mimics exhibit many of the same characteristics of natural or engineered antibodies, including high affinity and specificity for any targeted ligand. Any technique for evolving new or improved binding proteins can be used with these antibody mimics.

The structure of these fibronectin-based antibody mimics is similar to the structure of the variable region of the IgG heavy chain. Therefore, these mimics display antigen binding properties similar in nature and affinity to those of native antibodies. Further, these fibronectin-based antibody mimics exhibit certain benefits over antibodies and antibody fragments. For example, these antibody mimics do not rely on disulfide bonds for native fold stability, and are, therefore, stable under conditions which would normally break down antibodies. In addition, since the structure of these fibronectin-based antibody mimics is similar to that of the IgG heavy chain, the process for loop randomization and shuffling can be employed in vitro that is similar to the process of affinity maturation of antibodies in vivo.

Beste et al. (Proc Natl Acad Sci USA 96(5):1898-1903 (1999)) describe an antibody mimic based on a lipocalin scaffold (Anticalin®). Lipocalins are composed of a beta-barrel with four hypervariable loops at the terminus of the protein. Beste (1999), subjected the loops to random mutagenesis and selected for binding with, for example, fluorescein. Three variants exhibited specific binding with fluorescein, with one variant showing binding similar to that of an anti-fluorescein antibody. Further analysis revealed that all of the randomized positions are variable, indicating that Anticalin® would be suitable to be used as an alternative to antibodies.

Anticalins® are small, single chain peptides, typically between 160 and 180 residues, which provides several advantages over antibodies, including decreased cost of production, increased stability in storage and decreased immunological reaction.

Hamilton et al. (U.S. Pat. No. 5,770,380) describe a synthetic antibody mimic using the rigid, non-peptide organic scaffold of calixarene, attached with multiple variable peptide loops used as binding sites. The peptide loops all project from the same side geometrically from the calixarene, with respect to each other. Because of this geometric conformation, all of the loops are available for binding, increasing the binding affinity to a ligand. However, in comparison to other antibody mimics, the calixarene-based antibody mimic does not consist exclusively of a peptide, and therefore it is less vulnerable to attack by protease enzymes. Neither does the scaffold consist purely of a peptide, DNA or RNA, meaning this antibody mimic is relatively stable in extreme environmental conditions and has a long life span. Further, since the calixarene-based antibody mimic is relatively small, it is less likely to produce an immunogenic response.

Murali et al. (Cell Mol Biol. 49(2):209-216 (2003)) describe a methodology for reducing antibodies into smaller peptidomimetics, they term “antibody like binding peptidomimetics” (ABiP) which can also be useful as an alternative to antibodies.

Silverman et al. (Nat Biotechnol. (2005), 23: 1556-1561) describe fusion proteins that are single-chain polypeptides including multiple domains termed “avimers.” Developed from human extracellular receptor domains by in vitro exon shuffling and phage display the avimers are a class of binding proteins somewhat similar to antibodies in their affinities and specificities for various target molecules. The resulting multidomain proteins can include multiple independent binding domains that can exhibit improved affinity (in some cases sub-nanomolar) and specificity compared with single-epitope binding proteins. Additional details concerning methods of construction and use of avimers are disclosed, for example, in US Pat. App. Pub. Nos. 20040175756, 20050048512, 20050053973, 20050089932 and 20050221384.

In addition to non-immunoglobulin protein frameworks, antibody properties have also been mimicked in compounds including, but not limited to, RNA molecules and unnatural oligomers (e.g., protease inhibitors, benzodiazepines, purine derivatives and beta-turn mimics) all of which are suitable for use with the present invention.
(vii) Antibody Neutralizing NPTX1 Activity:
The term “neutralizing” in reference to an anti-NPTX1 antibody of the invention or the phrase “antibody that neutralizes NPTX1 activity” is intended to refer to an antibody whose binding to or contact with NPTX1 results in inhibition of a cell proliferative activity by NPTX1. Because the NPTX1 is secreted to extracellular and functions as an essential factor of proliferation of lung cancer cells, some anti-NPTX1 antibodies may neutralize this activity.
(viii) Selecting the Antibody or Antibody Fragment:

The antibody or antibody fragment prepared by an aforementioned method may be selected by detecting affinity of the CDKN3 binding region of EF-1delta (at the position of 72-160aa) expressing cells like cancers cell. Unspecific binding to these cells is blocked by treatment with PBS containing 3% BSA for 30 min at room temperature. Cells are incubated for 60 min at room temperature with candidate antibody or antibody fragment. After washing with PBS, the cells are stained by FITC-conjugated secondary antibody for 60 min at room temperature and detected by using fluorometer. Alternatively, a biosensor using the surface plasmon resonance phenomenon may be used as a mean for detecting or quantifying the antibody or antibody fragment in the present invention. The antibody or antibody fragment which can detect the CDKN3 binding region (at the position of 72-160aa) of EF-1delta on the cell surface is selected in the presence invention.

Rabbit polyclonal antibodies (pAbs) specific for NPTX1 (BB017) were raised by immunizing rabbits with GST-fused human NPTX1 protein (codons 20-145: SEQ ID NO: 88 and 297-430: SEQ ID NO: 89), and purified using a standard protocol. Mouse monoclonal antibody (mAb) specific for human NPTX1 (mAb-75-1) was also generated by immunizing BALB/c mice (Chowdhury) intradermally with plasmid DNA encoding human NPTX1 protein using gene gun. NPTX1 mAb was purified by affinity chromatography from cell culture supernatant. NPTX1 mAb was proved to be specific for human NPTX1, by western-blot analysis using lysates of lung-cancer cell lines which expressed NPTX1 endogenously or not.

### (ix) Pharmaceutical Formulations:

Therapeutic formulations of present antibodies used in accordance with the present invention may be prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Lyophilized formulations adapted for subcutaneous administration are described in W097/04801. Such lyophilized formulations may be reconstituted with a suitable diluent to a high protein concentration and the reconstituted formulation may be administered subcutaneously to the mammal to be treated herein.

The formulation herein may also contain more than one active compound as necessary for the particular indication being treated, preferably those with complementary activities that do not adversely affect each other. For example, it may be desirable to further provide a chemotherapeutic agent, cytokine or immunosuppressive agent. The effective amount of such other agents depends on the amount of antibody present in the formulation, the type of disease or disorder or treatment, and other factors discussed above. These are generally used in the same dosages and with administration routes as used hereinbefore or about from 1 to 99% of the heretofore employed dosages.

The active ingredients may also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained release preparations include semipermeable matrices of solid hydrophobic polymers containing the agent, which matrices are in the form of shaped articles, e.g. films, or microcapsules. Examples of sustained-release matrices include polyesters, hydrogels (for example, poly (2-hydroxyethyl-methacrylate), or poly (vinylalcohol)), polylactides (U.S. Pat. No. 3,773,919), copolymers of L-glutamic acid and ethyl-L-glutamate, non degradable ethylene-vinyl acetate, degradable lactic acid-glycolic acid copolymers such as the LUPRON DEPOT (injectable microspheres composed of lactic acid-glycolic acid copolymer and leuprolide acetate), and poly-D(−)-3-hydroxybutyric acid. The formulations to be used for in vivo administration must be sterile. This is readily accomplished by filtration through sterile filtration membranes.

(x) Treatment with an Antibody:

A composition comprising present antibodies may be formulated, dosed, and administered in a fashion consistent with good medical practice. Preferably, the present antibody will be a human, chimeric or humanized antibody scFv, or antibody fragment. Factors for consideration in this context include the particular lung cancer being treated, the particular mammal being treated, the clinical condition of the individual patient, the cause of the disease or disorder, the site of delivery of the agent, the method of administration, the scheduling of administration, and other factors known to medical practitioners. The therapeutically effective amount of the antibody to be administered will be governed by such considerations.

As a general proposition, the therapeutically effective amount of the antibody administered parenterally per dose will be in the range of about 0.1 to 20 mg/kg of patient body weight per day, with the typical initial range of antibody used being in the range of about 2 to 10 mg/kg.

As noted above, however, these suggested amounts of antibody are subject to a great deal of therapeutic discretion. The key factor in selecting an appropriate dose and scheduling is the result obtained, as indicated above.

For example, relatively higher doses may be needed initially for the treatment of ongoing and acute diseases. To obtain the most efficacious results, depending on the disease or disorder, the antibody may be administered as close to the first sign, diagnosis, appearance, or occurrence of the disease or disorder as possible or during remissions of the disease or disorder.

The antibody may be administered by any suitable means, including parenteral, subcutaneous, intraperitoneal, intrapulmonary, and intranasal, and, if desired for local immunosuppressive treatment, intralesional administration. Parenteral infusions include intramuscular, intravenous, intraarterial, intraperitoneal, or subcutaneous administration.

In addition, the antibody may suitably be administered by pulse infusion, e.g., with declining doses of the antibody. Preferably the dosing is given by injections, most preferably intravenous or subcutaneous injections, depending in part on whether the administration is brief or chronic.

One additionally may administer other compounds, such as cytotoxic agents, chemotherapeutic agents, immunosuppressive agents and/or cytokines with the antibody herein. The combined administration includes co-administration, using separate formulations or a single pharmaceutical formulation, and consecutive administration in either order, wherein preferably there is a time period while both (or all) active agents simultaneously exert their biological activities.

Aside from administration of the antibody to the patient, the present invention contemplates administration of the antibody by gene therapy. Such administration of a nucleic acid encoding an antibody is encompassed by the expression “administering a therapeutically effective amount of an antibody”. See, for example, W096/07321 published Mar. 14, 1996 concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

The currently preferred in vivo nucleic acid transfer techniques include transfection with viral vectors (such as adenovirus, Herpes simplex I virus, or adeno-associated virus) and lipid-based systems (useful lipids for lipid mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262: 4429-4432 (1987); and Wagner et al., Proc. Nad. Acad. Sci. USA 87: 3410-3414 (1990). For review of the currently known gene marking and gene therapy protocols see Anderson et al., Science 256: 808-813 (1992). See also WO 93/25673 and the references cited therein.

### Double-Stranded Molecules:

As used herein, the term “isolated double-stranded molecule” refers to a nucleic acid molecule that inhibits expression of a target gene and includes, for example, short interfering RNA (siRNA; e.g., double-stranded ribonucleic acid (dsRNA) or small hairpin RNA (shRNA)) and short interfering DNA/RNA (siD/R-NA; e.g. double-stranded chimera of DNA and RNA (dsD/R-NA) or small hairpin chimera of DNA and RNA (shD/R-NA)).

As use herein, the term “siRNA” refers to a double-stranded RNA molecule which prevents translation of a target mRNA. Standard techniques of introducing siRNA into the cell are used, including those in which DNA is a template from which RNA is transcribed. The siRNA includes an EBI3, CDKN3 or EF-1delta sense nucleic acid sequence (also referred to as “sense strand”), an EBI3, CDKN3 or EF-1delta antisense nucleic acid sequence (also referred to as “antisense strand”) or both. The siRNA may be constructed such that a single transcript has both the sense and complementary antisense nucleic acid sequences of the target gene, e.g., a hairpin. The siRNA may either be a dsRNA or shRNA.

As used herein, the term “dsRNA” refers to a construct of two RNA molecules composed of complementary sequences to one another and that have annealed together via the complementary sequences to form a double-stranded RNA molecule. The nucleotide sequence of two strands may include not only the “sense” or “antisense” RNAs selected from a protein coding sequence of target gene sequence, but also RNA molecule having a nucleotide sequence selected from non-coding region of the target gene.

The term “shRNA”, as used herein, refers to an siRNA having a stem-loop structure, composed of first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shRNA is a single-stranded region intervening between the sense and antisense strands and may also be referred to as “intervening single-strand”.

As use herein, the term “siD/R-NA” refers to a double-stranded polynucleotide molecule which is composed of both RNA and DNA, and includes hybrids and chimeras of RNA and DNA and prevents translation of a target mRNA. Herein, a hybrid indicates a molecule wherein a polynucleotide composed of DNA and a polynucleotide composed of RNA hybridize to each other to form the double-stranded molecule; whereas a chimera indicates that one or both of the strands composing the double stranded molecule may contain RNA and DNA. Standard techniques of introducing siD/R-NA into the cell are used. The siD/R-NA includes an EBI3, CDKN3 or EF-1delta sense nucleic acid sequence (also referred to as “sense strand”), an EBI3, CDKN3 or EF-1delta antisense nucleic acid sequence (also referred to as “antisense strand”) or both. The siD/R-NA may be constructed such that a single transcript has both the sense and complementary antisense nucleic acid sequences from the target gene, e.g., a hairpin. The siD/R-NA may either be a dsD/R-NA or shD/R-NA.

As used herein, the term “dsD/R-NA” refers to a construct of two molecules composed of complementary sequences to one another and that have annealed together via the complementary sequences to form a double-stranded polynucleotide molecule. The nucleotide sequence of two strands may comprise not only the “sense” or “antisense” polynucleotides sequence selected from a protein coding sequence of target gene sequence, but also polynucleotide having a nucleotide sequence selected from non-coding region of the target gene. One or both of the two molecules constructing the dsD/R-NA are composed of both RNA and DNA (chimeric molecule), or alternatively, one of the molecules is composed of RNA and the other is composed of DNA (hybrid double-strand).

The term “shD/R-NA”, as used herein, refers to an siD/R-NA having a stem-loop structure, composed of a first and second regions complementary to one another, i.e., sense and antisense strands. The degree of complementarity and orientation of the regions being sufficient such that base pairing occurs between the regions, the first and second regions being joined by a loop region, the loop resulting from a lack of base pairing between nucleotides (or nucleotide analogs) within the loop region. The loop region of an shD/R-NA is a single-stranded region intervening between the sense and antisense strands and may also be referred to as “intervening single-strand”.

As used herein, an “isolated nucleic acid” is a nucleic acid removed from its original environment (e.g., the natural environment if naturally occurring) and thus, synthetically altered from its natural state. In the present invention, examples of isolated nucleic acid includes DNA, RNA, and derivatives thereof.

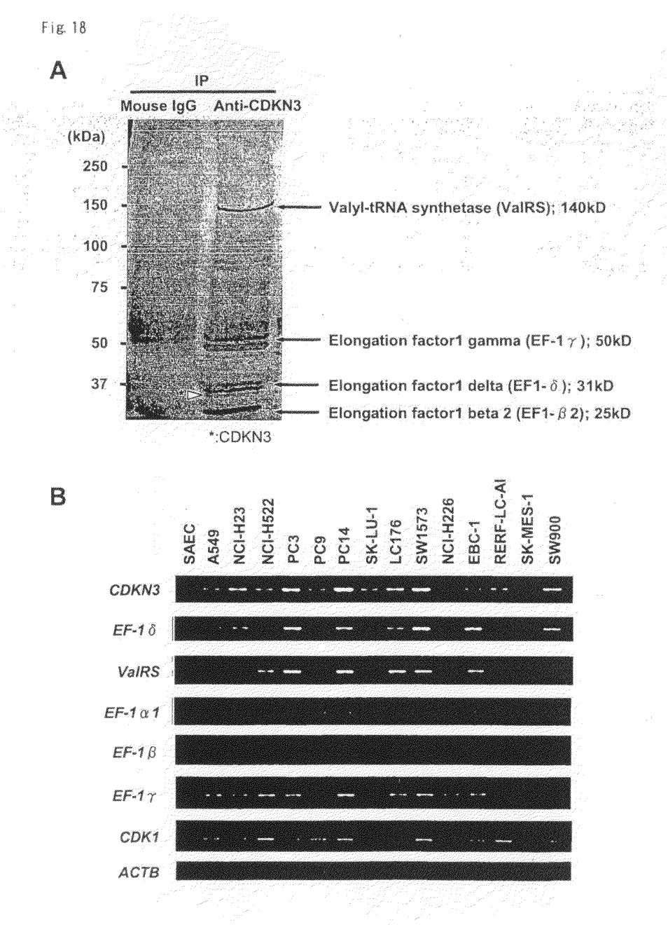

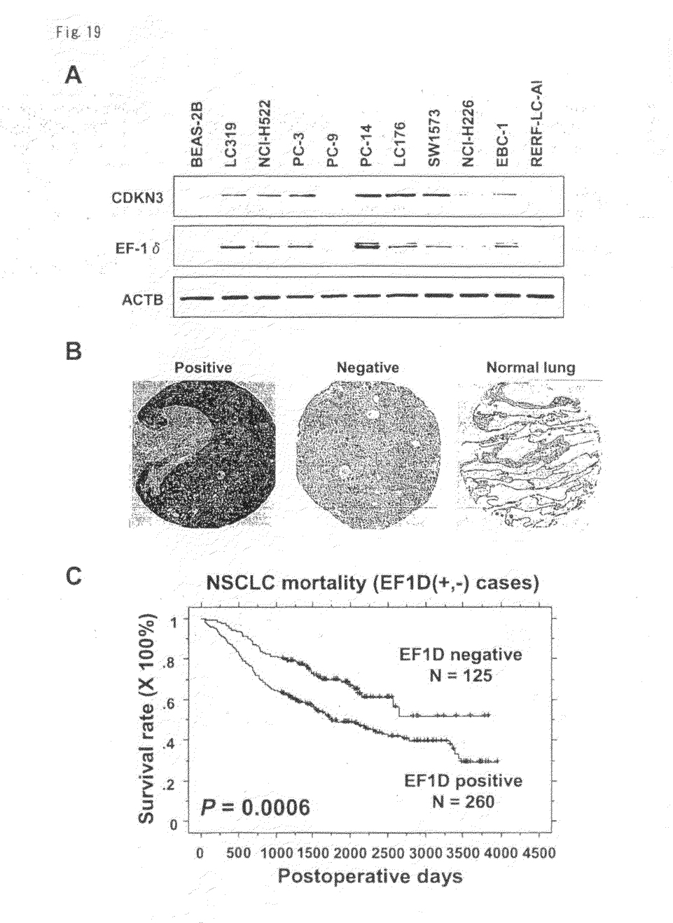

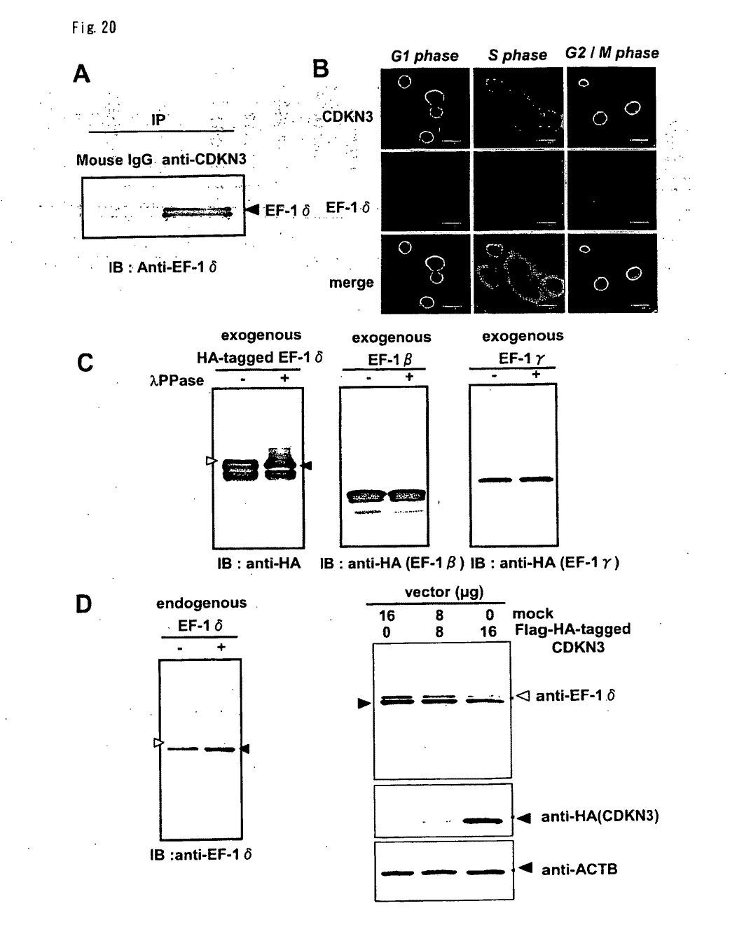

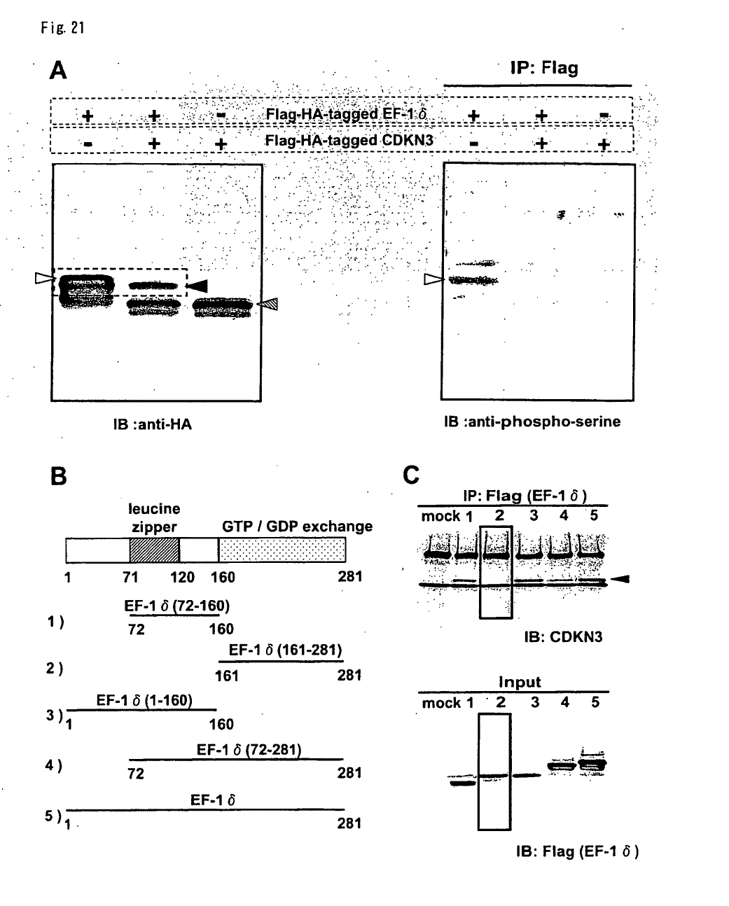

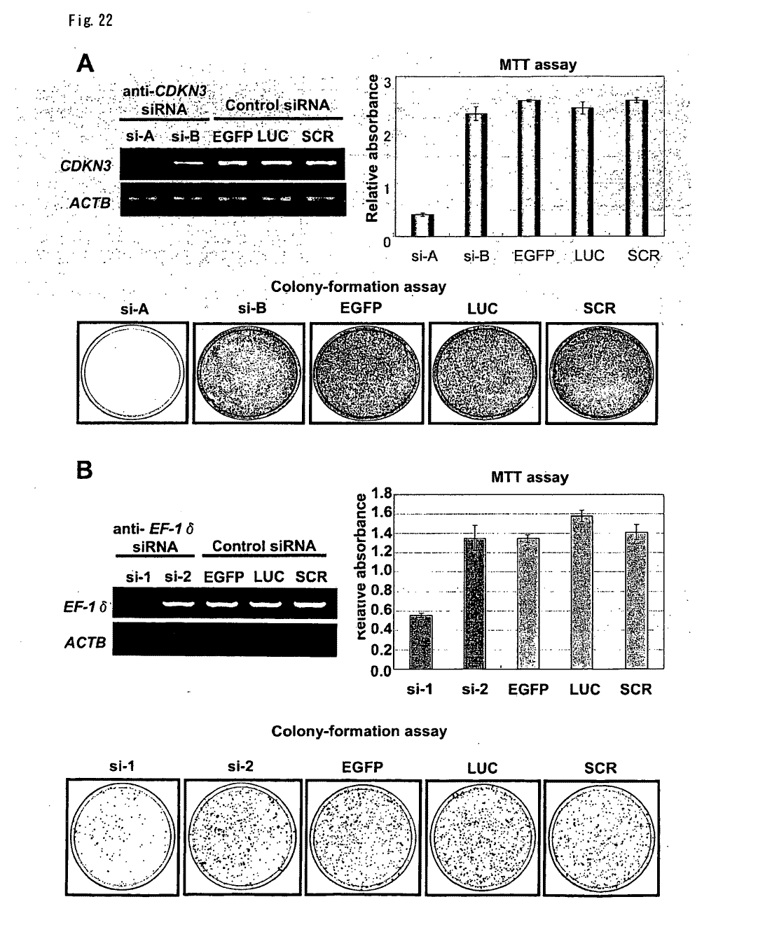

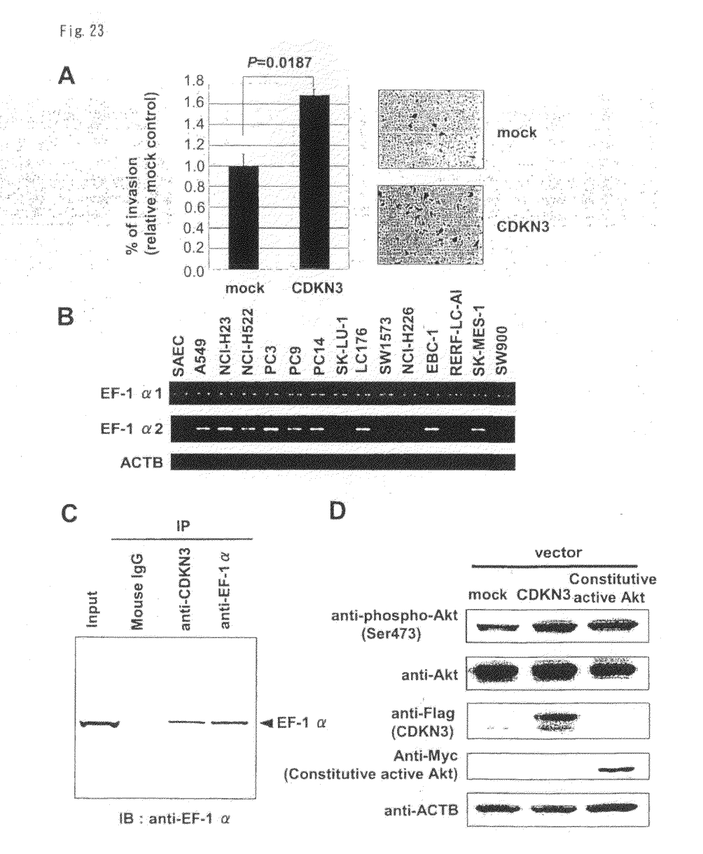

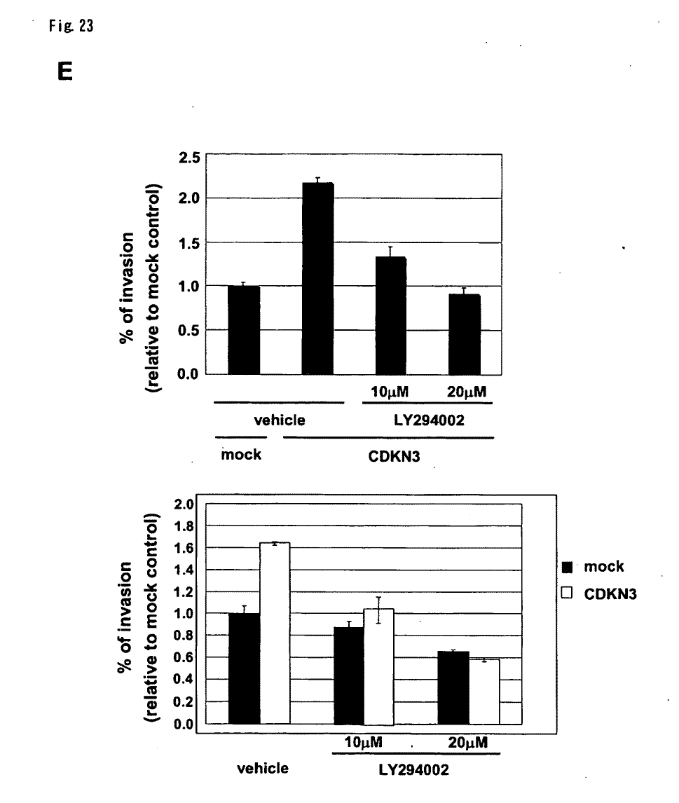

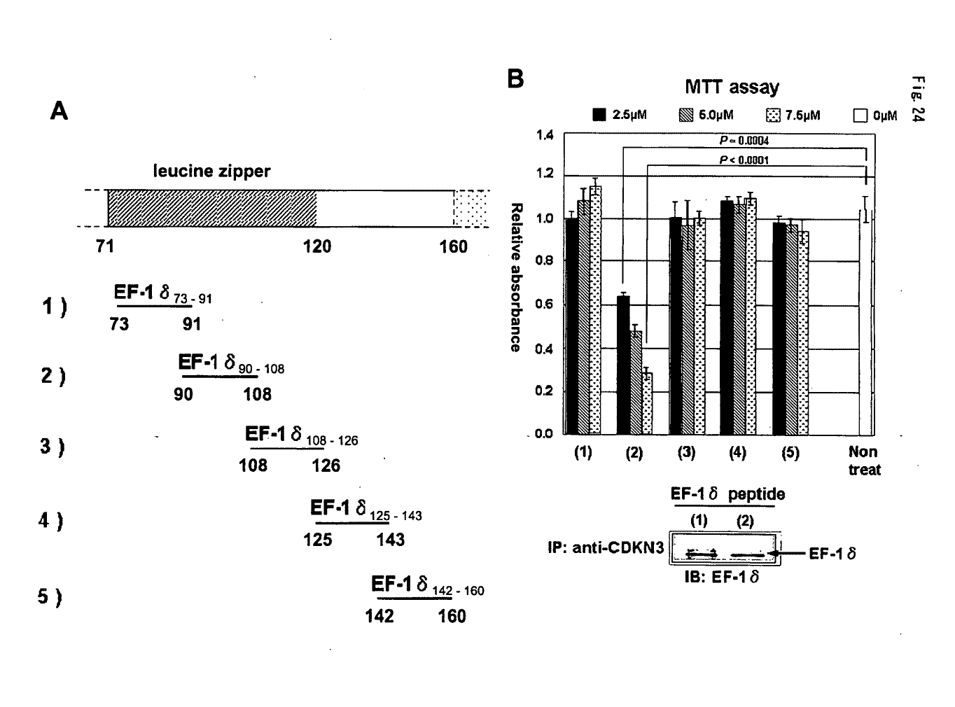

A double-stranded molecule against EBI3, CDKN3, EF-1delta or NPTXR, which molecule hybridizes to target mRNA, decreases or inhibits production of EBI3, CDKN3, EF-1delta or NPTXR protein encoded by EBI3, CDKN3, EF-1delta or NPTXR gene by associating with the normally single-stranded mRNA transcript of the gene, thereby interfering with translation and thus, inhibiting expression of the protein. As demonstrated herein, the expression of EBI3 in lung cancer cell lines was inhibited by dsRNA (FIG. 4D); the expression of CDKN3 in lung cancer cell lines was inhibited by dsRNA (FIG. 22A); the expression of NPTXR in lung cancer cell lines was inhibited by dsRNA (FIG. 13D); the expression of EF-1delta in lung cancer cell lines was inhibited by dsRNA (FIG. 22B).

Therefore the present invention provides isolated double-stranded molecules that are capable of inhibiting the inhibit expression of EBI3, CDKN3 or EF-1delta gene when introduced into a cell expressing the gene. The target sequence of double-stranded molecule may be designed by an siRNA design algorithm such as that mentioned below.

EBI3 target sequence includes, for example, nucleotides
SEQ ID NO: 18 (at the position 679-697nt of SEQ ID NO: 1)
SEQ ID NO: 20 (at the position 280-298nt of SEQ ID NO: 1)

CDKN3 target sequence includes, for example, nucleotides
SEQ ID NO: 49 (at the position of 310-328nt of SEQ ID NO: 5)

EF-1delta target sequence includes, for example, nucleotides
SEQ ID NO: 51 (at the position of 225-243nt of SEQ ID NO: 7)

NPTXR target sequence includes, for example, nucleotides
SEQ ID NO: 84 (at the position 1280-1298nt of SEQ ID NO: 86)
SEQ ID NO: 85 (at the position 1393-1411nt of SEQ ID NO: 86)

Specifically, the present invention provides the following double-stranded molecules [1] to [20]:

[1] An isolated double-stranded molecule that, when introduced into a cell, inhibits in vivo expression of EBI3, CDKN3, EF-1delta or NPTXR and cell proliferation, such molecules composed of a sense strand and an antisense strand complementary thereto, hybridized to each other to form the double-stranded molecule;

[2] The double-stranded molecule of [1], wherein said double-stranded molecule acts on mRNA, matching a target sequence selected from among SEQ ID NO: 18 (at the position of 679-697nt of SEQ ID NO: 1), SEQ ID NO: 20 (at the position of 280-298nt of SEQ ID NO: 1), SEQ ID NO: 49 (at the position of 310-328nt of SEQ ID NO: 5), SEQ ID NO: 51 (at the position of 225-243nt of SEQ ID NO: 7), SEQ ID NO: 84 (at the position 1280-1298nt of SEQ ID NO: 86) and SEQ ID NO: 85 (at the position 1393-1411nt of SEQ ID NO: 86);

[3] The double-stranded molecule of [2], wherein the sense strand contains a sequence corresponding to a target sequence selected from among SEQ ID NOs: 18, 20, 49, 51 84 and 85;

[4] The double-stranded molecule of [3], having a length of less than about 100 nucleotides;

[5] The double-stranded molecule of [4], having a length of less than about 75 nucleotides;

[6] The double-stranded molecule of [5], having a length of less than about 50 nucleotides;

[7] The double-stranded molecule of [6] having a length of less than about 25 nucleotides;

[8] The double-stranded molecule of [7], having a length of between about 19 and about 25 nucleotides;

[9] The double-stranded molecule of [3], composed of a single polynucleotide having both the sense and antisense strands linked by an intervening single-strand;

[10] The double-stranded molecule of [9], having the general formula 5′-[A]-[B]-[A′]-3′, wherein [A] is the sense strand containing a sequence corresponding to a target sequence selected from among SEQ ID NOs: 18, 20, 49, 51, 84 and 85, [B] is the intervening single-strand composed of 3 to 23 nucleotides, and [A′] is the antisense strand containing a sequence complementary to [A];

[11] The double-stranded molecule of [1], composed of RNA;

[12] The double-stranded molecule of [1], composed of both DNA and RNA;

[13] The double-stranded molecule of [12], wherein the molecule is a hybrid of a DNA polynucleotide and an RNA polynucleotide;

[14] The double-stranded molecule of [13] wherein the sense and the antisense strands are composed of DNA and RNA, respectively;

[15] The double-stranded molecule of [12], wherein the molecule is a chimera of DNA and RNA;

[16] The double-stranded molecule of [15], wherein a region flanking to the 3′-end of the antisense strand, or both of a region flanking to the 5′-end of sense strand and a region flanking to the 3′-end of antisense strand are RNA;

[17] The double-stranded molecule of [16], wherein the flanking region is composed of 9 to 13 nucleotides; and

[18] The double-stranded molecule of [2], wherein the molecule contains 3′ overhang;

[19] A vector expressing the double-stranded molecule of [2];

[20] The vector of [19], wherein the double-stranded molecule has the general formula 5′-[A]-[B]-[A′]-3′, wherein [A] is the sense strand contains a sequence corresponding to a target sequence selected from among SEQ ID NOs: 18, 20, 49, 51, 84 and 85, [B] is an intervening single-strand is composed of 3 to 23 nucleotides, and [A′] is the antisense strand contains a sequence complementary to [A].

The double-stranded molecule of the present invention will be described in more detail below.

Methods for designing double-stranded molecules having the ability to inhibit target gene expression in cells are known. (See, for example, U.S. Pat. No. 6,506,559, herein incorporated by reference in its entirety). For example, a computer program for designing siRNAs is available from the Ambion website (http://www.ambion.com/techlib/misc/siRNA_finder.html).

The computer program selects target nucleotide sequences for double-stranded molecules based on the following protocol.

Selection of Target Sites:

1. Beginning with the AUG start codon of the transcript, scan downstream for AA di-nucleotide sequences. Record the occurrence of each AA and the 3′ adjacent 19 nucleotides as potential siRNA target sites. Tuschl et al. recommend to avoid designing siRNA to the 5′ and 3′ untranslated regions (UTRs) and regions near the start codon (within 75 bases) as these may be richer in regulatory protein binding sites, and UTR-binding proteins and/or translation initiation complexes may interfere with binding of the siRNA endonuclease complex.

2. Compare the potential target sites to the appropriate genome database (human, mouse, rat, etc.) and eliminate from consideration any target sequences with significant homology to other coding sequences. Basically, BLAST, which can be found on the NCBI server at: www.ncbi.nlm.nih.gov/BLAST/, is used (Altschul S F et al., Nucleic Acids Res 1997 Sep. 1, 25(17): 3389-402).

3. Select qualifying target sequences for synthesis. Selecting several target sequences along the length of the gene to evaluate is typical.

Using the above protocol, the target sequence of the isolated double-stranded molecules of the present invention were designed as

SEQ ID NO: 18 and 20 for EBI3 gene,

SEQ ID NO: 49 and 50 for CDKN3 gene,

SEQ ID NO: 51 and 52 for EF-1delta gene or

SEQ ID NO: 84 and 85 for NPTXR gene.

Double-stranded molecules targeting the above-mentioned target sequences were respectively examined for their ability to suppress the growth of cells expressing the target genes. Therefore, the present invention provides double-stranded molecules targeting any of the sequences selected from the group of

SEQ ID NO: 18 (at the position 679-697nt of SEQ ID NO: 1) or 20 (at the position 280-298nt of SEQ ID NO: 1) for EBI3 gene,

SEQ ID NO: 49 (at the position of 310-328nt of SEQ ID NO: 5) for CDKN3 gene,

SEQ ID NO: 51 (at the position of 225-243nt of SEQ ID NO: 7) for EF-1delta gene and

SEQ ID NO: 84 (at the position of 1280-1298nt of SEQ ID NO: 86) or SEQ ID NO: 85 (at the position of 1393-1411nt of SEQ ID NO: 86).

The double-stranded molecule of the present invention may be directed to a single target EBI3, CDKN3, EF-1delta or NPTXR gene sequence or may be directed to a plurality of target EBI3, CDKN3, EF-1delta and/or NPTXR gene sequences.

A double-stranded molecule of the present invention targeting the above-mentioned targeting sequence of EBI3, CDKN3, EF-1delta and/or NPTXR gene include isolated polynucleotides that contain any of the nucleic acid sequences of target sequences and/or complementary sequences to the target sequences. Examples of polynucleotides targeting EBI3 gene include those containing the sequence of SEQ ID NO: 18 or 20 and/or complementary sequences to these nucleotides; polynucleotides targeting CDKN3 gene include those containing the sequence of SEQ ID NO: 49 and/or complementary sequences to these nucleotides; polynucleotides targeting EF-1delta gene include those containing the sequence of SEQ ID NO: 51 and/or complementary sequences to these nucleotides; polynucleotides targeting NPTXR gene include those containing the sequence of SEQ ID NO: 84 or 85 and/or complementary sequences to these nucleotides. However, the present invention is not limited to these examples, and minor modifications in the aforementioned nucleic acid sequences are acceptable so long as the modified molecule retains the ability to suppress the expression of EBI3, CDKN3, EF-1delta or NPTXR gene. Herein, the phrase “minor modification” as used in connection with a nucleic acid sequence indicates one, two or several substitution, deletion, addition or insertion of nucleic acids to the sequence.

In the context of the present invention, the term “several” as applies to nucleic acid substitutions, deletions, additions and/or insertions may mean 3-7, preferably 3-5, more preferably 3-4, even more preferably 3 nucleic acid residues.

According to the present invention, a double-stranded molecule of the present invention can be tested for its ability using the methods utilized in the Examples. In the Examples herein below, double-stranded molecules composed of sense strands of various portions of mRNA of EBI3, CDKN3, EF-1delta or NPTXR genes or antisense strands complementary thereto were tested in vitro for their ability to decrease production of EBI3, CDKN3, EF-1delta or NPTXR gene product in lung cancer cell lines (e.g., using A549 for EBI3, LC319 for CDKN3 or EF-1delta) according to standard methods. Furthermore, for example, reduction in EBI3, CDKN3, EF-1delta or NPTXR gene product in cells contacted with the candidate double-stranded molecule compared to cells cultured in the absence of the candidate molecule can be detected by, e.g. RT-PCR using primers for EBI3, CDKN3, EF-1delta or NPTXR mRNA mentioned under Example 1, 11 and 18 item “Semi-quantitative RT-PCR”. Sequences which decrease the production of EBI3, CDKN3, EF-1delta or NPTXR gene product in in vitro cell-based assays can then be tested for there inhibitory effects on cell growth. Sequences which inhibit cell growth in in vitro cell-based assay can then be tested for their in vivo ability using animals with cancer, e.g. nude mouse xenograft models, to confirm decreased production of EBI3, CDKN3, EF-1delta or NPTXR product and decreased cancer cell growth.

When the isolated polynucleotide is RNA or derivatives thereof, base “t” should be replaced with “u” in the nucleotide sequences. As used herein, the term “complementary” refers to Watson-Crick or Hoogsteen base pairing between nucleotides units of a polynucleotide, and the term “binding” means the physical or chemical interaction between two polynucleotides. When the polynucleotide includes modified nucleotides and/or non-phosphodiester linkages, these polynucleotides may also bind each other as same manner. Generally, complementary polynucleotide sequences hybridize under appropriate conditions to form stable duplexes containing few or no mismatches. Furthermore, the sense strand and antisense strand of the isolated polynucleotide of the present invention can form double-stranded molecule or hairpin loop structure by the hybridization. In a preferred embodiment, such duplexes contain no more than 1 mismatch for every 10 matches. In an especially preferred embodiment, where the strands of the duplex are fully complementary, such duplexes contain no mismatches.

The polynucleotide is preferably less than 1149 nucleotides in length for EBI3, less than 844 nucleotides in length for CDKN3, less than 1031 nucleotides in length for EF-1delta and less than 5815 nucleotides in length for NPTXR. For example, the polynucleotide is less than 500, 200, 100, 75, 50, or 25 nucleotides in length for all of the genes. The isolated polynucleotides of the present invention are useful for forming double-stranded molecules against EBI3, CDKN3, EF-1delta or NPTXR gene or preparing template DNAs encoding the double-stranded molecules. When the polynucleotides are used for forming double-stranded molecules, the polynucleotide may be longer than 19 nucleotides, preferably longer than 21 nucleotides, and more preferably has a length of between about 19 and 25 nucleotides.

The double-stranded molecules of the invention may contain one or more modified nucleotides and/or non-phosphodiester linkages. Chemical modifications well known in the art are capable of increasing stability, availability, and/or cell uptake of the double-stranded molecule. The skilled person will be aware of other types of chemical modification which may be incorporated into the present molecules (WO03/070744; WO2005/045037). In one embodiment, modifications can be used to provide improved resistance to degradation or improved uptake. Examples of such modifications include, but are not limited to, phosphorothioate linkages, 2′-O-methyl ribonucleotides (especially on the sense strand of a double-stranded molecule), 2′-deoxy-fluoro ribonucleotides, 2′-deoxy ribonucleotides, “universal base” nucleotides, 5′-C-methyl nucleotides, and inverted deoxybasic residue incorporation (US20060122137).

In another embodiment, modifications can be used to enhance the stability or to increase targeting efficiency of the double-stranded molecule. Examples of such modifications include, but are not limited to, chemical cross linking between the two complementary strands of a double-stranded molecule, chemical modification of a 3′ or 5′ terminus of a strand of a double-stranded molecule, sugar modifications, nucleobase modifications and/or backbone modifications, 2-fluoro modified ribonucleotides and 2′-deoxy ribonucleotides (WO2004/029212). In another embodiment, modifications can be used to increased or decreased affinity for the complementary nucleotides in the target mRNA and/or in the complementary double-stranded molecule strand (WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyl, or 7-alkenyl purine. In another embodiment, when the double-stranded molecule is a double-stranded molecule with a 3′ overhang, the 3′-terminal nucleotide overhanging nucleotides may be replaced by deoxyribonucleotides (Elbashir S M et al., Genes Dev 2001 Jan. 15, 15(2): 188-200). For further details, published documents such as US20060234970 are available. The present invention is not limited to these examples and any known chemical modifications may be employed for the double-stranded molecules of the present invention so long as the resulting molecule retains the ability to inhibit the expression of the target gene.

Furthermore, the double-stranded molecules of the invention may comprise both DNA and RNA, e.g., dsD/R-NA or shD/R-NA. Specifically, a hybrid polynucleotide of a DNA strand and an RNA strand or a DNA-RNA chimera polynucleotide shows increased stability. Mixing of DNA and RNA, i.e., a hybrid type double-stranded molecule composed of a DNA strand (polynucleotide) and an RNA strand (polynucleotide), a chimera type double-stranded molecule containing both DNA and RNA on any or both of the single strands (polynucleotides), or the like may be formed for enhancing stability of the double-stranded molecule.

The hybrid of a DNA strand and an RNA strand may be either where the sense strand is DNA and the antisense strand is RNA, or the opposite so long as it can inhibit expression of the target gene when introduced into a cell expressing the gene. Preferably, the sense strand polynucleotide is DNA and the antisense strand polynucleotide is RNA. Also, the chimera type double-stranded molecule may be either where both of the sense and antisense strands are composed of DNA and RNA, or where any one of the sense and antisense strands is composed of DNA and RNA so long as it has an activity to inhibit expression of the target gene when introduced into a cell expressing the gene. In order to enhance stability of the double-stranded molecule, the molecule preferably contains as much DNA as possible, whereas to induce inhibition of the target gene expression, the molecule is required to be RNA within a range to induce sufficient inhibition of the expression.

As a preferred example of the chimera type double-stranded molecule, an upstream partial region (i.e., a region flanking to the target sequence or complementary sequence thereof within the sense or antisense strands) of the double-stranded molecule is RNA. Preferably, the upstream partial region indicates the 5′ side (5′-end) of the sense strand and the 3′ side (3′-end) of the antisense strand. Alternatively, regions flanking to 5′-end of sense strand and/or 3′-end of antisense strand are referred to upstream partial region. That is, in preferable embodiments, a region flanking to the 3′-end of the antisense strand, or both of a region flanking to the 5′-end of sense strand and a region flanking to the 3′-end of antisense strand are composed of RNA. For instance, the chimera or hybrid type double-stranded molecule of the present invention include following combinations.

| | |
|---|---|
| sense strand: | 5′-[DNA]-3′ |
| | 3′-(RNA)-[DNA]-5′: antisense strand, |
| sense strand: | 5′-(RNA)-[DNA]-3′ |
| | 3′-(RNA)-[DNA]-5′: antisense strand, |

and

| | |
|---|---|
| sense strand: | 5′-(RNA)-[DNA]-3′ |
| | 3′-(RNA)-5′: antisense strand. |

The upstream partial region preferably is a domain composed of 9 to 13 nucleotides counted from the terminus of the target sequence or complementary sequence thereto within the sense or antisense strands of the double-stranded molecules. Moreover, preferred examples of such chimera type double-stranded molecules include those having a strand length of 19 to 21 nucleotides in which at least the upstream half region (5′ side region for the sense strand and 3′ side region for the antisense strand) of the polynucleotide is RNA and the other half is DNA. In such a chimera type double-stranded molecule, the effect to inhibit expression of the target gene is much higher when the entire antisense strand is RNA (US20050004064).

In the present invention, the double-stranded molecule may form a hairpin, such as a short hairpin RNA (shRNA) and short hairpin consisting of DNA and RNA (shD/R-NA). The shRNA or shD/R-NA is a sequence of RNA or mixture of RNA and DNA making a tight hairpin turn that can be used to silence gene expression via RNA interference. The shRNA or shD/R-NA comprises the sense target sequence and the antisense target sequence on a single strand wherein the sequences are separated by a loop sequence. Generally, the hairpin structure is cleaved by the cellular machinery into dsRNA or dsD/R-NA, which is then bound to the RNA-induced silencing complex (RISC). This complex binds to and cleaves mRNAs which match the target sequence of the dsRNA or dsD/R-NA.

A loop sequence composed of an arbitrary nucleotide sequence can be located between the sense and antisense sequence in order to form the hairpin loop structure. Thus, the present invention also provides a double-stranded molecule having the general formula 5′-[A]-[B]-[A′]-3′, wherein [A] is the sense strand containing a sequence corresponding to a target sequence, [B] is an intervening single-strand and [A′] is the antisense strand containing a complementary sequence to [A]. The target sequence may be selected from among, for example, nucleotides of SEQ ID NO: 18 and 20 for EBI3, SEQ ID NO: 49 for CDKN3, SEQ ID NO: 51 for EF-1delta or SEQ ID NO: 84 and 85 for NPTXR.

The present invention is not limited to these examples, and the target sequence in [A] may be modified sequences from these examples so long as the double-stranded molecule retains the ability to suppress the expression of the targeted EBI3, CDKN3, EF-1delta or NPTXRgene. The region [A] hybridizes to [A′] to form a loop composed of the region [B]. The intervening single-stranded portion [B], i.e., loop sequence may be preferably 3 to 23 nucleotides in length. The loop sequence, for example, can be selected from among the following sequences (http://www.ambion.com/techlib/tb/tb_{—}506.html). Furthermore, loop sequence consisting of 23 nucleotides also provides active siRNA (Jacque J M et al., Nature 2002 Jul. 25, 418(6896): 435-8, Epub 2002 Jun. 26):

CCC, CCACC, or CCACACC: Jacque J M et al., Nature 2002 Jul. 25, 418(6896): 435-8, Epub 2002 Jun. 26;

UUCG: Lee N S et al., Nat Biotechnol 2002 May, 20(5): 500-5; Fruscoloni P et al., Proc Natl Acad Sci USA 2003 Feb. 18, 100(4): 1639-44, Epub 2003 Feb. 10; and

UUCAAGAGA: Dykxhoorn D M et al., Nat Rev Mol Cell Biol 2003 June, 4(6): 457-67.

Examples of preferred double-stranded molecules of the present invention having hairpin loop structure are shown below. In the following structure, the loop sequence can be selected from among AUG, CCC, UUCG, CCACC, CTCGAG, AAGCUU, CCACACC, and UUCAAGAGA; however, the present invention is not limited thereto:

(for target sequence SEQ ID NO: 18)
CAAUGAGCCUGGGCAAGUA-[B]-UACUUGCCCAGGCUCAUUG;
(for target sequence SEQ ID NO: 20)
UCACGGAUGUCCAGCUGUU-[B]-AACAGCUGGACAUCCGUGA;
(for target sequence SEQ ID NO: 49)
UAUAGAGUCCCAAACCUUC-[B]-GAAGGUUUGGGACUCUAUA;
(for target sequence SEQ ID NO: 51)
GUGGAGAACCAGAGUCUGC-[B]-GCAGACUCUGGUUCUCCAC;
(for target sequence SEQ ID NO: 84)
GACAAUGGCUGGCACCACA-[B]-UGUGGUGCCAGCCAUUGUC;
and
(for target sequence SEQ ID NO: 85)
CAUCAAGCCUCAUGGGAUC-[B]-GAUCCCAUGAGGCUUGAUG

Furthermore, in order to enhance the inhibition activity of the double-stranded molecules, nucleotide “u” can be added to 3′ end of the antisense strand of the target sequence, as 3′ overhangs. The number of “u”s to be added is at least 2, generally 2 to 10, preferably 2 to 5. The added “u”s form single strand at the 3′ end of the antisense strand of the double-stranded molecule.

The method for preparing the double-stranded molecule is not particularly limited though it is preferable to use a chemical synthetic method known in the art. According to the chemical synthesis method, sense and antisense single-stranded polynucleotides are separately synthesized and then annealed together via an appropriate method to obtain a double-stranded molecule. Specific example for the annealing includes wherein the synthesized single-stranded polynucleotides are mixed in a molar ratio of preferably at least about 3:7, more preferably about 4:6, and most preferably substantially equimolar amount (i.e., a molar ratio of about 5:5). Next, the mixture is heated to a temperature at which double-stranded molecules dissociate and then is gradually cooled down. The annealed double-stranded polynucleotide can be purified by usually employed methods known in the art. Example of purification methods include methods utilizing agarose gel electrophoresis or wherein remaining single-stranded polynucleotides are optionally removed by, e.g., degradation with appropriate enzyme.

The regulatory sequences flanking EBI3, CDKN3, EF-1delta or NPTXR sequences may be identical or different, such that their expression can be modulated independently, or in a temporal or spatial manner. The double-stranded molecules can be transcribed intracellularly by cloning EBI3, CDKN3, EF-1delta or NPTXR gene templates into a vector containing, e.g., a RNA pol III transcription unit from the small nuclear RNA (snRNA) U6 or the human H1 RNA promoter.

### Vectors Containing a Double-Stranded Molecule of the Present Invention:

Also included in the present invention are vectors containing one or more of the double-stranded molecules described herein, and a cell containing such a vector. A vector of the present invention preferably encodes a double-stranded molecule of the present invention in an expressible form. Herein, the phrase “in an expressible form” indicates that the vector, when introduced into a cell, will express the molecule. In a preferred embodiment, the vector includes regulatory elements necessary for expression of the double-stranded molecule. Such vectors of the present invention may be used for producing the present double-stranded molecules, or directly as an active ingredient for treating cancer.

Vectors of the present invention can be produced, for example, by cloning EBI3, CDKN3, EF-1delta or NPTXR sequence into an expression vector so that regulatory sequences are operatively-linked to EBI3, CDKN3, EF-1delta or NPTXRsequence in a manner to allow expression (by transcription of the DNA molecule) of both strands (Lee N S et al., Nat Biotechnol 2002 May, 20(5): 500-5). For example, RNA molecule that is the antisense to mRNA is transcribed by a first promoter (e.g., a promoter sequence flanking to the 3′ end of the cloned DNA) and RNA molecule that is the sense strand to the mRNA is transcribed by a second promoter (e.g., a promoter sequence flanking to the 5′ end of the cloned DNA). The sense and antisense strands hybridize in vivo to generate a double-stranded molecule constructs for silencing of the gene. Alternatively, two vectors constructs respectively encoding the sense and antisense strands of the double-stranded molecule are utilized to respectively express the sense and anti-sense strands and then forming a double-stranded molecule construct. Furthermore, the cloned sequence may encode a construct having a secondary structure (e.g., hairpin); namely, a single transcript of a vector contains both the sense and complementary antisense sequences of the target gene.

The vectors of the present invention may also be equipped so to achieve stable insertion into the genome of the target cell (see, e.g., Thomas K R & Capecchi M R, Cell 1987, 51: 503-12 for a description of homologous recombination cassette vectors). See, e.g., Wolff et al., Science 1990, 247: 1465-8; U.S. Pat. Nos. 5,580,859; 5,589,466; 5,804,566; 5,739,118; 5,736,524; 5,679,647; and WO 98/04720. Examples of DNA-based delivery technologies include “naked DNA”, facilitated (bupivacaine, polymers, peptide-mediated) delivery, cationic lipid complexes, and particle-mediated (“gene gun”) or pressure-mediated delivery (see, e.g., U.S. Pat. No. 5,922,687).

The vectors of the present invention include, for example, viral or bacterial vectors. Examples of expression vectors include attenuated viral hosts, such as vaccinia or fowlpox (see, e.g., U.S. Pat. No. 4,722,848). This approach involves the use of vaccinia virus, e.g., as a vector to express nucleotide sequences that encode the double-stranded molecule. Upon introduction into a cell expressing the target gene, the recombinant vaccinia virus expresses the molecule and thereby suppresses the proliferation of the cell. Another example of useable vector includes Bacille Calmette Guerin (BCG). BCG vectors are described in Stover et al., Nature 1991, 351: 456-60. A wide variety of other vectors are useful for therapeutic administration and production of the double-stranded molecules; examples include adeno and adeno-associated virus vectors, retroviral vectors, Salmonella typhi vectors, detoxified anthrax toxin vectors, and the like. See, e.g., Shata et al., Mol Med Today 2000, 6: 66-71; Shedlock et al., J Leukoc Biol 2000, 68: 793-806; and Hipp et al., In Vivo 2000, 14: 571-85.

### Methods of Inhibiting or Reducing Growth of a Cancer Cell and Treating Cancer Using a Double-Stranded Molecule of the Present Invention:

The ability of certain siRNA to inhibit NSCLC has been previously described in WO 2005/89735, incorporated by reference herein. In present invention, two different dsRNA for EBI3, two different dsRNA for CDKN3 and two different dsRNA for EF-1delta were tested for their ability to inhibit cell growth. The two dsRNA for EBI3 (FIG. 4D), the one dsRNA for CDKN3 (FIG. 22A), the one dsRNA for EF-1delta (FIG. 22B) or the two dsRNA for NPTXR (FIG. 13D), effectively knocked down the expression of the gene in lung cancer cell lines coincided with suppression of cell proliferation.

Therefore, the present invention provides methods for inhibiting cell growth, i.e., lung cancer cell growth, by inducing dysfunction of EBI3, CDKN3, EF-1delta or NPTXRgene via inhibiting the expression of EBI3, CDKN3 or EF-1delta or NPTXR. EBI3, CDKN3 or EF-1delta or NPTXR gene expression can be inhibited by any of the aforementioned double-stranded molecules of the present invention which specifically target of EBI3, CDKN3, EF-1delta or NPTXR gene or the vectors of the present invention that can express any of the double-stranded molecules.

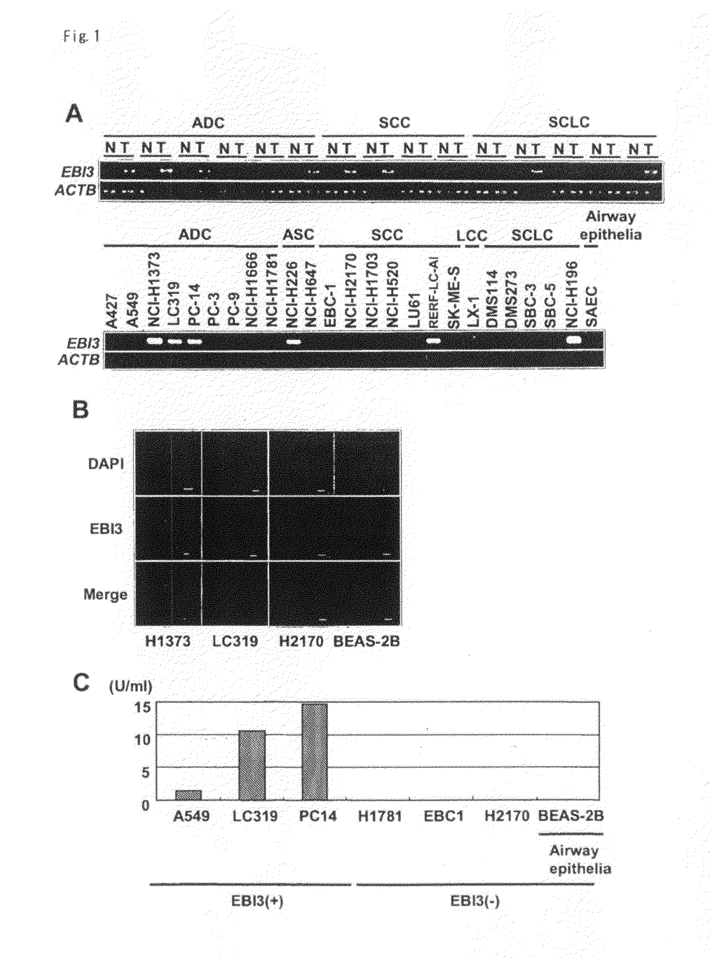

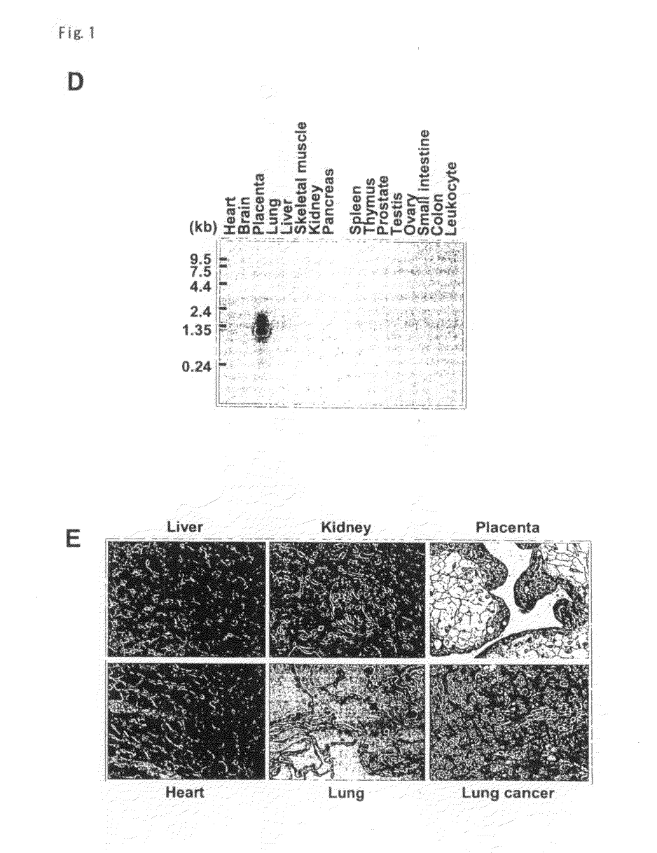

Such ability of the present double-stranded molecules and vectors to inhibit cell growth of cancerous cell indicates that they can be used for methods for treating cancer. Thus, the present invention provides methods to treat patients with lung cancer by administering a double-stranded molecule against EBI3, CDKN3, EF-1delta or NPTXR gene or a vector expressing the molecule without adverse effect because that genes were hardly detected in normal organs (FIGS. 1, 7E, 16, 17, 18B and 19).

Specifically, the present invention provides the following methods [1] to [25]:

[1] A method for inhibiting a growth of cancer cell and treating a cancer, wherein the cancer cell or the cancer expresses at least one gene selected from among EBI3, CDKN3, EF-1delta or NPTXR gene, which method includes the step of administering at least one isolated double-stranded molecule inhibiting the expression of EBI3, CDKN3, EF-1 and/or NPTXR in a cell over-expressing the gene and the cell proliferation, wherein the molecule is composed of a sense strand and an antisense strand complementary thereto, hybridized to each other to form the double-stranded molecule.

[2] The method of [1], wherein the double-stranded molecule acts at mRNA which matches a target sequence selected from among SEQ ID NO: 18 (at the position of 679-697 nt of SEQ ID NO: 1), SEQ ID NO: 20 (at the position of 280-298nt of SEQ ID NO: 1), SEQ ID NO: 49 (at the position of 310-328nt of SEQ ID NO: 5), SEQ ID NO: 51 (at the position of 225-243nt of SEQ ID NO: 7) SEQ ID NO: 84 (at the position of 1280-1298nt of SEQ ID NO: 86) and SEQ ID NO: 85 (at the position of 1393-1411nt of SEQ ID NO: 86);

[3] The double-stranded molecule of [2], wherein the sense strand contains the sequence corresponding to a target sequence selected from among SEQ ID NOs: 18, 20, 49, 51, 84 and 85.

[4] The method of [1], wherein the cancer to be treated is lung cancer;

[5] The method of [1], wherein the lung cancer is NSCLC or SCLC;

[6] The method of [1], wherein plural kinds of the double-stranded molecules are administered;

[7] The method of [6], wherein plural kinds of double-stranded molecules target the same gene;

[8] The method of [3], wherein the double-stranded molecule has a length of less than about 100 nucleotides;

[9] The method of [8], wherein the double-stranded molecule has a length of less than about 75 nucleotides;

[10] The method of [9], wherein the double-stranded molecule has a length of less than about 50 nucleotides;

[11] The method of [10], wherein the double-stranded molecule has a length of less than about 25 nucleotides;

[12] The method of [11], wherein the double-stranded molecule has a length of between about 19 and about 25 nucleotides in length;

[13] The method of [1], wherein the double-stranded molecule is composed of a single polynucleotide containing both the sense strand and the antisense strand linked by an interventing single-strand;

[14] The method of [13], wherein the double-stranded molecule has the general formula 5′-[A]-[B]-[A′]-3′, wherein [A] is the sense strand containing a sequence corresponding to a target sequence selected from among SEQ ID NOs: 18, 20, 49, 51, 84 and 85, [B] is the intervening single strand composed of 3 to 23 nucleotides, and [A′] is the antisense strand containing a sequence complementary to [A];

[15] The method of [1], wherein the double-stranded molecule is an RNA;

[16] The method of [1], wherein the double-stranded molecule contains both DNA and RNA;

[17] The method of [16], wherein the double-stranded molecule is a hybrid of a DNA polynucleotide and an RNA polynucleotide;

[18] The method of [17] wherein the sense and antisense strand polynucleotides are composed of DNA and RNA, respectively;

[19] The method of [16], wherein the double-stranded molecule is a chimera of DNA and RNA;

[20] The method of [19], wherein a region flanking to the 3′-end of the antisense strand, or both of a region flanking to the 5′-end of sense strand and a region flanking to the 3′-end of antisense strand are composed of RNA;

[21] The method of [20], wherein the flanking region is composed of 9 to 13 nucleotides;

[22] The method of [1], wherein the double-stranded molecule contains 3′ overhangs;

[23] The method of [1], wherein the double-stranded molecule is encoded by a vector;

[24] The method of [23], wherein the double-stranded molecule encoded by the vector has the general formula 5′-[A]-[B]-[A′]-3′, wherein [A] is the sense strand containing a sequence corresponding to a target sequence selected from among SEQ ID NOs: 18, 20, 49, 51, 84 and 85, [B] is a intervening single-strand is composed of 3 to 23 nucleotides, and [A′] is the antisense strand containing a sequence complementary to [A]; and

[25] The method of [1], wherein the double-stranded molecule is contained in a composition which includes, in addition to the molecule, a transfection-enhancing agent and pharmaceutically acceptable carrier.

The method of the present invention will be described in more detail below.

The growth of cells expressing EBI3, CDKN3, EF-1delta or NPTXR gene may be inhibited by contacting the cells with a double-stranded molecule against EBI3, CDKN3, EF-1delta or NPTXR gene, a vector expressing the molecule or a composition containing the same. The cell may be further contacted with a transfection agent. Suitable transfection agents are known in the art. The phrase “inhibition of cell growth” indicates that the cell proliferates at a lower rate or has decreased viability as compared to a cell not exposed to the molecule. Cell growth may be measured by methods known in the art, e.g., using the MTT cell proliferation assay.

The growth of any kind of cell may be suppressed according to the present method so long as the cell expresses or over-expresses the target gene of the double-stranded molecule of the present invention. Exemplary cells include lung cancer cells, including both NSCLC and SCLC.

Thus, patients suffering from or at risk of developing disease related to EBI3, CDKN3, EF-1delta or NPTXR may be treated by administering at least one of the present double-stranded molecules, at least one vector expressing at least one of the molecules or at least one composition containing at least one of the molecules. For example, patients of lung cancer may be treated according to the present methods. The type of cancer may be identified by standard methods according to the particular type of tumor to be diagnosed. Lung cancer may be diagnosed, for example, with Carcinoembryonic antigen (CEA), CYFRA, pro-GRP and so on, as lung cancer marker, or with Chest X-Ray and/or Sputum Cytology. More preferably, patients treated by the methods of the present invention are selected by detecting the expression of EBI3, CDKN3, EF-1delta or NPTXR in a biopsy from the patient by RT-PCR or immunoassay. Preferably, before the treatment of the present invention, the biopsy specimen from the subject is confirmed for EBI3, CDKN3, EF-1delta or NPTXR gene over-expression by methods known in the art, for example, immunohistochemical analysis or RT-PCR.

According to the present method to inhibit cell growth and thereby treating cancer, when administering plural kinds of the double-stranded molecules (or vectors expressing or compositions containing the same), each of the molecules may have different structures but acts at mRNA which matches the same target sequence of EBI3, CDKN3, EF-1delta and/or NPTXR. Alternatively plural kinds of the double-stranded molecules may acts at mRNA which matches different target sequence of same gene or acts at mRNA which matches different target sequence of different gene. For example, the method may utilize double-stranded molecules directed to EBI3, CDKN3, EF-1delta or NPTXR. Alternatively, for example, the method may utilize double-stranded molecules directed to one, two or more target sequences selected from EBI3, CDKN3, EF-1delta and NPTXR.

For inhibiting cell growth, a double-stranded molecule of present invention may be directly introduced into the cells in a form to achieve binding of the molecule with corresponding mRNA transcripts. Alternatively, as described above, a DNA encoding the double-stranded molecule may be introduced into cells as a vector. For introducing the double-stranded molecules and vectors into the cells, transfection-enhancing agent, such as FuGENE (Roche diagnostics), Lipofectamine 2000 (Invitrogen), Oligofectamine (Invitrogen), and Nucleofector (Wako pure Chemical), may be employed.

A treatment is deemed “efficacious” if it leads to clinical benefit such as, reduction in expression of EBI3, CDKN3, EF-1delta or NPTXRgene, or a decrease in size, prevalence, or metastatic potential of the cancer in the subject. When the treatment is applied prophylactically, “efficacious” means that it retards or prevents cancers from forming or prevents or alleviates a clinical symptom of cancer. Efficaciousness is determined in association with any known method for diagnosing or treating the particular tumor type.

It is understood that the double-stranded molecule of the invention degrades the target mRNA (EBI3, CDKN3, EF-1delta or NPTXR) in substoichiometric amounts. Without wishing to be bound by any theory, it is believed that the double-stranded molecule of the invention causes degradation of the target mRNA in a catalytic manner. Thus, compared to standard cancer therapies, significantly less a double-stranded molecule needs to be delivered at or near the site of cancer to exert therapeutic effect.

One skilled in the art can readily determine an effective amount of the double-stranded molecule of the invention to be administered to a given subject, by taking into account factors such as body weight, age, sex, type of disease, symptoms and other conditions of the subject; the route of administration; and whether the administration is regional or systemic. Generally, an effective amount of the double-stranded molecule of the invention is an intercellular concentration at or near the cancer site of from about 1 nanomolar (nM) to about 100 nM, preferably from about 2 nM to about 50 nM, more preferably from about 2.5 nM to about 10 nM. It is contemplated that greater or smaller amounts of the double-stranded molecule can be administered. The precise dosage required for a particular circumstance may be readily and routinely determined by one of skill in the art.

The present methods can be used to inhibit the growth or metastasis of cancer expressing at least one EBI3, CDKN3, EF-1delta or NPTXR; for example lung cancer, especially NSCLC or SCLC. In particular, a double-stranded molecule containing a target sequence of EBI3 (i.e., SEQ ID NOs: 18 or 20), CDKN3 (i.e., SEQ ID NO: 49), EF-1delta (i.e., SEQ ID NO: 51) or NPTXR (i.e., SEQ ID NOs: 84 or 85) is particularly preferred for the treatment of lung cancer.

For treating cancer, the double-stranded molecule of the invention can also be administered to a subject in combination with a pharmaceutical agent different from the double-stranded molecule. Alternatively, the double-stranded molecule of the invention can be administered to a subject in combination with another therapeutic method designed to treat cancer. For example, the double-stranded molecule of the invention can be administered in combination with therapeutic methods currently employed for treating cancer or preventing cancer metastasis (e.g., radiation therapy, surgery and treatment using chemotherapeutic agents, such as cisplatin, carboplatin, cyclophosphamide, 5-fluorouracil, adriamycin, daunorubicin or tamoxifen).

In the present methods, the double-stranded molecule can be administered to the subject either as a naked double-stranded molecule, in conjunction with a delivery reagent, or as a recombinant plasmid or viral vector which expresses the double-stranded molecule.

Suitable delivery reagents for administration in conjunction with the present a double-stranded molecule include the Mirus Transit TKO lipophilic reagent; lipofectin; lipofectamine; cellfectin; or polycations (e.g., polylysine), or liposomes. A preferred delivery reagent is a liposome.

Liposomes can aid in the delivery of the double-stranded molecule to a particular tissue, such as retinal or tumor tissue, and can also increase the blood half-life of the double-stranded molecule. Liposomes suitable for use in the invention are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al., Ann Rev Biophys Bioeng 1980, 9: 467; and U.S. Pat. Nos. 4,235,871; 4,501,728; 4,837,028; and 5,019,369, the entire disclosures of which are herein incorporated by reference.

Preferably, the liposomes encapsulating the present double-stranded molecule comprises a ligand molecule that can deliver the liposome to the cancer site. Ligands which bind to receptors prevalent in tumor or vascular endothelial cells, such as monoclonal antibodies that bind to tumor antigens or endothelial cell surface antigens, are preferred.

Particularly preferably, the liposomes encapsulating the present double-stranded molecule are modified so as to avoid clearance by the mononuclear macrophage and reticuloendothelial systems, for example, by having opsonization-inhibition moieties bound to the surface of the structure. In one embodiment, a liposome of the invention can comprise both opsonization-inhibition moieties and a ligand.

Opsonization-inhibiting moieties for use in preparing the liposomes of the invention are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example, solid tumors, will efficiently accumulate these liposomes; see Gabizon et al., Proc Natl Acad Sci USA 1988, 18: 6949-53. In addition, the reduced uptake by the RES lowers the toxicity of stealth liposomes by preventing significant accumulation in liver and spleen. Thus, liposomes of the invention that are modified with opsonization-inhibition moieties can deliver the present double-stranded molecule to tumor cells.

Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM.sub.1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

Preferably, the opsonization-inhibiting moiety is a PEG, PPG, or derivatives thereof. Liposomes modified with PEG or PEG-derivatives are sometimes called “PEGylated liposomes”.

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH. sub. 3 and a solvent mixture such as tetrahydrofuran and water in a 30:12 ratio at 60 degrees C.

Vectors expressing a double-stranded molecule of the invention are discussed above. Such vectors expressing at least one double-stranded molecule of the invention can also be administered directly or in conjunction with a suitable delivery reagent, including the Minis Transit LT1 lipophilic reagent; lipofectin; lipofectamine; cellfectin; polycations (e.g., polylysine) or liposomes. Methods for delivering recombinant viral vectors, which express a double-stranded molecule of the invention, to an area of cancer in a patient are within the skill of the art.

The double-stranded molecule of the invention can be administered to the subject by any means suitable for delivering the double-stranded molecule into cancer sites. For example, the double-stranded molecule can be administered by gene gun, electroporation, or by other suitable parenteral or enteral administration routes.

Suitable enteral administration routes include oral, rectal, or intranasal delivery.

Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition including subcutaneous infusion (such as by osmotic pumps); direct application to the area at or near the site of cancer, for example by a catheter or other placement device (e.g., a retinal pellet or a suppository or an implant comprising a porous, non-porous, or gelatinous material); and inhalation. It is preferred that injections or infusions of the double-stranded molecule or vector be given at or near the site of cancer.

The double-stranded molecule of the invention can be administered in a single dose or in multiple doses. Where the administration of the double-stranded molecule of the invention is by infusion, the infusion can be a single sustained dose or can be delivered by multiple infusions. Injection of the agent directly into the tissue is at or near the site of cancer preferred. Multiple injections of the agent into the tissue at or near the site of cancer are particularly preferred.

One skilled in the art can also readily determine an appropriate dosage regimen for administering the double-stranded molecule of the invention to a given subject. For example, the double-stranded molecule can be administered to the subject once, for example, as a single injection or deposition at or near the cancer site. Alternatively, the double-stranded molecule can be administered once or twice daily to a subject for a period of from about three to about twenty-eight days, more preferably from about seven to about ten days. In a preferred dosage regimen, the double-stranded molecule is injected at or near the site of cancer once a day for seven days. Where a dosage regimen comprises multiple administrations, it is understood that the effective amount of a double-stranded molecule administered to the subject can comprise the total amount of a double-stranded molecule administered over the entire dosage regimen.

### Compositions Containing a Double-Stranded Molecule of the Present Invention:

In addition to the above, the present invention also provides pharmaceutical compositions that include at least one of the present double-stranded molecules or the vectors coding for the molecules. Specifically, the present invention provides the following compositions [1] to [25]:

[1] A composition for inhibiting a growth of cancer cell and treating a cancer, wherein the cancer cell and the cancer expresses at least one gene selected from among EBI3, CDKN3, EF-1delta or NPTXR gene, including at least one isolated double-stranded molecule inhibiting the expression of EBI3, CDKN3, EF-1delta or NPTXR and the cell proliferation, which molecule is composed of a sense strand and an antisense strand complementary thereto, hybridized to each other to form the double-stranded molecule.

[2] The composition of [1], wherein the double-stranded molecule acts at mRNA which matches a target sequence selected from among SEQ ID NO: 18 (at the position of 679-697nt of SEQ ID NO: 1), SEQ ID NO: 20 (at the position of 280-298nt of SEQ ID NO: 1), SEQ ID NO: 49 (at the position of 310-328nt of SEQ ID NO: 5), SEQ ID NO: 51 (at the position of 225-243nt of SEQ ID NO: 7) SEQ ID NO: 84 (at the position of 1280-1298nt of SEQ ID NO: 86) and SEQ ID NO: 85 (at the position of 1393-1411nt of SEQ ID NO: 86);

[3] The composition of [2], wherein the double-stranded molecule, wherein the sense strand contains a sequence corresponding to a target sequence selected from among SEQ ID NOs: 18, 20, 49, 51, 84 and 85.

[4] The composition of [1], wherein the cancer to be treated is lung cancer;

[5] The composition of [4], wherein the lung cancer is NSCLC or SCLC;

[6] The composition of [1], wherein the composition contains plural kinds of the double-stranded molecules;

[7] The composition of [6], wherein the plural kinds of the double-stranded molecules target the same gene;

[8] The composition of [3], wherein the double-stranded molecule has a length of less than about 100 nucleotides;

[9] The composition of [8], wherein the double-stranded molecule has a length of less than about 75 nucleotides;

[10] The composition of [9], wherein the double-stranded molecule has a length of less than about 50 nucleotides;

[11] The composition of [10], wherein the double-stranded molecule has a length of less than about 25 nucleotides;

[12] The composition of [11], wherein the double-stranded molecule has a length of between about 19 and about 25 nucleotides;

[13] The composition of [1], wherein the double-stranded molecule is composed of a single polynucleotide containing the sense strand and the antisense strand linked by an intervening single-strand;

[14] The composition of [13], wherein the double-stranded molecule has the general formula 5′-[A]-[B]-[A′]-3′, wherein [A] is the sense strand sequence contains a sequence corresponding to a target sequence selected from among SEQ ID NOs: 18, 20, 49, 51, 84 and 85, [B] is the intervening single-strand consisting of 3 to 23 nucleotides, and [A′] is the antisense strand contains a sequence complementary to [A];

[15] The composition of [1], wherein the double-stranded molecule is an RNA;

[16] The composition of [1], wherein the double-stranded molecule is DNA and/or RNA;

[17] The composition of [16], wherein the double-stranded molecule is a hybrid of a DNA polynucleotide and an RNA polynucleotide;

[18] The composition of [17], wherein the sense and antisense strand polynucleotides are composed of DNA and RNA, respectively;

[19] The composition of [16], wherein the double-stranded molecule is a chimera of DNA and RNA;

[20] The composition of [19], wherein a region flanking to the 3′-end of the antisense strand, or both of a region flanking to the 5′-end of sense strand and a region flanking to the 3′-end of antisense strand are composed of RNA;

[21] The composition of [20], wherein the flanking region is composed of 9 to 13 nucleotides;

[22] The composition of [1], wherein the double-stranded molecule contains 3′ overhangs;

[23] The composition of [1], wherein the double-stranded molecule is encoded by a vector and contained in the composition;

[24] The composition of [23], wherein the double-stranded molecule has the general formula 5′-[A]-[B]-[A′]-3′, wherein [A] is the sense strand containing a sequence corresponding to a target sequence selected from among SEQ ID NOs: 18, 20, 49, 51, 84 and 85, [B] is a intervening single-strand composed of 3 to 23 nucleotides, and [A′] is the antisense strand containing a sequence complementary to [A]; and

[25] The composition of [1], wherein the composition includes a transfection-enhancing agent and pharmaceutically acceptable carrier.

Suitable compositions of the present invention are described in additional detail below.

The double-stranded molecules of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations contain at least one of the double-stranded molecules or vectors encoding them of the present invention (e.g., 0.1 to 90% by weight), or a physiologically acceptable salt of the molecule, mixed with a physiologically acceptable carrier medium. Preferred physiologically acceptable carrier media are water, buffered water, normal saline, 0.4% saline, 0.3% glycine, hyaluronic acid and the like.

According to the present invention, the composition may contain plural kinds of the double-stranded molecules, each of the molecules may be directed to the same target sequence, or different target sequences of EBI3, CDKN3, EF-1delta and/or NPTXR. For example, the composition may contain double-stranded molecules directed to EBI3, CDKN3, EF-1delta or NPTXR. Alternatively, for example, the composition may contain double-stranded molecules directed to one, two or more target sequences selected from EBI3, CDKN3, EF-1delta and NPTXR.

Furthermore, the present composition may contain a vector coding for one or plural double-stranded molecules. For example, the vector may encode one, two or several kinds of the present double-stranded molecules. Alternatively, the present composition may contain plural kinds of vectors, each of the vectors coding for a different double-stranded molecule.

Moreover, the present double-stranded molecules may be contained as liposomes in the present composition. See under the item of “Methods of treating cancer using the double-stranded molecule” for details of liposomes.

Pharmaceutical compositions of the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like.

For example, a solid pharmaceutical composition for oral administration can include any of the carriers and excipients listed above and 10-95%, preferably 25-75%, of one or more double-stranded molecule of the invention. A pharmaceutical composition for aerosol (inhalational) administration can comprise 0.01-20% by weight, preferably 1-10% by weight, of one or more double-stranded molecule of the invention encapsulated in a liposome as described above, and propellant. A carrier can also be included as desired; e.g., lecithin for intranasal delivery.

In addition to the above, the present composition may contain other pharmaceutical active ingredients so long as they do not inhibit the in vivo function of the present double-stranded molecules. For example, the composition may contain chemotherapeutic agents conventionally used for treating cancers.

In another embodiment, the present invention also provides the use of the double-stranded nucleic acid molecules of the present invention in manufacturing a pharmaceutical composition for treating a lung cancer characterized by the expression of EBI3, CDKN3, EF-1delta or NPTXR. For example, the present invention relates to a use of double-stranded nucleic acid molecule inhibiting the expression of gene selected from among EBI3, CDKN3, EF-1delta and NPTXR in a cell, which molecule includes a sense strand and an antisense strand complementary thereto, hybridized to each other to form the double-stranded nucleic acid molecule and targets to a sequence selected from among SEQ ID NOs: 18, 20, 49, 51, 84 and 85, for manufacturing a pharmaceutical composition for treating lung cancer expressing EBI3, CDKN3, EF-1delta or NPTXR.

Alternatively, the present invention further provides a method or process for manufacturing a pharmaceutical composition for treating a lung cancer characterized by the expression of EBI3, CDKN3, EF-1delta or NPTXR, wherein the method or process includes a step for formulating a pharmaceutically or physiologically acceptable carrier with a double-stranded nucleic acid molecule inhibiting the expression of EBI3, CDKN3, EF-1delta or NPTXR in a cell, which over-expresses the gene, which molecule includes a sense strand and an antisense strand complementary thereto, hybridized to each other to form the double-stranded nucleic acid molecule and targets to a sequence selected from among SEQ ID NOs: 18, 20, 49, 51, 84 and 85 as active ingredients.

In another embodiment, the present invention also provides a method or process for manufacturing a pharmaceutical composition for treating a lung cancer characterized by the expression of EBI3, CDKN3, EF-1delta or NPTXR, wherein the method or process includes a step for admixing an active ingredient with a pharmaceutically or physiologically acceptable carrier, wherein the active ingredient is a double-stranded nucleic acid molecule inhibiting the expression of EBI3, CDKN3, EF-1delta or NPTXR in a cell, which over-expresses the gene, which molecule includes a sense strand and an antisense strand complementary thereto, hybridized to each other to form the double-stranded nucleic acid molecule and targets to a sequence selected from among SEQ ID NOs: 18, 20, 49, 51, 84 and 85.

### A Method for Diagnosing Lung Cancer:

The expression of EBI3, DLX5, NPTX1, CDKN3 or EF-1delta was found to be specifically elevated in lung cancer cells (FIGS. 1, 5, 7, 8 and 16). Therefore, the genes identified herein as well as their transcription and translation products find diagnostic utility as markers for lung cancer and by measuring the expression of EBI3, DLX5, NPTX1, CDKN3 or EF-1delta in a cell sample, lung cancer can be diagnosed. Specifically, the present invention provides a method for diagnosing lung cancer by determining the expression level of EBI3, DLX5, NPTX1, CDKN3 or EF-1delta in the subject. Lung cancers that can be diagnosed by the present method include NSCLC and SCLC. Furthermore, NSCLC, including lung adenocarcinoma and lung squamous cell carcinoma (SCC), can also be diagnosed or detected by the present invention.

According to the present invention, an intermediate result for examining the condition of a subject may be provided. Such intermediate result may be combined with additional information to assist a doctor, nurse, or other practitioner to diagnose that a subject suffers from the disease. Alternatively, the present invention may be used to detect cancerous cells in a subject-derived tissue, and provide a doctor with useful information to diagnose that the subject suffers from the disease.

Specifically, the present invention provides the following methods [1] to [10]:

[1] A method for diagnosing lung cancer, said method including the steps of:

(a) detecting the expression level of the gene encoding the amino acid sequence of EBI3, CDKN3 or EF-1delta in a biological sample; and

(b) correlating an increase in the expression level detected as compared to a normal control level of the gene to the presence of disease.

[2] The method of [1], wherein the expression level is at least 10% greater than the normal control level.

[3] The method of [1], wherein the expression level is detected by a methods selected from among:

(a) detecting an mRNA including the sequence of EBI3, DLX5, NPTX1, CDKN3 or EF-1delta,

(b) detecting a protein including the amino acid sequence of EBI3, DLX5, NPTX1, CDKN3 or EF-1delta, and

(c) detecting a biological activity of a protein including the amino acid sequence of EBI3, DLX5, NPTX1, CDKN3 or EF-1delta.

[4] The method of [1], wherein the lung cancer is NSCLC or SCLC.

[5] The method of [3], wherein the expression level is determined by detecting hybridization of a probe to a gene transcript of the gene.

[6] The method of [3], wherein the expression level is determined by detecting the binding of an antibody against the protein encoded by a gene as the expression level of the gene.

[7] The method of [1], wherein the biological sample includes biopsy, sputum or blood.

[8] The method of [1], wherein the subject-derived biological sample includes an epithelial cell.

[9] The method of [1], wherein the subject-derived biological sample includes a cancer cell.

[10] The method of [1], wherein the subject-derived biological sample includes a cancerous epithelial cell.

The method of diagnosing lung cancer will be described in more detail below.

A subject to be diagnosed by the present method is preferably a mammal. Exemplary mammals include, but are not limited to, e.g., human, non-human primate, mouse, rat, dog, cat, horse, and cow.

It is preferred to collect a biological sample from the subject to be diagnosed to perform the diagnosis. Any biological material can be used as the biological sample for the determination so long as it includes the objective transcription or translation product of EBI3, DLX5, NPTX1, CDKN3 or EF-1delta. The biological samples include, but are not limited to, bodily tissues and fluids, such as blood, sputum and urine. Preferably, the biological sample contains a cell population comprising an epithelial cell, more preferably a cancerous epithelial cell or an epithelial cell derived from tissue suspected to be cancerous. Further, if necessary, the cell may be purified from the obtained bodily tissues and fluids, and then used as the biological sample.

According to the present invention, the expression level of EBI3, DLX5, NPTX1, CDKN3 or EF-1delta in the subject-derived biological sample is determined. The expression level can be determined at the transcription (nucleic acid) product level, using methods known in the art. For example, the mRNA of EBI3, DLX5, NPTX1, CDKN3 or EF-1delta may be quantified using probes by hybridization methods (e.g., Northern hybridization). The detection may be carried out on a chip or an array. The use of an array is preferable for detecting the expression level of a plurality of genes (e.g., various cancer specific genes) including EBI3, DLX5, NPTX1, CDKN3 or EF-1delta. Those skilled in the art can prepare such probes utilizing the sequence information of the EBI3 (SEQ ID NO 1; GenBank accession number: NM_{—}005755) or DLX5 (SEQ ID NO 3; GenBank accession number: BC006226) or NPTX1 (SEQ ID NO; 78; GenBank accession number: NM_{—}002522) or CDKN3 (SEQ ID NO 5; GenBank accession number: L27711) or EF-1delta (SEQ ID NO 7; GenBank accession number: BC009907). For example, the cDNA of EBI3, DLX5, NPTX1, CDKN3 or EF-1delta may be used as the probes. If necessary, the probe may be labeled with a suitable label, such as dyes, fluorescent and isotopes, and the expression level of the gene may be detected as the intensity of the hybridized labels.

Furthermore, the transcription product of EBI3, DLX5, NPTX1, CDKN3 or EF-1delta may be quantified using primers by amplification-based detection methods (e.g., RT-PCR). Such primers can also be prepared based on the available sequence information of the gene. For example, the primers (SEQ ID NO 9 and 10, 21 and 22, 34 and 35, or 36, 37, 80 and 81) used in the Example may be employed for the detection by RT-PCR or Northern blot, but the present invention is not restricted thereto.

Specifically, a probe or primer used for the present method hybridizes under stringent, moderately stringent, or low stringent conditions to the mRNA of EBI3, DLX5, NPTX1, CDKN3 or EF-1delta. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

Alternatively, the translation product may be detected for the diagnosis of the present invention. For example, the quantity of EBI3, DLX5, NPTX1, CDKN3 or EF-1delta protein may be determined. A method for determining the quantity of the protein as the translation product includes immunoassay methods that use an antibody specifically recognizing the protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to EBI3, DLX5, NPTX1, CDKN3 or EF-1delta protein. Methods to prepare these kinds of antibodies for the detection of proteins are well known in the art, and any method may be employed in the present invention to prepare such antibodies and equivalents thereof.

As another method to detect the expression level of EBI3, DLX5, NPTX1, CDKN3 or EF-1delta gene based on its translation product, the intensity of staining may be observed via immunohistochemical analysis using an antibody against EBI3, DLX5, NPTX1, CDKN3 or EF-1delta protein. Namely, the observation of strong staining indicates increased presence of the protein and at the same time high expression level of EBI3, DLX5, NPTX1, CDKN3 or EF-1delta gene.

Moreover, in addition to the expression level of EBI3, DLX5, NPTX1, CDKN3 or EF-1delta gene, the expression level of other cancer-associated genes, for example, genes known to be differentially expressed in lung cancer may also be determined to improve the accuracy of the diagnosis.

The expression level of cancer marker gene including EBI3, DLX5, NPTX1, CDKN3 or EF-1delta gene in a biological sample can be considered to be increased if it increases from the control level of the corresponding cancer marker gene by, for example, 10%, 25%, or 50%; or increases to more than 1.1 fold, more than 1.5 fold, more than 2.0 fold, more than 5.0 fold, more than 10.0 fold, or more.

The control level may be determined at the same time with the test biological sample by using a sample(s) previously collected and stored from a subject/subjects whose disease state (cancerous or non-cancerous) is/are known. Alternatively, the control level may be determined by a statistical method based on the results obtained by analyzing previously determined expression level(s) of EBI3, DLX5, NPTX1, CDKN3 or EF-1delta gene in samples from subjects whose disease state are known. Furthermore, the control level can be a database of expression patterns from previously tested cells. Moreover, according to an aspect of the present invention, the expression level of EBI3, DLX5, NPTX1, CDKN3 or EF-1delta gene in a biological sample may be compared to multiple control levels, which control levels are determined from multiple reference samples. It is preferred to use a control level determined from a reference sample derived from a tissue type similar to that of the patient-derived biological sample. Moreover, it is preferred, to use the standard value of the expression levels of EBI3, DLX5, NPTX1, CDKN3 or EF-1delta gene in a population with a known disease state. The standard value may be obtained by any method known in the art. For example, a range of mean+/−2 S.D. or mean+/−3 S.D. may be used as standard value.

In the context of the present invention, a control level determined from a biological sample that is known not to be cancerous is referred to as a “normal control level”. On the other hand, if the control level is determined from a cancerous biological sample, it is referred to as a “cancerous control level”.

When the expression level of EBI3, DLX5, NPTX1, CDKN3 or EF-1delta gene is increased as compared to the normal control level or is similar to the cancerous control level, the subject may be diagnosed to be suffering from or at a risk of developing cancer. Furthermore, in the case where the expression levels of multiple cancer-related genes are compared, a similarity in the gene expression pattern between the sample and the reference which is cancerous indicates that the subject is suffering from or at a risk of developing cancer.

Difference between the expression levels of a test biological sample and the control level can be normalized to the expression level of control nucleic acids, e.g., housekeeping genes, whose expression levels are known not to differ depending on the cancerous or non-cancerous state of the cell. Exemplary control genes include, but are not limited to, beta-actin, glyceraldehyde 3 phosphate dehydrogenase, and ribosomal protein P1.

### Method for Assessing the Prognosis of Cancer:

The present invention relates to the novel discovery that EBI3, DLX5, NPTX1, CDKN3 and EF-1delta expression is significantly associated with poorer prognosis of patients. Thus, the present invention provides a method for determining or assessing the prognosis of a patient with cancer, in particular lung cancer, by detecting the expression level of the EBI3, DLX5, NPTX1, CDKN3 and/or EF-1delta gene in a biological sample of the patient; comparing the detected expression level to a control level; and determining a increased expression level to the control level as indicative of poor prognosis (poor survival).

Herein, the term “prognosis” refers to a forecast as to the probable outcome of the disease as well as the prospect of recovery from the disease as indicated by the nature and symptoms of the case. Accordingly, a less favorable, negative, poor prognosis is defined by a lower post-treatment survival term or survival rate. Conversely, a positive, favorable, or good prognosis is defined by an elevated post-treatment survival term or survival rate.

The terms “assessing the prognosis” refer to the ability of predicting, forecasting or correlating a given detection or measurement with a future outcome of cancer of the patient (e.g., malignancy, likelihood of curing cancer, survival, and the like). For example, a determination of the expression level of EBI3, DLX5, NPTX1, CDKN3 and/or EF-1delta over time enables a predicting of an outcome for the patient (e.g., increase or decrease in malignancy, increase or decrease in grade of a cancer, likelihood of curing cancer, survival, and the like).

In the context of the present invention, the phrase “assessing (or determining) the prognosis” is intended to encompass predictions and likelihood analysis of cancer, progression, particularly cancer recurrence, metastatic spread and disease relapse. The present method for assessing prognosis is intended to be used clinically in making decisions concerning treatment modalities, including therapeutic intervention, diagnostic criteria such as disease staging, and disease monitoring and surveillance for metastasis or recurrence of neoplastic disease.

The patient-derived biological sample used for the method may be any sample derived from the subject to be assessed so long as the EBI3, DLX5, NPTX1, CDKN3 and/or EF-1delta gene can be detected in the sample. Preferably, the biological sample is a lung cell (a cell obtained from the lung). Furthermore, the biological sample may include bodily fluids such as sputum, blood, serum, or plasma. Moreover, the sample may be cells purified from a tissue. The biological samples may be obtained from a patient at various time points, including before, during, and/or after a treatment.

According to the present invention, it was shown that the higher the expression level of the EBI3, DLX5, NPTX1, CDKN3 and/or EF-1delta gene measured in the patient-derived biological sample, the poorer the prognosis for post-treatment remission, recovery, and/or survival and the higher the likelihood of poor clinical outcome. Thus, according to the present method, the “control level” used for comparison may be, for example, the expression level of the EBI3, DLX5, NPTX1, CDKN3 and/or EF-1delta gene detected before any kind of treatment in an individual or a population of individuals who showed good or positive prognosis of cancer, after the treatment, which herein will be referred to as “good prognosis control level”. Alternatively, the “control level” may be the expression level of the EBI3, DLX5, NPTX1, CDKN3 and/or EF-1delta gene detected before any kind of treatment in an individual or a population of individuals who showed poor or negative prognosis of cancer, after the treatment, which herein will be referred to as “poor prognosis control level”. The “control level” is a single expression pattern derived from a single reference population or from a plurality of expression patterns. Thus, the control level may be determined based on the expression level of the EBI3, DLX5, NPTX1, CDKN3 and/or EF-1delta gene detected before any kind of treatment in a patient of cancer, or a population of the patients whose disease state (good or poor prognosis) is known. Preferably, cancer is lung cancer. It is preferred, to use the standard value of the expression levels of the EBI3, DLX5, NPTX1, CDKN3 and EF-1delta gene in a patient group with a known disease state. The standard value may be obtained by any method known in the art. For example, a range of mean+/−2 S.D. or mean+/−3 S.D. may be used as standard value.

The control level may be determined at the same time with the test biological sample by using a sample(s) previously collected and stored before any kind of treatment from cancer patient(s) (control or control group) whose disease state (good prognosis or poor prognosis) are known.

Alternatively, the control level may be determined by a statistical method based on the results obtained by analyzing the expression level of the EBI3, DLX5, NPTX1, CDKN3 and/or EF-1delta gene in samples previously collected and stored from a control group. Furthermore, the control level can be a database of expression patterns from previously tested cells.

Moreover, according to an aspect of the present invention, the expression level of the EBI3, DLX5, NPTX1, CDKN3 or EF-1delta gene in a biological sample may be compared to multiple control levels, which control levels are determined from multiple reference samples. It is preferred to use a control level determined from a reference sample derived from a tissue type similar to that of the patient-derived biological sample.

According to the present invention, a similarity in the expression level of the EBI3, DLX5, NPTX1, CDKN3 and/or EF-1delta gene to a good prognosis control level indicates a more favorable prognosis of the patient and an increase in the expression level to the good prognosis control level indicates less favorable, poorer prognosis for post-treatment remission, recovery, survival, and/or clinical outcome. On the other hand, a decrease in the expression level of the EBI3, DLX5, NPTX1, CDKN3 or EF-1delta gene to the poor prognosis control level indicates a more favorable prognosis of the patient and a similarity in the expression level to the poor prognosis control level indicates less favorable, poorer prognosis for post-treatment remission, recovery, survival, and/or clinical outcome.

The expression level of the EBI3, DLX5, NPTX1, CDKN3 and/or EF-1delta gene in a biological sample can be considered altered when the expression level differs from the control level by more than 1.0, 1.5, 2.0, 5.0, 10.0, or more fold.

The difference in the expression level between the test biological sample and the control level can be normalized to a control, e.g., housekeeping gene. For example, polynucleotides whose expression levels are known not to differ between the cancerous and non-cancerous cells, including those coding for beta-actin, glyceraldehyde 3-phosphate dehydrogenase, and ribosomal protein P1, may be used to normalize the expression levels of the EBI3, DLX5, NPTX1, CDKN3 and/or EF-1delta genes.

The expression level may be determined by detecting the gene transcript in the patient-derived biological sample using techniques well known in the art. The gene transcripts detected by the present method include both the transcription and translation products, such as mRNA and protein.

For instance, the transcription product of the EBI3, DLX5, NPTX1, CDKN3 and/or EF-1delta gene can be detected by hybridization, e.g., Northern blot hybridization analyses, that use a EBI3, DLX5, NPTX1, CDKN3 and/or EF-1delta gene probe to the gene transcript. The detection may be carried out on a chip or an array. The use of an array is preferable for detecting the expression level of a plurality of genes including the EBI3, DLX5, NPTX1, CDKN3 and/or EF-1delta gene. As another example, amplification-based detection methods, such as reverse-transcription based polymerase chain reaction (RT-PCR) which use primers specific to the EBI3, DLX5, NPTX1, CDKN3 and/or EF-1delta gene may be employed for the detection (see Example). The EBI3, DLX5, NPTX1, CDKN3 and/or EF-1delta gene-specific probe or primers may be designed and prepared using conventional techniques by referring to the whole sequence of the EBI3, DLX5, NPTX1, CDKN3 and/or EF-1delta gene (SEQ ID NO: 1, 3, 5 and 7, respectively). For example, the primers (SEQ ID NOs: 9 and 10 (EBI3), 21 and 22 (DLX5), 82 and 83 (NPTX1), 34 and 35 (CDKN3), 36 and 37 (EF-1delta)) used in the Example may be employed for the detection by RT-PCR, but the present invention is not restricted thereto.

Specifically, a probe or primer used for the present method hybridizes under stringent, moderately stringent, or low stringent conditions to the mRNA of the EBI3, DLX5, NPTX1, CDKN3 and/or EF-1delta gene. As used herein, the phrase “stringent (hybridization) conditions” refers to conditions under which a probe or primer will hybridize to its target sequence, but to no other sequences. Stringent conditions are sequence-dependent and will be different under different circumstances. Specific hybridization of longer sequences is observed at higher temperatures than shorter sequences. Generally, the temperature of a stringent condition is selected to be about 5 degree Centigrade lower than the thermal melting point (Tm) for a specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 degree Centigrade for short probes or primers (e.g., 10 to 50 nucleotides) and at least about 60 degree Centigrade for longer probes or primers. Stringent conditions may also be achieved with the addition of destabilizing agents, such as formamide.

Alternatively, the translation product may be detected for the assessment of the present invention. For example, the quantity of the EBI3, DLX5, NPTX1, CDKN3 and/or EF-1delta protein may be determined. A method for determining the quantity of the protein as the translation product includes immunoassay methods that use an antibody specifically recognizing the EBI3, DLX5, NPTX1, CDKN3 and/or EF-1delta protein. The antibody may be monoclonal or polyclonal. Furthermore, any fragment or modification (e.g., chimeric antibody, scFv, Fab, F(ab′)2, Fv, etc.) of the antibody may be used for the detection, so long as the fragment retains the binding ability to the EBI3, DLX5, NPTX1, CDKN3 and/or EF-1delta protein. Methods to prepare these kinds of antibodies for the detection of proteins are well known in the art, and any method may be employed in the present invention to prepare such antibodies and equivalents thereof.

As another method to detect the expression level of the EBI3, DLX5, NPTX1, CDKN3 and/or EF-1delta gene based on its translation product, the intensity of staining may be observed via immunohistochemical analysis using an antibody against EBI3, DLX5, NPTX1, CDKN3 and/or EF-1delta protein. Namely, the observation of strong staining indicates increased presence of the EBI3, DLX5, NPTX1, CDKN3 and/or EF-1delta protein and at the same time high expression level of the EBI3, DLX5, NPTX1, CDKN3 and/or EF-1delta gene.

Furthermore, the EBI3, DLX5, NPTX1, CDKN3 and/or EF-1delta protein is known to have a cell proliferating activity. Therefore, the expression level of the EBI3, DLX5, NPTX1, CDKN3 and/or EF-1delta gene can be determined using such cell proliferating activity as an index. For example, cells which express EBI3, DLX5, NPTX1, CDKN3 and/or EF-1delta are prepared and cultured in the presence of a biological sample, and then by detecting the speed of proliferation, or by measuring the cell cycle or the colony forming ability the cell proliferating activity of the biological sample can be determined.

Moreover, in addition to the expression level of the EBI3, DLX5, NPTX1, CDKN3 and/or EF-1delta gene, the expression level of other lung cancer-associated genes, for example, genes known to be differentially expressed in lung cancer may also be determined to improve the accuracy of the assessment. Examples of such other lung cell-associated genes include those described in WO 2004/031413 and WO 2005/090603, the contents of which are incorporated by reference herein.

Alternatively, according to the present invention, an intermediate result may also be provided in addition to other test results for assessing the prognosis of a subject. Such intermediate result may assist a doctor, nurse, or other practitioner to assess, determine, or estimate the prognosis of a subject. Additional information that may be considered, in combination with the intermediate result obtained by the present invention, to assess prognosis includes clinical symptoms and physical conditions of a subject.

The patient to be assessed for the prognosis of cancer according to the method is preferably a mammal and includes human, non-human primate, mouse, rat, dog, cat, horse, and cow.

### A Kit for Diagnosing Cancer or Assessing the Prognosis of Cancer:

The present invention provides a kit for diagnosing cancer or assessing the prognosis of cancer. Preferably, the cancer is lung cancer. Specifically, the kit includes at least one reagent for detecting the expression of the EBI3, DLX5, NPTX1, CDKN3 and/or EF-1delta gene in a patient-derived biological sample, which reagent may be selected from the group of:

(a) a reagent for detecting mRNA of the EBI3, DLX5, NPTX1, CDKN3 and/or EF-1delta gene;

(b) a reagent for detecting the EBI3, DLX5, NPTX1, CDKN3 and/or EF-1delta protein; and

(c) a reagent for detecting the biological activity of the EBI3, DLX5, NPTX1, CDKN3 and/or EF-1delta protein.

Suitable reagents for detecting mRNA of the EBI3, DLX5, NPTX1, CDKN3 and/or EF-1delta gene include nucleic acids that specifically bind to or identify the EBI3, DLX5, NPTX1, CDKN3 and/or EF-1delta mRNA, such as oligonucleotides which have a complementary sequence to a part of the EBI3, DLX5, NPTX1, CDKN3 and/or EF-1delta mRNA. These kinds of oligonucleotides are exemplified by primers and probes that are specific to the EBI3, DLX5, NPTX1, CDKN3 and/or EF-1delta mRNA. These kinds of oligonucleotides may be prepared based on methods well known in the art. If needed, the reagent for detecting the EBI3, DLX5, NPTX1, CDKN3 and/or EF-1delta mRNA may be immobilized on a solid matrix. Moreover, more than one reagent for detecting the EBI3, DLX5, NPTX1, CDKN3 and/or EF-1delta mRNA may be included in the kit.

On the other hand, suitable reagents for detecting the EBI3, DLX5, NPTX1, CDKN3 and/or EF-1delta protein include antibodies to the EBI3, DLX5, NPTX1, CDKN3 and/or EF-1delta protein. The antibody may be monoclonal or polyclonal. Furthermore, any fragment or modification (e.g., chimeric antibody, scFv, Fab, F(ab′)2, Fv, etc.) of the antibody may be used as the reagent, so long as the fragment retains the binding ability to the EBI3, DLX5, NPTX1, CDKN3 and/or EF-1delta protein. Methods to prepare these kinds of antibodies for the detection of proteins are well known in the art, and any method may be employed in the present invention to prepare such antibodies and equivalents thereof. Furthermore, the antibody may be labeled with signal generating molecules via direct linkage or an indirect labeling technique. Labels and methods for labeling antibodies and detecting the binding of antibodies to their targets are well known in the art and any labels and methods may be employed for the present invention. Moreover, more than one reagent for detecting the EBI3, DLX5, NPTX1, CDKN3 and/or EF-1delta protein may be included in the kit.

Furthermore, the biological activity can be determined by, for example, measuring the cell proliferating activity due to the expressed EBI3, DLX5, NPTX1, CDKN3 and/or EF-1delta protein in the biological sample. For example, the cell is cultured in the presence of a patient-derived biological sample, and then by detecting the speed of proliferation, or by measuring the cell cycle or the colony forming ability the cell proliferating activity of the biological sample can be determined. If needed, the reagent for detecting the EBI3, DLX5, NPTX1, CDKN3 and/or EF-1delta mRNA may be immobilized on a solid matrix. Moreover, more than one reagent for detecting the biological activity of the EBI3, DLX5, NPTX1, CDKN3 and/or EF-1delta protein may be included in the kit.

The kit may contain more than one of the aforementioned reagents. Furthermore, the kit may include a solid matrix and reagent for binding a probe against the EBI3, DLX5, NPTX1, CDKN3 and/or EF-1delta gene or antibody against the EBI3, DLX5, NPTX1, CDKN3 and/or EF-1delta protein, a medium and container for culturing cells, positive and negative control reagents, and a secondary antibody for detecting an antibody against the EBI3, DLX5, NPTX1, CDKN3 and/or EF-1delta protein. For example, tissue samples obtained from patient with good prognosis or poor prognosis may serve as useful control reagents. A kit of the present invention may further include other materials desirable from a commercial and user standpoint, including buffers, diluents, filters, needles, syringes, and package inserts (e.g., written, tape, CD-ROM, etc.) with instructions for use. These reagents and such may be comprised in a container with a label. Suitable containers include bottles, vials, and test tubes. The containers may be formed from a variety of materials, such as glass or plastic.

As an embodiment of the present invention, when the reagent is a probe against the EBI3, DLX5, NPTX1, CDKN3 and/or EF-1delta mRNA, the reagent may be immobilized on a solid matrix, such as a porous strip, to form at least one detection site. The measurement or detection region of the porous strip may include a plurality of sites, each containing a nucleic acid (probe). A test strip may also contain sites for negative and/or positive controls. Alternatively, control sites may be located on a strip separated from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of EBI3, DLX5, NPTX1, CDKN3 and/or EF-1delta mRNA present in the sample. The detection sites may be configured in any suitably detectable shape and are typically in the shape of a bar or dot spanning the width of a test strip.

The kit of the present invention may further include a positive control sample or EBI3, DLX5, NPTX1, CDKN3 and/or EF-1delta standard sample. The positive control sample of the present invention may be prepared by collecting EBI3, DLX5, NPTX1, CDKN3 and/or EF-1delta positive blood samples and then those EBI3, DLX5, NPTX1, CDKN3 and/or EF-1delta level are assayed. Alternatively, purified EBI3, DLX5, NPTX1, CDKN3 or EF-1delta protein or polynucleotide may be added to EBI3, DLX5, NPTX1, CDKN3 and/or EF-1delta free serum to form the positive sample or the EBI3, DLX5, NPTX1, CDKN3 and/or EF-1delta standard. In the present invention, purified KDD1 may be recombinant protein. The EBI3, DLX5, NPTX1, CDKN3 and/or EF-1delta level of the positive control sample is, for example more than cut off value.

### Serological Diagnosis of Lung Cancer:

By measuring the level of EBI3 in subject-derived blood samples, the occurrence of or a predisposition to develop cancer expressing EBI3 in a subject can be determined. The cancer can be lung cancer, e.g. NSCLC and SCLC. Moreover, SCLC includes lung adenocarcinoma and lung squamous cell carcinoma (SCC). Accordingly, the present invention involves determining (e.g., measuring) the level of EBI3 in blood samples. In the present invention, a method for diagnosing lung cancer also includes a method for testing or detecting lung cancer. Alternatively, in the present invention, diagnosing lung cancer also refers to showing a suspicion, risk, or possibility of lung cancer in a subject.

Alternatively, by measuring the level of NPTX1 in subject-derived blood samples, the occurrence of or a predisposition to develop SCC expressing NPTX1 in a subject can be determined. Accordingly, the present invention involves determining (e.g., measuring) the level of NPTX1 in blood samples. In the present invention, a method for diagnosing SCC also includes a method for testing or detecting SCC. Alternatively, in the present invention, diagnosing SCC also refers to showing a suspicion, risk, or possibility of SCC in a subject.

Any blood samples may be used for determining the level of EBI3 or NPTX1 so long as either the gene or the protein of EBI3 or NPTX1 can be detected in the samples. Preferably, the blood samples comprise whole blood, serum, and plasma.

In the present invention, the “level of EBI3 or NPTX1 in blood samples” refers to the concentration of EBI3 or NPTX1 present in the blood after correcting the corpuscular volume in the whole blood. One of skill will recognize that the percentage of corpuscular volume in the blood varies greatly between individuals. For example, the percentage of erythrocytes in the whole blood is very different between men and women. Furthermore, differences between individuals cannot be ignored. Therefore, the apparent concentration of a substance in the whole blood which comprises corpuscular components varies greatly depending on the percentage of corpuscular volume. For example, even if the concentration in the serum is the same, the measured value for a sample with a large amount of corpuscular component will be lower than the value for a sample with a small amount of corpuscular component. Therefore, to compare the measured values of components in the blood, values for which the corpuscular volume has been corrected are usually used.

For example, by measuring components in the blood using, as samples, serum or plasma obtained by separating blood cells from the whole blood, measured values from which the effect from the corpuscular volume has been removed can be obtained. Therefore, the level of EBI3 or NPTX1 in the present invention can usually be determined as a concentration in the serum or plasma. Alternatively, it may first be measured as a concentration in the whole blood, and then the effect from the corpuscular volume may be corrected. Methods for measuring a corpuscular volume in a whole blood sample are known.

Subjects diagnosed for lung cancer or SCC according to the present methods are preferably mammals and include humans, non-human primates, mice, rats, dogs, cats, horses and cows. A preferable subject of the present invention is a human.

In the present invention, a subject may be a patient suspected of having the lung cancer or healthy individuals. The patient may be diagnosed by the present invention to facilitate clinical decision-making. In another embodiment, the present invention may also be applied to healthy individuals for screening of the lung cancer or SCC.

Furthermore, an intermediate result for examining the condition of a subject may be provided. Such intermediate result may be combined with additional information to assist a doctor, nurse, or other practitioner to diagnose that a subject suffers from the disease. Alternatively, the present invention may be used to detect cancerous cells in a subject-derived tissue, and provide a doctor with useful information to diagnose that the subject suffers from the disease.

In one embodiment of the present invention, the level of EBI3 is determined by measuring the quantity or concentration of EBI3 protein in blood samples. Methods for determining the quantity of the EBI3 protein in blood samples include immunoassay methods.

In the methods of diagnosis of the present invention, the blood concentration of CEA or pro-GRP may be determined, in addition to the blood concentration of EBI3, to detect lung cancer. Therefore, the present invention provides methods for diagnosing lung cancer, in which lung cancer is detected when either the blood concentration of EBI3 or the blood concentration of CEA or pro-GRP, or both of them, are higher as compared with healthy individuals.

Carcinoembryonic antigen (CEA) is a frequently studied tumor marker of cancer including lung cancer.

Pro-gastrin-releasing peptide (pro-GRP) is a useful marker in small cell lung carcinomas. As described above, CEA or pro-GRP has already been used as serological marker for diagnosing or detecting lung cancer. However, the sensitivity of CEA or pro-GRP as a marker for lung cancer is somewhat insufficient for detecting lung cancer, completely. Accordingly, it is required that the sensitivity of diagnosing lung cancer would be improved.

In the present invention, a novel serological marker for lung cancer, EBI3, is provided. Improvement in the sensitivity of diagnostic or detection methods for lung cancer may be achieved by the present invention. Namely, the present invention provides a method for diagnosing lung cancer in a subject, including the steps of:

(a) collecting a blood sample from a subject to be diagnosed;

(b) determining a level of EBI3 in the blood sample;

(c) comparing the EBI3 level determined in step (b) with that of a normal control, wherein a high EBI3 level in the blood sample, as compared to the normal control, indicates that the subject suffers from lung cancer. Alternatively, the present invention provide a method for diagnosing SCC in a subject, including the steps of:

(a) determining a level of EBI3 in the blood sample collected from a subject to be diagnosed;

(b) comparing the EBI3 level determined in step (a) with that of a normal control, wherein a high EBI3 level in the blood sample, as compared to the normal control, indicates that the subject suffers from lung cancer.

In preferable embodiments, in the case of NSCLC the diagnostic or detection method of the present invention may further include the steps of:

(d) determining a level of CEA in the blood sample;

(e) comparing the CEA level determined in step (d) with that of a normal control; and

(f) judging that either or both of high EBI3 and high CEA levels in the blood sample, as compared to the normal control, indicate that the subject suffers from lung cancer, especially NSCLC.

By the combination between EBI3 and CEA, the sensitivity for detection of lung cancer, especially NSCLC may be significantly improved. For example, in the group analyzed in the working example discussed below, positive rate of CEA for lung cancer is about 40.0%. In comparison, that of combination between CEA and EBI3 increases to 64.9% (FIG. 4C left panel). In the present invention, “combination of CEA and EBI3” refers to either or both levels of CEA and EBI3 being used as marker. In the preferable embodiments, a patient with positive either of CEA and EBI3 may be judged to have a high risk of lung cancer. The use of combination of EBI3 and CEA as serological marker for lung cancer is novel.

ROC analyses for the patients with SCC determined the cut off value of CYFRA as 2.0 ng/ml, with a sensitivity of 48.6% (18 of 37) and a specificity of 2.3% (3 of 130; FIG. 4C, middle top panel). The correlation coefficient between serum EBI3 and CYFRA was not significant (Spearman rank correlation coefficient: ρ (rho)=−0.117; P=0.4817), indicating that measuring both markers in serum can improve overall sensitivity for detection of SCC to 78.5%; for diagnosing SCC, the sensitivity of CYFRA alone is 48.6% (18 of 37) and that of EBI3 is 54.1% (20 of 37). False-positive rates for either of the two tumor markers among normal volunteers (control group) were 4.6% (6 of 130), although the false-positive rates for each of CYFRA and EBI3 in the same control group were 2.3% (3 of 130) and 2.3% (3 of 130; FIG. 4C, middle bottom panel).

In the case of SCC the diagnostic or detection method of the present invention may further include the steps of:

(d) determining a level of CYFRA in the blood sample;

(e) comparing the CYFRA level determined in step (d) with that of a normal control; and

(f) judging that either or both of high EBI3 and high CYFRA levels in the blood sample,

as compared to the normal control, indicate that the subject suffers from lung cancer, especially SCC.

Alternatively, in the case of SCLC the diagnostic or detection method of the present invention may further include the steps of:

(d) determining a level of pro-GRP in the blood sample;

(e) comparing the pro-GRP level determined in step (d) with that of a normal control; and

(f) judging that either or both of high EBI3 and high pro-GRP levels in the blood sample, as compared to the normal control, indicate that the subject suffers from lung cancer, especially SCLC.

By combining EBI3 and pro-GRP, the sensitivity for detection of lung cancer, especially SCLC, may be significantly improved. For example, in the group analyzed in the working example discussed below, positive rate of pro-GRP for lung cancer is about 67.5%. In comparison, that of combination between pro-GRP and EBI3 increases to 76.3% (FIG. 4C, right panel). In the present invention, “combination of pro-GRP and EBI3” refers to either or both levels of pro-GRP and EBI3 being used as marker. In the preferable embodiments, a patient with positive either of pro-GRP and EBI3 may be judged to have a high risk of lung cancer. The use of combination of EBI3 and pro-GRP as serological marker for lung cancer is a novel discovery of the present invention.

Therefore, the present invention can greatly improve the sensitivity for detecting lung cancer patients, compared to determinations based on results of measuring CEA or pro-GRP alone. Behind this improvement is the fact that the group of CEA- or pro-GRP-positive patients and the group of EBI3-positive patients do not match completely.

For example, among patients who, as a result of CEA or pro-GRP measurements, were determined to have a lower value than a standard value (i.e. not to have lung cancer), there is actually a certain percentage of patients that have lung cancer. Such patients are referred to as CEA- or pro-GRP-false negative patients. By combining a determination based on CEA or pro-GRP with a determination based on EBI3, patients whose EBI3 value is above the standard value can be found from among the CEA- or pro-GRP-false-negative patients. That is, from among patients falsely determined to be “negative” due to a low blood concentration of CEA or pro-GRP, the present invention provides a means to identify patients actually having lung cancer. The sensitivity for detecting lung cancer patients is thus improved by the present invention. Generally, simply combining the results from determinations using multiple markers may increase the detection sensitivity, but on the other hand, it often causes a decrease in specificity. However, by determining the best balance between sensitivity and specificity, the present invention has determined a characteristic combination that can increase the detection sensitivity without compromising the specificity.

In the present invention, in order to consider the results of CEA or pro-GRP measurements at the same time, for example, the blood concentration of CEA or pro-GRP may be measured and compared with standard values, in the same way as for the aforementioned comparison between the measured values and standard values of EBI3. For example, how to measure the blood concentration of CEA or pro-GRP and compare it to standard values are already known. Moreover, ELISA kits for CEA or pro-GRP are also commercially available. These methods described in known reports can be used in the method of the present invention for diagnosing or detecting lung cancer.

Similarly, in further embodiment of the present invention, the level of NPTX1 is determined by measuring the quantity or concentration of NPTX1 protein in blood samples. Methods for determining the quantity of the NPTX1 protein in blood samples include immunoassay methods.

In the methods of diagnosis of the present invention, the blood concentration of CYFRA may be determined, in addition to the blood concentration of NPTX, to detect SCC. Therefore, the present invention provides methods for diagnosing SCC, in which SCC is detected when either the blood concentration of NPTX1 or the blood concentration of CYFRA, or both of them, are higher as compared with healthy individuals.

Cytokeratin 19 fragment (CYFRA, or CYFRA 21-1) is a frequently studied tumor marker of cancer same as Carcinoembryonic antigen (CEA). CYFRA is a useful marker in non-small cell lung carcinomas. As described above, CYFRA has already been used as serological marker for diagnosing or detecting NSCLC. However, the sensitivity of CYFRA as a marker for SCC is somewhat insufficient for detecting SCC, completely, especially at early stage. Accordingly, it is required that the sensitivity of diagnosing SCC would be improved.

In the present invention, a novel serological marker for SCC, NPTX, is provided. Improvement in the sensitivity of diagnostic or detection methods for SCC may be achieved by the present invention. Namely, the present invention provides a method for diagnosing SCC in a subject, including the steps of:

(a) collecting a blood sample from a subject to be diagnosed;

(b) determining a level of NPTX1 in the blood sample;

(c) comparing the NPTX1 level determined in step (b) with that of a normal control, wherein a high NPTX1 level in the blood sample, as compared to the normal control, indicates that the subject suffers from lung cancer. Alternatively, the present invention provide a method for diagnosing SCC in a subject, including the steps of:

(a) determining a level of NPTX1 in the blood sample collected from a subject to be diagnosed;

(b) comparing the NPTX1 level determined in step (a) with that of a normal control, wherein a high NPTX1 level in the blood sample, as compared to the normal control, indicates that the subject suffers from lung cancer.

In preferable embodiments, in the case of SCC the diagnostic or detection method of the present invention may further include the steps of:

(d) determining a level of CYFRA in the blood sample;

(e) comparing the CYFRA level determined in step (d) with that of a normal control; and

(f) judging that either or both of high NPTX1 and high CYFRA levels in the blood sample, as compared to the normal control, indicate that the subject suffers from SCC.

By the combination between NPTX1 and CYFRA, the sensitivity for detection of SCC may be significantly improved. For example, in the group analyzed in the working example discussed below, positive rate of CYFRA for SCC is about 29.4%. In comparison, that of combination between CYFRA and NPTX1 increases to 62.3%. In the present invention, “combination of CYFRA and NPTX” refers to either or both levels of CYFRA and NPTX1 being used as marker. In the preferable embodiments, a patient with positive either of CYFRA and NPTX1 may be judged to have a high risk of SCC. The use of combination of NPTX1 and CYFRA as serological marker for SCC is novel.

Therefore, the present invention can greatly improve the sensitivity for detecting SCC patients, compared to determinations based on results of measuring CYFRA alone. Behind this improvement is the fact that the group of CYFRA-positive patients and the group of NPTX-positive patients do not match completely.

For example, among patients who, as a result of CYFRA measurements, were determined to have a lower value than a standard value (i.e. not to have SCC), there is actually a certain percentage of patients that have SCC. Such patients are referred to as CYFRA-false negative patients. By combining a determination based on CYFRA with a determination based on NPTX, patients whose NPTX1 value is above the standard value can be found from among the CYFRA-false-negative patients. That is, from among patients falsely determined to be “negative” due to a low blood concentration of CYFRA, the present invention provides a means to identify patients actually having SCC. The sensitivity for detecting SCC patients is thus improved by the present invention. Generally, simply combining the results from determinations using multiple markers may increase the detection sensitivity, but on the other hand, it often causes a decrease in specificity. However, by determining the best balance between sensitivity and specificity, the present invention has determined a characteristic combination that can increase the detection sensitivity without compromising the specificity.

In the present invention, in order to consider the results of CYFRA measurements at the same time, for example, the blood concentration of CYFRA may be measured and compared with standard values, in the same way as for the aforementioned comparison between the measured values and standard values of NPTX. For example, how to measure the blood concentration of CYFRA and compare it to standard values are already known. Moreover, ELISA kits for CYFRA are also commercially available. These methods described in known reports can be used in the method of the present invention for diagnosing or detecting SCC.

In the present invention, the standard value of the blood concentration of EBI3 and/or NPTX1 can be determined statistically. For example, the blood concentration of EBI3 and/or NPTX1 in healthy individuals can be measured to determine the standard blood concentration of EBI3 and/or NPTX1 statistically. When a statistically sufficient population is gathered, a value in the range of twice or three times the standard deviation (S.D.) from the mean value is often used as the standard value. Therefore, values corresponding to the mean value +2×S.D. or mean value +3×S.D. may be used as standard values. The standard values set as described theoretically comprise 90% and 99.7% of healthy individuals, respectively.

Alternatively, standard values can also be set based on the actual blood concentration of EBI3 and/or NPTX1 in lung cancer or SCC patients, respectively. Generally, standard values set this way minimize the percentage of false positives, and are selected from a range of values satisfying conditions that can maximize detection sensitivity. Herein, the percentage of false positives refers to a percentage, among healthy individuals, of patients whose blood concentration of EBI3 and/or NPTX1 is judged to be higher than a standard value. On the contrary, the percentage, among healthy individuals, of patients whose blood concentration of EBI3 and/or NPTX1 is judged to be lower than a standard value indicates specificity. That is, the sum of the false positive percentage and the specificity is always 1. The detection sensitivity refers to the percentage of patients whose blood concentration of EBI3 and/or NPTX1 is judged to be higher than a standard value, among all lung cancer patients within a population of individuals for whom the presence of lung cancer has been determined.

Furthermore, in the present invention, the percentage of lung cancer or SCC patients among patients whose EBI3 and/or NPTX1 concentration was judged to be higher than a standard value represents the positive predictive value. On the other hand, the percentage of healthy individuals among patients whose EBI3 and/or NPTX1 concentration was judged to be lower than a standard value represents the negative predictive value. The relationship between these values is summarized in Table 1. As the relationship shown below indicates, each of the values for sensitivity, specificity, positive predictive value, and negative predictive value, which are indexes for evaluating the diagnostic accuracy for lung cancer or SCC, varies depending on the standard value for judging the level of the blood concentration of EBI3 and/or NPTX.

TABLE 1

| Blood | Lung | | |
|---|---|---|---|
| concentration | cancer | Healthy | |
| of EBI3 | patients | individuals | |
| High | a: True | b: False | Positive predictive value |
| | positive | positive | a/(a + b) |
| Low | c: False | d: True | Negative predictive value |
| | negative | negative | d/(c + d) |
| | Sensitivity | Specificity | |
| | a/(a + c) | d/(b + d) | |

As mentioned previously, a standard value is usually set such that the false positive ratio is low and the sensitivity is high. However, as also apparent from the relationship shown above, there is a trade-off between the false positive ratio and sensitivity. That is, if the standard value is decreased, the detection sensitivity increases. However, since the false positive ratio also increases, it is difficult to satisfy the conditions to have a “low false positive ratio”. Considering this situation, for example, values that give the following predicted results may be selected as the preferable standard values in the present invention.

Standard values for which the false positive ratio is 50% or less (that is, standard values for which the specificity is not less than 50%).

Standard values for which the sensitivity is not less than 20%.

In the present invention, the standard values can be set using a receiver operating characteristic (ROC) curve. An ROC curve is a graph that shows the detection sensitivity on the vertical axis and the false positive ratio (that is, “1-specificity”) on the horizontal axis. In the present invention, an ROC curve can be obtained by plotting the changes in the sensitivity and the false positive ratio, which were obtained after continuously varying the standard value for determining the high/low degree of the blood concentration of EBI3 and/or NPTX.

The “standard value” for obtaining the ROC curve is a value temporarily used for the statistical analyses. The “standard value” for obtaining the ROC curve can generally be continuously varied within a range that is allowed to cover all selectable standard values. For example, the standard value can be varied between the smallest and largest measured EBI3 and/or NPTX1 values in an analyzed population.

Based on the obtained ROC curve, a preferable standard value to be used in the present invention can be selected from a range that satisfies the above-mentioned conditions. Alternatively, a standard value can be selected based on an ROC curve produced by varying the standard values from a range that includes most of the measured EBI3 and/or NPTX1 values.

EBI3 and/or NPTX1 in the blood can be measured by any method that can quantitate proteins. For example, immunoassay, liquid chromatography, surface plasmon resonance (SPR), mass spectrometry, or the like can be used in the present invention. In mass spectrometry, proteins can be quantitated by using a suitable internal standard. For example, isotope-labeled EBI3 and/or NPTX1 can be used as the internal standard. The concentration of EBI3 and/or NPTX1 in the blood can be determined from the peak intensity of EBI3 and/or NPTX1 in the blood and that of the internal standard. Generally, the matrix-assisted laser desorption/ionization (MALDI) method is used for mass spectrometry of proteins. With an analysis method that uses mass spectrometry or liquid chromatography, EBI3 can also be analyzed simultaneously with other tumor markers (e.g. CEA or pro-GRP). Alternatively, with an analysis method that uses mass spectrometry or liquid chromatography, NPTX1 can also be analyzed simultaneously with other tumor markers (e.g. CYFRA).

A preferable method for measuring EBI3 and/or NPTX1 in the present invention is the immunoassay. The amino acid sequence of EBI3 is known (GenBank Accession Number NM_{—}005755). The amino acid sequence of EBI3 is shown in SEQ ID NO: 2, and the nucleotide sequence of the cDNA encoding it is shown in SEQ ID NO: 1. Similarly, the amino acid sequence of NPTX1 is known (GenBank Accession Number NP_{—}002513). The amino acid sequence of NPTX1 is shown in SEQ ID NO: 79, and the nucleotide sequence of the cDNA encoding it is shown in SEQ ID NO: 78 (GenBank Accession Number NM_{—}002522). Therefore, those skilled in the art can prepare antibodies by synthesizing necessary immunogens based on the amino acid sequence of EBI3 or NPTX1. The peptide used as immunogen can be easily synthesized using a peptide synthesizer. The synthetic peptide can be used as an immunogen by linking it to a carrier protein.

Keyhole limpet hemocyanin, myoglobin, albumin, and the like can be used as the carrier protein. Preferable carrier proteins are KLH, bovine serum albumin, and such. The maleimidobenzoyl-N-hydrosuccinimide ester method (hereinafter abbreviated as the MBS method) and the like are generally used to link synthetic peptides to carrier proteins.

Specifically, a cysteine is introduced into the synthetic peptide and the peptide is crosslinked to KLH by MBS using the cysteine's SH group. The cysteine residue may be introduced at the N-terminus or C-terminus of the synthesized peptide.

Alternatively, EBI3 and NPTX1 can be prepared using the nucleotide sequence of EBI3 (GenBank Accession Number NM_{—}005755) and NPTX1 (GenBank Accession Number NM_{—}002522), respectively, or a portion thereof. DNAs comprising the necessary nucleotide sequence can be cloned using mRNAs prepared from EBI3 or NPTX1-expressing tissues. Alternatively, commercially available cDNA libraries can be used as the cloning source. The obtained genetic recombinants of EBI3 and/or NPTX1, or fragments thereof, can also be used as the immunogen. EBI3 and/or NPTX1 recombinants expressed in this manner are preferable as the immunogen for obtaining the antibodies used in the present invention.

Immunogens obtained in this manner are mixed with a suitable adjuvant and used to immunize animals. Known adjuvants include Freund's complete adjuvant (FCA) and incomplete adjuvant. The immunization procedure is repeated at appropriate intervals until an increase in the antibody titer is confirmed. There are no particular limitations on the immunized animals in the present invention. Specifically, animals commonly used for immunization such as mice, rats, or rabbits can be used.

When obtaining the antibodies as monoclonal antibodies, animals that are advantageous for their production may be used. For example in mice, many myeloma cell lines for cell fusion are known, and techniques for establishing hybridomas with a high probability are already well known. Therefore, mice are a desirable immunized animal to obtain monoclonal antibodies.

Furthermore, the immunization treatments are not limited to in vitro treatments. Methods for immunologically sensitizing cultured immunocompetent cells in vitro can also be employed. Antibody-producing cells obtained by these methods are transformed and cloned. Methods for transforming antibody-producing cells to obtain monoclonal antibodies are not limited to cell fusion. For example, methods for obtaining cloneable transformants by virus infection are known.

Hybridomas that produce the monoclonal antibodies used in the present invention can be screened based on their reactivity to EBI3 and/or NPTX1. Specifically, antibody-producing cells are first selected by using as an index the binding activity toward EBI3 and/or NPTX1, or a domain peptide thereof, that was used as the immunogen. Positive clones that are selected by this screening are subcloned as necessary.

The monoclonal antibodies to be used in the present invention can be obtained by culturing the established hybridomas under suitable conditions and collecting the produced antibodies. When the hybridomas are homohybridomas, they can be cultured in vivo by inoculating them intraperitoneally in syngeneic animals. In this case, monoclonal antibodies are collected as ascites fluid. When heterohybridomas are used, they can be cultured in vivo using nude mice as a host.

In addition to in vivo cultures, hybridomas are also commonly cultured ex vivo, in a suitable culture environment. For example, basal media such as RPMI 1640 and DMEM are generally used as the medium for hybridomas. Additives such as animal sera can be added to these media to maintain the antibody-producing ability to a high level. When hybridomas are cultured ex vivo, the monoclonal antibodies can be collected as a culture supernatant. Culture supernatants can be collected by separating from cells after culturing, or by continuously collecting while culturing using a culture apparatus that uses a hollow fiber.

Monoclonal antibodies used in the present invention are prepared from monoclonal antibodies collected as ascites fluid or culture supernatants, by separating immunoglobulin fractions by saturated ammonium sulfate precipitation and further purifying by gel filtration, ion exchange chromatography, or such. In addition, if the monoclonal antibodies are IgGs, purification methods based on affinity chromatography with a protein A or protein G column are effective.

On the other hand, to obtain antibodies used in the present invention as polyclonal antibodies, blood is drawn from animals whose antibody titer increased after immunization, and the serum is separated to obtain an anti-serum. Immunoglobulins are purified from anti-sera by known methods to prepare the antibodies used in the present invention. EBI3-specific antibodies can be prepared by combining immunoaffinity chromatography which uses EBI3 and/or NPTX1 as a ligand with immunoglobulin purification.

When antibodies against EBI3 and/or NPTX1 contact EBI3 and/or NPTX1, the antibodies bind to the antigenic determinant (epitope) that the antibodies recognize through an antigen-antibody reaction. The binding of antibodies to antigens can be detected by various immunoassay principles. Immunoassays can be broadly categorized into heterogeneous analysis methods and homogeneous analysis methods. To maintain the sensitivity and specificity of immunoassays to a high level, the use of monoclonal antibodies is desirable. Methods of the present invention for measuring EBI3 and/or NPTX1 by various immunoassay formats are explained in further detail herein.

First, methods for measuring substance (EBI3 and/or NPTX1) using a heterogeneous immunoassay are described. In heterogeneous immunoassays, a mechanism for detecting antibodies that bind to the substance after separating them from those that do not bind to the substance is required.

To facilitate the separation, immobilized reagents are generally used. For example, a solid phase onto which antibodies recognizing the substance have been immobilized is first prepared (immobilized antibodies). The substance is made to bind to these, and secondary antibodies are further reacted thereto.

When the solid phase is separated from the liquid phase and further washed, as necessary, secondary antibodies remain on the solid phase in proportion to the concentration of the substance. By labeling the secondary antibodies, the substance can be quantitated by measuring the signal derived from the label.

Any method may be used to bind the antibodies to the solid phase. For example, antibodies can be physically adsorbed to hydrophobic materials such as polystyrene. Alternatively, antibodies can be chemically bound to a variety of materials having functional groups on their surfaces. Furthermore, antibodies labeled with a binding ligand can be bound to a solid phase by trapping them using a binding partner of the ligand. Combinations of a binding ligand and its binding partner include avidin-biotin and such. The solid phase and antibodies can be conjugated at the same time or before the reaction between the primary antibodies and the substance.

Similarly, the secondary antibodies do not need to be directly labeled. That is, they can be indirectly labeled using antibodies against antibodies or using binding reactions such as that of avidin-biotin.

The concentration of the substance in a sample is determined based on the signal intensities obtained using standard samples with known concentrations of the substance.

Any antibody can be used as the immobilized antibody and secondary antibody for the heterogeneous immunoassays mentioned above, so long as it is an antibody, or a fragment including an antigen-binding site thereof, that recognizes the substance. Therefore, it may be a monoclonal antibody, a polyclonal antibody, or a mixture or combination of both. For example, a combination of monoclonal antibodies and polyclonal antibodies is a preferable combination in the present invention. Alternatively, when both antibodies are monoclonal antibodies, combining monoclonal antibodies recognizing different epitopes is preferable.

Since the antigens to be measured are sandwiched by antibodies, such heterogeneous immunoassays are called sandwich methods. Since sandwich methods excel in the measurement sensitivity and the reproducibility, they are a preferable measurement principle in the present invention.

The principle of competitive inhibition reactions can also be applied to the heterogeneous immunoassays. Specifically, they are immunoassays based on the phenomenon where the substance in a sample competitively inhibits the binding between the substance with a known concentration and an antibody. The concentration of the substance in the sample can be determined by labeling substance with a known concentration and measuring the amount of substance that reacted (or did not react) with the antibody.

A competitive reaction system is established when antigens with a known concentration and antigens in a sample are simultaneously reacted to an antibody. Furthermore, analyses by an inhibitory reaction system are possible when antibodies are reacted with antigens in a sample, and antigens with a known concentration are reacted thereafter. In both types of reaction systems, reaction systems that excel in the operability can be constructed by setting either one of the antigens with a known concentration used as a reagent component or the antibody as the labeled component, and the other one as the immobilized reagent.

Radioisotopes, fluorescent substances, luminescent substances, substances having an enzymatic activity, macroscopically observable substances, magnetically observable substances, and such are used in these heterogeneous immunoassays. Specific examples of these labeling substances are shown below.

Substances having an enzymatic activity:
peroxidase,
alkaline phosphatase,
urease, catalase,
glucose oxidase,
lactate dehydrogenase, or amylase, etc.

Fluorescent substances:
fluorescein isothiocyanate,
tetramethylrhodamine isothiocyanate,
substituted rhodamine isothiocyanate, or
dichlorotriazine isothiocyanate, etc.

Radioisotopes:
tritium,
¹²⁵I, or
¹³¹I, etc.

Among these, non-radioactive labels such as enzymes are an advantageous label in terms of safety, operability, sensitivity, and such. Enzymatic labels can be linked to antibodies or to EBI3 by known methods such as the periodic acid method or maleimide method.

As the solid phase, beads, inner walls of a container, fine particles, porous carriers, magnetic particles, or such are used. Solid phases formed using materials such as polystyrene, polycarbonate, polyvinyltoluene, polypropylene, polyethylene, polyvinyl chloride, nylon, polymethacrylate, latex, gelatin, agarose, glass, metal, ceramic, or such can be used. Solid materials in which functional groups to chemically bind antibodies and such have been introduced onto the surface of the above solid materials are also known. Known binding methods, including chemical binding such as poly-L-lysine or glutaraldehyde treatment and physical adsorption, can be applied for solid phases and antibodies (or antigens).

Although the steps of separating the solid phase from the liquid phase and the washing steps are required in all heterogeneous immunoassays exemplified herein, these steps can easily be performed using the immunochromatography method, which is a variation of the sandwich method.

Specifically, antibodies to be immobilized are immobilized onto porous carriers capable of transporting a sample solution by the capillary phenomenon, then a mixture of a sample comprising substance (EBI3 and/or NPTX1) and labeled antibodies is deployed therein by this capillary phenomenon. During deployment, substance reacts with the labeled antibodies, and when it further contacts the immobilized antibodies, it is trapped at that location. The labeled antibodies that do not react with the substance pass through, without being trapped by the immobilized antibodies.

As a result, the presence of the substance can be detected using, as an index, the signals of the labeled antibodies that remain at the location of the immobilized antibodies. If the labeled antibodies are maintained upstream in the porous carrier in advance, all reactions can be initiated and completed by just dripping in the sample solutions, and an extremely simple reaction system can be constructed. In the immunochromatography method, labeled components that can be distinguished macroscopically, such as colored particles, can be combined to construct an analytical device that does not even require a special reader.

Furthermore, in the immunochromatography method, the detection sensitivity for the substance can be adjusted. For example, by adjusting the detection sensitivity near the cutoff value described below, the aforementioned labeled components can be detected when the cutoff value is exceeded. By using such a device, whether a subject is positive or negative can be judged very simply. By adopting a constitution that allows a macroscopic distinction of the labels, necessary examination results can be obtained by simply applying blood samples to the device for immunochromatography.

Various methods for adjusting the detection sensitivity of the immunochromatography method are known in the art. For example, a second immobilized antibody for adjusting the detection sensitivity can be placed between the position where samples are applied and the immobilized antibodies (Japanese Patent Application Kokai Publication No. (JP-A) H06-341989 (unexamined, published Japanese patent application)). The substance in the sample is trapped by the second immobilized antibody while deploying from the position where the sample was applied to the position of the first immobilized antibody for label detection. After the second immobilized antibody is saturated, the substance can reach the position of the first immobilized antibody located downstream. As a result, when the concentration of the substance comprised in the sample exceeds a predetermined concentration, the substance bound to the labeled antibody is detected at the position of the first immobilized antibody.

Next, homogeneous immunoassays are described. As opposed to heterogeneous immunological assay methods that require a separation of the reaction solutions as described above, substance (EBI3 and/or NPTX1) can also be measured using homogeneous analysis methods. Homogeneous analysis methods allow the detection of antigen-antibody reaction products without their separation from the reaction solutions.

A representative homogeneous analysis method is the immunoprecipitation reaction, in which antigenic substances are quantitatively analyzed by examining precipitates produced following an antigen-antibody reaction. Polyclonal antibodies are generally used for the immunoprecipitation reactions. When monoclonal antibodies are applied, multiple types of monoclonal antibodies that bind to different epitopes of the substance are preferably used. The products of precipitation reactions that follow the immunological reactions can be macroscopically observed or can be optically measured for conversion into numerical data.

The immunological particle agglutination reaction, which uses as an index the agglutination by antigens of antibody-sensitized fine particles, is a common homogeneous analysis method. As in the aforementioned immunoprecipitation reaction, polyclonal antibodies or a combination of multiple types of monoclonal antibodies can be used in this method as well. Fine particles can be sensitized with antibodies through sensitization with a mixture of antibodies, or they can be prepared by mixing particles sensitized separately with each antibody. Fine particles obtained in this manner gives matrix-like reaction products upon contact with the substance. The reaction products can be detected as particle aggregation. Particle aggregation may be macroscopically observed or can be optically measured for conversion into numerical data.

Immunological analysis methods based on energy transfer and enzyme channeling are known as homogeneous immunoassays. In methods utilizing energy transfer, different optical labels having a donor/acceptor relationship are linked to multiple antibodies that recognize adjacent epitopes on an antigen. When an immunological reaction takes place, the two parts approach and an energy transfer phenomenon occurs, resulting in a signal such as quenching or a change in the fluorescence wavelength. On the other hand, enzyme channeling utilizes labels for multiple antibodies that bind to adjacent epitopes, in which the labels are a combination of enzymes having a relationship such that the reaction product of one enzyme is the substrate of another. When the two parts approach due to an immunological reaction, the enzyme reactions are promoted; therefore, their binding can be detected as a change in the enzyme reaction rate.

In the present invention, blood for measuring EBI3 and/or NPTX1 can be prepared from blood drawn from patients. Preferable blood samples are the serum or plasma. Serum or plasma samples can be diluted before the measurements. Alternatively, the whole blood can be measured as a sample and the obtained measured value can be corrected to determine the serum concentration. For example, concentration in whole blood can be corrected to the serum concentration by determining the percentage of corpuscular volume in the same blood sample.

In a preferred embodiment, the immunoassay comprises an ELISA. The present inventors established sandwich ELISA to detect serum EBI3 and/or NPTX1 in patients with lung cancer.

The EBI3 level and/or NPTX1 level in the blood samples is then compared with an EBI3 level and/or NPTX1 level associated with a reference sample such as a normal control sample. The phrase “normal control level” refers to the level of EBI3 and/or NPTX1 typically found in a blood sample of a population not suffering from lung cancer or SCC, respectively. The reference sample is preferably of a similar nature to that of the test sample. For example, if the test samples includes patient serum, the reference sample should also be serum. The EBI3 level and/or NPTX1 level in the blood samples from control and test subjects may be determined at the same time or, alternatively, the normal control level may be determined by a statistical method based on the results obtained by analyzing the level of EBI3 and/or NPTX in samples previously collected from a control group.

The EBI3 level and/or NPTX1 level may also be used to monitor the course of treatment of lung cancer or SCC. In this method, a test blood sample is provided from a subject undergoing treatment for lung cancer or SCC. Preferably, multiple test blood samples are obtained from the subject at various time points, including before, during, and/or after the treatment. The level of EBI3 and/or NPTX1 in the post-treatment sample may then be compared with the level of EBI3 and/or NPTX1 in the pre-treatment sample or, alternatively, with a reference sample (e.g., a normal control level). For example, if the post-treatment EBI3 level or NPTX1 level is lower than the pre-treatment EBI3 level and/or NPTX1 level, one can conclude that the treatment was efficacious Likewise, if the post-treatment EBI3 level and/or NPTX1 level is similar to the normal control EBI3 level and/or NPTX1 level, one can also conclude that the treatment was efficacious.

An “efficacious” treatment is one that leads to a reduction in the level of EBI3 and/or NPTX1 or a decrease in size, prevalence, or metastatic potential of lung cancer in a subject. When a treatment is applied prophylactically, “efficacious” means that the treatment retards or prevents occurrence of lung cancer (or SCC) or alleviates a clinical symptom of lung cancer (or SCC). The assessment of lung cancer (or SCC) can be made using standard clinical protocols. Furthermore, the efficaciousness of a treatment can be determined in association with any known method for diagnosing or treating lung cancer or SCC. For example, lung cancer is routinely diagnosed histopathologically or by identifying symptomatic anomalies.

### Kit for the Serological Diagnosis of Lung Cancer:

Components used to carry out the diagnosis of lung cancer according to the present invention can be combined in advance and supplied as a testing kit. Accordingly, the present invention provides a kit for detecting a lung cancer, including:

(i) an immunoassay reagent for determining a level of EBI3 in a blood sample; and

(ii) a positive control sample for EBI3.

In the preferable embodiments, the kit of the present invention may further comprise:

(iii) an immunoassay reagent for determining a level of CEA or pro-GRP in a blood sample; and

(iv) a positive control sample for CEA and/or pro-GRP.

Alternatively, components used to carry out the diagnosis of SCC according to the present invention can be combined in advance and supplied as a testing kit. Accordingly, the present invention provides a kit for detecting a lung cancer, including:

(i) an immunoassay reagent for determining a level of NPTX1 in a blood sample; and

(ii) a positive control sample for NPTX1.

In the preferable embodiments, the kit of the present invention may further comprise:

(iii) an immunoassay reagent for determining a level of CYFRA in a blood sample; and

(iv) a positive control sample for CYFRA.

The reagents for the immunoassays which constitute a kit of the present invention may include reagents necessary for the various immunoassays described above. Specifically, the reagents for the immunoassays include an antibody that recognizes the substance to be measured. The antibody can be modified depending on the assay format of the immunoassay. ELISA can be used as a preferable assay format of the present invention. In ELISA, for example, a first antibody immobilized onto a solid phase and a second antibody having a label are generally used.

Therefore, the immunoassay reagents for ELISA can include a first antibody immobilized onto a solid phase carrier. Fine particles or the inner walls of a reaction container can be used as the solid phase carrier. Magnetic particles can be used as the fine particles. Alternatively, multi-well plates such as 96-well microplates are often used as the reaction containers. Containers for processing a large number of samples, which are equipped with wells having a smaller volume than in 96-well microplates at a high density, are also known. In the present invention, the inner walls of these reaction containers can be used as the solid phase carriers.

The immunoassay reagents for ELISA may further include a second antibody having a label. The second antibody for ELISA may be an antibody onto which an enzyme is directly or indirectly linked. Methods for chemically linking an enzyme to an antibody are known. For example, immunoglobulins can be enzymatically cleaved to obtain fragments comprising the variable regions. By reducing the —SS— bonds comprised in these fragments to —SH groups, bifunctional linkers can be attached. By linking an enzyme to the bifunctional linkers in advance, enzymes can be linked to the antibody fragments.

Alternatively, to indirectly link an enzyme, for example, the avidin-biotin binding can be used. That is, an enzyme can be indirectly linked to an antibody by contacting a biotinylated antibody with an enzyme to which avidin has been attached. In addition, an enzyme can be indirectly linked to a second antibody using a third antibody which is an enzyme-labeled antibody recognizing the second antibody. For example, enzymes such as those exemplified above can be used as the enzymes to label the antibodies.

Kits of the present invention include a positive control for EBI3. A positive control for EBI3 includes EBI3 whose concentration has been determined in advance. Preferable concentrations are, for example, a concentration set as the standard value in a testing method of the present invention. Alternatively, a positive control having a higher concentration can also be combined. The positive control for EBI3 in the present invention can additionally comprise CEA and/or pro-GRP whose concentration has been determined in advance. A positive control comprising EBI3, CEA and/or pro-GRP is preferable as the positive control of the present invention.

Therefore, the present invention provides a positive control for detecting lung cancer, which includes EBI3 and CEA and/or pro-GRP at concentrations above a normal value. Alternatively, the present invention relates to the use of a blood sample including EBI3 and CEA and/or pro-GRP at concentrations above a normal value in the production of a positive control for the detection of lung cancer. It has been known that CEA and/or pro-GRP can serve as an index for lung cancer; however, that EBI3 can serve as an index for lung cancer is a novel finding obtained by the present invention. Therefore, positive controls including EBI3 in addition to CEA and/or pro-GRP are novel. The positive controls of the present invention can be prepared by adding CEA and/or pro-GRP and EBI3 at concentrations above a standard value to blood samples. For example, sera comprising CEA and/or pro-GRP and EBI3 at concentrations above a standard value are preferable as the positive controls of the present invention.

Alternatively, kits of the present invention can include a positive control for NPTX1. A positive control for NPTX1 includes NPTX1 whose concentration has been determined in advance. Preferable concentrations are, for example, a concentration set as the standard value in a testing method of the present invention. Alternatively, a positive control having a higher concentration can also be combined. The positive control for NPTX1 in the present invention can additionally include CYFRA whose concentration has been determined in advance. A positive control including NPTX1 and/or CYFRA is preferable as the positive control for detecting SCC of the present invention.

Therefore, the present invention provides a positive control for detecting SCC, which includes NPTX1 and CYFRA at concentrations above a normal value. Alternatively, the present invention relates to the use of a blood sample including NPTX1 and CYFRA at concentrations above a normal value in the production of a positive control for the detection of SCC. It has been known that CYFRA can serve as an index for NSCLC; however, that NPTX1 can serve as an index for SCC is a novel finding obtained by the present invention. Therefore, positive controls including NPTX1 in addition to CYFRA are novel. The positive controls of the present invention can be prepared by adding CYFRA and NPTX1 at concentrations above a standard value to blood samples. For example, sera including CYFRA and nptx1 at concentrations above a standard value are preferable as the positive controls of the present invention.

The positive controls in the present invention are preferably in a liquid form. In the present invention, blood samples are used as samples. Therefore, samples used as controls also need to be in a liquid form. Alternatively, by dissolving a dried positive control with a predefined amount of liquid at the time of use, a control that gives the tested concentration can be prepared. By packaging, together with a dried positive control, an amount of liquid necessary to dissolve it, the user can obtain the necessary positive control by just mixing them. EBI3 or NPTX1 used as the positive control can be a naturally-derived protein or it may be a recombinant protein. Not only positive controls, but also negative controls can be combined in the kits of the present invention. The positive controls or negative controls are used to verify that the results indicated by the immunoassays are correct.

### Screening for an Anti-Lung Cancer Compound:

In the context of the present invention, agents to be identified through the present screening methods may be any compound or composition including several compounds. Furthermore, the test agent exposed to a cell or protein according to the screening methods of the present invention may be a single compound or a combination of compounds. When a combination of compounds is used in the methods, the compounds may be contacted sequentially or simultaneously.

Any test agent, for example, cell extracts, cell culture supernatant, products of fermenting microorganism, extracts from marine organism, plant extracts, purified or crude proteins, peptides, non-peptide compounds, synthetic micromolecular compounds (including nucleic acid constructs, such as antisense RNA, siRNA, Ribozymes, and aptamer etc.) and natural compounds can be used in the screening methods of the present invention. The test agent of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds may be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

A compound in which a part of the structure of the compound screened by any of the present screening methods is converted by addition, deletion and/or replacement, is included in the agents obtained by the screening methods of the present invention.

Furthermore, when the screened test agent is a protein, for obtaining a DNA encoding the protein, either the whole amino acid sequence of the protein may be determined to deduce the nucleic acid sequence coding for the protein, or partial amino acid sequence of the obtained protein may be analyzed to prepare an oligo DNA as a probe based on the sequence, and screen cDNA libraries with the probe to obtain a DNA encoding the protein. The obtained DNA is confirmed it's usefulness in preparing the test agent which is a candidate for treating or preventing cancer.

Test agents useful in the screenings described herein can also be antibodies that specifically bind to EBI3, DLX5, NPTX1, CDKN3 or EF-1delta protein or partial peptides thereof that lack the biological activity of the original proteins in vivo.

Although the construction of test agent libraries is well known in the art, herein below, additional guidance in identifying test agents and construction libraries of such agents for the present screening methods are provided.

### (i) Molecular Modeling:

Construction of test agent libraries is facilitated by knowledge of the molecular structure of compounds known to have the properties sought, and/or the molecular structure of the target molecules to be inhibited, i.e., EBI3, DLX5, NPTX1, CDKN3 and EF-1delta. One approach to preliminary screening of test agents suitable for further evaluation is computer modeling of the interaction between the test agent and its target.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above includes the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. Acta Pharmaceutica Fennica 1988, 97: 159-66; Ripka, New Scientist 1988, 54-8; McKinlay & Rossmann, Annu Rev Pharmacol Toxiciol 1989, 29: 111-22; Perry & Davies, Prog Clin Biol Res 1989, 291: 189-93; Lewis & Dean, Proc R Soc Lond 1989, 236: 125-40, 141-62; and, with respect to a model receptor for nucleic acid components, Askew et al., J Am Chem Soc 1989, 111: 1082-90.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al., J Med Chem 1988, 31: 722-9; Meng et al., J Computer Chem 1992, 13: 505-24; Meng et al., Proteins 1993, 17: 266-78; Shoichet et al., Science 1993, 259: 1445-50.

Once a putative inhibitor has been identified, combinatorial chemistry techniques can be employed to construct any number of variants based on the chemical structure of the identified putative inhibitor, as detailed below. The resulting library of putative inhibitors, or “test agents” may be screened using the methods of the present invention to identify test agents treating or preventing the lung cancer.

### (ii) Combinatorial Chemical Synthesis:

Combinatorial libraries of test agents may be produced as part of a rational drug design program involving knowledge of core structures existing in known inhibitors. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of Combinatorial Chemical Libraries is Well Known to Those of Skill in the art, and may be generated by either chemical or biological synthesis. Combinatorial chemical libraries include, but are not limited to, peptide libraries (see, e.g., U.S. Pat. No. 5,010,175; Furka, Int J Pept Prot Res 1991, 37: 487-93; Houghten et al., Nature 1991, 354: 84-6). Other chemistries for generating chemical diversity libraries can also be used. Such chemistries include, but are not limited to: peptides (e.g., PCT Publication No. WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (DeWitt et al., Proc Natl Acad Sci USA 1993, 90:6909-13), vinylogous polypeptides (Hagihara et al., J Amer Chem Soc 1992, 114: 6568), nonpeptidal peptidomimetics with glucose scaffolding (Hirschmann et al., J Amer Chem Soc 1992, 114: 9217-8), analogous organic syntheses of small compound libraries (Chen et al., J. Amer Chem Soc 1994, 116: 2661), oligocarbamates (Cho et al., Science 1993, 261: 1303), and/or peptidylphosphonates (Campbell et al., J Org Chem 1994, 59: 658), nucleic acid libraries (see Ausubel, Current Protocols in Molecular Biology 1995 supplement; Sambrook et al., Molecular Cloning: A Laboratory Manual, 1989, Cold Spring Harbor Laboratory, New York, USA), peptide nucleic acid libraries (see, e.g., U.S. Pat. No. 5,539,083), antibody libraries (see, e.g., Vaughan et al., Nature Biotechnology 1996, 14(3):309-14 and PCT/US96/10287), carbohydrate libraries (see, e.g., Liang et al., Science 1996, 274: 1520-22; U.S. Pat. No. 5,593,853), and small organic molecule libraries (see, e.g., benzodiazepines, Gordon E M. Curr Opin Biotechnol. 1995 Dec. 1; 6(6):624-31; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

(iii) Phage Display:

Another approach uses recombinant bacteriophage to produce libraries. Using the “phage method” (Scott & Smith, Science 1990, 249: 386-90; Cwirla et al., Proc Natl Acad Sci USA 1990, 87: 6378-82; Devlin et al., Science 1990, 249: 404-6), very large libraries can be constructed (e.g., 106-108 chemical entities). A second approach uses primarily chemical methods, of which the Geysen method (Geysen et al., Molecular Immunology 1986, 23: 709-15; Geysen et al., J Immunologic Method 1987, 102: 259-74); and the method of Fodor et al. (Science 1991, 251: 767-73) are examples. Furka et al. (14th International Congress of Biochemistry 1988, Volume #5, Abstract FR:013; Furka, Int J Peptide Protein Res 1991, 37: 487-93), Houghten (U.S. Pat. No. 4,631,211) and Rutter et al. (U.S. Pat. No. 5,010,175) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Screening for an EBI3, DLX5, NPTX1, CDKN3 and/or EF-1Delta Binding Compound:

In present invention, over-expression of EBI3, DLX5, NPTX1, CDKN3 and EF-1delta was detected in lung cancer, in spite of no expression in normal organs (FIGS. 1, 5, 7, 16 and 19). Therefore, using the EBI3, DLX5, CDKN3 and/or EF-1delta genes, proteins encoded by the genes, the present invention provides a method of screening for a compound that binds to EBI3, DLX5, NPTX1, CDKN3 and/or EF-1delta. Due to the expression of EBI3, DLX5, NPTX1, CDKN3 and EF-1delta in lung cancer, a compound binds to EBI3, DLX5, NPTX1, CDKN3 and/or EF-1delta is expected to suppress the proliferation of lung cancer cells, and thus be useful for treating or preventing lung cancer. Therefore, the present invention also provides a method for screening a compound that suppresses the proliferation of lung cancer cells, and a method for screening a compound for treating or preventing lung cancer using the EBI3, DLX5, NPTX1, CDKN3 and/or EF-1delta polypeptide. Specially, an embodiment of this screening method includes the steps of:

(a) contacting a test compound with a polypeptide encoded by a polynucleotide of EBI3, DLX5, NPTX1, CDKN3 and/or EF-1delta;

(b) detecting the binding activity between the polypeptide and the test compound; and

(c) selecting the test compound that binds to the polypeptide.

The method of the present invention will be described in more detail below.

The EBI3, DLX5, NPTX1, CDKN3 and/or EF-1delta polypeptide to be used for screening may be a recombinant polypeptide or a protein derived from the nature or a partial peptide thereof. The polypeptide to be contacted with a test compound can be, for example, a purified polypeptide, a soluble protein, a form bound to a carrier or a fusion protein fused with other polypeptides.

As a method of screening for proteins, for example, that bind to the EBI3, DLX5, NPTX1, CDKN3 and/or EF-1delta polypeptide using the EBI3, DLX5, NPTX1, CDKN3 and/or EF-1delta polypeptide, many methods well known by a person skilled in the art can be used. Such a screening can be conducted by, for example, immunoprecipitation method, specifically, in the following manner. The gene encoding the EBI3, DLX5, NPTX1, CDKN3 and/or EF-1delta polypeptide is expressed in host (e.g., animal) cells and so on by inserting the gene to an expression vector for foreign genes, such as pSV2neo, pcDNA I, pcDNA3.1, pCAGGS and pCD8.

The promoter to be used for the expression may be any promoter that can be used commonly and include, for example, the SV40 early promoter (Rigby in Williamson (ed.), Genetic Engineering, vol. 3. Academic Press, London, 83-141 (1982)), the EF-alpha promoter (Kim et al., Gene 91: 217-23 (1990)), the CAG promoter (Niwa et al., Gene 108: 193 (1991)), the RSV LTR promoter (Cullen, Methods in Enzymology 152: 684-704 (1987)) the SR alpha promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and so on.

The introduction of the gene into host cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1641-3 (1984)), the Lipofectin method (Derijard B., Cell 76: 1025-37 (1994); Lamb et al., Nature Genetics 5: 22-30 (1993): Rabindran et al., Science 259: 230-4 (1993)) and so on.

The polypeptide encoded by EBI3, DLX5, CDKN3 and/or EF-1delta gene can be expressed as a fusion protein including a recognition site (epitope) of a monoclonal antibody by introducing the epitope of the monoclonal antibody, whose specificity has been revealed, to the N- or C-terminus of the polypeptide. A commercially available epitope-antibody system can be used (Experimental Medicine 13: 85-90 (1995)). Vectors which can express a fusion protein with, for example, beta-galactosidase, maltose binding protein, glutathione S-transferase, green florescence protein (GFP) and so on by the use of its multiple cloning sites are commercially available. Also, a fusion protein prepared by introducing only small epitopes consisting of several to a dozen amino acids so as not to change the property of the EBI3, DLX5, NPTX1, CDKN3 and/or EF-1delta polypeptide by the fusion is also reported. Epitopes, such as polyhistidine (His-tag), influenza aggregate HA, human c-myc, FLAG, Vesicular stomatitis virus glycoprotein (VSV-GP), T7 gene 10 protein (T7-tag), human simple herpes virus glycoprotein (HSV-tag), E-tag (an epitope on monoclonal phage) and such, and monoclonal antibodies recognizing them can be used as the epitope-antibody system for screening proteins binding to the EBI3, DLX5, NPTX1, CDKN3 and/or EF-1delta polypeptide (Experimental Medicine 13: 85-90 (1995)).

In immunoprecipitation, an immune complex is formed by adding these antibodies to cell lysate prepared using an appropriate detergent. The immune complex consists of the EBI3, DLX5, NPTX1, CDKN3 and/or EF-1delta polypeptide, a polypeptide including the binding ability with the polypeptide, and an antibody. Immunoprecipitation can be also conducted using antibodies against the EBI3, DLX5, NPTX1, CDKN3 and/or EF-1delta polypeptide, besides using antibodies against the above epitopes, which antibodies can be prepared as described above. An immune complex can be precipitated, for example by Protein A sepharose or Protein G sepharose when the antibody is a mouse IgG antibody. If the polypeptide encoded by EBI3, DLX5, NPTX1, CDKN3 and/or EF-1delta gene is prepared as a fusion protein with an epitope, such as GST, an immune complex can be formed in the same manner as in the use of the antibody against the EBI3, DLX5, NPTX1, CDKN3 and/or EF-1delta polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the EBI3, DLX5, NPTX1, CDKN3 and/or EF-1delta polypeptide is difficult to detect by a common staining method, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

As a method of screening for proteins binding to the EBI3, DLX5, NPTX1, CDKN3 and/or EF-1delta polypeptide using the polypeptide, for example, West-Western blotting analysis (Skolnik et al., Cell 65: 83-90 (1991)) can be used. Specifically, a protein binding to the EBI3, DLX5, NPTX1, CDKN3 and/or EF-1delta polypeptide can be obtained by preparing a cDNA library from cultured cells (e.g., LC176, LC319, A549, NCI-H23, NCI-H226, NCI-H522, PC3, PC9, PC14, SK-LU-1, EBC-1, RERF-LC-AI, SK-MES-1, SW900, and SW 1573) expected to express a protein binding to the EBI3, DLX5, CDKN3 and/or EF-1delta polypeptide using a phage vector (e.g., ZAP), expressing the protein on LB-agarose, fixing the protein expressed on a filter, reacting the purified and labeled EBI3, DLX5, NPTX1, CDKN3 and/or EF-1delta polypeptide with the above filter, and detecting the plaques expressing proteins bound to the EBI3, DLX5, NPTX1, CDKN3 and/or EF-1delta polypeptide according to the label. The polypeptide of the invention may be labeled by utilizing the binding between biotin and avidin, or by utilizing an antibody that specifically binds to the EBI3, DLX5, CDKN3 and/or EF-1delta polypeptide, or a peptide or polypeptide (for example, GST) that is fused to the EBI3, DLX5, NPTX1, CDKN3 and/or EF-1delta polypeptide. Methods using radioisotope or fluorescence and such may be also used.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, the polypeptide of the invention is fused to the SRF-binding region or GAL4-binding region and expressed in yeast cells. A cDNA library is prepared from cells expected to express a protein binding to the polypeptide of the invention, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the invention is expressed in yeast cells, the binding of the two activates a reporter gene, making positive clones detectable). A protein encoded by the cDNA can be prepared by introducing the cDNA isolated above to E. coli and expressing the protein. As a reporter gene, for example, Ade2 gene, lacZ gene, CAT gene, luciferase gene and such can be used in addition to the HIS3 gene.

A compound binding to the polypeptide encoded by EBI3, DLX5, NPTX1, CDKN3 and/or EF-1delta gene can also be screened using affinity chromatography. For example, the polypeptide of the invention may be immobilized on a carrier of an affinity column, and a test compound, containing a protein capable of binding to the polypeptide of the invention, is applied to the column. A test compound herein may be, for example, cell extracts, cell lysates, etc. After loading the test compound, the column is washed, and compounds bound to the polypeptide of the invention can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the polypeptide of the invention and a test compound can be observed real-time as a surface plasmon resonance signal, using only a minute amount of polypeptide and without labeling (for example, BIAcore, Pharmacia). Therefore, it is possible to evaluate the binding between the polypeptide of the invention and a test compound using a biosensor such as BIAcore.

The methods of screening for molecules that bind when the immobilized EBI3, DLX5, NPTX1, CDKN3 and/or EF-1delta polypeptide is exposed to synthetic chemical compounds, or natural substance banks or a random phage peptide display library, and the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the EBI3, DLX5, NPTX1, CDKN3 and/or EF-1delta protein (including agonist and antagonist) are well known to one skilled in the art.

Screening for a Compound Suppressing the Biological Activity of EBI3, DLX5, NPTX1, CDKN3 and/or EF-1Delta:

In the present invention the EBI3, DLX5, NPTX1, CDKN3 and EF-1delta protein have the activity of promoting cell proliferation of lung cancer cells (FIGS. 4D, 6D, 10A, 10B, 22A and 22B), cell invasion activity (FIG. 23A), extracellular secretion (FIGS. 1C and 7D), phospatase activity (FIG. 21A) and Akt phosphorylation (FIG. 23D). Using these biological activities, the present invention provides a method for screening a compound that suppresses the proliferation of lung cancer cells, and a method for screening a compound for treating or preventing lung cancer. Thus, the present invention provides a method of screening for a compound for treating or preventing lung cancer using the polypeptide encoded by EBI3, DLX5, NPTX1, CDKN3 and/or EF-1delta gene including the steps as follows:

(a) contacting a test compound with a polypeptide encoded by a polynucleotide of EBI3, DLX5, NPTX1, CDKN3 and/or EF-1delta;

(b) detecting the biological activity of the polypeptide of step (a); and

(c) selecting the test compound that suppresses the biological activity of the polypeptide encoded by the polynucleotide of EBI3, DLX5, NPTX1, CDKN3 and/or EF-1delta as compared to the biological activity of said polypeptide detected in the absence of the test compound.

The method of the present invention will be described in more detail below.

Any polypeptides can be used for screening so long as they include the biological activity of the EBI3, DLX5, NPTX1, CDKN3 and/or EF-1delta protein. Such biological activity includes cell-proliferating activity of the EBI3, DLX5, NPTX1, CDKN3 and/or EF-1delta protein. For example, EBI3, DLX5, NPTX1, CDKN3 and/or EF-1delta protein can be used and polypeptides functionally equivalent to these proteins can also be used. Such polypeptides may be expressed endogenously or exogenously by cells.

The compound isolated by this screening is a candidate for antagonists of the polypeptide encoded by EBI3, DLX5, NPTX1, CDKN3 and/or EF-1delta gene. The term “antagonist” refers to molecules that inhibit the function of the polypeptide by binding thereto. Said term also refers to molecules that reduce or inhibit expression of the gene encoding EBI3, DLX5, NPTX1, CDKN3 and/or EF-1delta. Moreover, a compound isolated by this screening is a candidate for compounds which inhibit the in vivo interaction of the EBI3, DLX5, NPTX1, CDKN3 and/or EF-1delta polypeptide with molecules (including DNAs and proteins).

When the biological activity to be detected in the present method is cell proliferation, it can be detected, for example, by preparing cells which express the EBI3, DLX5, NPTX1, CDKN3 and/or EF-1delta polypeptide, culturing the cells in the presence of a test compound, and determining the speed of cell proliferation, measuring the cell cycle and such, as well as by measuring the colony forming activity, for example, shown in FIGS. 4D, 6D, 10A, 10B, 22A and 22B. The compounds that reduce the speed of proliferation of cells expressed the EBI3, DLX5, NPTX1, CDKN3 and/or EF-1delta polypeptide compared with that of no compound treated cells and keep the speed of that compared with no or little those polypeptides expressed cells are selected as candidate compound for treating or preventing lung cancer.

When the biological activity to be detected in the present method is cell invasion activity, it can be detected, for example, by preparing cells which express CDKN3 polypeptide and determining the amount of invasion cells, measuring with matrigel invasion assay, for example, shown in FIG. 23A. The compounds that reduce the amount of invasion cells expressed CDKN3 polypeptide compared with that of no compound treated cells and keep the amount of that compared with no or little CDKN3 polypeptides expressed cells are selected as candidate compound for treating or preventing lung cancer.

When the biological activity to be detected in the present method is extracellular secretion, it can be detected, for example, by preparing cells which express EBI3 or NPTX1 polypeptide, culturing the cells in the presence of a test compound, and determining the amount of secreted protein of those polypeptides in culture medium, measuring with ELISA, for example, shown in FIGS. 1C and 7D. The compounds that reduce the amount of secreted protein from the cells expressed EBI3 or NPTX1 polypeptide compared with that of no compound treated cells or EBI3 and keep the amount of that compared with no or little NPTX1 polypeptides expressed cells are selected as candidate compound for treating or preventing lung cancer.

When the biological activity to be detected in the present method is phospatase activity, it can be detected, for example, by contacting CDKN3 polypeptide or functional equivalent thereof with EF-1delta polypeptide or functional equivalent thereof, in the presence of a test compound and determining the phosphorylation level of EF-1delta polypeptide, for example, measuring with western bloting shown in FIG. 21A. The compounds that reduce the phosphorylation level of EF-1delta polypeptide compared with that of no compound treated cells are selected as candidate compound for treating or preventing lung cancer. In the preferably method, the detection of phosphorylation level of EF-1delta polypeptide is measured by phospho-serine.

When the biological activity to be detected in the present method is Akt phosphorylation, it can be detected, for example, by preparing cells which express CDKN3 polypeptide and determining the level of Akt phosphorylation, measuring with western blot, for example, shown in FIG. 23D. The compounds that reduce the level of Akt phosphorylation in cells expressed CDKN3 polypeptide compared with that of no compound treated cells and keep the amount of that compared with no or little CDKN3 polypeptides expressed cells are selected as candidate compound for treating or preventing lung cancer.

For example, it was confirmed that EF-1delta was co-expressed with CDKN3 in lung cancer cells, and is likely to be a physiological substrate of CDKN3 phosphatase in vivo suggesting that CDKN3 could have a growth-promoting function in lung tumors through dephosphorylation of EF-1delta (FIGS. 20-21). Accordingly, compounds that inhibit the dephosphorylation of EF-1delta through the inhibition of CDKN3 function is expected to suppress the proliferation of lung cancer cells, and thus is useful for treating or preventing lung cancer, including NSCLC or SCLC. Therefore, the present invention also provides a method for screening a compound that suppresses the proliferation of lung cancer cells, and a method for screening a compound for treating or preventing lung cancer, including NSCLC and/or SCLC.

More specifically, the method includes the steps of:
(a) contacting a candidate compound with cells which overexpress CDKN3;
(b) measuring a phosphorylation level of EF-1delta; and
(c) selecting a candidate compound that reduces the dephosphorylation as compared to a control.

Preferably, the phosphorylation and dephosphorylation of EF-1delta may be detected by determining molecular weight of EF-1delta. Method for determining molecular weight of proteins is well known. For example, by using western blot analysis described in following EXAMPLES section, phosphorylation and dephosphorylation can be detected as increase and decrease of the molecular weight, respectively. Alternatively, phosphorylation level of EF-1delta can also be evaluated by immunological technique using antibody recognizing phosphorylated EF-1delta. For example, antibody recognizing phosphorylated serine on EF-1delta, or pan-phospho-specific antibody can be used for such purpose. In preferred embodiments, control level to be compared may be phosphorylation level of EF-1delta detected in absence of the candidate compound under the condition same as test condition (in presence of the candidate compound).

Alternatively, in the present invention, it was revealed that the Akt phosphorylation (Ser473) is enhanced by CDKN3 overexpression (FIG. 23). Accordingly, compounds that decrease the Akt phosphorylation through the inhibition of CDKN3 function is also expected to suppress the proliferation of lung cancer cells, and thus is useful for treating or preventing lung cancer, including NSCLC and/or SCLC. Therefore, the present invention also provides a method for screening a compound that suppresses the proliferation of lung cancer cells, and a method for screening a compound for treating or preventing lung cancer, including NSCLC and/or SCLC.

More specifically, the method includes the steps of:
(a) contacting a candidate compound with cells which overexpress CDKN3;
(b) measuring the phosphorylation of Akt Ser473; and
(c) selecting a candidate compound that reduces the phosphorylation as compared to a control.

In preferred embodiments, a test compound selected by the method of the present invention may be candidate for further screening to evaluate the therapeutic effect thereof.

Preferably, the phosphorylation level of Akt may be detected at the 473 serine residue of the amino acid sequence of SEQ ID NO: 60 encoded by nucleotide sequence of SEQ ID NO: 59 (NP_{—}001014431). The detection method of Akt phosphorylation well known by one skilled in the art can be used. For example, western blot analysis described in following EXAMPLES section can be used.

In the context of the present invention, the conditions suitable for the phosphorylation of Akt by CDKN3 may be provided with an incubation of Akt and CDKN3 in the presence of phosphate donor, e.g. ATP. The conditions suitable for the Akt phosphorylation by CDKN3 also include culturing cells expressing CDKN3 and Akt. For example, the cell may be a transformant cell harboring an expression vector containing a polynucleotide that encodes the polypeptide. After the incubation, the phosphorylation level of the Akt can be detected with an antibody recognizing phosphorylated Akt. In preferred embodiments, control level to be compared may be phosphorylation level of Akt detected in absence of the candidate compound under the condition same as test condition (in presence of the candidate compound).

Prior to the detection of phosphorylated Akt, Akt may be separated from other elements, or cell lysate of Akt expressing cells. For instance, gel electrophoresis may be used for the separation of Akt from remaining components. Alternatively, Akt may be captured by contacting Akt with a carrier having an anti-Akt antibody. When the labeled phosphate donor is used, the phosphorylation level of the Akt can be detected by tracing the label. For example, when radio-labeled ATP (e.g. ³²P-ATP) is used as a phosphate donor, radio activity of the separated Akt correlates with the phosphorylation level of the Akt. Alternatively, an antibody specifically recognizing phosphorylated Akt from unphosphorylated Akt may be used to detect phosphorylated Akt. Preferably, the antibody recognizes phosphorylated Akt at Ser-473 residues.

Methods for preparing polypeptides functionally equivalent to a given protein are well known by a person skilled in the art and include known methods of introducing mutations into the protein. Generally, it is known that modifications of one or more amino acid in a protein do not influence the function of the protein (Mark D F et al., Proc Natl Acad Sci USA 1984, 81: 5662-6; Zoller M J & Smith M, Nucleic Acids Res 1982, 10: 6487-500; Wang A et al., Science 1984, 224:1431-3; Dalbadie-McFarland G et al., Proc Natl Acad Sci USA 1982, 79: 6409-13). In fact, mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)). Accordingly, one of skill in the art will recognize that individual additions, deletions, insertions, or substitutions to an amino acid sequence which alter a single amino acid or a small percentage of amino acids, or those considered to be “conservative modifications”, wherein the alteration of a protein results in a protein with similar functions, are contemplated in the context of the instant invention.

For example, one skilled in the art can prepare polypeptides functionally equivalent to EBI3, DLX5, NPTX1, CDKN3, EF-1delta and/or Akt protein by introducing an appropriate mutation in the amino acid sequence of either of these proteins using, for example, site-directed mutagenesis (Hashimoto-Gotoh et al., Gene 152:271-5 (1995); Zoller and Smith, Methods Enzymol 100: 468-500 (1983); Kramer et al., Nucleic Acids Res. 12:9441-56 (1984); Kramer and Fritz, Methods Enzymol 154: 350-67 (1987); Kunkel, Proc Natl Acad Sci USA 82: 488-92 (1985); Kunkel T A, et al., Methods Enzymol. 1991; 204:125-39.). The polypeptides of the present invention includes those having the amino acid sequences of EBI3, DLX5, NPTX1, CDKN3, EF-1delta and/or Akt in which one or more amino acids are mutated, provided the resulting mutated polypeptides are functionally equivalent to EBI3, DLX5, NPTX1, CDKN3, EF-1delta and/or Akt, respectively. So long as the activity the protein is maintained, the number of amino acid mutations is not particularly limited. However, it is generally preferred to alter 5% or less of the amino acid sequence. Accordingly, in a preferred embodiment, the number of amino acids to be mutated in such a mutant is generally 30 amino acids or less, typically 20 amino acids or less, more typically 10 amino acids or less, preferably 5-6 amino acids or less, and more preferably 1-3 amino acids.

The amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Note, the parenthetic letters indicate the one-letter codes of amino acids. Furthermore, conservative substitution tables providing functionally similar amino acids are well known in the art. For example, the following eight groups each contain amino acids that are conservative substitutions for one another:
1) Alanine (A), Glycine (G);
2) Aspartic acid (D), Glutamic acid (E);
3) Aspargine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M) (see, e.g., Creighton, Proteins 1984).

Such conservatively modified polypeptides are included in the present EBI3, DLX5, NPTX1, CDKN3, EF-1delta or Akt protein. However, the present invention is not restricted thereto and the EBI3, DLX5, NPTX1, CDKN3, EF-1delta or Akt proteins include non-conservative modifications so long as the binding activity of the original proteins is retained. Furthermore, the modified proteins do not exclude polymorphic variants, interspecies homologues, and those encoded by alleles of these proteins.

An example of a polypeptide to which one or more amino acids residues are added to the amino acid sequence of EBI3, DLX5, NPTX1, CDKN3, EF-1delta or Akt is a fusion protein containing EBI3, DLX5, NPTX1, CDKN3, EF-1delta or Akt, respectively. Accordingly, fusion proteins, i.e., fusions of EBI3, DLX5, NPTX1, CDKN3, EF-1delta or Akt and other peptides or proteins, are included in the present invention. Fusion proteins can be made by techniques well known to a person skilled in the art, such as by linking the DNA encoding EBI3, DLX5, NPTX1, CDKN3, EF-1delta or Akt with DNA encoding other peptides or proteins, so that the frames match, inserting the fusion DNA into an expression vector and expressing it in a host. There is no restriction as to the peptides or proteins fused to the protein of the present invention.

Known peptides that can be used as peptides to be fused to the EBI3, DLX5, NPTX1, CDKN3, EF-1delta or Akt proteins include, for example, FLAG (Hopp T P et al., Biotechnology 1988 6: 1204-10), 6×His containing six His (histidine) residues, 10×His, Influenza agglutinin (HA), human c-myc fragment, VSP-GP fragment, p18HIV fragment, T7-tag, HSV-tag, E-tag, SV40T antigen fragment, lck tag, alpha-tubulin fragment, B-tag, Protein C fragment, and the like. Examples of proteins that may be fused to a protein of the invention include GST (glutathione-S-transferase), Influenza agglutinin (HA), immunoglobulin constant region, beta-galactosidase, MBP (maltose-binding protein), and such.

Fusion proteins can be prepared by fusing commercially available DNA, encoding the fusion peptides or proteins discussed above, with the DNA encoding the EBI3, DLX5, NPTX1, CDKN3, EF-1delta or Akt proteins and expressing the fused DNA prepared.

An alternative method known in the art to isolate functionally equivalent polypeptides involves, for example, hybridization techniques (Sambrook et al., Molecular Cloning 2nd ed. 9.47-9.58, Cold Spring Harbor Lab. Press (1989)). One skilled in the art can readily isolate a DNA having high homology with EBI3, DLX5, NPTX1, CDKN3, EF-1delta or Akt, and isolate polypeptides functionally equivalent to the EBI3, DLX5, NPTX1, CDKN3, EF-1delta or Akt from the isolated DNA. The proteins of the present invention include those that are encoded by DNA that hybridize with a whole or part of the DNA sequence encoding EBI3, DLX5, NPTX1, CDKN3, EF-1delta or Akt and are functionally equivalent to EBI3, DLX5, NPTX1, CDKN3, EF-1delta or Akt. These polypeptides include mammalian homologues corresponding to the protein derived from humans (for example, a polypeptide encoded by a monkey, rat, rabbit and bovine gene). In isolating a cDNA highly homologous to the DNA encoding EBI3, DLX5, NPTX1, CDKN3, EF-1delta or Akt from animals, it is particularly preferable to use prostate cancer tissues.

The condition of hybridization for isolating a DNA encoding a protein functional equivalent to the human EBI3, DLX5, NPTX1, CDKN3, EF-1delta or Akt protein can be routinely selected by a person skilled in the art. The phrase “stringent (hybridization) conditions” refers to conditions under which a nucleic acid molecule will hybridize to its target sequence, typically in a complex mixture of nucleic acids, but not detectably to other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). In the context of the present invention, suitable hybridization conditions can be routinely selected by a person skilled in the art

Generally, stringent conditions are selected to be about 5-10 degrees C. lower than the thermal melting point (Tₘ) for the specific sequence at a defined ionic strength pH. The Tₙ, is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may also be achieved with the addition of destabilizing agents such as formamide. For selective or specific hybridization, a positive signal is preferably at least two times of background, more preferably 10 times of background hybridization.

Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 50° C. Suitable hybridization conditions may also include prehybridization at 68 degrees C. for 30 min or longer using “Rapid-hyb buffer” (Amersham LIFE SCIENCE), adding a labeled probe, and warming at 68 degrees C. for 1 h or longer.

The washing step can be conducted, for example, under conditions of low stringency. Thus, an exemplary low stringency condition may include, for example, 42° C., 2×SSC, 0.1% SDS, or preferably 50° C., 2×SSC, 0.1% SDS. Alternatively, an exemplary high stringency condition may include, for example, washing 3 times in 2×SSC, 0.01% SDS at room temperature for 20 min, then washing 3 times in 1×SSC, 0.1% SDS at 37 degrees C. for 20 min, and washing twice in 1×SSC, 0.1% SDS at 50 degrees C. for 20 min. However, several factors such as temperature and salt concentration can influence the stringency of hybridization and one skilled in the art can suitably select the factors to achieve the requisite stringency.

Preferably, the functionally equivalent polypeptide has an amino acid sequence with at least about 80% homology (also referred to as sequence identity) to the native EBI3, DLX5, NPTX1, CDKN3, EF-1delta or Akt sequence disclosed here, more preferably at least about 85%, 90%, 95%, 96%, 97%, 98%, or 99% homology. The homology of a polypeptide can be determined by following the algorithm in “Wilbur and Lipman, Proc Natl Acad Sci USA 80: 726-30 (1983)”. In other embodiments, the functional equivalent polypeptide can be encoded by a polynucleotide that hybridizes under stringent conditions (as defined below) to a polynucleotide encoding such a functional equivalent polypeptide.

In place of hybridization, a gene amplification method, for example, the polymerase chain reaction (PCR) method, can be utilized to isolate a DNA encoding a polypeptide functionally equivalent to EBI3, DLX5, NPTX1, CDKN3, EF-1delta or Akt, using a primer synthesized based on the sequence information for EBI3, DLX5, NPTX1, CDKN3, EF-1delta or Akt.

An EBI3, DLX5, NPTX1, CDKN3, EF-1delta or Akt functional equivalent useful in the context of the present invention may have variations in amino acid sequence, molecular weight, isoelectric point, the presence or absence of sugar chains, or form, depending on the cell or host used to produce it or the purification method utilized. Nevertheless, so long as it is a function equivalent of any one of the EBI3, DLX5, NPTX1, CDKN3, EF-1delta or Akt polypeptide, it is within the scope of the present invention.

“Suppress the biological activity” as defined herein are preferably at least 10% suppression of the biological activity of EBI3, DLX5, NPTX1, CDKN3 and/or EF-1delta in comparison with in absence of the compound, more preferably at least 25%, 50% or 75% suppression and most preferably at 90% suppression.

Screening for a Compound Altering the Expression of EBI3, DLX5, NPTX1, CDKN3 and/or EF-1Delta:

In the present invention, the decrease of the expression of EBI3, DLX5, NPTX1, CDKN3 and/or EF-1delta by siRNA causes inhibiting cancer cell proliferation (FIGS. 4D, 6D, 10A, 10B, 22A and 22B). Therefore, the present invention provides a method of screening for a compound that inhibits the expression of EBI3, DLX5, NPTX1, CDKN3 and/or EF-1delta. A compound that inhibits the expression of EBI3, DLX5, NPTX1, CDKN3 and/or EF-1delta is expected to suppress the proliferation of lung cancer cells, and thus is useful for treating or preventing lung cancer. Therefore, the present invention also provides a method for screening a compound that suppresses the proliferation of lung cancer cells, and a method for screening a compound for treating or preventing lung cancer. In the context of the present invention, such screening may include, for example, the following steps:

(a) contacting a candidate compound with a cell expressing EBI3, DLX5, NPTX1, CDKN3 and/or EF-1delta; and

(b) selecting the candidate compound that reduces the expression level of EBI3, DLX5, NPTX1, CDKN3 and/or EF-1delta as compared to a control.

The method of the present invention will be described in more detail below.

Cells expressing the EBI3, DLX5, NPTX1, CDKN3 and/or EF-1delta include, for example, cell lines established from lung cancer; such cells can be used for the above screening of the present invention (e.g., A427, LC 176, LC319, A549, NCI-H23, NCI-H1317, NCI-H1666, NCI-H1781, NCI-H226, NCI-H522, PC3, PC9, PC14, EBC01, LU61, NCI-H520, NCI-H1703, NCI-H2170, NCI-H647, LX1, DMS114, DMS273, SBC-3, SBC-5, SK− LU-1, ESC-1, RERF-LC-AI, SK-MES-1, SW900, and SW1573). The expression level can be estimated by methods well known to one skilled in the art, for example, RT-PCR, Northern bolt assay, Western bolt assay, immunostaining and flow cytometry analysis. “reduce the expression level” as defined herein are preferably at least 10% reduction of expression level of EBI3, DLX5, NPTX1, CDKN3 and/or EF-1delta in comparison to the expression level in absence of the compound, more preferably at least 25%, 50% or 75% reduced level and most preferably at 95% reduced level. The compound herein includes chemical compound, double-strand nucleotide, and so on. The preparation of the double-strand nucleotide is in aforementioned description. In the method of screening, a compound that reduces the expression level of EBI3, DLX5, NPTX1, CDKN3 and/or EF-1delta can be selected as candidate compounds to be used for the treatment or prevention of lung cancer.

Alternatively, the screening method of the present invention may include the following steps:

(a) contacting a candidate compound with a cell into which a vector, including the transcriptional regulatory region of EBI3, DLX5, NPTX1, CDKN3 and/or EF-1delta and a reporter gene that is expressed under the control of the transcriptional regulatory region, has been introduced;

(b) measuring the expression or activity of said reporter gene; and

(c) selecting the candidate compound that reduces the expression or activity of said reporter gene.

Suitable reporter genes and host cells are well known in the art. For example, reporter genes are luciferase, green florescence protein (GFP), Discosoma sp. Red Fluorescent Protein (DsRed), Chrolamphenicol Acetyltransferase (CAT), lacZ and beta-glucuronidase (GUS), and host cell is COST, HEK293, HeLa and so on. The reporter construct required for the screening can be prepared by connecting reporter gene sequence to the transcriptional regulatory region of EBI3, DLX5, NPTX1, CDKN3 and/or EF-1delta. The transcriptional regulatory region of EBI3, DLX5, NPTX1, CDKN3 and/or EF-1delta herein is the region from start codon to at least 500 bp upstream, preferably 1000 bp, more preferably 5000 or 10000 bp upstream. A nucleotide segment containing the transcriptional regulatory region can be isolated from a genome library or can be propagated by PCR. The reporter construct required for the screening can be prepared by connecting reporter gene sequence to the transcriptional regulatory region of any one of these genes. Methods for identifying a transcriptional regulatory region, and also assay protocol are well known (Molecular Cloning third edition chapter 17, 2001, Cold Springs Harbor Laboratory Press).

The vector containing the said reporter construct is infected to host cells and the expression or activity of the reporter gene is detected by method well known in the art (e.g., using luminometer, absorption spectrometer, flow cytometer and so on). “reduces the expression or activity” as defined herein are preferably at least 10% reduction of the expression or activity of the reporter gene in comparison with in absence of the compound, more preferably at least 25%, 50% or 75% reduction and most preferably at 95% reduction.

### Screening for a Compound Decreasing the Binding Between CDKN3 and VRS, EF-1Alpha, EF-1Beta, EF-1Gamma or EF-1Delta or Between NPTX1 and NPTXR:

In the present invention, the interaction between CDKN3 (SEQ ID NO 5; GenBank accession number: L27711) and Valyl-tRNA synthetase (VRS) (SEQ ID NO 26 or 28; GenBank accession number: NM_{—}006295 or BC012808) or EF-1beta (SEQ ID NO 30; GenBank accession number: NM_{—}001959) or EF-1gamma (SEQ ID NO 7; GenBank accession number: BC009907) or EF-1delta (SEQ ID NO 32; GenBank accession number: BC009865) is shown by immunoprecipitation (FIG. 18A) or the interaction between NPTX1 and NPTXR is shown in FIG. 15B. Moreover, CDKN3 binds the region corresponding to 72 to 160 amino acid of EF-1gamma (SEQ ID NO: 48) (FIGS. 21B and 21C). Additionally, CDKN3 dephosphorylates the EF-1delta (FIGS. 20D and 21A). Therefore, the present invention provides a method of screening for a compound that inhibits the binding between CDKN3 and the interaction partner selected from among VRS, EF-1 alpha, EF-1beta, EF-1gamma, and EF-1delta or between NPTX1 and NPTXR. A compound that inhibits the binding between CDKN3 and these interaction partners or between NPTX1 and NPTXR is expected to suppress the proliferation of lung cancer cells, and thus is useful for treating or preventing lung cancer. Therefore, the present invention also provides a method for screening a compound that suppresses the proliferation of lung cancer cells, and a method for screening a compound for treating or preventing lung cancer.

More specifically, the method includes the steps of:

(a) contacting CDKN3 polypeptide or functional equivalent thereof with a interaction partner, wherein the interaction partner is selected from among VRS polypeptide, EF-1alpha polypeptide, EF-1beta polypeptide, EF-1gamma polypeptide, EF-1delta polypeptide and functional equivalent thereof, in the presence of a test compound;

(b) detecting the binding between the polypeptides; and

(c) selecting the test compound that inhibits the binding between the polypeptides; or

(a) contacting NPTX1 polypeptide or functional equivalent thereof with a interaction NPTXR polypeptide or functional equivalent thereof, in the presence of a test compound;

(b) detecting the binding between the polypeptides; and

(c) selecting the test compound that inhibits the binding between the polypeptides.

In the present invention, “interaction partner” refers to a substance or compound that involves biological activity of CDKN3. Accordingly, for example, when CDKN3 requires a polypeptide for expressing its function, the polypeptide may be “interaction partner”. Generally, CDKN3 and the interaction partner bind each other to maintain the function. In preferred embodiments, interaction partner is polypeptide. It is herein revealed that CDKN3 interacts with VRS polypeptide, EF-1alpha polypeptide, EF-1beta polypeptide, EF-1gamma polypeptide, EF-1delta polypeptide. Therefore, these molecules and functional equivalent are preferred interaction partners. Herein, for example, a “functional equivalent” of interaction partner includes a polypeptide that has a biological activity equivalent to the interaction partner.

Namely, any polypeptide that retains at least one biological activity of such interaction partner may be used as such a functional equivalent in the present invention. Exemplary, the functional equivalent of interaction partner retains promoting activity of cell proliferation. In addition, the biological activity of interaction partner contains binding activity to CDKN3 and/or CDKN3-mediated cell migration or proliferation. Functional equivalents of interaction partner include those wherein one or more amino acids are substituted, deleted, added, or inserted to the natural occurring amino acid sequence of the these interaction partner protein.

The phrase “functional equivalent of EF-1gamma polypeptide” as used herein refers to the polypeptide which includes amino acid sequence of CDKN3 binding domain; (SEQ ID NO: 48). Similarly, the term “functional equivalent of CDKN3 polypeptide” refers to the polypeptide which includes amino acid sequence of VRS or EF-1beta or EF-1gamma or EF-1delta binding domain and the term “functional equivalent of VRS or EF-1beta or EF-1gamma polypeptide” refers to the polypeptide which includes amino acid sequence of CDKN3 binding domain.

The method of the present invention is described in further detail below.

As a method of screening for compounds that inhibit binding between CDKN3 and VRS, EF-1beta, EF-1gamma, or EF-1delta, or between NPTX1 and NPTXR many methods well known by one skilled in the art can be used. Such a screening can be carried out as an in vitro assay system. More specifically, first, CDKN3 or NPTX1 polypeptide is bound to a support, and VRS, EF-1beta, EF-1gamma, EF-1delta polypeptide, or NPTXR is added together with a test compound thereto. Next, the mixture is incubated, washed and VRS, EF-1beta, EF-1gamma, EF-1delta polypeptide, or NPTXR bound to the support is detected and/or measured. Promising candidate compound can reduce the amount of detecting VRS, EF-1beta, EF-1gamma, EF-1delta polypeptide, or NPTXR. On the contrary, VRS, EF-1beta, EF-1gamma, EF-1delta polypeptide, or NPTXR may be bound to a support and CDKN3 polypeptide or NPTX1 may be added. Here, CDKN3 or NPTX1 and the VRS, EF-1beta, EF-1gamma, EF-1delta, or NPTXR polypeptide can be prepared not only as a natural protein but also as a recombinant protein prepared by the gene recombination technique. The natural protein can be prepared, for example, by affinity chromatography. On the other hand, the recombinant protein may be prepared by culturing cells transformed with DNA encoding CDKN3, VRS, EF-1beta, EF-1gamma, EF-1delta, NPTX1 or NPTXR to express the protein therein and then recovering it.

Examples of supports that may be used for binding proteins include insoluble polysaccharides, such as agarose, cellulose and dextran; and synthetic resins, such as polyacrylamide, polystyrene and silicon; preferably commercial available beads and plates (e.g., multi-well plates, biosensor chip, etc.) prepared from the above materials may be used. When using beads, they may be filled into a column. Alternatively, the use of magnetic beads of also known in the art, and enables to readily isolate proteins bound on the beads via magnetism.

The binding of a protein to a support may be conducted according to routine methods, such as chemical bonding and physical adsorption. Alternatively, a protein may be bound to a support via antibodies specifically recognizing the protein. Moreover, binding of a protein to a support can be also conducted by means of avidin and biotin. The binding between proteins is carried out in buffer, for example, but are not limited to, phosphate buffer and Tris buffer, as long as the buffer does not inhibit binding between the proteins.

In the present invention, a biosensor using the surface plasmon resonance phenomenon may be used as a mean for detecting or quantifying the bound protein. When such a biosensor is used, the interaction between the proteins can be observed real-time as a surface plasmon resonance signal, using only a minute amount of polypeptide and without labeling (for example, BIAcore, Pharmacia). Therefore, it is possible to evaluate binding between CDKN3 and VRS, EF-1beta, EF-1gamma, or EF-1delta, or between NPTX1 and NPTXR using a biosensor such as BIAcore.

Alternatively, CDKN3, VRS, EF-1beta, EF-1gamma, or EF-1delta, NPTX1 or NPTXR may be labeled, and the label of the polypeptide may be used to detect or measure the binding activity. Specifically, after pre-labeling one of the polypeptide, the labeled polypeptide is contacted with the other polypeptide in the presence of a test compound, and then bound polypeptide are detected or measured according to the label after washing. Labeling substances such as radioisotope (e.g., 3H, ¹⁴C, ³²P, ³³P, ³⁵S, ¹²⁵I, ¹³¹I), enzymes (e.g., alkaline phosphatase, horseradish peroxidase, b-galactosidase, b-glucosidase), fluorescent substances (e.g., fluorescein isothiosyanete (FITC), rhodamine) and biotin/avidin, may be used for the labeling of a protein in the present method. When the protein is labeled with radioisotope, the detection or measurement can be carried out by liquid scintillation. Alternatively, proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Furthermore, binding between CDKN3 and VRS, EF-1beta, EF-1gamma, or EF-1delta, or between NPTX1 and NPTXR can be also detected or measured using antibodies to CDKN3, VRS, EF-1beta, EF-1gamma, EF-1delta, NPTX1 or NPTXR. For example, after contacting CDKN3 polypeptide or NPTX1 polypeptide immobilized on a support with a test compound and VRS, EF-1beta, EF-1gamma, or EF-1delta polypeptide or NPTXR polypeptide, the mixture is incubated and washed, and detection or measurement can be conducted using an antibody against VRS, EF-1beta, EF-1gamma, or EF-1delta polypeptide or NPTXR polypeptide.

Alternatively, VRS, EF-1beta, EF-1gamma, EF-1delta polypeptide, or NPTXR polypeptide may be immobilized on a support, and an antibody against CDKN3 or NPTX1 may be used as the antibody. In case of using an antibody in the present screening, the antibody is preferably labeled with one of the labeling substances mentioned above, and detected or measured based on the labeling substance. Alternatively, the antibody against CDKN3, VRS, EF-1beta, EF-1gamma, EF-1delta, NPTX1 or NPTXR polypeptide may be used as a primary antibody to be detected with a secondary antibody that is labeled with a labeling substance. Furthermore, the antibody bound to the protein in the screening of the present invention may be detected or measured using protein G or protein A column.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, for example, CDKN3 polypeptide or NPTX1 polypeptide is fused to the SRF-binding region or GAL4-binding region and expressed in yeast cells. VRS, EF-1beta, EF-1gamma, or EF-1delta polypeptide that binds to CDKN3 polypeptide or NPTXR polypeptide that binds to NPTX1 polypeptide is fused to the VP16 or GAL4 transcriptional activation region and also expressed in the yeast cells in the existence of a test compound. Alternatively, CDKN3 polypeptide or NPTX1 polypeptide may be fused to the SRF-binding region or GAL4-binding region, and VRS, EF-1beta, EF-1gamma, EF-1delta polypeptide or NPTXR polypeptide to the VP16 or GAL4 transcriptional activation region. The binding of the two activates a reporter gene, making positive clones detectable. As a reporter gene, for example, Ade2 gene, lacZ gene, CAT gene, luciferase gene and such can be used besides HIS3 gene.

Moreover, in the case of using CDKN3 and EF-1gamma, the screening method of this invention is detecting the phosphorylation level of EF-1gamma by using anti-phospho-serine antibody.

### Further Screening for a Compound Treating or Preventing Lung Cancer:

In the present invention, it is revealed that suppressing one or more of the following events reduces cell proliferation of lung cancer including NSCLC and SCLC.

Expression of EBI3, DLX5, NPTX1, CDKN3 and/or EF-1delta,

Biological activity of EBI3, DLX5, NPTX1, CDKN3 and/or EF-1delta, and

Interaction between CDKN3 and EF-1alpha, EF-1beta, EF-1gamma and/or EF-1delta,

Thus, by screening for test compounds that inhibit at least one event among them, candidate compounds that have the potential to treat or prevent lung cancers can be identified. Potential of these candidate compounds to treat or prevent lung cancers may be evaluated by second and/or further screening to identify therapeutic agent for cancers.

### EF-1Delta Mutant:

Dominant negative mutants of the proteins disclosed here can be used to treat or prevent lung cancer. For example, the present invention provides methods for treating or preventing lung cancer in a subject by administering an EF-1delta mutant having a dominant negative effect, or a polynucleotide encoding such a mutant. The EF-1delta mutant may include an amino acid sequence that includes a CDKN3 binding region, e.g. a part of EF-1delta protein and included a part of leucine zipper of EF-1delta (see FIG. 20A). The EF-1delta mutant may have the amino acid sequence of SEQ ID NO: 61 corresponding to positions 90-108 of SEQ ID NO: 8.

The present invention also provides a polypeptide including the sequence ENQSLRGVVQELQQAISKL (SEQ ID NO: 61); or an amino acid sequence of a polypeptide functionally equivalent to the polypeptide, wherein the polypeptide lacks the biological function of a peptide consisting of SEQ ID NO: 8. In a preferred embodiment, the biological function to be deleted is an activity to promote a cell proliferation of lung cancer cell. Length of the polypeptide of the present invention may be less than the full length EF-1delta (SEQ ID NO: 8; 281 residues). Generally, polypeptides of the present invention may have less than 200 amino acid residues, preferably less than 100 amino acid residues, more preferably 10-50, alternatively 8-30 amino acid residues.

The polypeptides of the present invention include modified polypeptides. In the present invention, the term “modified” refers, for example, to binding with other substances. Accordingly, in the present invention, the polypeptides of the present invention may further include other substances such as cell-membrane permeable substance. The other substances include organic compounds such as peptides, lipids, saccharides, and various naturally-occurring or synthetic polymers. The polypeptides of the present invention may have any modifications so long as the polypeptides retain the desired activity of inhibiting the binding of EF-1delta to CDKN3. In some embodiments, the inhibitory polypeptides can directly compete with EF-1delta binding to CDKN3. Modifications can also confer additive functions on the polypeptides of the invention. Examples of the additive functions include targetability, deliverability, and stabilization.

In some preferred embodiments, the EF-1delta mutant may be linked to a membrane transducing agent. A number of peptide sequences have been characterized for their ability to translocate into live cells and can be used for this purpose in the present invention. Such membrane transducing agents (typically peptides) are defined by their ability to reach the cytoplasmic and/or nuclear compartments in live cells after internalization. Examples of proteins from which transducing agents may be derived include HIV Tat transactivator 1, 2, the Drosophila melanogaster transcription factor Antennapedia3. In addition, nonnatural peptides with transducing activity have been used. These peptides are typically small peptides known for their membrane-interacting properties which are tested for translocation. The hydrophobic region within the secretion signal sequence of K-fibroblast growth factor (FGF), the venom toxin mastoparan (transportan)13, and Buforin I14 (an amphibian antimicrobial peptide) have been shown to be useful as transducing agents. For a review of transducing agents useful in the present invention see Joliot et al. Nature Cell Biology 6:189-96 (2004).

The EF-1delta mutant may have the general formula:

[R]-[D],

wherein [R] is a membrane transducing agent, and [D] is a polypeptide having the amino acid sequence of SEQ ID NO: 61. In the general formula, [R] may directly link with [D], or indirectly link with [D] through a linker. Peptides or compounds having plural functional groups may be used as the linker. Specifically, an amino acid sequence of -GGG- may be used as the linker. Alternatively, the membrane transducing agent and the polypeptide having the amino acid sequence of SEQ ID NO: 61 can bind to the surface of micro-particle. In the present invention, [R] may link with arbitral region of [D]. For example, [R] may link with N-terminus or C-terminus of [D], or side chain of the amino acid residues constituting [D]. Furthermore, plural molecules of [R] may also link with one molecule of [D]. In some embodiments, plural molecules of [R]s may link with different site of [D]. In another embodiments, [D] may be modified with some [R]s linked together.

The membrane transducing agent can be selected from group listed below;

[poly-arginine]; Matsushita, M. et al, J. Neurosci. 21, 6000-7 (2003).

[Tat/RKKRRQRRR] (SEQ ID NO: 63) Frankel, A. et al, Cell 55, 1189-93 (1988).
Green, M. & Loewenstein, P. M. Cell 55, 1179-88 (1988).

[Penetratin/RQIKIWFQNRRMKWKK] (SEQ ID NO: 64)
Derossi, D. et al, J. Biol. Chem. 269, 10444-50 (1994).

[Buforin II/TRSSRAGLQFPVGRVHRLLRK] (SEQ ID NO: 65)
Park, C. B. et al. Proc. Natl. Acad. Sci. USA 97, 8245-50 (2000).

[Transportan/GWTLNSAGYLLGKINLKALAALAKKIL] (SEQ ID NO: 66)
Pooga, M. et al. FASEB J. 12, 67-77 (1998).

[MAP (model amphipathic peptide)/KLALKLALKALKAALKLA] (SEQ ID NO: 67)
Oehlke, J. et al. Biochim. Biophys. Acta. 1414, 127-39 (1998).

[K-FGF/AAVALLPAVLLALLAP] (SEQ ID NO: 68)
Lin, Y. Z. et al. J. Biol. Chem. 270, 14255-14258 (1995).

[Ku70/VPMLK] (SEQ ID NO: 69)
Sawada, M. et al. Nature Cell Biol. 5, 352-7 (2003).

[Ku70/PMLKE] (SEQ ID NO: 70)
Sawada, M. et al. Nature Cell Biol. 5, 352-7 (2003).

[Prion/MANLGYWLLALFVTMWTDVGLCKKRPKP] (SEQ ID NO: 71)
Lundberg, P. et al. Biochem. Biophys. Res. Commun. 299, 85-90 (2002).

[pVEC/LLIILRRRIRKQAHAHSK] (SEQ ID NO: 72)
Elmquist, A. et al. Exp. Cell Res. 269, 237-44 (2001).

[Pep-1/KETWWETWWTEWSQPKKKRKV] (SEQ ID NO: 73)
Morris, M. C. et al. Nature Biotechnol. 19, 1173-6 (2001).

[SynB1/RGGRLSYSRRRFSTSTGR] (SEQ ID NO: 74)
Rousselle, C. et al. Mol. Pharmacol. 57, 679-86 (2000).

[Pep-7/SDLWEMMMVSLACQY] (SEQ ID NO: 75)
Gao, C. et al. Bioorg. Med. Chem. 10, 4057-65 (2002).

[HN-1/TSPLNIHNGQKL] (SEQ ID NO: 76)
Hong, F. D. & Clayman, G L. Cancer Res. 60, 6551-6 (2000).

In the present invention, number of arginine residues that constitute the poly-arginine is not limited. In some preferred embodiments, 5 to 20 contiguous arginine residues may be exemplified. In a preferred embodiment, the number of arginine residues of the poly-arginine is 11 (SEQ ID NO: 77).

As used herein, the phrase “dominant negative fragment of EF-1delta” refers to a mutated form of EF-1delta that is capable of complexing with CDKN3. Thus, a dominant negative fragment is one that is not functionally equivalent to the full length EF-1delta polypeptide. Preferred dominant negative fragments are those that include an CDKN3 binding region, e.g. a part of EF-1delta protein and included a part of leucine zipper of EF-1deltas.

In another embodiment, the present invention provides for the use of a polypeptide having the sequence ENQSLRGVVQELQQAISKL (SEQ ID NO: 61); a polypeptide functionally equivalent to the polypeptide; or polynucleotide encoding those polypeptides in manufacturing a pharmaceutical composition for treating or preventing lung cancer, wherein the polypeptide lacks the biological function of a peptide consisting of SEQ ID NO: 8. Moreover, in another embodiments, the present invention also provides an agent for either or both of treating and preventing lung cancer including as an active ingredient a polypeptide which includes the sequence ENQSLRGVVQELQQAISKL (SEQ ID NO: 61); a polypeptide functionally equivalent to the polypeptide; or polynucleotide encoding those polypeptides, wherein the polypeptide lacks the biological function of a peptide consisting of SEQ ID NO:8. Alternatively, the present invention also provides a pharmaceutical composition for treating or preventing lung cancer, including a polypeptide composed of the sequence ENQSLRGVVQELQQAISKL (SEQ ID NO: 61); or a polypeptide functionally equivalent to the polypeptide; and a pharmaceutically acceptable carrier, wherein the polypeptide lacks the biological function of a peptide of SEQ ID NO: 8.

One skilled in the art can readily determine an effective amount of the polypeptide of the invention to be administered to a given subject, by taking into account factors such as body weight, age, sex, type of disease, symptoms and other conditions of the subject; the route of administration; and whether the administration is regional or systemic.

Although dosages may vary according to the symptoms, an exemplary dose of an antibody or fragments thereof for treating or preventing NSCLC is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the condition of the patient, symptoms of the disease and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kg of body-weight.

It is contemplated that greater or smaller amounts of the peptide can be administered. The precise dosage required for a particular circumstance may be readily and routinely determined by one of skill in the art.

The present invention further provides a method or process for manufacturing a pharmaceutical composition for treating lung cancer expressing EF-1delta, wherein the method or process includes step for admixing an active ingredient with a pharmaceutically or physiologically acceptable carrier, wherein the active ingredient is a polypeptide including the sequence ENQSLRGVVQELQQAISKL (SEQ ID NO: 61); or a polypeptide functionally equivalent to the polypeptide.

Aspects of the present invention are described in the following examples, which are not intended to limit the scope of the invention described in the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be further described in the following examples, which do not limit the scope of the invention described in the claims.

### EXAMPLES

The invention will be further described in the following examples, which do not limit the scope of the invention described in the claims.

### Part I

### EBI3 Related Experiments

### Example 1

### General Methods

### 1. Cell Lines and Tissue Samples

The 23 human lung cancer cell lines used in this study included nine adenocarcinomas (ADC; A427, A549, LC319, PC-3, PC-9, PC-14, NCI-H1373, NCI-H1666, and NCI-H1781), two adenosquamous carcinomas (ASC; NCI-H226 and NCI-H647), seven SCCs (EBC-1, LU61, NCI-H520, NCI-H1703, NCI-H2170, RERF-LC-AI, and SK-MES-1), one large cell carcinoma (LX1), and four small cell lung cancers (SCLC; DMS114, DMS273, SBC-3, and SBC-5). All cells were grown in monolayer in appropriate medium supplemented with 10% FCS and maintained at 37 degree Centigrade in humidified air with 5% CO₂. Human small airway epithelial cells (SAEC) used as a control were grown in optimized medium (small airway growth medium) from Cambrex Bioscience, Inc. (East Rutherford, N.J.). Primary lung cancer samples had been obtained earlier with informed consent (Yamabuki T, et al., Int J Oncol 28: 1375-84 (2006), Kikuchi T, et al., Oncogene 22: 2192-205 (2003), Taniwaki M, et al., Int J Oncol 29: 567-75 (2006)). Clinical stage was judged according to the International Union Against Cancer TNM classification (Sobin L, et al., 6th ed. New York: Wiley-Liss; (2002)). A total of 423 formalin-fixed samples of primary NSCLCs (stage I-IIIA) including 271 ADCs, 110 SCCs, 28 LCCs, 14 ASCs and adjacent normal lung tissues, had been obtained earlier along with clinicopathological data from patients undergoing surgery at Saitama Cancer Center (Saitama, Japan). This study and the use of all clinical materials mentioned were approved by individual institutional Ethical Committees.

### 2. Serum Samples

Serum samples were obtained with written informed consent from 120 healthy control individuals (96 males and 24 females; median age of 51.6 with a range of 27 to 60 years) and from 63 non-neoplastic lung disease patients with chronic obstructive pulmonary disease (COPD) (53 males and 10 females; median age of 67.0 with a range of 54 to 73 years). All of these COPD patients were current and/or former smokers [the mean (+/−1 SD) of pack-year index (PYI) was 70.0+/−42.7; PYI was defined as the number of cigarette packs (20 cigarettes per pack) consumed a day multiplied by years]. Serum samples were also obtained with informed consent from 95 lung cancer patients (49 males and 46 females; median age of 64.4 with a range of 38 to 83 years) admitted to and from 194 patients with lung cancer (142 males and 52 females; median age of 68.0 with a range of 38 to 89 years). These 289 lung cancer cases included 170 ADCs, 37 SCCs, and 82 SCLCs. These serum samples from a total of 289 lung cancer patients were selected for the study based on the following criteria: (a) patients were newly diagnosed and previously untreated and (b) their tumors were pathologically diagnosed as lung cancers (stages I-IV). Serum was obtained at the time of diagnosis and stored at −150 degree Centigrade

### 3. Semiquantitative Reverse Transcription-PCR

A total of 3 micro g aliquot of mRNA from each sample was reversely transcribed to single-stranded cDNAs using random primer (Roche Diagnostics, Basel, Switzerland) and SuperScript II (Invitrogen, Carlsbad, Calif.). Semiquantitative reverse transcription-PCR(RT-PCR) experiments were carried out with the following sets of synthesized primers specific to EBI3 or beta-actin (ACTB) specific primers as an internal control:

| | |
|---|---|
| EBI3, 5′-TGTTCTCCATGGCTCCCTAC-3′ | (SEQ ID No: 9) |
| and | |
| 5′-AGCTCCCTGACGCTTGTAAC-3′; | (SEQ ID No: 10) |
| ACTB, 5′-GAGGTGATAGCATTGCTTTCG-3′ | (SEQ ID No: 11) |
| and | |
| 5′-CAAGTCAGTGTACAGGTAAGC-3′. | (SEQ ID No: 12) |

PCRs were optimized for the number of cycles to ensure product intensity to be within the linear phase of amplification.

### 4. Northern Blot Analysis

Human multiple tissue blots covering 16 tissues (BD Biosciences, Palo Alto, Calif.) were hybridized with an [alpha-³²P]-dCTP-labeled, 404-bp PCR product of EBI3 that was prepared as a probe using primers

| 5′-TGTTCTCCATGGCTCCCTAC-3′ | (SEQ ID No: 13) |
|---|---|
| and | |
| 5′-CTACTTGCCCAGGCTCATTG-3′. | (SEQ ID No: 14) |

Prehybridization, hybridization, and washing were done following the manufacturer's specifications. The blots were autoradiographed with intensifying screens at −80 degrees C. for 7 days.

### 5. Immunocytochemical Analysis

Cells were plated on glass coverslips (Becton Dickinson Labware, Franklin Lakes, N.J.), fixed with 4% paraformaldehyde, and permeabilized with 0.1% Triton X-100 in PBS for 3 min at room temperature. Nonspecific binding was blocked by Casblock (ZYMED, San Francisco, Calif.) for 10 min at room temperature. Cells were then incubated for 60 min at room temperature with primary antibodies diluted in PBS containing 3% BSA. After being washed with PBS, the cells were stained by Alexa488-conjugated secondary antibody (Invitrogen) for 60 min at room temperature. After another wash with PBS, each specimen was mounted with Vectashield (Vector Laboratories, Inc., Burlingame, Calif.) containing 4′,6-diamidino-2-phenylindole and visualized with Spectral Confocal Scanning Systems (TSC SP2 AOBS; Leica Microsystems, Wetzlar, Germany).

### 6. Immunohistochemistry and Tissue Microarray

To investigate the EBI3 protein in clinical samples that had been embedded in paraffin blocks, the sections were stained by the following manner. Briefly, 3.3 mg/mL of a goat polyclonal anti-human EBI3 antibody (Santa Cruz Biotechnology, Santa Cruz, Calif.) were added to each slide after blocking of endogenous peroxidase and proteins, and the sections were incubated with HRP-labeled anti-goat IgG [Histofine Simple Stain MAX PO (G), Nichirei, Tokyo, Japan] as the secondary antibody. Substrate-chromogen was added, and the specimens were counterstained with hematoxylin. Tumor tissue microarrays were constructed with formalin-fixed 423 primary lung cancers as described elsewhere (Chin S F, et al., Mol Pathol 56: 275-9 (2003), Callagy G, et al., Diagn Mol Pathol 12: 27-34 (2003), Callagy G, et al., J Pathol 205: 388-96 (2005)). The tissue area for sampling was selected based on visual alignment with the corresponding H&E-stained section on a slide. Three, four, or five tissue cores (diameter, 0.6 mm; depth, 3-4 mm) taken from a donor tumor block were placed into a recipient paraffin block with a tissue microarrayer (Beecher Instruments, Sun Prairie, Wis.). A core of normal tissue was punched from each case, and 5-micro m sections of the resulting microarray block were used for immunohistochemical analysis. Three independent investigators semiquantitatively assessed EBI3 positivity without prior knowledge of clinicopathologic data as reported previously (Suzuki C, et al., Cancer Res 65: 11314-25 (2005), Ishikawa N, et al., Clin Cancer Res 10: 8363-70 (2004), Kato T, et al., Cancer Res 65: 5638-46 (2005), Hayama S, et al., Cancer Res 67: 4113-22 (2007)). The intensity of EBI3 staining was evaluated using the following criteria: strong positive (scored as 2+), brown staining in >50% of tumor cells completely obscuring cytoplasm; weak positive (1+), any lesser degree of brown staining appreciable in tumor cell cytoplasm; and absent (scored as 0), no appreciable staining in tumor cells. Cases were accepted as strongly positive only if reviewers independently defined them as such.

### 7. Statistical Analysis

Statistical analyses were done using the StatView statistical program (SAS, Cary, N.C.). Tumor-specific survival curves were calculated from the date of surgery to the time of death related to NSCLC or to the last follow-up observation. Kaplan-Meier curves were calculated for each relevant variable and for EBI3 expression; differences in survival times among patient subgroups were analyzed using the log-rank test. Univariate and multivariate analyses were done with the Cox proportional hazard regression model to determine associations between clinicopathologic variables and cancer-related mortality. First, the associations between death and possible prognostic factors, including age, gender, pathologic tumor classification, and pathologic node classification, taking into consideration one factor at a time, were analyzed. Second, multivariate Cox analysis was applied on backward (stepwise) procedures that always forced strong EBI3 expression into the model, along with any and all variables that satisfied an entry level of a P value of <0.05. As the model continued to add factors, independent factors did not exceed an exit level of P<0.05.

### 8. ELISA

Serum levels of EBI3 were measured by ELISA system, which had been originally constructed. First, a goat polyclonal antibody specific to EBI3 was added to a 96-well microplate (Nunc, Roskilde, Denmark) as a capture antibody and incubated for 2 h at room temperature. After washing away any unbound antibody, 5% BSA was added to the wells and incubated for 16 h at 4 degree Centigrade for blocking. After a wash, 3-fold diluted sera were added to the wells and incubated for 2 h at room temperature. After washing away any unbound substances, a biotinylated polyclonal antibody specific for EBI3 using Biotin Labeling Kit-NH2 (DOJINDO, Kumamoto, Japan) was added to the wells as a detection antibody and incubated for 2 h at room temperature. After a wash to remove any unbound antibody-enzyme reagent, HRP-streptavidin was added to the wells and incubated for 20 min. After a wash, a substrate solution (R&D Systems, Inc., Minneapolis, Minn.) was added to the wells and allowed to react for 30 min. The reaction was stopped by adding 100 micro L of 2N sulfuric acid. Color intensity was determined by a photometer at a wavelength of 450 nm, with a reference wavelength of 570 nm. Levels of CEA in serum were measured by ELISA with a commercially available enzyme test kit (Hope Laboratories, Belmont, Calif.) according to the supplier's recommendations. Levels of ProGRP in serum were measured by ELISA with a commercially available enzyme test kit (TFB, Tokyo, Japan) according to the manufacturer's protocol. Differences in the levels of EBI3, CEA, and ProGRP between tumor groups and a healthy control group were analyzed by Mann-Whitney U tests. The levels of EBI3, CEA, and ProGRP were evaluated by receiver operating characteristic (ROC) curve analysis to determine cutoff levels with optimal diagnostic accuracy and likelihood ratios. The correlation coefficients between EBI3 and CEA/ProGRP were calculated with Spearman rank correlation. Significance was defined as P<0.05.

### 9. RNA Interference Assay

Small interfering RNA (siRNA) duplexes (Dharmacon, Inc., Lafayette, Colo.) (600 μM) were transfected into a NSCLC cell line A549 and LC319, using 30 micro 1 of Lipofectamine 2000 (Invitrogen, Carlsbad, Calif.) following the manufacturer's protocol. The transfected cells were cultured for 7 days, and viability of cells was evaluated by 3-(4,5-dimethylthiazol-2-yl)-2,5-diphenyltetrazolium bromide (MTT) assay (cell counting kit-8 solution; Dojindo Laboratories, Kumanoto, Japan). To confirm suppression of EBI3 expression, semiquantitative RT-PCR was carried out with synthesized primers specific to EBI3 described above. The sequences of the synthetic oligonucleotides for RNAi were as follows:

control 1 (On-Target plus; Dharmacon, Inc.; pool
of 5′-UGGUUUACAUGUCGACUAA-3′ (RNA corresponding
to SEQ ID NO: 53); 5′-UGGUUUACAUGUUUUCUGA-3′
(RNA corresponding to SEQ ID NO: 54);
5′-UGGUUUACAUGUUUUCCUA-3′ (RNA corresponding to
SEQ ID NO: 55); 5′-UGGUUUACAUGUUGUGUGA-3′
(RNA corresponding to SEQ ID NO: 56));
control 2 (Luciferase/LUC: Photinus pyralis
luciferase gene),
(RNA corresponding to SEQ ID No: 16)
5′-NNCGUACGCGGAAUACUUCGA-3′;
siRNAs against EBI3-1 (si-EBI3-#1),
(SEQ ID NO: 17)
5′-UACUUGCCCAGGCUCAUUGUU-3′
(SEQ ID NO: 18)
5′-CAATGAGCCTGGGCAAGTA-3′
as the target sequence of si-EBI3-#1;
si-EBI3-#2,
(SEQ ID NO: 19)
5′-AACAGCUGGACAUCCGUGAUU-3′
(SEQ ID NO: 20)
5′-TCACGGATGTCCAGCTGTT-3′.
as the target sequence of si-EBI3-#1

### 10. EBI3-Expressing COS-7 Transfectants

Transfectants stably expressing EBI3 were established according to a standard protocol. The entire coding region of EBI3 was amplified by RT-PCR using the primer sets (5′-CCGCTCGAGGGAATTCCAGCCATGACCCCGCAGCTT-3′ and 5′-TGCTCTAGAGCACTTGCCCAGGCTCATTGTGGC-3′). The product was digested with EcoRI and XbaI, and cloned into appropriate sites of a pcDNA3.1-myc/His A(+) vector (Invitrogen) that contained c-myc-His epitope sequences (LDEESILKQEHHHHHH) at the COOH-terminal of the EBI3 protein. Using FuGENE 6 Transfection Reagent (Roche Diagnostics, Basel, Switherland) according to the manufacturer's protocol, we transfected COS-7 cells, which do not express endogenous EBI3, with plasmids expressing either EBI3 (pcDNA3.1-EBI3-myc/His), or mock plasmids (pcDNA3.1-myc/His). Transfected cells were cultured in DMEM containing 10% FBS and geneticin (0.4 mg/ml) for 14 days; then 50 individual colonies were trypsinized and screened for stable transfectants by a limiting-dilution assay. Expression of EBI3 was determined in each clone by Western blotting and immunostaining.

### 11. MTT and Colony Formation Assays

COS-7 transfectants that could stably express EBI3 were seeded onto six-well plates (1×10⁴ cells/well), and maintained in medium containing 10% FCS and 0.4 mg/ml geneticin. After 120 hours cell proliferation was evaluated by the MTT assay using Cell Counting Kits (Wako, Osaka, Japan). Colonies were stained and counted at the same time. All experiments were done in triplicate

### Example 2

### EBI3 Expression in Lung Cancers and Normal Tissues

To identify novel molecules that can be applicable to detect presence of cancer at an early stage and to develop novel treatments based on the biological characteristics of cancer cells, genome-wide expression profile analysis of 101 lung carcinomas was performed using a cDNA microarray (Kikuchi T, et al., Oncogene 22: 2192-205 (2003), Taniwaki M, et al., Int Oncol 29: 567-75 (2006), Kikuchi T, et al., Int J Oncol 28: 799-805 (2006), Kakiuchi S, et al., Mol Cancer Res 1: 485-99 (2003), Kakiuchi S, et al., Hum Mol Genet 13: 3029-43 (2004)). Among 32,256 genes screened, elevated expression (3-fold or higher) of EBI3 transcript was identified in cancer cells in the great majority of the lung cancer samples examined. The overexpression was confirmed by means of semiquantitative RT-PCR experiments in 11 of 15 lung cancer tissues, in 12 of 23 lung cancer cell lines (FIG. 1A). Immunofluorescence analysis was performed to examine the subcellular localization of endogenous EBI3 in lung cancer cells. EBI3 was detected at cytoplasm of tumor cells with granular appearance at a high level in LC319 and NCI-H1373 cells in which EBI3 transcript was detected by semiquantitative RT-PCR experiments (FIG. 1A), but not in NCI-H2170 cells as well as bronchial epithelia derived BEAS-2B cells, both of which showed no expression of EBI3. The results also indicated that the antibody specifically bound to EBI3 (FIG. 1B). Since EBI3 encodes a secreted protein, we also evaluated culture supernatant levels of EBI3 by ELISA and confirmed that EBI3 was secreted by LC319 and PC 14, whereas no secreted EBI3 was detected by NCI-H2170 or BEAS-2B (FIG. 1C).

Northern blot analysis using an EBI3 cDNA fragment as a probe identified a transcript of 1.3 kb that was highly expressed only in placenta, and its transcript was hardly detectable in any other normal tissues (FIG. 1D). The expression of EBI3 protein was also examined with polyclonal antibody specific to EBI3 on five normal tissues (liver, heart, kidney, lung, and placenta) and lung cancer tissues. EBI3 staining was mainly observed at cytoplasm of tumor cells and syncytiotrophoblasts and cytotrophblast in placenta, but not detected in other four normal tissues (FIG. 1E). The expression level of EBI3 protein in lung cancer was higher than in placenta.

### Example 3

### Association of EBI3 Expression with Poor Prognosis for NSCLC Patients

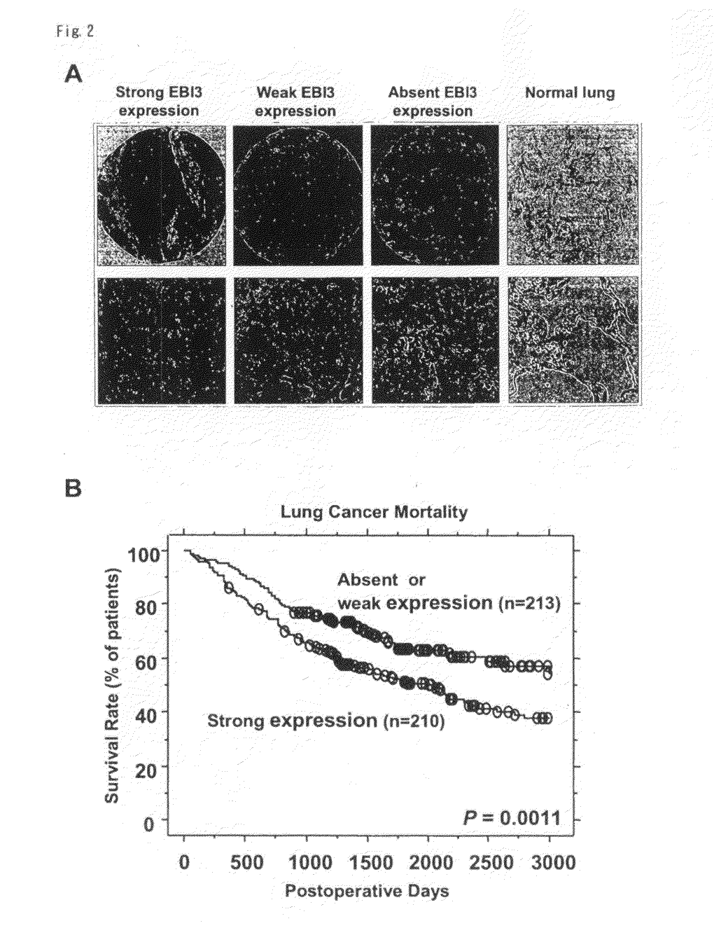

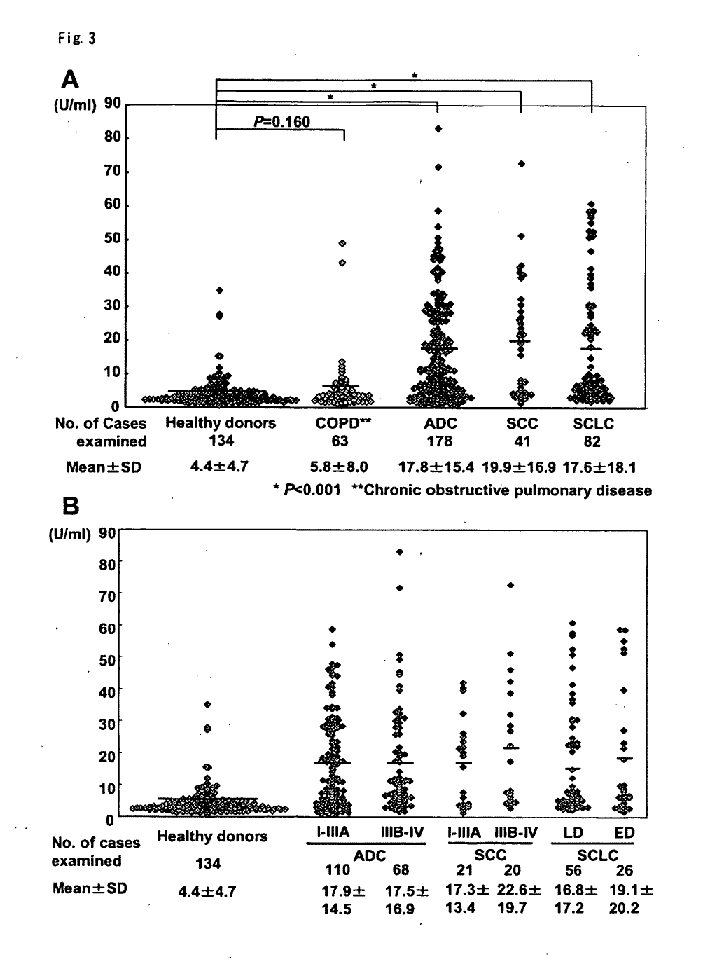

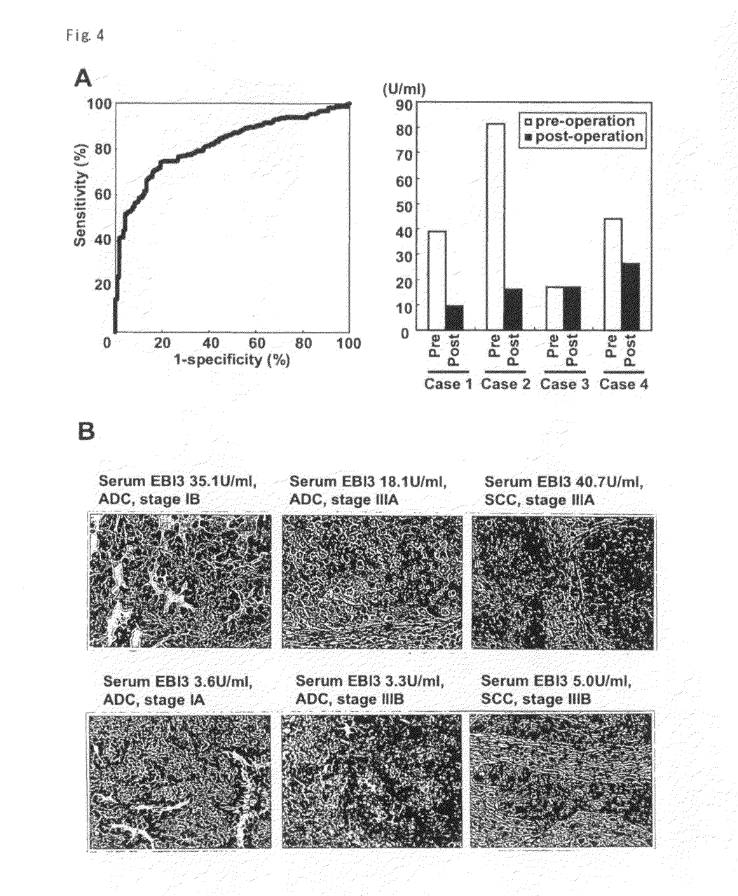

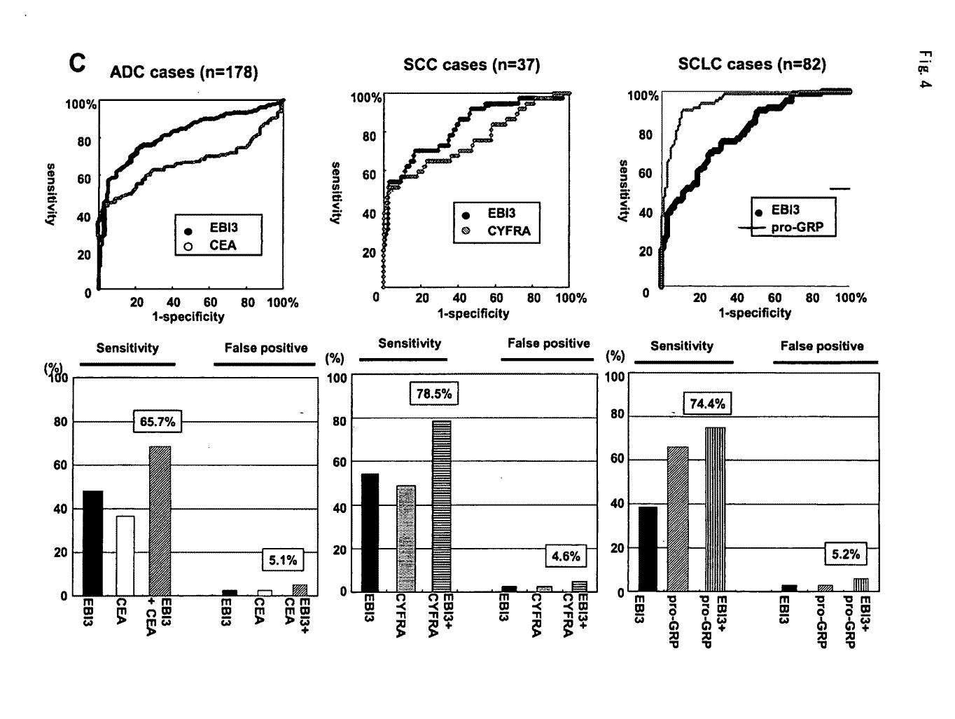

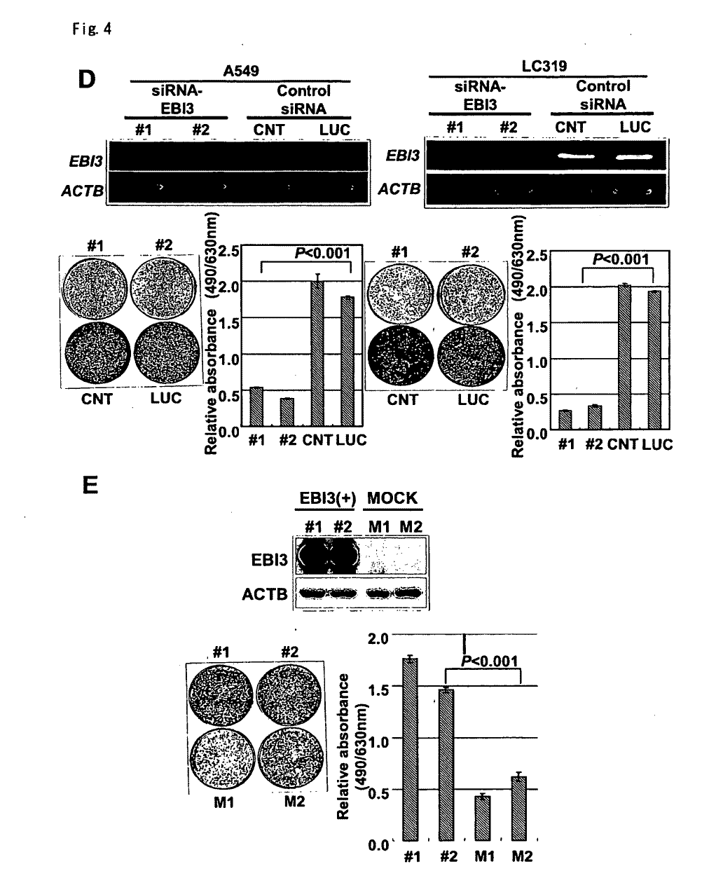

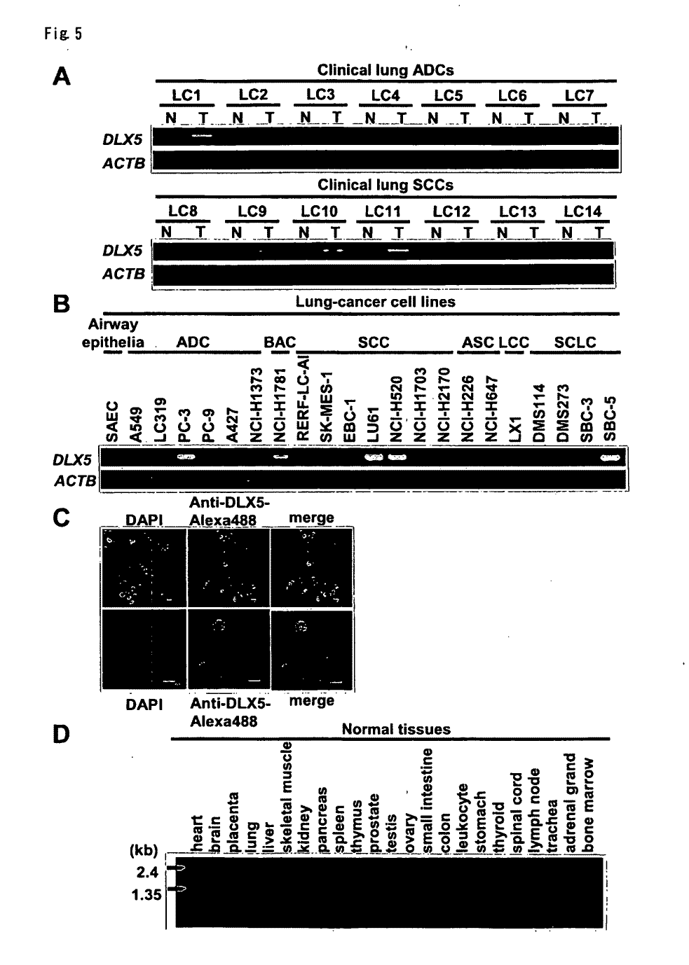

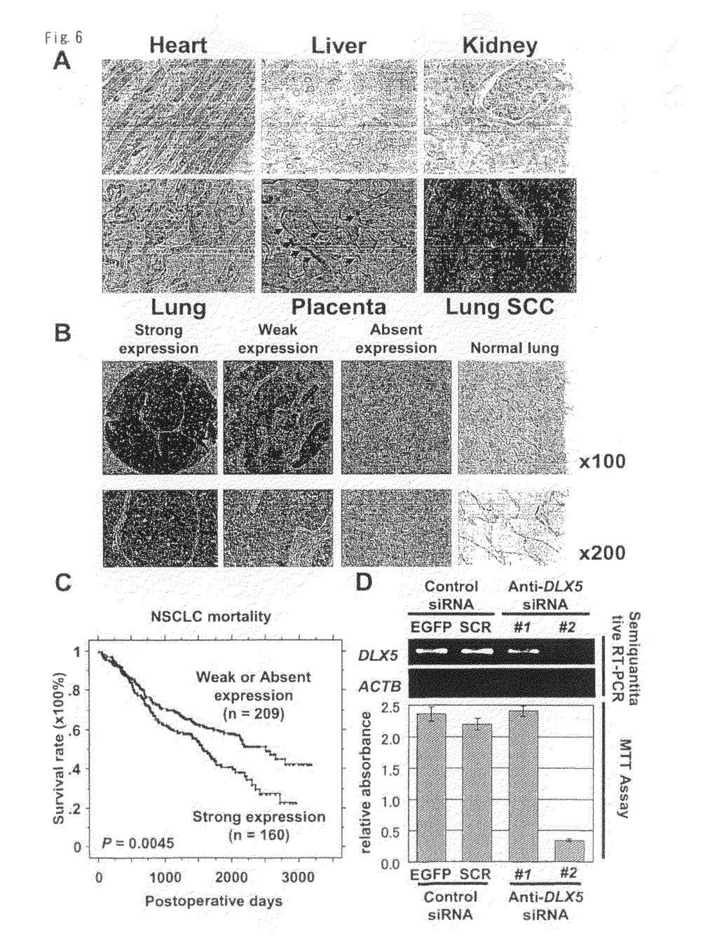

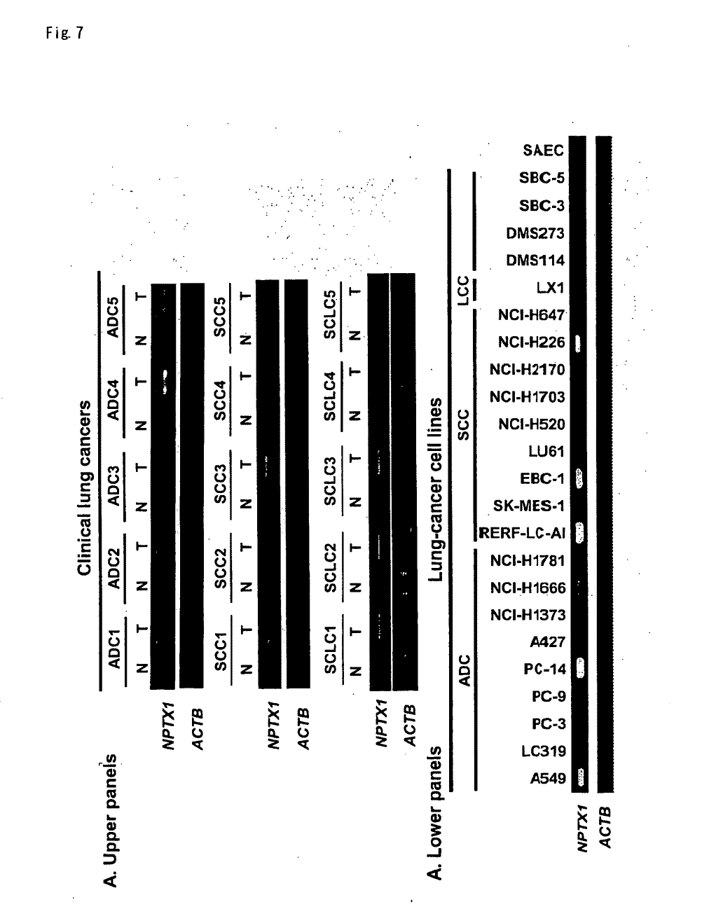

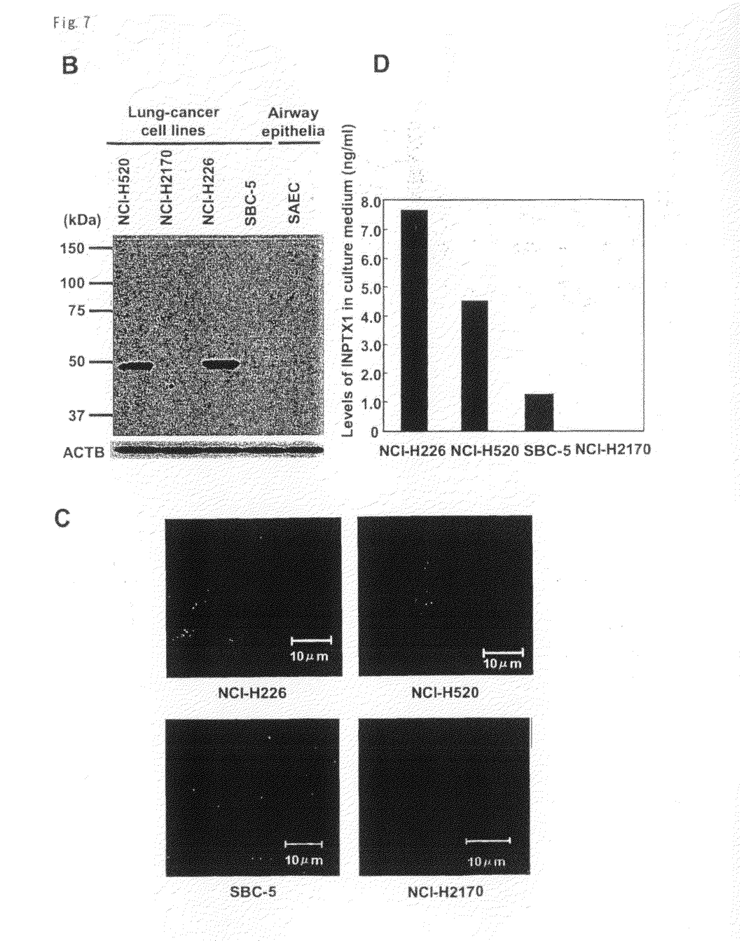

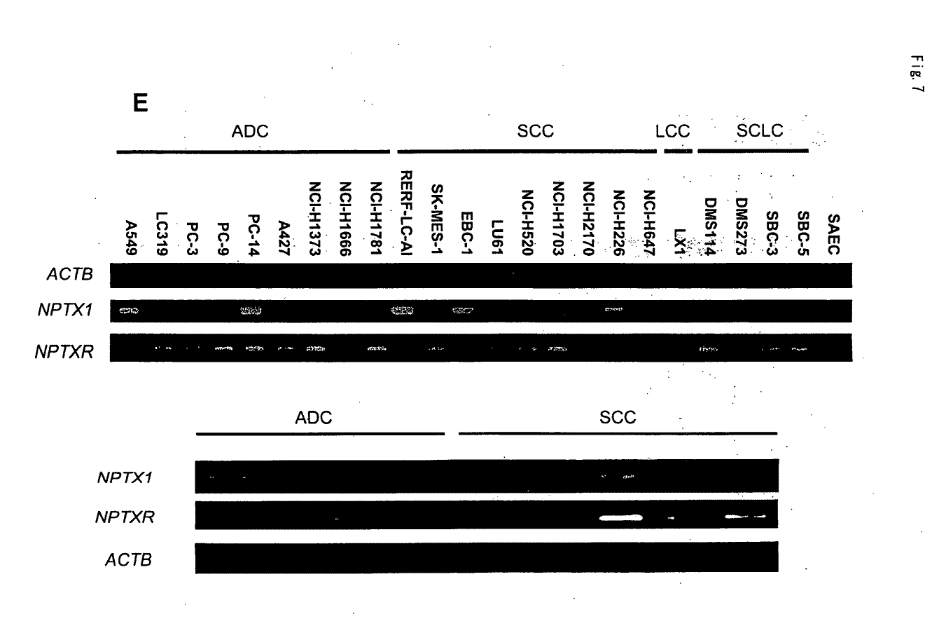

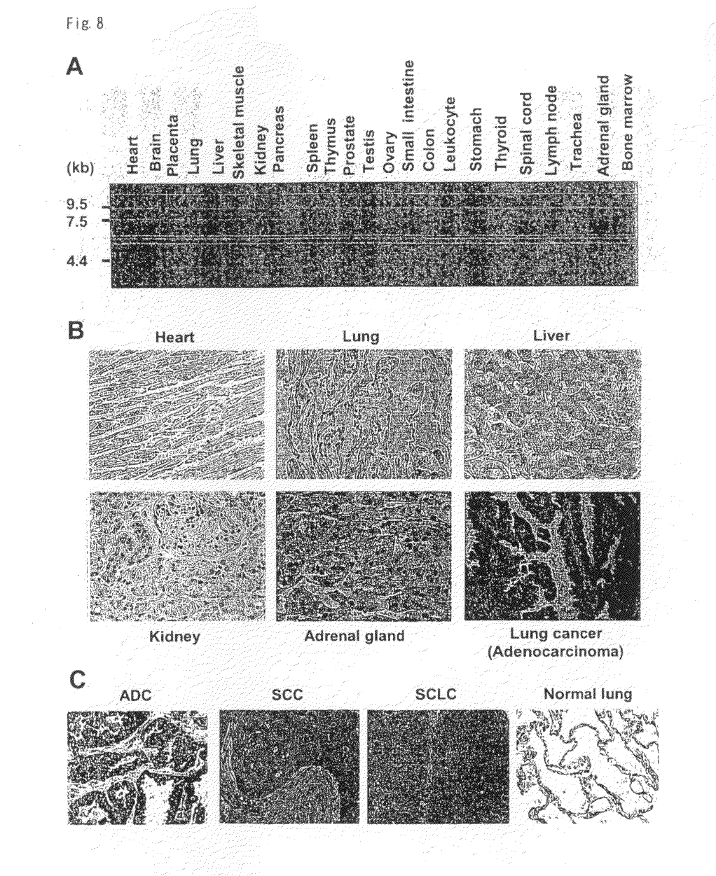

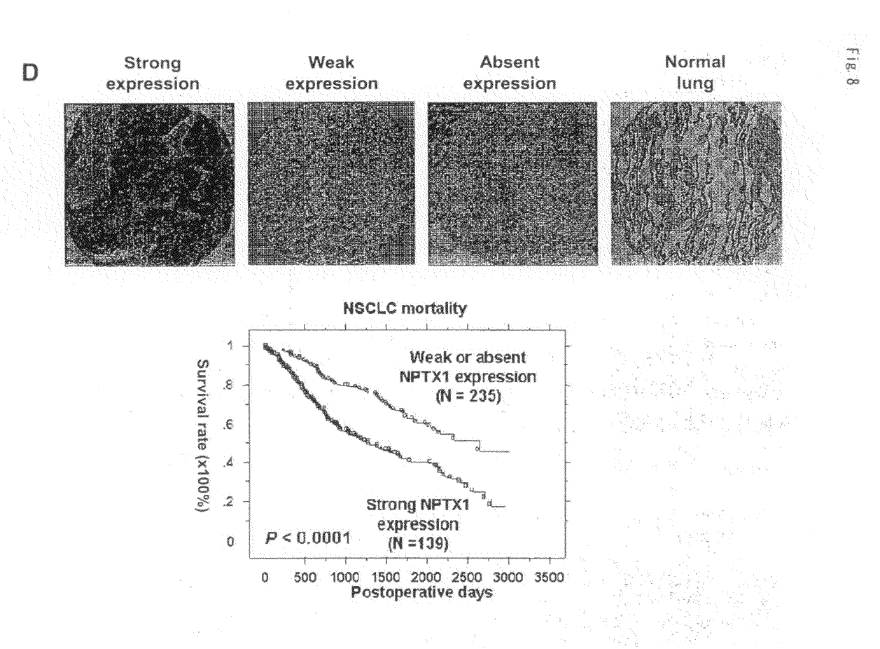

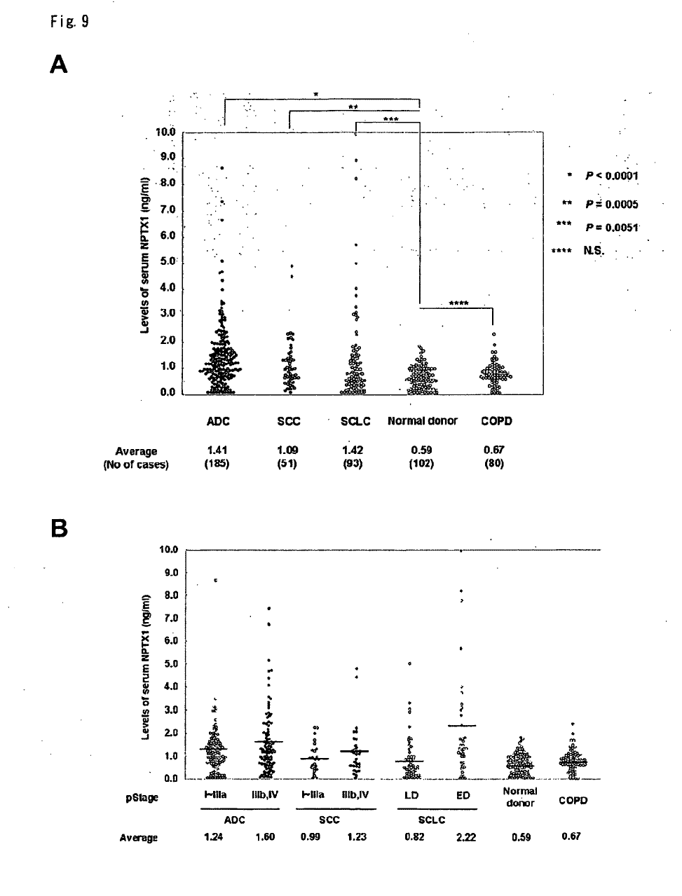

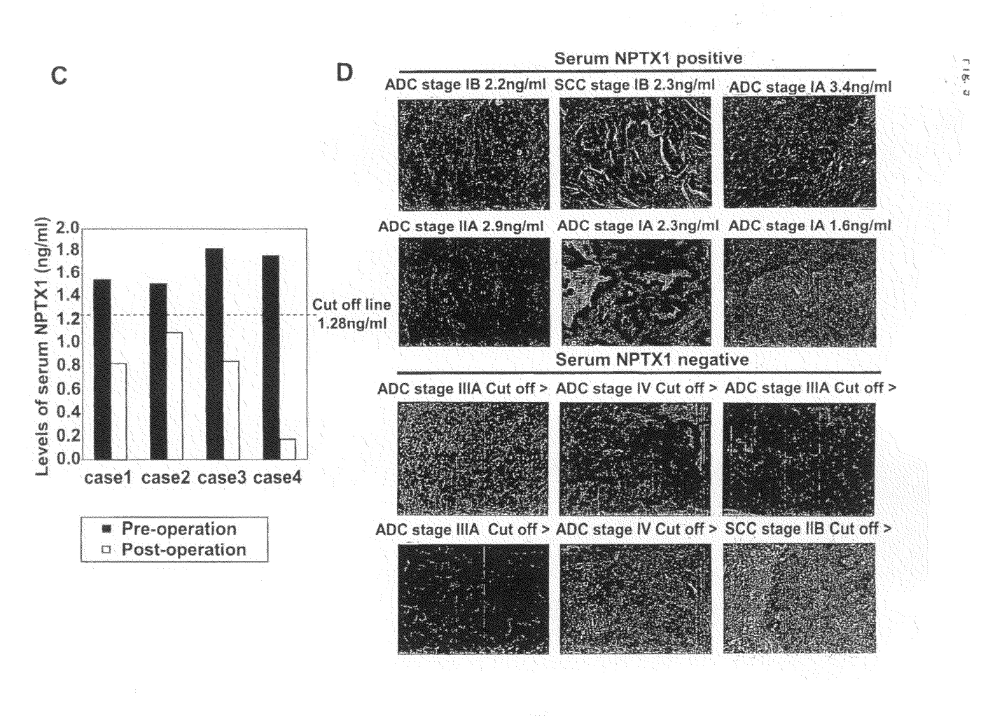

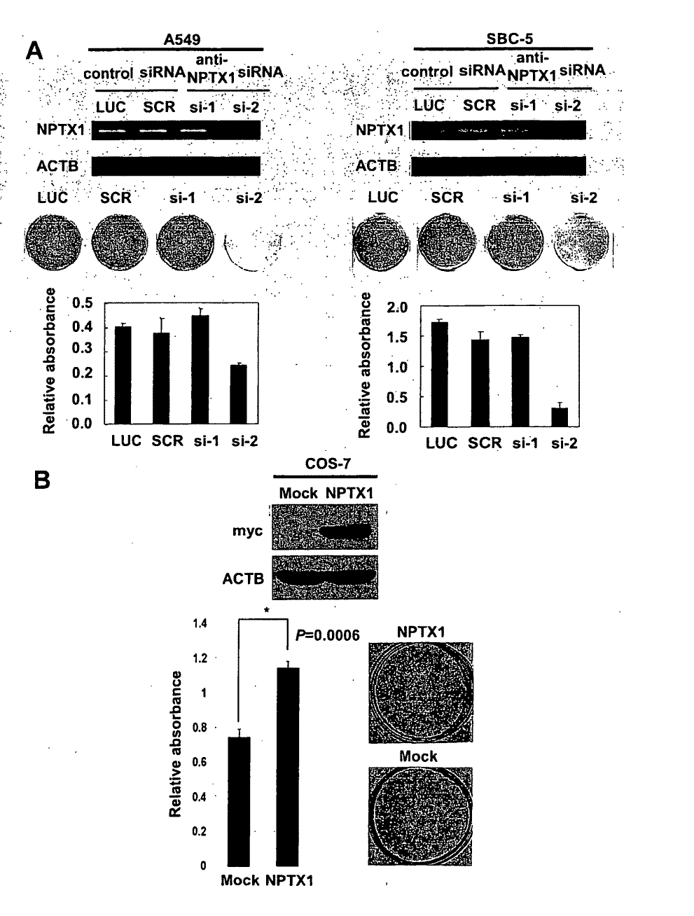

To investigate the biological and clinicopathological significance of EBI3 in pulmonary carcinogenesis, immunohistochemical staining was carried out on tissue microarray containing tissue sections from 423 NSCLC cases that underwent curative surgical resection. EBI3 staining detected with polyclonal antibody specific to EBI3 was mainly observed at cytoplasm of tumor cells but was not in normal lung cells (FIG. 2A). A pattern of EBI3 expression was classified on the tissue array ranging from absent (scored as 0) to weak/strong positive (scored as 1+ to 2+). Of the 423 NSCLCs, EBI3 was strongly stained in 210 (49.6%) cases (score 2+), weakly stained in 159 (37.6%) cases (score 1+), and not stained in 54 (12.8%) cases (score 0) (Table 2A). Then, a correlation of EBI3 expression (strong positive vs weak positive/absent) was found its significant correlation with gender (higher in male; P<0.0001 by Fisher's exact test), histological type (higher in non-ADC; P=0.0004 by Fisher's exact test), tumor size (higher in pT2-4; P=0.0009 by Fisher's exact test), and lymph-node metastasis (higher in pN1-2; P=0.0039 by Fisher's exact test) (Table 2A). The median survival time of NSCLC patients was significantly shorter in accordance with the higher expression levels of EBI3 (P=0.0011, log-rank test; FIG. 2B). In addition, univariate analysis was applied to evaluate associations between patient prognosis and several factors, including age, sex, pathologic tumor stage (tumor size; T1 vs T2-4), pathologic node stage (node status; N0 vs N1, N2), histology (ADC vs other histology types), and EBI3 status (score 0, 1+ vs score 2+). All those variables were significantly associated with poor prognosis. Multivariate analysis using a Cox proportional hazard model determined that EBI3 (P=0.0435) as well as other three factors (age, pathologic tumor stage, and pathologic node stage) were independent prognostic factors for surgically treated NSCLC patients (Table 2B).

TABLE 2A
Association between EBI3-positivity in NSCLC tissues and patients'
characteristics (n = 423)

| | | EBI3 expression | | | | P value |
|---|---|---|---|---|---|---|
| | | Strong | Low | Absent | | Strong vs |
| | Total | expression | expression | expression | Chi- | Low or |
| | n = 423 | n = 210 | n = 159 | n = 54 | square | absent |
| Sex | | | | | | |
| Female | 132 | 46 | 57 | 29 | 15.958 | <0.0001* |
| Male | 291 | 164 | 102 | 25 | | |
| Age(year) | | | | | | |
| >=65 | 211 | 107 | 72 | 32 | 0.116 | NS |
| <65 | 212 | 103 | 87 | 22 | | |
| T factor | | | | | | |
| T1 | 136 | 51 | 58 | 27 | 11.124 | 0.0009* |
| T2 + T3 + T4 | 287 | 159 | 101 | 27 | | |
| N factor | | | | | | |
| N0 | 264 | 120 | 107 | 37 | 4.499 | 0.0339* |
| N1 + N2 | 159 | 90 | 52 | 17 | | |
| Histological type | | | | | | |
| ADC | 272 | 117 | 110 | 45 | 12.674 | 0.0004* |
| non-ADC | 151 | 93 | 49 | 9 | | |

*P < 0.05 (Fisher's exact test)
NS, no significance
ADC, adenocarcinoma
non-ADC, squamous cell carcinoma plus large cell carcinoma and adenosquamous cell carcinoma

TABLE 2B
Cox's proportional hazards model analysis of prognostic
factors in patients with NSCLCs

| Variables | Hazards ratio | 95% CI | Unfavorable/Favorable | P-value |
|---|---|---|---|---|
| Univariate analysis | | | | |
| EBI3 | 1.617 | 1.208-2.164 | Positive/Negative | 0.0012* |
| Age (years) | 1.492 | 1.116-1.994 | >=65/65> | 0.007* |
| Gender | 1.669 | 1.193-2.334 | Male/Female | 0.0028* |
| pT factor | 2.761 | 1.895-4.023 | T2 + T3 + T4/T1 | <0.0001* |
| pN factor | 2.389 | 1.791-3.185 | N1 + N2/N0 | <0.0001* |
| Histological type | 1.390 | 1.040-1.858 | non-ADC/ADC | 0.026* |
| Multivariate analysis | | | | |
| EBI3 | 1.361 | 1.009-1.835 | Positive/Negative | 0.0435* |
| Age (years) | 1.678 | 1.245-2.261 | >=65/65> | 0.0007* |
| Gender | 1.398 | 0.967-2.021 | Male/Female | NS |
| pT factor | 2.075 | 1.403-3.067 | T2 + T3 + T4/T1 | 0.0003* |
| pN factor | 2.313 | 1.712-3.125 | N1 + N2/N0 | <0.0001* |
| Histological type | 0.921 | 0.670-1.266 | non-ADC/ADC | NS |

ADC, adenocarcinoma
non-ADC, squamous-cell carcinoma plus large-cell carcinoma and adenosquamous-cell carcinoma
NS, no significance
*P < 0.05

### Example 4

### Serum Levels of EBI3 in Patients with Lung Cancer

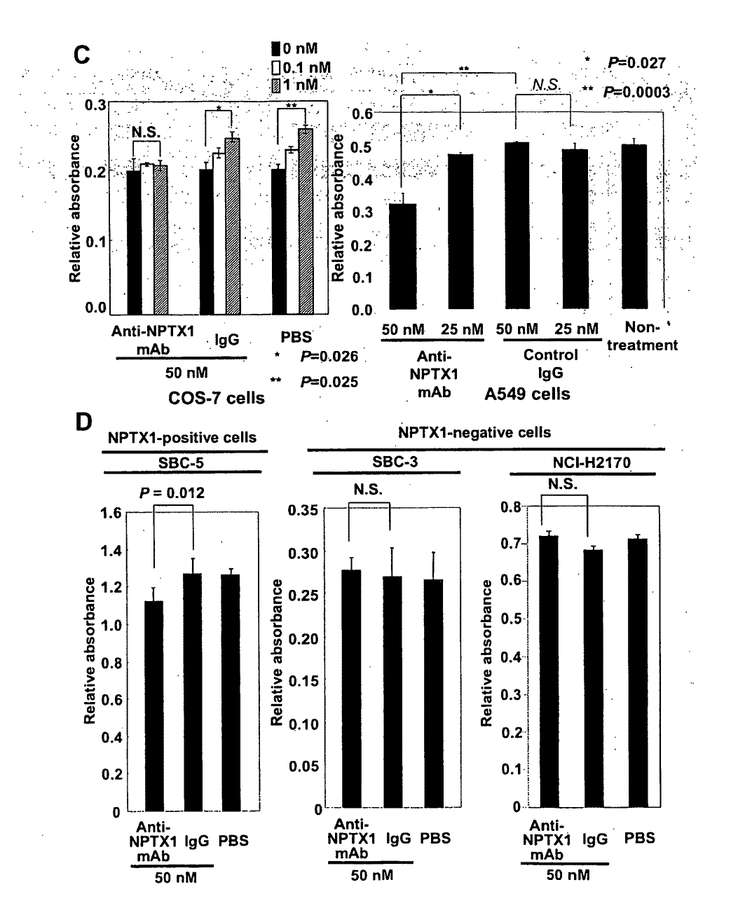

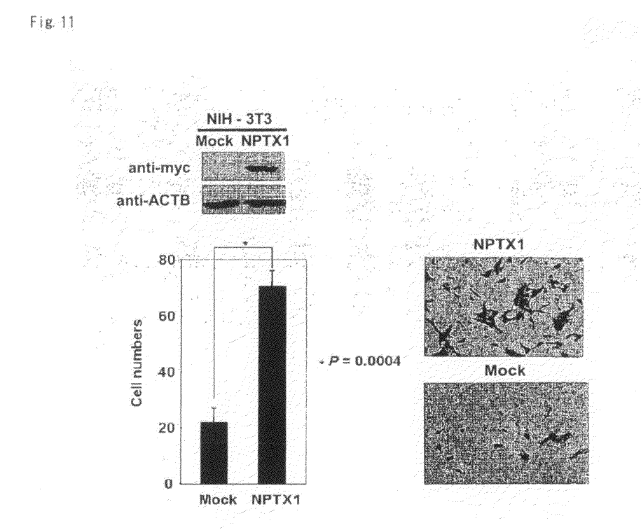

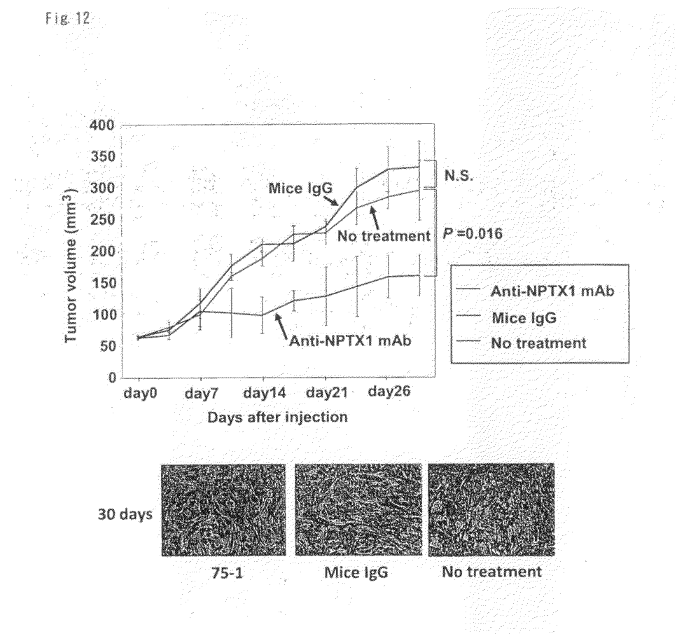

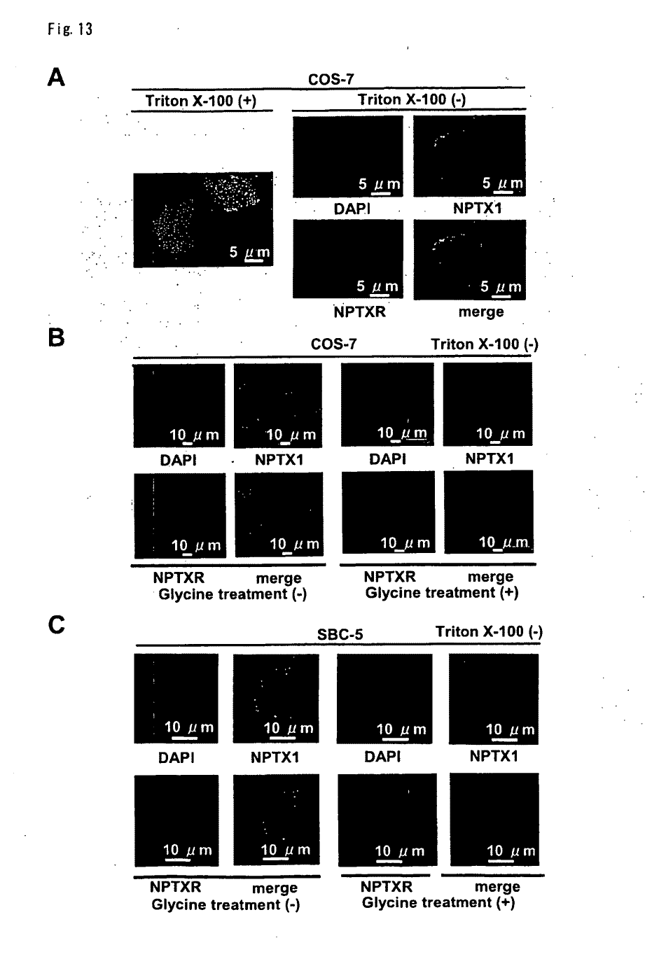

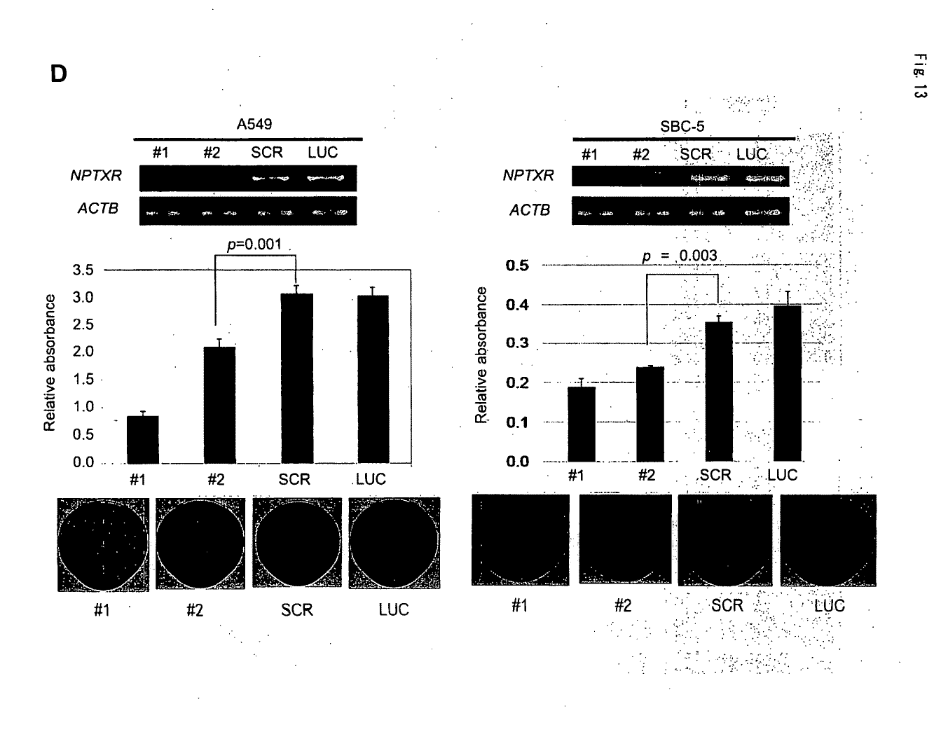

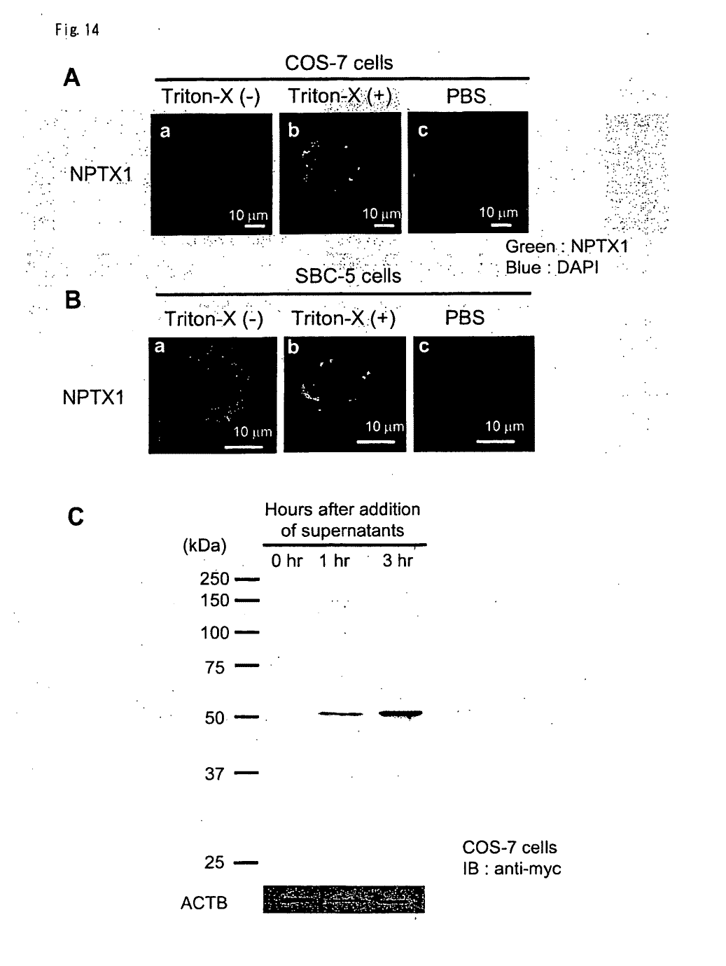

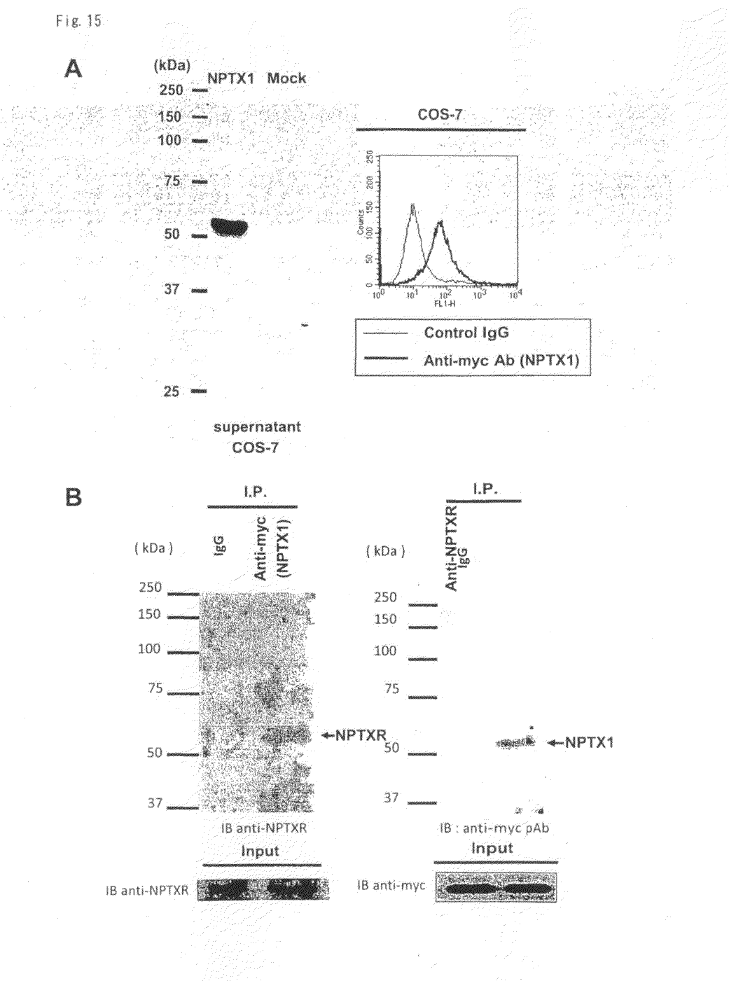

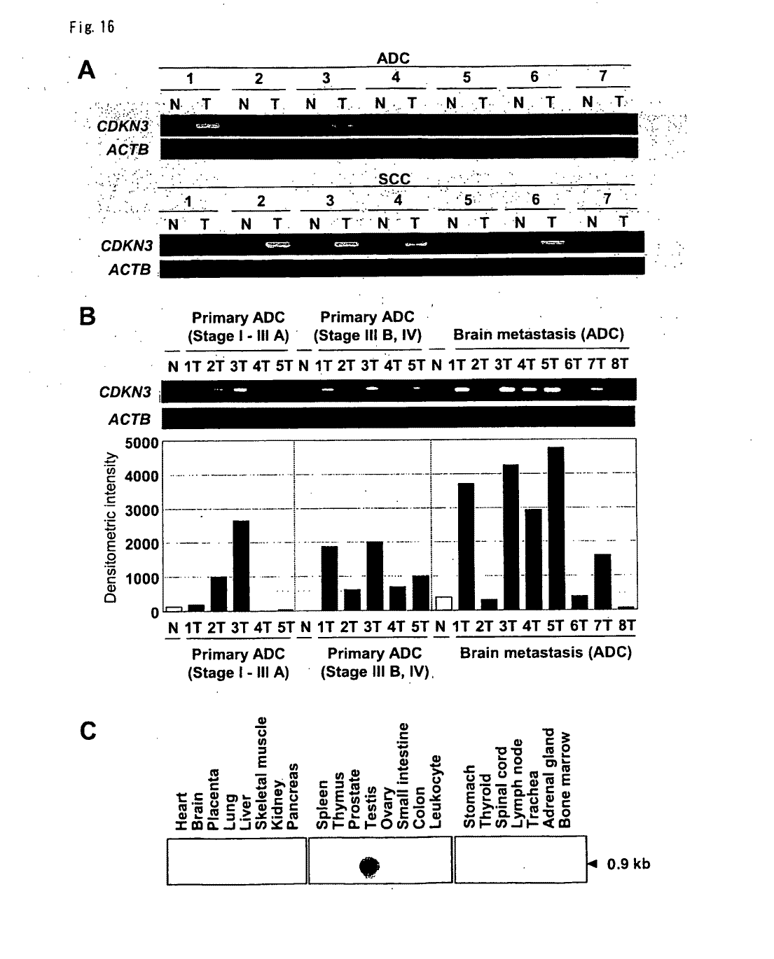

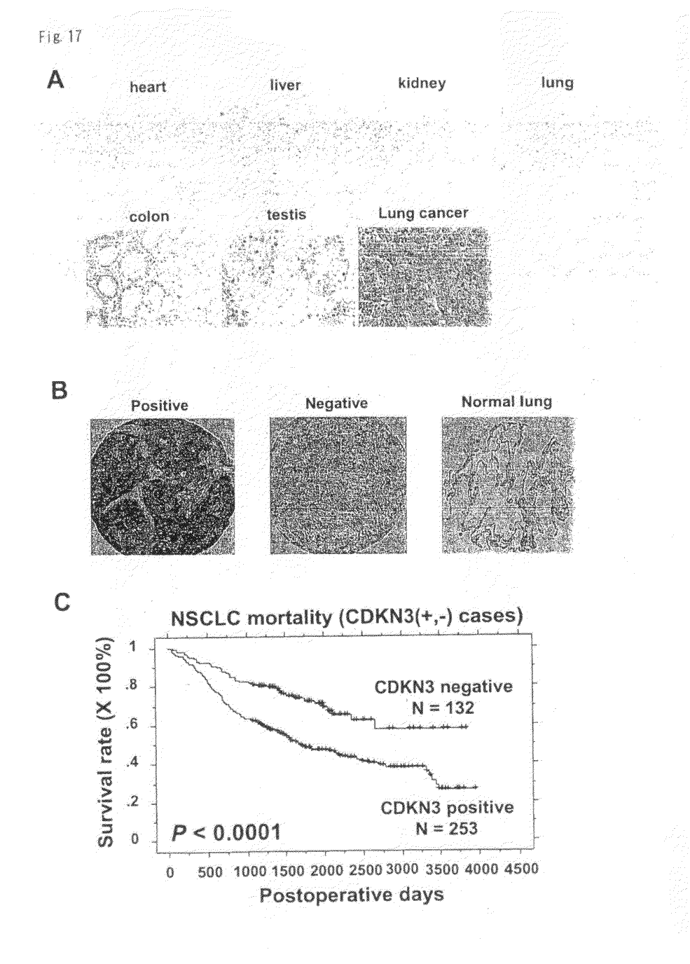

Because EBI3 encodes a secreted protein, we investigated whether the EBI3 protein is secreted into sera of patients with lung cancer. ELISA experiments detected EBI3 protein in serologic samples from the great majority of the 301 lung cancer patients. The mean (±1SD) of serum levels of EBI3 in lung cancer patients was 18.0±16.4 units/mL. In contrast, the mean (±1SD) serum levels of EBI3 in 134 healthy individuals were 4.4±4.7 units/mL and those in 63 patients with COPD, who were current and/or former smokers, were 5.8±8.0 units/mL. The levels of serum EBI3 protein were significantly higher in lung cancer patients than in healthy donors or in COPD patients (P<0.0001, Mann-Whitney U test); the difference between healthy individuals and COPD patients was not significant (P=0.160). According to histologic types of lung cancer, the serum levels of EBI3 were 17.8±15.4 units/mL in 178 adenocarcinoma patients, 19.9±16.9 units/mL in 41 SCC patients, and 17.6±18.1 units/mL in 82 SCLC patients (FIG. 3A); the differences among the three histologic types were not significant. The present inventors then evaluated the relationship between levels of EBI3 and clinical stage of lung cancer patients whose information was available. High levels of serum EBI3 were detected even in patients with earlier-stage tumors (FIG. 3B). Using ROC curves drawn with the data of these 301 cancer patients and 134 healthy controls (FIG. 4A, left panel), the cutoff level in this assay was set to provide optimal diagnostic accuracy and likelihood ratios (minimal false-negative and false-positive results) for EBI3 [i.e., 15.4 units/mL with a sensitivity of 45.2% (136 of 301) and a specificity of 97.8% (131 of 134)]. According to tumor histology, the proportions of the serum EBI3-positive cases were 47.9% for NSCLC (105 of 219) and 37.8% for SCLC (31 of 82). The proportions of the serum EBI3-positive cases were 3.2% (2 of 63) for COPD. It was performed ELISA experiments using paired preoperative and postoperative (2 months after the surgery) serum samples from NSCLC patients to monitor the levels of serum EBI3 in the same patients. The concentration of serum EBI3 was dramatically reduced after surgical resection of primary tumors (FIG. 4A, right panel). The present inventors further compared the serum EBI3 values with the expression levels of EBI3 in primary tumors in the same set of 6 NSCLC cases whose serum had been collected before surgery (three patients with EBI3-positive tumors and three with EBI3-negative tumors). The levels of serum EBI3 showed good correlation with the expression levels of EBI3 in primary tumor (FIG. 4B). The results independently support the high specificity and the great potentiality of serum EBI3 as a biomarker for detection of cancer at an early stage and for monitoring of the relapse of the disease.

### Example 5

### Combination Assay of EBI3 and CEA/CYFRA/ProGRP as Tumor Markers

To evaluate the clinical usefulness of serum EBI3 level as a tumor detection biomarker, the serum levels of two conventional tumor markers (CEA for ADC, CYFRA for SCC, and ProGRP for SCLC patients) were measured by ELISA in the same set of serum samples from cancer patients and control individuals. ROC analyses determined the cutoff value of CEA for NSCLC detection to be 2.2 ng/mL [with a sensitivity of 36.0% (64 of 178) and a specificity of 97.5% (115 of 118); FIG. 4C, left top panel]. The correlation coefficient between serum EBI3 and CEA values was not significant (Spearman rank correlation coefficient: ρ(rho)=0.063; P=0.4016), indicating that measuring both markers in serum can improve overall sensitivity for detection of ADC to 65.7% (117 of 178); for diagnosing ADC, the sensitivity of CEA alone is 36.0% (64 of 178) and that of EBI3 is 46.1% (82 of 205). False-positive rates for either of the two tumor markers among normal volunteers (control group) were 5.1% (6 of 118), although the false-positive rates for each of CEA and EBI3 in the same control group were 2.5% (3 of 118) and 2.5% (3 of 118; FIG. 4C, left bottom panel), respectively. ROC analyses for the patients with SCC determined the cut off value of CYFRA as 2.0 ng/ml, with a sensitivity of 48.6% (18 of 37) and a specificity of 2.3% (3 of 130; FIG. 4C, middle top panel). The correlation coefficient between serum EBI3 and CYFRA was not significant (Spearman rank correlation coefficient: ρ (rho)=−0.117; P=0.4817), indicating that measuring both markers in serum can improve overall sensitivity for detection of SCC to 78.5%; for diagnosing SCC, the sensitivity of CYFRA alone is 48.6% (18 of 37) and that of EBI3 is 54.1% (20 of 37). False-positive rates for either of the two tumor markers among normal volunteers (control group) were 4.6% (6 of 130), although the false-positive rates for each of CYFRA and EBI3 in the same control group were 2.3% (3 of 130) and 2.3% (3 of 130; FIG. 4C, middle bottom panel). ROC analyses for the patients with SCLC determined the cutoff value of ProGRP as 39.5 pg/mL, with a sensitivity of 64.6% (53 of 82) and a specificity of 97.4% (3 of 116; FIG. 4C, right top panel). The correlation coefficient between serum EBI3 and ProGRP values was not significant (Spearman rank correlation coefficient: p (rho). 0.074; P=0.5075), also indicating that measurement of serum levels of both markers can improve overall sensitivity for detection of SCLC to 74.4% (61 of 82); for diagnosing SCLC, the sensitivity of ProGRP alone was 64.6% (53 of 82) and that of EBI3 was 37.8% (31 of 82). False-positive cases for either of the two tumor markers among normal volunteers (control group) were 5.2% (6 of 116), although the false-positive rates for ProGRP and EBI3 in the same control group were 2.6% (3 of 116) and 2.6% (3 of 116; FIG. 4C, right bottom panel), respectively.

### Example 6

### Inhibition of the Growth of Lung Cancer Cells by siRNA Against EBI3

To assess whether up-regulation of EBI3 plays a role in growth or survival of lung cancer cells, we evaluated the inhibition of endogenous EBI3 expression by siRNA, along with two different control siRNAs (siRNAs for ON-Target and LUC). Treatment of two different NSCLC cells, A549 or LC319 with the effective siRNA could reduce expression of EBI3 (FIG. 4D), and resulted in significant inhibition of cell viability and colony numbers measured by MTT and colony formation assays (FIG. 4D). The result suggest that up-regulation of EBI3 is related to growth or survival of cancer cells.

### Example 7

### Growth-Promoting Effect of EBI3

To disclose the potential role of EBI3 in tumorigenesis, we prepared plasmids designed to express EBI3 (pcDNA3.1-EBI3-myc/His). This plasmids or mock plasmids were transfected into COS-7 cells and established stable clones expressing EBI3. It was confirmed the expression of EBI3 protein in cytoplasm by immunocytochemical staining using anti-EBI3 antibody (data not shown). To determine the effect of EBI3 on the growth of mammalian cells, the present inventors carried out a colony formation assay of COS-7-derived transfectants that stably expressed EBI3. The present inventors established two independent COS-7 cell lines expressing exogenous EBI3 (COS-7-EBI3-#1 and -#2; FIG. 4E, top panels), and compared their growth with control cells transfected with mock vector (COS-7-MOCK-M1 and -M2). Growth of both of two COS-7-EBI3 cells was promoted at a significant degree in accordance with the expression level of EBI3 (FIG. 4E, bottom panels). There was also a remarkable tendency in COST-EBI3 cells to form larger colonies than the control cells (FIG. 4E, bottom panels). In accordance with the result of siRNA assays, these data strongly suggest that EBI3 plays a significant role in the tumor growth and/or survival.

### Analysis and Discussion:

Despite recent advances in diagnostic imaging of tumors, combination chemotherapy and radiation therapy, little improvement has been achieved within the last decade in terms of prognosis and quality of life for patients with lung cancer. Therefore, it is now urgently required to develop novel diagnostic biomarkers for early detection of cancer and for the better choice of adjuvant treatment modalities to individual patients. Genome-wide expression profile analyses of 101 lung cancers after enrichment of cancer cells by laser microdissection were performed using a cDNA microarray containing more than 32,256 genes (Kikuchi T, et al., Oncogene 22: 2192-205 (2003), Taniwaki M, et al., Int J Oncol 29: 567-75 (2006), Kikuchi T, et al., Int J Oncol 28: 799-805 (2006), Kakiuchi S, et al., Mol Cancer Res 1: 485-99 (2003), Kakiuchi S, et al., Hum Mol Genet 13: 3029-43 (2004)). Through the analyses, it was revealed that several genes have potential as candidates for development of novel diagnostic markers, therapeutic drugs, and/or immunotherapy (Suzuki C, et al., Cancer Res 65: 11314-25 (2005), Ishikawa N, et al., Clin Cancer Res 10: 8363-70 (2004), Kato T, et al., Cancer Res 65: 5638-46 (2005), Hayama S, et al., Cancer Res 67: 4113-22 (2007)).

Among them, the genes encoding putative tumor-specific transmembrane or secretory proteins are considered to have significant advantages because they are present on the cell surface or within the extracellular space, and/or in serum, making them easily accessible as molecular markers and therapeutic targets. In the context of the present invention, one of such genes, EBI3, encoding a secretory protein, was examined the protein expression status by means of tissue microarray and ELISA for evaluating it for usefulness as diagnostic and prognostic biomarker(s) for lung cancer.

EBI3 was identified by the induction of its expression in B lymphocytes by Epstein-Barr virus infection (Devergne O, et al., J Virol 70: 1143-1153 (1996)). This 34-kDa glycoprotein is a member of the hematopoietin receptor family related to the p40 subunit of IL-12, and is suggested to play a role in regulating cell-mediated immune responses.

EBI3 is a 34-kDa glycoprotein that first described as its strong expression in EBV-immortalized lymphoblastoid cell lines in vitro (Devergne O, et al., J Virol 70: 1143-1153 (1996)). Recent studies disclose that EBI3 forms a novel cytokine called IL-27 by heterodimerizing with p28, a new IL-12 p35-related subunit and that plays an important role for initiation of Th 1 immunoresponse (Pflanz S, et al., Immunity 16: 779-90 (2002)). On the contrary, recent reports have suggested that EBI3 may form IL-35 with IL-12 alpha and modulate the immunoresponse to immunosuppression by reacting with regulatory T (T_{reg}) cells (Niedbala W, et al., Eur J Immunol 37: 1-9 (2007), Collison L W, et al., Nature 450: 566-9 (2007)). Moreover, it has been reported that during human pregnancy, expression of EBI3 could be seen in placental vill, suggesting that EBI3 may modulate the immunoresponse between maternal body and placenta, such as maternal immunotolerance (Devergne O, et al., Am J Pathol 159: 1763-76 (2001)).

In the context of the present invention, high level of EBI3 protein expression was found in tissue samples from lung cancer patients. Concordantly, it was also demonstrated that inhibition of endogenous expression of EBI3 by siRNA resulted in marked reduction of viability of lung cancer cells, while mammalian cells expressing exogenous EBI3 exhibited significant growth promotion. Although the detailed function of EBI3 in lung carcinogenesis is unknown, the present results implied that EBI3 expression could promote the cancer cell proliferation/survival.

High level of EBI3 protein was also found in serologic samples from lung cancer patients. As a half of the serum samples used for this study were derived from patients with early-stage cancers, EBI3 should be useful for diagnosis of even early-stage cancers. To examine the feasibility for applying EBI3 as the diagnostic tool, the serum levels of EBI3 was compared with those of CEA, CYFRA or ProGRP, three conventional diagnostic markers for NSCLCs and SCLCs, from the view point of there sensitivity and specificity for diagnosis. An assay combining both markers (EBI3+CEA, EBI3+CYFRA, or EBI3+ProGRP) increased the sensitivity to about 65-75% for lung cancer (NSCLC as well as SCLC), significantly higher than that of CEA or ProGRP alone, whereas 5% to 7% of healthy volunteers were falsely diagnosed as positive. Although further validation using a larger set of serum samples covering various clinical stages will be required, present data presented here sufficiently show a potential clinical usefulness of EBI3 itself as a serologic/histochemical biomarker for lung cancers.

In conclusion, EBI3 is identified herein as a potential biomarker for serum diagnosis and immunohistochemical prediction of prognosis for lung cancer patients. This molecule is also a likely candidate for development of therapeutic approaches such as antibody therapy, small molecular compounds, and cancer vaccines.

### Part II

### DLX5 Related Experiments

### Example 8

### General Methods

### 1. Lung-Cancer Cell Lines and Tissue Samples.

The human lung-cancer cell lines used in this study were as follows: lung adenocarcinomas (ADC), A427, A549, LC319, PC3, PC9, and NCI-H1373; a bronchiolo-alveolar carcinoma (BAC), NCI-H1781; lung squamous-cell carcinomas (SCC), RERF-LC-AI, SK-MES-1, EBC-1, LU61, NCI-H520, NCI-H1703, and NCI-H2170; lung adenosquamous carcinomas (ASC), NCI-H226 and NCI-H647; a lung large-cell carcinoma (LCC), LX1; and small cell lung cancers (SCLC), DMS114, DMS273, SBC-3, and SBC-5. All cells were grown in monolayer in appropriate medium supplemented with 10% fetal calf serum (FCS) and were maintained at 37 degree Centigrade in atmospheres of humidified air with 5% CO2. Human small airway epithelial cells (SAEC) were grown in optimized medium (SAGM) purchased from Cambrex Bio Science Inc. (Walkersville, Md.). 14 primary NSCLCs (seven ADCs and seven SCCs) had been obtained from patients with written informed consent, as described previously (Kato T, et al., Cancer Res 65: 5638-46 (2005)). A total of 369 NSCLCs and adjacent normal lung-tissue samples for immunostaining on tissue microarray were obtained from patients who underwent curative surgery at Saitama Cancer Center (Saitama, Japan). This study and the use of all clinical materials were approved by the Institutional Research Ethics Committees.

### 2. Semiquantitative RT-PCR

Total RNA was extracted from cultured cells and clinical tissues using TRIzol reagent (Life Technologies, Inc., Gaithersburg, Md.) according to the manufacturer's protocol. Extracted RNAs and normal human tissue poly(A) RNAs were treated with DNase I (Nippon Gene, Tokyo, Japan) and reversely-transcribed using oligo (dT) primer and SuperScript II reverse transcriptase (Invitrogen, Carlsbad, Calif.). Semiquantitative RT-PCR experiments were carried out with the following DLX5-specific primers or with ACTB-specific primers as an internal control:

(SEQ ID NO: 21)
DLX5, 5′-CTCGCTCAGCCACCACCCTCAT-3′,
and
(SEQ ID NO: 22)
5′-AGTTGAGGTCATAGATTTCAAGGCAC-3′;
(SEQ ID NO: 11)
ACTB, 5′-GAGGTGATAGCATTGCTTTCG-3′
and
(SEQ ID NO: 12)
5′-CAAGTCAGTGTACAGGTAAGC-3′.

PCR reactions were optimized for the number of cycles to ensure product intensity within the logarithmic phase of amplification.

### 3. Northern-Blot Analysis.

Human multiple-tissue blots (BD Biosciences Clontech, Palo Alto, Calif.) were hybridized with a 32P-labeled PCR product of DLX5. The cDNA probes of DLX5 were prepared by RT-PCR using the primers described above. Pre-hybridization, hybridization, and washing were performed according to the supplier's recommendations. The blots were autoradiographed at room temperature for 30 hours with intensifying BAS screens (BIO-RAD, Hercules, Calif.).

### 4. Anti-DLX5 Antibodies

Plasmids expressing full length fragments of DLX5 that contained His-tagged epitopes at their NH2-terminals were prepared using pET28 vector (Novagen, Madison, Wis.). The recombinant peptides were expressed in Escherichia coli, BL21 codon-plus strain (Stratagene, LaJolla, Calif.), and purified using TALON resin (BD Bioscience) according to the supplier's protocol. The protein, extracted on an SDS-PAGE gel, was inoculated into rabbits; the immune sera were purified on affinity columns according to standard methodology. Affinity-purified anti-DLX5 antibodies were used for immunohistochemical study. It was confirmed that the antibody was specific to DLX5, on western blots using lysates from cell lines that had been transfected with DLX5 expression vector as well as by immunocytochemical staining of cell lines, either of which expressed DLX5 endogenously or not.

### 5. Immunocytochemistry

SBC-5 cells were seeded on coverslips and cells were fixed in 4% formamide and permeabilized with cold methanol acetone (50:50) for 5 min at room temperature. After washing in PBS once, cells were incubated with the anti-DLX5 antibody for 1 hour at room temperature, followed by incubation with Alexa488 conjugated goat anti-rabbit antibodies (Molecular Probes) (1:1000 dilution) for 1 hour in the dark. Images were captured on a confocal microscope (TCS SP2-AOBS, Leica Microsystems).

### 6. Immunohistochemistry and Tissue-Microarray Analysis

To investigate the presence of DLX5 protein in clinical materials, tissue sections were stained by ENVISION+ Kit/HRP (DakoCytomation, Glostrup, Denmark). Affinity-purified anti-DLX5 antibodies were added after blocking of endogenous peroxidase and proteins, and each section was incubated with HRP-labeled anti-rabbit IgG as the secondary antibody. Substrate-chromogen was added and the specimens were counterstained with hematoxylin. Tumor-tissue microarrays were constructed as published elsewhere, using formalin-fixed NSCLCs (Ishikawa N, et al., Clin Cancer Res 10: 8363-70 (2004)). Tissue areas for sampling were selected based on visual alignment with the corresponding HE-stained sections on slides. Three, four, or five tissue cores (diameter 0.6 mm; height 3-4 mm) taken from donor-tumor blocks were placed into recipient paraffin blocks using a tissue microarrayer (Beecher Instruments, Sun Prairie, Wis.). A core of normal tissue was punched from each case. Five-micro m sections of the resulting microarray block were used for immunohistochemical analysis. Positivity for DLX5 was assessed semiquantitatively by three independent investigators without prior knowledge of the clinical follow-up data, each of who recorded staining intensity as absent (scored as 0), weak (1+) or strongly positive (2+). Lung-cancers were scored as strongly positive (2+) only if all reviewers defined them as such.

### 7. Statistical Analysis

All analyses were performed using statistical analysis software (StatView, version 5.0; SAS Institute, Inc. Cary, N.C., USA). Correlations between its expression levels and clinicopathological variables such as age, gender, pathological TNM stage, and histological type were then examined. Strong DLX5 immunoreactivity was assessed for association with clinicopathologic variables using the Fisher's exact test. Univariate and multivariate analyses were performed with the Cox proportional-hazard regression model to determine associations between clinicopathological variables and cancer-related mortality. First, associations between death and possible prognostic factors including age, gender, histological type, pT-classification, and pN-classification, taking into consideration one factor at a time were analyzed. Second, multivariate Cox analysis was applied on backward (stepwise) procedures that always forced DLX5 expression into the model, along with any and all variables that satisfied an entry level of a P value less than 0.05. As the model continued to add factors, independent factors did not exceed an exit level of P<0.05.

### 8. RNA Interference Assay

A vector-based RNA interference (RNAi) system, psiH1BX3.0 that was designed to generate siRNAs in mammalian cells has been previously established (Suzuki C, et al., Cancer Res 63: 7038-41 (2003)). Using 30 micro L of Lipofectamine 2000 (Invitrogen), 10 micro g of DLX5-specific siRNA-expression vector was transfected into SBC-5 and NCI-H1781 cell lines that endogenously overexpressed DLX5. The transfected cells were cultured for seven days in the presence of appropriate concentrations of geneticin (G418), and the numbers of colonies and viable cells were counted by Giemsa staining in triplicate MTT assays. The target sequences of the synthetic oligonucleotides for RNAi were as follows:

control 1 (EGFP: enhanced green fluorescent
protein gene, a mutant of Aequorea victoria GFP),
(SEQ ID NO: 23)
5′-GAAGCAGCACGACTTCTTC-3′;
control 2 (Scramble: chloroplast Euglena gracilis
gene coding for 5S and 16S rRNAs),
(SEQ ID NO: 16)

5′-GCGCGCTTTGTAGGATTCG-3′;

siRNA-DLX5-#1,
5′-CCAGCCAGAGAAAGAAGTG-3′;
siRNA-DLX5-#2,
5′-GTGCAGCCAGCTCAATCAA-3′.

To validate present RNAi system, down-regulation of DLX5 expression by functional siRNA, but not by controls or non-effective siRNA, was confirmed in the cell lines used for this assay.

### Example b 9

### Expression of DLX5 Gene in Lung Cancers and Normal Tissues

To identify target molecules for development of novel therapeutic agents and/or biomarkers for lung cancer, first screening through a cDNA microarray for genes that showed 5-fold or higher expression in more than 50% of 86 NSCLCs or 15 SCLCs analyzed (Kikuchi T, et al. Oncogene. 2003 Apr. 10; 22(14):2192-205; Taniwaki M, et al, Int J Oncol. 2006 September; 29(3):567-75; Kakiuchi S, et al. Mol Cancer Res. 2003 May; 1(7):485-99) was performed. Among 27,648 genes screened, the DLX5 gene was identified to be overexpressed in the majority of lung cancers, and confirmed its overexpression by semiquantitative RT-PCR experiments in 9 of 14 additional NSCLC cases (2 of 7 ADCs and all of 7 SCCs) (FIG. 5A) as well as in 10 of 23 lung cancer cell lines, whereas its expression was hardly detectable in SAEC cells derived from normal bronchial epithelium (FIG. 5B). To determine the subcellular localization of endogenous DLX5 in lung cancer cells, rabbit polyclonal antibody specific to human DLX5 was subsequently generated and found to stain strongly in the nucleus and weakly in the cytoplasm of SBC-5 cells (FIG. 5C). Northern-blot analysis using DLX5 cDNA as a probe identified a strong signal corresponding to a 1.8-kb transcript only in the placenta among 23 tissues examined (FIG. 5D). Furthermore, DLX5 protein expressions in 5 normal tissues (heart, liver, kidney, lung, and placenta) were compared with those in lung cancers using anti-DLX5 polyclonal antibodies by immunohistochemical analysis. In concordant with the result of northern analysis, DLX5 expression was observed in the placenta and lung cancers, but was hardly detectable in the four other normal tissues (FIG. 6A).

### Example 10

### Association of DLX5 Expression with Poor Prognosis for NSCLC Patients

To verify the clinicopathological significance of DLX5, the expression of DLX5 protein was additionally examined by means of tissue microarrays containing lung-cancer tissues from 369 patients who underwent curative surgical resection. A pattern of DLX5 expression was classified on the tissue array ranging from absent/weak (scored as 0˜1+) to strong (2+) (FIG. 6B). Positive staining was found in 191 of 234 ADC tumors (81.6%), 80 of 95 SCC tumors (84.2%), 24 of 27 LCC tumors (88.9%), and 10 of 13 ASC tumors (76.9%). A correlation of DLX5 expression (strong positive vs. weak positive/absent) with various clinicopathological parameters was then examined and significant correlation with pT classification was found (higher in larger tumor; P=0.0053 by Fisher's exact test) (Table 3A).

Of the 369 NSCLC cases examined, DLX5 was strongly stained in 160 cases (43.4%; score 2+), weakly stained in 145 cases (39.3%; score 1+), and not stained in 64 cases (17.3%; score 0) (details are shown in Table 3A). NSCLC patients whose tumors showed strong DLX5 expression revealed shorter tumor-specific survival periods compared to those with absent/weak DLX5 expression (P=0.0045 by the Log-rank test; FIG. 6C). Univariate analysis was also applied to evaluate associations between patient prognosis and other factors including age (<65 vs. 65<=), gender (female vs. male), histological type (ADC vs. non-ADC), pT classification (T1 vs. T2, T3, 4), pN classification (N0 vs. N1, N2), and DLX5 status (0, 1+ vs. 2+).

Among those parameters, DLX5 status (P=0.0048), elderly (P=0.0028), male (P=0.001), non-ADC histological classification (P=0.01), advanced pT stage (P<0.0001), and advanced pN stage (P<0.0001) were significantly associated with poor prognosis (Table 3B). In multivariate analysis of the prognostic factors, strong DLX5 expression, elderly, higher pT stage, and higher pN stage were indicated to be independent prognostic factors (P=0.0415, 0.0007, 0.0004, and <0.0001, respectively; Table 3B).

TABLE 3A
Association between DLX5-positivity in NSCLC tissues and
patients' characteristics (n = 369)

| | | DLX5 | DLX5 | | P-value |
|---|---|---|---|---|---|
| | | strong | weak | DLX5 | strong vs |
| | Total | positive | positive | absent | weak/ |
| | n = 369 | n = 160 | n = 145 | n = 64 | absent |
| Gender | | | | | |
| Male | 255 | 109 | 99 | 47 | NS |
| Female | 114 | 51 | 46 | 17 | |
| Age (years) | | | | | |
| <65 | 189 | 90 | 64 | 35 | NS |
| >=65 | 180 | 70 | 81 | 29 | |
| Histological type | | | | | |
| ADC | 234 | 96 | 95 | 43 | NS* |
| SCC | 95 | 44 | 36 | 15 | |
| Others | 40 | 20 | 14 | 6 | |
| pT factor | | | | | |
| T1 | 121 | 40 | 59 | 22 | 0.0053** |
| T2 − T4 | 248 | 120 | 86 | 42 | |
| pN factor | | | | | |
| N0 | 226 | 90 | 97 | 39 | NS |
| N1 + N2 | 143 | 70 | 48 | 25 | |

ADC, adenocarcinoma;
SCC, squamous-cell carcinoma
Others, large-cell carcinoma (LCC) plus adenosquamous-cell carcinoma (ASC)
*ADC versus non-ADC
**P < 0.05 (Fisher's exact test)
NS, no significance

TABLE 3B
Cox's proportional hazards model analysis of prognostic
factors in patients with NSCLCs

| | Hazards | | | |
|---|---|---|---|---|
| Variables | ratio | 95% CI | Unfavorable/Favorable | P-value |
| Univariate analysis | | | | |
| DLX5 | 1.517 | 1.136-2.026 | Strong(+)/Weak(+) or (−) | 0.0048* |
| Age (years) | 1.665 | 1.192-2.324 | 65<=/<65 | 0.0028* |
| Gender | 1.62 | 1.157-2.269 | Male/Female | 0.001* |
| Histological type | 1.466 | 1.096-1.963 | Non-ADC/ADC¹ | 0.01* |
| pT factor | 2.699 | 1.867-3.902 | T2 + T3 + T4/T1 | <0.0001* |
| pN factor | 2.674 | 1.999-3.576 | N1 + N2/N0 | <0.0001* |
| Multivariate analysis | | | | |
| DLX5 | 1.354 | 1.012-1.811 | Strong(+/Weak(+) or (−) | 0.0415* |
| Age (years) | 1.674 | 1.244-2.254 | 65<=/<65 | 0.0007* |
| Gender | 1.387 | 0.960-2.004 | Male/Female | NS |
| Histological type | 1.099 | 0.799-1.512 | non-ADC/ADC | NS |
| pT factor | 2.206 | 1.357-2.912 | T2 + T3 + T4/T1 | 0.0004* |
| pN factor | 2.536 | 1.879-3.421 | N1 + N2/N0 | <0.0001* |

¹ADC, adenocarcinoma
*P < 0.05
NS, no significance

### Example 11

### Growth Inhibition of NSCLC Cells by Specific siRNA Against DLX5

To assess whether DLX5 is essential for growth or survival of lung-cancer cells, plasmids were constructed to express siRNAs against DLX5 (si-DLX5-#1 and -#2) as well as two control plasmids (siRNAs for EGFP and Scramble), and transfected into lung-cancer cell lines, SBC-5 and NCI-H1781. The mRNA levels in cells transfected with si-DLX5-#2 were significantly decreased in comparison with those transfected with either of the two control siRNAs or si-DLX5-#1. The significant decreases were observed in the number of colonies and in the numbers of viable cells measured by MTT assay, suggesting that up-regulation of DLX5 is related to growth or survival of cancer cells (representative data of SBC-5 was shown in FIG. 6D).

### Discussion:

Although advances have been made in development of molecular-targeting drugs for cancer therapy, the proportion of patients showing good response to available treatments is still very limited (Imai K, et al., Nat Rev Cancer 6: 714-27 (2006)). Hence, it is urgent to develop new anti-cancer agents that will be highly specific to malignant cells, with minimal or no adverse reactions. Toward this direction, the present inventors have been pursuing a strategy to identify good molecular targets for drug development as follows; 1) screening for up-regulated genes in cancer cells on the basis of cDNA microarray analysis; 2) investigating loss-of-function phenotypes using RNAi systems and defining biological functions of the proteins; and 3) systematic analysis of protein expression among hundreds of clinical samples on tissue microarrays. Taking this approach, it is demonstrated herein that DLX5, a member of distal-less homeobox protein family, is frequently overexpressed in the great majority of clinical lung-cancer samples and cell lines, and that the gene product is necessary for survival/growth of lung-cancer cells.

The vertebrate Dlx genes, which encode a family of homeobox-containing transcription factors related in sequence to the Drosophila Distal-less (Dll) gene product, constitute one example of functional diversification of paralogs. All vertebrates investigated thus far have at least six Dlx genes that are generally arranged as three bigene clusters: Dlx1/Dlx2, Dlx5/Dlx6, and Dlx3/Dlx4(Dlx7) (24, 28-30). The Dlx5 protein is first expressed in the anterior region of mouse embryos during early embryonic development (Simeone A, et al., Proc Natl Acad Sci USA 91: 2250-4 (1994)). It has been reported that homozygous Dlx5/Dlx6 double-knockout mice exhibit split hand/foot malformation (SHFM) phenotypes, a heterogeneous limb disorder characterized by missing central digits and claw-like distal extremities, suggesting that DLX5 gene is one of critical regulators for mammalian limb development (Merlo G R, et al., Genesis 33: 97-101 (2002)). In fact, DLX5 was indicated to be a master regulatory transcriptional factor essential for initiating the cascade involved in osteoblast differentiation in mammals (Lee J Y, et al., Mol Cells 22: 182-8 (2006), Ryoo H M, et al., Mol Endocrinol 11: 1681-94 (1997)).

In the present study, it was demonstrated that DLX5 gene was frequently overexpressed in lung cancer, and might play an important role in the development/progression of lung cancers. In this study, knockdown of DLX5 expression by siRNA in lung cancer cells resulted in suppression of cell growth. Moreover, clinicopathological evidence obtained through present tissue-microarray experiments indicated that NSCLC patients with DLX5-strong positive tumors had shorter cancer-specific survival periods than those with DLX5-weak positive/negative tumors. The results obtained by in vitro and in vivo assays strongly suggested that DLX5 is likely to be an important growth factor and be associated with a more malignant phenotype of lung-cancer cells. Since the DLX5 protein is present mainly in the nucleus and includes a homeodomain, it should play an important role in the transcriptional regulation, and directly or indirectly transactivate various downstream genes in lung cancer cells. Further investigations of DLX5 pathway could lead to a better understanding of the mechanisms of oncogenes activation in pulmonary carcinogenesis. Because DLX5 is not expressed in any of normal adult tissues except the placenta, selective inhibition of DLX5 activity could be a promising therapeutic strategy that is expected to have a powerful biological activity against cancer with a minimal risk of adverse events.

In summary, the DLX5 gene appears to play an important role in the growth/progression of lung cancers. DLX5 overexpression in resected specimens may be a useful index for application of adjuvant therapy to the patients who are likely to have poor prognosis. In addition, the data herein strongly suggest the potential of designing new anti-cancer drugs and cancer vaccines to specifically target the DLX5 for human cancer treatment.

### Part III

### NPTX1 Related Experiments

### Example 12

### General Methods

1. Cell lines and tissue samples. The 23 human lung-cancer cell lines used in this study included nine adenocarcinomas (ADCs; A427, A549, LC319, PC-3, PC-9, PC-14, NCI-H1373, NCI-H1666, and NCI-H1781), nine squamous-cell carcinomas (SCCs; EBC-1, LU61, NCI-H226, NCI-H520, NCI-H647, NCI-H1703, NCI-H2170, RERF-LC-AI, and SK-MES-1), one large-cell carcinoma (LCC; LX1), and four small-cell lung cancers (SCLCs; DMS114, DMS273, SBC-3, and SBC-5). All cells were grown in monolayers in appropriate media supplemented with 10% fetal calf serum (FCS) and were maintained at 37 degree Centigrade in an atmosphere of humidified air with 5% CO₂. Human small airway epithelial cells (SAEC) were grown in optimized medium (SAGM) purchased from Cambrex Bio Science Inc (Walkersville, Md.). Primary lung-cancer tissue samples had been obtained with written informed consent as described previously (Kikuchi 2003; Taniwaki 2006). A total of 374 formalin-fixed samples of primary NSCLCs including 238 ADCs, 95 SCCs, 28 LCCs, and 13 ASCs, and adjacent normal lung tissue, had been obtained earlier along with clinicopathological data from patients who had undergone surgery at Saitama Cancer Center (Saitama, Japan). 13 SCLCs were obtained from individuals who underwent autopsy at Hiroshima University (Hiroshima, Japan). The histological classification of the tumor specimens was based on WHO criteria (Travis WD). NSCLC specimen and five tissues (heart, liver, lung, kidney, and adrenal gland) from post-mortem materials (2 individuals with ADC) were also obtained from Hiroshima University. This study and the use of all clinical materials mentioned were approved by individual institutional Ethical Committees.
2. Serum samples. Serum samples were obtained with informed consent from 102 healthy individuals as controls (84 males and 18 females; median age 49.0+/−7.46 SD, range 31-60) and from 80 non-neoplastic lung disease patients with chronic obstructive pulmonary disease (COPD) enrolled as a part of the Japanese Project for Personalized Medicine (BioBank Japan) or admitted to Hiroshima University Hospital (68 males and 12 females; median age 66.4+/−5.92 SD, range 54-73). All of these patients were current and/or former smokers (The mean [+/−1 SD] of pack-year index (PYI) was 64.2+/−41.6; PYI was defined as the number of cigarette packs [20 cigarette per pack] consumed a day multiplied by years). The healthy individuals showed no abnormalities in complete blood cell counts, C-reactive proteins (CRP), erythrocyte sedimentation rates, liver function tests, renal function tests, urinalyses, fecal examinations, chest X-rays, or electrocardiograms. Serum samples were also obtained with informed consent from 223 lung-cancer patients admitted to Hiroshima University Hospital, as well as Kanagawa Cancer Center Hospital, and from 106 patients with lung cancer enrolled as a part of the Japanese Project for Personalized Medicine BioBank Japan; (227 males and 102 females; median age 66.6+/−11.2 SD, range 30-86). Samples were selected for the study on the basis of the following criteria: (1) patients were newly diagnosed and previously untreated and (2) their tumors were pathologically diagnosed as lung cancers (stages I-IV). These 329 cases included 185 ADCs, 51 SCCs, and 93 SCLCs. Clinicopathological records were fully documented. Serum was obtained at the time of diagnosis and stored at −80 degree Centigrade.

### 3. Semiquantitative RT-PCR Analysis.

Total RNA was extracted from cultured cells and clinical tissues using Trizol reagent (Life Technologies, Inc. Gaithersburg, Md.) according to the manufacturer's protocol. Extracted RNAs and normal human-tissue polyA RNAs were treated with DNase I (Roche Diagnostics, Basel, Switzerland) and then reverse-transcribed using oligo (dT)₁₂₋₁₈ primer and SuperScript II reverse transcriptase (Life Technologies, Inc.). Semiquantitative RT-PCR experiments were carried out with synthesized NPTX1 gene-specific primers (5′-GTTGGGGACCGGAGGTAAA-3′ and 5′-AAACCACGACTTCGTCAAGC-3′), with synthesized NPTXR gene-specific primers (5′-TCTGCCAGATCTTCCCATCT-3′ and 5′-GGCTTCAGCTTCCTCATCTG-3′), or with beta-actin (ACTB)-specific primers (5′-ATCAAGATCATTGCTCCTCCT-3′ and 5′-CTGCGCAAGTTAGGTTTTGT-3′) as an internal control. All PCR reactions involved initial denaturation at 94 degrees C. for 2 min followed by 22 (for ACTB) or 35 cycles (for NPTX1) of 94 degree Centigrade 30 s, 54 or 60 degree Centigrade for 30 s, and 72 degree Centigrade for 60 s on a GeneAmp PCR system 9700 (Applied Biosystems, Foster City, Calif.).
4. Northern-blot analysis. Human multiple-tissue blots (BD Biosciences, Palo Alto, Calif.) were hybridized with ³²P-labeled PCR products. PCR product of NPTX1 was prepared as a probe by RT-PCR using the same primers above. Prehybridization, hybridization, and washing were performed according to the supplier's recommendations. The blots were autoradiographed with intensifying screens at −80 degree Centigrade for one week.
5. Preparation of anti-NPTX1 antibodies. Rabbit polyclonal antibodies (pAbs) specific for NPTX1 (BB017) were raised by immunizing rabbits with GST-fused human NPTX1 protein (codons 20-145 and 297-430), and purified using a standard protocol. Mouse monoclonal antibody (mAb) specific for human NPTX1 (mAb-75-1) was also generated by immunizing BALB/c mice (Chowdhury) intradermally with plasmid DNA encoding human NPTX1 protein using gene gun. NPTX1 mAb was purified by affinity chromatography from cell culture supernatant. NPTX1 mAb was proved to be specific for human NPTX1, by western-blot analysis using lysates of lung-cancer cell lines which expressed NPTX1 endogenously or not.
6. Western blotting.
Cells were lysed with radioimmunoprecipitation assay buffer [50 mmol/L Tris-HCl (pH 8.0), 150 mmol/L NaCl, 1% NP40, 0.5% deoxychorate-Na, 0.1% SDS] containing Protease Inhibitor Cocktail Set III (Calbiochem, Darmstadt, Germany). Protein samples were separated by SDS-polyacrylamide gels and electroblotted onto Hybond-ECL nitrocellulose membranes (GE Healthcare Bio-Sciences, Piscataway, N.J.). Blots were incubated with a mouse monoclonal anti-NPTX1 antibody (mAb-75-1). Antigen-antibody complexes were detected using secondary antibodies conjugated to horseradish peroxidase (GE Healthcare Bio-Sciences). Protein bands were visualized by enhanced chemiluminescence Western blotting detection reagents (GE Healthcare Bio-Sciences).

### 7. Immunofluorescence Analysis.

Cells were plated on glass coverslips (Becton Dickinson Labware, Franklin Lakes, N.J.), fixed with 4% paraformaldehyde, and permeabilized with 0.1% Triton X-100 in PBS for 3 minutes at room temperature. Non-specific binding was blocked by CASBLOCK (ZYMED, South San Francisco, Calif.) for 10 minutes at room temperature. Cells were then incubated for 60 minutes at room temperature with primary antibodies for human NPTX1 antibody (mAb-75-1) diluted in PBS containing 3% BSA. After being washed with PBS, the cells were stained by Alexa Fluor 488-conjugated secondary antibody (Molecular Probes) for 60 minutes at room temperature. After another wash with PBS, each specimen was mounted with Vectashield (Vector Laboratories, Inc, Burlingame, Calif.) containing 4′,6′-diamidine-2′-phenylindolendihydrochrolide (DAPI) and visualized with Spectral Confocal Scanning Systems (TSC SP2 AOBS: Leica Microsystems, Wetzlar, Germany).

### 8. Immunohistochemistry and Tissue Microarray.

To investigate the presence of NPTX1 protein in clinical samples embedded in paraffin blocks, sections were stained in the following manner. Briefly, 100 mg/ml of mouse monoclonal anti-human NPTX1 antibody (mAb-75-1) was added after blocking of endogenous peroxidase and proteins. The sections were incubated with HRP-labeled anti-mouse IgG as the secondary antibody. Substrate-chromogen was added and the specimens were counterstained with hematoxylin.

Tumor-tissue microarrays were constructed using 387 formalin-fixed primary lung cancers (374 NSCLCs and 13 SCLCs), as described elsewhere (Callagy, 2003, 2005; Chin). The tissue area for sampling was selected based on visual alignment with the corresponding HE-stained section on a slide. Three, four, or five tissue cores (diameter 0.6 mm; height 3-4 mm) taken from a donor tumor block were placed into a recipient paraffin block using a tissue microarrayer (Beecher Instruments, Sun Prairie, Wis.). A core of normal tissue was punched from each case, and 5-m m sections of the resulting microarray block were used for immunohistochemical analysis. Three independent investigators semi-quantitatively assessed NPTX1 positivity without prior knowledge of clinicopathological data. The intensity of NPTX1 staining was evaluated using following criteria: strong positive (scored as 2+), dark brown staining in more than 50% of tumor cells completely obscuring cytoplasm; weak positive (1+), any lesser degree of brown staining appreciable in tumor cell cytoplasm; absent (scored as 0), no appreciable staining in tumor cells. Cases were accepted as strongly positive only if reviewers independently defined them as such.

9. Statistical analysis. Statistical analyses were performed using the StatView statistical program (SaS, Cary, N.C.). Associations between clinicopathological variables and positivity for NPTX1 were compared by Fisher's exact test. Tumor-specific survival was evaluated with the Kaplan-Meier method, and differences between the two groups were evaluated with the log-rank test. Risk factors associated with the prognosis were evaluated using Cox's proportional-hazard regression model with a step-down procedure. Proportional-hazard assumptions were checked and satisfied; only those variables with statistically significant results in univariate analysis were included in a multivariate analysis. The criterion for removing a variable from the model was the likelihood ratio statistic, which was based on the maximum partial likelihood estimate (default P value of 0.05 for removal).
10. ELISA. Serum levels of NPTX1 were measured by ELISA system which had been originally constructed. First of all, 100 ml per well of a mouse monoclonal antibody specific to NPTX1 (mAb-75-1; 4 mg/ml) was added to a 96-well microplate (Nunc Maxisorp Bioscience, Inc., Naperville, Ill.) as a capture antibody and incubated for 2 hours at room temperature. After washing away any unbound antibody using PBST (PBS containing 1% bovine serum albumin (BSA) and 0.05% Tween) at room temperature, 200 ml per well of 5% BSA was added to the wells and incubated for 2 hours at room temperature for blocking. After three times wash, 100 ml per well of 3-fold diluted sera in PBS with 1% BSA were added to the wells and incubated for 2 hours at room temperature. After washing away any unbound substances, 100 ml per well of a rabbit polyclonal antibody specific for NPTX1 (BB017; 0.01 mg/ml) biotinylated using Biotin Labeling Kit-NH₂ (DOJINDO, Kumamoto, Japan) was added to the wells as a detection antibody and incubated for 2 hours at room temperature. After three times wash to remove any unbound antibody-enzyme reagent, Streptoavidin-Horseradish Peroxidase (SAv-HRP) was added to the wells and incubated for 20 minutes. After three times wash, 100 ml per well of a substrate solution (R&D Systems, Inc., Minneapolis, Minn.) was added to the wells and allowed to react for 30 minutes. The reaction was stopped by adding 50 ml of 2 N sulfuric acid. Color intensity was determined by a photometer at a wavelength of 450 nm, with a reference wavelength of 570 nm. Levels of CEA in serum were measured by ELISA with a commercially available enzyme test kit (HOPE Laboratories, Belmont, Calif.), according to the supplier's recommendations. Levels of CYFRA in serum were measured by ELISA with a commercially available enzyme test kit (DRG International Inc USA, Mountainside, N.J.), according to the supplier's recommendations. Levels of proGRP in serum were measured by ELISA with a commercially available enzyme test kit (TFB Tokyo Japan), according to the supplier's recommendations. Differences in the levels of NPTX1, CEA, CYFRA and proGRP between tumor groups and a healthy control group were analyzed by Mann-Whitney U tests. The levels of NPTX1, CEA, CYFRA and proGRP were evaluated by receiver-operating characteristic (ROC) curve analysis to determine cutoff levels with optimal diagnostic accuracy and likelihood ratios. The correlation coefficients between NPTX1 and CEA were calculated with Spearman rank correlation. Significance was defined as P<0.05.
11. RNA interference assay. As noted above, a vector-based RNA interference (RNAi) system, psiH1BX3.0, to direct the synthesis of siRNAs in mammalian cells has been previously established (Suzuki, 2003). Herein, 10 micro g of siRNA-expression vector were transfected, using 30 micro L of Lipofectamine 2000 (Invitrogen), into a NSCLC cell line, A549 and a SCLC cell line, SBC-5, which overexpressed NPTX1. The transfected cells were cultured for five days in the presence of appropriate concentrations of geneticin (G418), after which cell numbers and viability were measured by Giemsa staining and triplicate MTT assays; briefly, cell-counting kit-8 solution (DOJINDO) was added to each dish at a concentration of 1/10 volume, and the plates were incubated at 37 degree Centigrade for additional 2 hours. Absorbance was then measured at 450 nm with a Microplate Reader 550 (BIO-RAD, Hercules, Calif.). To confirm suppression of NPTX1 mRNA expression, semiquantitative RT-PCR experiments were carried out with the synthesized NPTX1-specific primers. The target sequences of the synthetic oligonucleotides for RNAi were as follows:

control 1 (Luciferase, LUC: Photinus pyralis
luciferase gene),
5′-CGTACGCGGAATACTTCGA-3′;
control 2 (Scramble, SCR: chloroplast Euglena
gracilis gene coding for 5S and 16S rRNAs),
5′-GCGCGCTTTGTAGGATTCG-3′;
NPTX1 siRNA-1 (si-NPTX1-1),
5′-CTCGGGCAAACTTTGCAAT-3′;
NPTX1 siRNA-2 (si-NPTX1-2),
5′-GGTGAAGAAGAGCCTGCCA-3′.

12. Cell-growth assay. The entire coding sequence of NPTX1 was cloned into the appropriate site of pcDNA3.1 myc-His plasmid vector (Invitrogen, Carlsbad, Calif.). COS-7 cells transfected either with plasmids expressing myc-His-tagged NPTX1 or with mock plasmids were grown for eight days in DMEM containing 10% FCS in the presence of appropriate concentrations of geneticin (G418). Viability of cells was evaluated by MTT assay; briefly, cell-counting kit-8 solution (DOJINDO) was added to each dish at a concentration of 1/10 volume, and the plates were incubated at 37 degree Centigrade for additional 2 hours. Absorbance was then measured at 450 nm as a reference, with a Microplate Reader 550 (BIO-RAD, Hercules, Calif.)
13. Matrigel invasion assay. NIH-3T3 cells transfected either with pcDNA3.1-myc/His plasmids expressing human NPTX1 or with mock plasmids were grown to near confluence in DMEM containing 10% FCS. The cells were harvested by trypsinization, washed in DMEM without addition of serum or proteinase inhibitor, and suspended in DMEM at concentration of 1×10⁵ cells/ml. Before preparing the cell suspension, the dried layer of Matrigel matrix (Becton Dickinson Labware, Franklin Lakes, N.J.) was rehydrated with DMEM for 2 hours at room temperature. DMEM (0.75 ml) containing 10% FCS was added to each lower chamber in 24-well Matrigel invasion chambers, and 0.5 ml (5×10⁴ cells) of cell suspension was added to each insert of the upper chamber. The plates of inserts were incubated for 22 hours at 37 degree Centigrade. After incubation the chambers were processed; cells invading through the Matrigel were fixed and stained by Giemsa as directed by the supplier (Becton Dickinson Labware).

### Example 13

### NPTX1 Expression in Lung Tumors and Normal Tissues

To search for novel target molecules for development of therapeutic agents and/or diagnostic biomarkers for lung cancer, genes were first screened that showed more than a 3-fold higher level of expression in cancer cells than in normal cells, in half or more of 101 lung cancer samples analyzed by cDNA microarray (Kikuchi, 2003, 2006, Kakiuchi, 2004, Taniwaki). Among 27,648 genes screened, the overexpression of NPTX1 was identified in the great majority of lung cancers examined, and confirmed its transactivation by semiquantitative RT-PCR experiments in 10 of 15 additional lung-cancer tissues and in 17 of 23 lung-cancer cell lines (FIG. 7A, upper and lower panels). A mouse monoclonal antibody specific for human NPTX1 was subsequently generated, and confirmed by Western-blot analysis as an expression of endogenous NPTX1 protein in four lung-cancer cell lines (three NPTX1-positive cells: NCI-H226, NCI-H520, and SBC-5 vs. one NPTX1-negative line, NCI-H2170) and small airway epithelia derived cells (SAEC) (FIG. 7B).

Immunofluorescence analysis was performed to examine the subcellular localization of endogenous NPTX1 in these four lung-cancer cell lines. NPTX1 was detected at cytoplasm of tumor cells with granular appearance at a high level in NCI-H226 cells, at a low level in NCI-H520 and SBC-5 cells, but not in NCI-H2170 cells, which was concordant with the result of western-blotting (FIG. 7C). Since the NPTX1 was a secretory protein (Schlimgen), the ELISA method was applied to examine its presence in the culture media of these lung-cancer cell lines. NPTX1 protein was detected in media of NCI-H226, NCI-H520 and SBC-5 cells, but not in medium of NCI-H2170 cells (FIG. 7D). The amounts of detectable NPTX1 in the cell lysate by Western blot and in the culture media by ELISA showed good correlation with those of NPTX1 detected by RT-PCR, indicating that the antibody specifically bound to NPTX1 protein.

Northern-blot analysis using human NPTX1 cDNA as a probe detected a very weak 6.5-kb band only in brain and adrenal gland; no expression was observed in any other tissues (FIG. 8A). The expression of NPTX1 protein was also examined with monoclonal antibody specific to NPTX1 on five normal tissues (liver, heart, kidney, lung, adrenal gland) and lung ADC tissues. NPTX1 staining was mainly observed at cytoplasm of tumor cells and cells (cortex) in adrenal gland, but not detected in normal cells (FIG. 8B). The expression levels of NPTX1 protein in lung cancer were significantly higher than those in adrenal gland.

### Example 14

### Association of NPTX1 Expression with Poor Prognosis

To verify the biological and clinicopathological significance of NPTX1, the expression of NPTX1 protein was examined by means of tissue microarrays containing primary NSCLC tissues from 374 NSCLC patients as well as SCLC tissues from 13 patients. Positive cytoplasmic staining for NPTX1 was observed in 56.1% of surgically-resected NSCLCs (210/374) and in 69.2% of SCLCs (9/13), while no staining was observed in any of normal lung tissues examined. (FIG. 8C). A correlation of its positive staining was then examined with various clinicopathological parameters in 374 NSCLC patients. A pattern of NPTX1 expression was classified on the tissue array, ranging from absent (scored as 0) to weak/strong positive (scored as 1+˜2+) (FIG. 8D, upper panels; see Methods).

Of the 374 NSCLC cases examined, NPTX1 was strongly stained in 139 (37.1%; score 2+), weakly stained in 71 (19.0%; score 1+), and not stained in 164 cases (43.9%; score j) (Table 1A). In this study, tumor size (pT₂₋₄ versus pT₁; P<0.0001 by Fisher's exact test) and lymph-node metastasis (pN₁₋₂ versus pN₀; P=0.0044 by Fisher's exact test) were significantly associated with the NPTX1 status (Table 1B). Kaplan-Meier analysis indicated that the median survival time of patients with strong NPTX1-staining (scored 2+) was significantly shorter than that of NSCLC patients with absent/weak NPTX1-staining (scored 0, 1+) (P<0.0001 by log-rank test; FIG. 8D, lower panel). In multivariate analysis of the prognostic factors, pT stage, pN stage, and strong NPTX1 positivity were indicated to be an independent prognostic factor (Table 1B).

TABLE 1A
Association between NPTX1-positivity in NSCLC tissues and
patients' characteristics (n = 374)

| | | NPTX1 | NPTX1 | | P-value |
|---|---|---|---|---|---|
| | | strong | weak | NPTX1 | strong vs |
| | Total | positive | positive | absent | weak/ |
| | n = 374 | n = 139 | n = 71 | n = 164 | absent |
| Gender | | | | | |
| Male | 259 | 104 | 47 | 108 | NS |
| Female | 115 | 35 | 24 | 56 | |
| Age (years) | | | | | |
| <65 | 188 | 72 | 35 | 81 | NS |
| >=65 | 186 | 67 | 36 | 83 | |
| Histological type | | | | | |
| ADC | 238 | 93 | 38 | 107 | NS |
| SCC | 95 | 26 | 24 | 45 | |
| Others | 41 | 20 | 9 | 12 | |
| pT factor | | | | | |
| T1 | 125 | 27 | 26 | 72 | <0.0001** |
| T2 − T4 | 249 | 107 | 45 | 92 | |
| pN factor | | | | | |
| N0 | 229 | 72 | 44 | 113 | 0.0044** |
| N1 + N2 | 145 | 67 | 27 | 51 | |

ADC, adenocarcinoma;
SCC, squamous-cell carcinoma
Others, large-cell carcinoma (LCC) plus adenosquamous-cell carcinoma (ASC)
*ADC versus non-ADC
**P < 0.05 (Fisher's exact test)
NS, no significance

TABLE 1B
Cox's proportional hazards model analysis of prognostic
factors in patients with NSCLCs

| | Hazards | | Unfavorable/ | |
|---|---|---|---|---|
| Variables | ratio | 95% CI | Favorable | P-value |
| Univariate analysis | | | | |
| NPTX1 | 2.224 | 1.672-2.958 | Strong(2+)/ | <0.0001* |
| | | | Weak(1+) or (−) | |
| Age (years) | 1.329 | 0.998-1.770 | 65<=/<65 | NS |
| Gender | 1.750 | 1.256-2.440 | Male/Female | 0.001* |
| Histological type | 1.474 | 1.106-1.965 | non-ADC/ADC¹ | 0.0081* |
| pT factor | 2.667 | 1.860-3.822 | T2 − T4/T1 | <0.0001* |
| pN factor | 2.565 | 1.928-3.414 | N1 + N2/N0 | <0.0001* |
| Multivariate analysis | | | | |
| NPTX1 | 1.898 | 1.412-2.552 | Strong(2+)/ | <0.0001* |
| | | | Weak(1+) or (−) | |
| Gender | 1.331 | 0.922-1.921 | Male/Female | NS |
| Histological type | 1.248 | 0.907-1.717 | non-ADC/ADC¹ | NS |
| pT factor | 1.910 | 1.309-2.789 | T2 − T4/T1 | 0.0008* |
| pN factor | 2.236 | 1.674-2.986 | N1 + N2 /N0 | <0.0001* |

¹ADC, adenocarcinoma
*P < 0.05
NS, no significance

### Example 15

### Serum Levels of NPTX1 in Lung Cancer Patients

Since NPTX1 encodes a secretory protein, it was investigated whether the NPTX1 protein was secreted into sera of patients with lung cancer. ELISA experiments detected NPTX1 in serologic samples from the majority of the 329 patients with lung cancer; serum levels of NPTX1 in lung cancer patients were 1.36+/−1.60 ng/ml (mean+/−1SD) and those in healthy individuals were 0.59+/−0.44 ng/ml (The difference was significant with P-value of <0.001 by Mann-Whitney U test; FIG. 9A). According to histological types of lung cancer, the serum levels of NPTX1 were 1.41+/−1.27 ng/ml in ADC patients, 1.09+/−0.95 ng/ml in SCC patients, and 1.42+/−2.33 ng/ml in SCLC patients; the differences among the three histologic types were not significant. Serum levels of NPTX1 were 0.67+/−0.48 ng/ml in benign lung disease of COPD patients. Serum levels of NPTX1 in lung cancer patients were significantly higher than those of normal volunteers and COPD patients (P<0.0001). High levels of serum NPTX1 were detected even in patients with earlier-stage tumors. Furthermore levels of NPTX1 were significantly more common in serum from patients with locally advanced lung cancer (stage IIIB) or distant organ metastasis (stage IV or ED) than in those with earlier stage diseases (stages I-IIIA or LD) (FIG. 9B). Using receiver-operating characteristic (ROC) curves drawn with the data of these 329 cancer patients and 102 healthy controls, the cut-off level in this assay was set to provide optimal diagnostic accuracy and likelihood ratios for NPTX1, i.e., 1.28 ng/ml for NPTX1 (with a sensitivity of 41.5% for NSCLC, 44.3% for ADC, 29.4% for SCC, and 31.2% for SCLC) and a specificity of 96.1% for NSCLC). Among the 80 patients with COPD, 7 (8.8%) had a positive NPTX1 level. It was then performed ELISA experiments using paired preoperative and postoperative (two months after the surgery) serum samples from four NSCLC patients to monitor the levels of serum NPTX1 in the same patients. The concentration of serum NPTX1 was dramatically reduced after surgical resection of primary tumors (FIG. 9C). The present inventors further compared the serum NPTX1 values with the expression levels of NPTX1 in primary tumors in the same set of 12 NSCLC cases whose serum had been collected before surgery (six patients with NPTX1-positive tumors and six with NPTX1-negative tumors). The levels of serum NPTX1 showed good correlation with the expression levels of NPTX1 in primary tumor (FIG. 9D). The results independently support the high specificity and the great potentiality of serum NPTX1 as a biomarker for detection of cancer at an early stage and for monitoring of the resection of tumors and relapse of the disease.

### Example 16

### Combination Assay of NPTX1 CEA, CYFRA and proGRP as Tumor Markers

To evaluate the clinical usefulness of serum NPTX1 level as a tumor detection biomarker in clinic, the serum levels of two conventional tumor markers (CEA for ADC patients, CYFRA for SCC patients and proGRP for SCLC patients) were also measured by ELISA, in the same set of serum samples from cancer patients and control individuals. Cutoff levels in this assay determined by ROC analyses were set to result in optimal diagnostic accuracy and likelihood ratios for CEA, i.e., 2.5 ng/ml (with a sensitivity of 38.4% and a specificity of 98.0% for ADC), CYFRA, i.e., 2.0 ng/ml (with a sensitivity of 29.4% and a specificity of 98.0% for SCC) and proGRP, i.e., 46.0 pg/ml (with a sensitivity of 62.4% and a specificity of 99.0% for SCLC). The correlation coefficient between serum NPTX1 and CEA values was not significant (Spearman rank correlation coefficient: p=0.109, P=0.1474).

Measuring both NPTX1 and CEA in serum can improve overall sensitivity for detection of lung ADC patients to 64.9%. False-positive rates for either of the two tumor markers among normal volunteers (control group) amounted to 4.9%. The correlation coefficient between serum NPTX1 and CYFRA values was not significant (Spearman rank correlation coefficient: p=0.013, P=0.9242). Measuring both NPTX1 and CYFRA in serum can improve overall sensitivity for detection of lung SCC patients to 62.3%. False-positive rates for either of the two tumor markers among normal volunteers (control group) amounted to 5.9%. The correlation coefficient between serum NPTX1 and proGRP values was not significant (Spearman rank correlation coefficient: p=0.161, P=0.1232). Measuring both NPTX1 and proGRP in serum can improve overall sensitivity for detection of lung SCLC patients to 72.0%. False-positive rates for either of the two tumor markers among normal volunteers (control group) amounted to 4.9%.

### Example 17

### Autocrine Growth-Promoting Effect of NPTX1 on Lung Cancer Cells

To assess whether up-regulation of NPTX1 plays a role in growth or survival of lung-cancer cells, plasmids were designed and constructed to express siRNA against NPTX1 (si-NPTX1-1, -2), along with two different control plasmids (siRNAs for Luciferase (LUC), and Scramble (SCR)), and transfected them into A549 and SBC-5 cells to suppress expression of endogenous NPTX1. The amount of NPTX1 in the cells transfected with si-NPTX1-2 was significantly reduced compared to cells transfected with any of the two control siRNAs (FIG. 10A, upper panels); si-NPTX1-1 showed almost no suppressive effect on NPTX1 expression. In accord with its suppressive effect on gene expression levels, transfected si-NPTX1-2 caused significant decreases in colony numbers and cell viability measured by colony-formation and MTT assays, but no such effects were observed by two control siRNAs or si-NPTX1-1 (FIG. 10A, middle and lower panels).

To further disclose a potential role of NPTX1 in tumorigenesis, the present inventors prepared plasmids designed to express either NPTX1 (pcDNA3.1-NPTX1-myc/His) or mock vector. It was transfected these plasmid DNAs into COS-7 cells, in which NPTX1 expression was detectable, and carried out the colony-formation and MTT assays. The cell viability was significantly increased in dishes containing COS-7 cells that had been transfected with the sense-strand of NPTX1 cDNA, in comparison to cells transfected with the mock vector (FIG. 10B).

Next, it was investigated whether the affinity purified anti-NPTX1 monoclonal antibody (mAb-75-1) could inhibit the growth of COS-7 cells cultured in the medium containing NPTX1. Expectedly, growth enhancement caused by the addition of NPTX1 was neutralized by the 50 nM concentration of the anti-NPTX1 antibody, and the viability of COS-7 cells became almost equivalent to the cells cultured without NPTX1 (FIG. 10B). Subsequently, autocrine assays were carried out using the recombinant NPTX1 protein. To investigate whether secreted NPTX1 would affect cell growth, COS-7 cells were incubated with NPTX1 at final concentration of 0.1 nM to 1 nM in the culture medium. COS-7 cells incubated with NPTX1 showed enhancement of the cell growth by MTT assays, compared with control, in a dose dependent manner (FIG. 10C).

These results suggested that the growth-promoting effect of NPTX1 was likely to be mediated through binding of NPTX1 to a receptor(s) on the cell surface of COS-7. Next, it was investigated whether anti-NPTX1 antibody (50 nM) could inhibit the growth of COS-7 cells cultured in the medium containing NPTX1. Expectedly, growth enhancement caused by the addition of NPTX1 was neutralized by the 50 nM concentration of anti-NPTX1 antibody, and the viability of COS-7 cells became almost equivalent to the cells cultured without NPTX1 (FIG. 10C). These results suggested that the growth-promoting effect of NPTX1 was likely to be mediated through binding of NPTX1 to a receptor(s) on the cell surface of COS-7.

Next, it was investigated the effect of anti-NPTX1 antibody on the growth of NPTX1-positive lung cancer cell lines, SBC-5 and A549, as well as NPTX1-negative SBC-3 and NCI-H2170 cells. The growth of both SBC-5 and A549 was suppressed in a dose dependent manner by the addition of anti-NPTX1 monoclonal antibody (25 or 50 nM; mAb-75-1) into the culture media (SBC-5: P=0.012; A549: 25 or 50 nM P=0.027 and P=0.0003, respectively; each paired t-test), whereas that of NPTX1-non-expressing SBC-3 cells was not affected (FIG. 10D). These data indicated that NPTX1 functions as an autocrine/paracrine growth factor for the proliferation of lung cancer cells and could be a potential immunotherapeutic target for antibody-based therapy.

### Example 18

### Activation of Cellular Invasion by NPTX1

As the immunohistochemical analysis on tissue microarray had indicated that NSCLC patients with NPTX1 strong-positive tumors showed shorter cancer-specific survival period than those with NPTX1-weak positive or -negative tumors, a possible role of NPTX1 in cellular invasion was examined using Matrigel assays, using NIH-3T3 cells. Transfection of NPTX1 cDNA into NIH-3T3 cells significantly enhanced its invasive activity through Matrigel, compared to cells transfected with mock vector (FIG. 11).

### Example 19

### Inhibition of Growth of Lung Cancer Cells by Anti-NPTX1 Monoclonal Antibody In Vivo

This invention further investigated the in vivo tumor suppressive effect of the anti-NPTX1 antibody as a therapeutic agent in mice model. The present inventors grafted the A549 cells to subcutaneous of 7-week-old female BALB/c nude mice (nu/nu), and administered 300 micro g/body of the affinity purified anti-NPTX1 monoclonal antibody (mAb-75-1), or normal mice IgG (control) into the tumor twice a week for 30 days. The anti-NPTX1 monoclonal antibody (mAb-75-1) caused a significant suppression of the growth of A549 lung carcinoma, while the same dose of normal mice IgG unaffected the tumor growth (P=0.016 by each paired t test; FIG. 12, top panels). HE staining using frozen section of the resected tumors detected significant fibromatic change and decrease of viable cancer cells in anti-NPTX1 antibody-treated tumor tissues (FIG. 12, bottom panels). Taken together, these results revealed that the anti-NPTX1 monoclonal antibody (mAb-75-1) had the growth suppressive effect on cancer cells in vitro and in vivo.

### Example 20

### NPTXR as a Receptor for NPTX1 in a Growth-Promoting Pathway

A known NPTX1 receptor, NPTXR was suggested to play a role in the transport of a presynaptic snake venom toxin taipoxin into synapses that may represent a novel neuronal uptake pathway involved in the clearance of synaptic debris (Kirkpatrick L L, et al., J Biol Chem 278: 17786-92 (2000), Dodds D C, et al., J Biol Chem 272(34): 21488-94 (1997)). To investigate whether NPTXR genes was expressed in lung cancers and responsible for growth promoting effect, the present inventors analyzed expression of NPTXR in lung cancer cell lines, and in clinical tissues by semiquantitative RT-PCR experiments. NPTXR was expressed at a relatively high level in lung cancer samples, but not in normal lung (FIG. 7E). The expression pattern of NPTXR showed good concordance with NPTX1 expression in these tumors. COS-7 cells examined on autocrine growth-promoting effect of NPTX1 as described above, were confirmed by semiquantitative RT-PCR analysis and immunocytochemical analysis to express endogenously NPTXR (data not shown). The data suggested that NPTX1 is likely to mediate its growth-promoting effect through interaction with NPTXR in lung cancer cells.

To investigate binding of NPTX1 to the endogenous NPTXR on the COS-7 and lung cancer cells, it was performed receptor-ligand binding assay using COS-7 and lung cancer SBC-5 cells that had endogenously expressed NPTXR and was transfected with NPTX1-expressing vector. It was confirmed secretion of exogenous NPTX1 in the culture media of these cells, and detected binding of NPTX1 to the surface of the cells by flow cytometric analysis (Representative data of COS-7 is shown in FIG. 15A). It was also observed colocalization of secreted NPTX1 with endogenous NPTXR on the surface of these two cell lines (COS-7 and SBC-5 cells) (FIG. 13A). To confirm the specific interaction of NPTX1 to COS-7 and SBC-5 cells, we added stripping buffer (glycine 100 mM, 500 mM NaCl, pH 2.5) in their media to remove anti-NPTX1 and anti-NPTXR antibodies as a primary antibody bound to the cell surface. After glycine treatment, NPTX1 as well as NPTXR were not detected on the cell surface of the cells, suggesting the interaction of NPTX1 to NPTXR on the cell surface (FIGS. 13B and 13C). To examine the direct association between NPTX1 and NPTXR, the inventors transiently expressed myc/His-tagged NPTX1 in COS-7 or SBC-5 cells. Cell lysates were immunoprecipitated by anti-myc or aniti NPTXR antibody, and were served for western-blot analysis using anti-NPTXR or anti-myc antibody. it was found co-precipitation of NPTX1 and NPTXR (FIG. 15B). These results confirm an interaction between NPTX1 and NPTXR, implying the existence of NPTX1/NPTXR complex.

It was examined the biological significance of the NPTX1-receptor interaction in pulmonary carcinogenesis using plasmids designed to express siRNA against NPTXR (si-NPTXR-1 and si-NPTXR-2). Transfection of either of these plasmids into A549 or SBC-5 cells suppressed expression of the endogenous receptor in comparison to cells containing any of the two control siRNAs (FIG. 13D, top panels). In accordance with the reduced expression of the receptors, A549 and SBC-5 cells showed significant decreases in cell viability and numbers of colonies (FIG. 13D, middle and bottom panels). These results strongly supported the possibility that NPTX1, by interaction with NPTXR, might play a very significant role in development/progression of lung cancer.

### Example 21

### Internalization of NPTX1 after Binding with NPTXR

To determine the mechanism involved in the regulation of NPTX1/NPTXR signaling, the present inventors examined whether NPTX1/NPTXR could be internalized when cells were exposed to secreted NPTX1, through confocal microscopy observation of the subcellular distribution of the NPTX1 and NPTXR. Recipient COS-7 or SBC-5 cells were grown on coverslips overnight at 37° C. in medium. It was also collected the supernatants of donor COS-7 or SBC-5 cells transfected with NPTX1 vector. Then, the recipient COS-7 or SBC-5 cells were incubated with the supernatant of donor cells for three hours. The present Mentors detected binding of NPTX1 to the surface of these cells by immunocytochemistry (FIGS. 14A and 14B). It was also observed colocalization of exogenous NPTX1 with endogenous NPTXR on the surface of these two cell lines (data not shown). Then, immunocytochemistry was done under membrane-permeabilizing conditions and we detected internalized exogenous NPTX1 (FIGS. 14A and 14B). 1 or 3 hours after treatment of the recipient COS-7 cells with conditioned medium from donor NPTX1-transfected (+) COS-7 cells, internalized NPTX1 was detected by western blotting using anti-myc antibodies. Recipient COS-7 cells appeared to uptake in a time-dependent manner the secreted NPTX1 in conditioned medium from donor NPTX1-transfected (+) COS-7 cells (FIG. 14C). All analyses were performed blind, without experimenter knowledge of the treatment conditions.

### Discussion:

In spite of many advances in diagnostic imaging of tumors, combination chemotherapy, modern surgical techniques and radiation therapy, little improvement has been achieved within the last decade in terms of prognosis and quality of life for most patients with lung cancer. In fact, two thirds of the patients are diagnosed at advanced stages which preclude curative surgical treatment. The efficacy of new chemotherapeutic regimens for advanced NSCLC has been improved, but the median survival for advanced NSCLC by conventional chemotherapy is still around 7-8 months (Breathnach 2001, Hanna 2004).

Therefore, it is now urgently required to develop practical diagnostic biomarkers for early detection of cancer and new types of drugs targeting specific cell signals important for malignant nature of cancer cells. As discussed above, genome-wide expression profile analyses of 101 lung cancers after enrichment of cancer cells by laser microdissection using a cDNA microarray containing 27,648 genes was performed. Through the analysis, several genes that could be potentially good candidates for development of novel diagnostic markers, therapeutic drugs, and/or immunotherapy were identified. Among them, the genes encoding putative tumor-specific transmembrane/secretory proteins are considered to have significant advantages because they are present on the cell surface or within the extracellular space, and/or in serum, making them easily accessible as molecular markers and therapeutic targets.

In the context of the present invention, NPTX1 encoding a secretory protein, is identified as a potential target for development of novel tools for diagnosis and treatment of lung cancer. NPTX1 is a member of a newly recognized subfamily of “long pentraxin” (Goodman). NPTX1 mediates uptake of synaptic macromolecules and involved in both synaptogenesis and synaptic plasticity in developing and adult brain (Breathnach, O. S, et al., J Clin Oncol. 19:1734-1742 (2001)). However the relevance of NPTX1 to carcinogenesis has never been described.

Herein, the NPTX1 protein was shown to be expressed in the great majority of lung cancer specimens, whereas it was scarcely expressed in normal tissues. Furthermore, the higher NPTX1 expression level was associated with shorter cancer specific survival periods. Concordantly, induction of exogenous expression of NPTX1 enhanced the growth/invasive activity of COS-7 cells and NIH-3T3 cells. Secreted NPTX1 could function as an autocrine/paracrine cell growth/invasion factor. NPTX1 have previously identified to bind to Neuronal pentraxin receptor (NPTXR) (Goodman, A R, et al., Cytokine Growth Factor Rev. August; 7(2):192-202 (1996)). However, when mRNA expression of NPTXR was analyzed in lung cancer cell lines and cancer tissues by semiquantitative RT-PCR, the expression pattern of NPTXR was not perfectly concordant with that of NPTX1 (data not shown). Although the precise molecular mechanism underlying the observations herein remains to be elucidated by identification of the NPTX1 receptor in cancer cells, the results obtained by in vitro and in vivo assays clearly suggest that over-expressed NPTX1 is likely to be an autocrine/paracrine growth factor associated with cancer cell growth and invasion, inducing a highly malignant phenotype of lung cancer cells. Furthermore the data demonstrated the potential of NPTX1 as a molecular target for lung cancer treatment.

Interestingly, hypoxia induced a significant increase of NPTX1 expression in lung cancer cells (data not shown). Clinical studies have clearly shown that the low p02 tension within a neoplastic lesion is an independent prognostic indicator of poor outcome and correlates with an increased risk to develop distant metastasis independently of therapeutic treatment (46-48). Hypoxia plays a key role in tumor cell survival, invasion, and metastasis. A series of genes and proteins that may increase the survival of tumor cells under hypoxia conditions, including vascular endothelial growth factor (VEGF), insulin-like growth factor, inducible nitric oxide synthase, platelet-derived endothelial growth factor, glucose transporter 1, erythropoietin and nitric oxide synthase gene, are regulated by Hypoxia Inducible Factor-1a (49-52). Other clinical studies have shown that reduced hypoxia in solid tumors adversely affects the outcome of radiotherapy. Therefore, the data herein suggests that targeting NPTX1 may serve as a promising therapeutic strategy for the treatment of invasive, metastatic, and radioresistant hypoxic lung cancers.

On the other hand, it was also discovered that high levels of NPTX1 protein is present in serologic samples from lung cancer patients. Serum markers could be applied to the differential diagnoses, early detection of cancer, prognostic predictions, monitoring of treatment efficacy, and surveillance of disease relapse. The studies herein reveal that high levels of serum NPTX1 are detected even in patients with earlier-stage tumors. Furthermore serologic concentration of NPTX1 dramatically reduced after surgical resection of primary tumors. Furthermore the levels of serum NPTX1 showed good correlation with the expression levels of NPTX1 in primary tumor tissue in the same patients.

To validate the feasibility of applying NPTX1 as a diagnostic tool, serum levels of NPTX1 were compared with those of CEA, CYFRA and proGRP, a conventional diagnostic marker for ADC, SCC and SCLC, in terms of sensitivity and specificity for diagnosis. An assay combining both markers (NPTX1+CEA, NPTX1+CYFRA or NPTX1+ proGRP) increased the sensitivity to about 64-72% for lung cancer (ADC, SCC or SCLC), higher than that of CEA, CYFRA or proGRP alone, whereas 5-6% of healthy volunteers were falsely diagnosed as positive. Although additional validation with a larger set of serum samples covering various clinical stages will be necessary, the data suggested here sufficiently demonstrate that NPTX1 as a serologic biomarker should be useful for diagnosis of even early-stage lung cancers, monitoring of treatment efficacy and surveillance of disease relapse.

In conclusion, NPTX1 was overexpressed in the great majority of lung cancers and its serum levels were elevated in sera of a large proportion of the patients. NPTX1, combined with other tumor marker(s), could significantly improve the sensitivity of cancer diagnosis, while it could be used at initial diagnosis as an immunohistochemical marker to identify patients who might benefit from early systemic treatment. Since up-regulation of NPTX1 is a frequent and important feature of lung carcinogenesis, targeting NPTX1 might be a new strategy to design anti-cancer drugs specific for lung cancer.

### Part IV

### CDKN3 and EF-1Delta Related Experiments

### Example 22

### General Methods

### 1. Cell Lines and Clinical Tissue Samples

The 15 human lung-cancer cell lines used in this study were as follows: 15 NSCLCs LC176, LC319, A549, NCI-H23, NCI-H226, NCI-H522, PC3, PC9, PC14, SK-LU-1, EBC-1, RERF-LC-AI, SK-MES-1, SW900, and SW1573. All cells were grown in monolayers in appropriate medium supplemented with 10% fetal calf serum (FCS) and were maintained at 37 degree Centigrade in an atmosphere of humidified air with 5% CO₂. Human small airway epithelial cells (SAEC) were grown in optimized medium (SAGM) purchased from Cambrex Bio Science Inc. (Walkersville, Md.). Primary NSCLCs, including seven ADCs and seven SCCs, were obtained as described elsewhere (Kikuchi et al., 2003). A total of 385 formalin-fixed samples of primary NSCLCs including 243 ADCs, 102 SCCs, 28 LCCs, 12 adenosquamous carcinomas (ASCs) and adjacent normal lung tissues, had been obtained earlier along with clinicopathological data from patients undergoing curative surgery at Saitama Cancer Center (Saitama, Japan). NSCLC specimen and five tissues (heart, liver, lung, kidney, and stomach) from post-mortem materials (2 individuals with SCC) were also obtained from Hiroshima University. This study and the use of all clinical materials were approved by the individual Institutional Research Ethics Committees.

2. Semiquantitative RT-PCR analysis

Total RNA was extracted from cultured cells and clinical tissues using TRIzol reagent (Life Technologies, Inc.) according to the manufacturer's protocol. Extracted RNAs and normal human tissue poly(A) RNAs were treated with DNase I (Nippon Gene) and were reverse-transcribed using oligo(dT)20 primer and SuperScript II reverse transcriptase (Invitrogen). Semiquantitative RT-PCR experiments were carried out with the following synthesized gene-specific primers or with beta-actin (ACTB)-specific primers as an internal control:

(SEQ ID NP: 34)
CDKN3, 5′-GTGAATTGTTCTCAGTTTCTCGG-3′
and
(SEQ ID NP: 35)
5′-TCTCTTGATGATAGATGTGCAGC-3′;
(SEQ ID NP: 36)
EF-1delta, 5′-TGGCTACAAACTTCCTAGCACAT-3′
and
(SEQ ID NP: 37)
5′-CTCCACCACACACTGAATCTGTA-3′;
(SEQ ID NP: 38)
ValRS, 5′-TAAGCATCACGCGAGCCGTG-3′
and
(SEQ ID NP: 39)
5′-GGATGGAGCAGCAGCGATCAGAA-3′;
(SEQ ID NP: 40)
EF-1alpha1, 5′-AGACTGGTTAATGATAACAATGC-3′
and
(SEQ ID NP: 41)
5′-GGTCTCAAAATTCTGTGACAAAT-3′;
(SEQ ID NP: 42)
EF-1beta, 5′-CAGAAGCATTCAAGCAGACG-3′
and
(SEQ ID NP: 43)
5′-ATGCCATGATCCAGGATGGA-3′;
(SEQ ID NP: 44)
EF-1gamma, 5′-GGTGGACTACGAGTCATACACAT-3′
and
(SEQ ID NP: 45)
5′-CAGTTTCCTTTAATGACCCCC-3′;
(SEQ ID NP: 46)
CDK1, 5′-AGCCTAGCATCCCATGTCAA-3′
and
(SEQ ID NP: 47)
5′-GAAGACGAAGTACAGCTGAAG-3′;
(SEQ ID NP: 11)
ACTB, 5′-GAGGTGATAGCATTGCTTTCG-3′
and
(SEQ ID NP: 12)
5′-CAAGTCAGTGTACAGGTAAGC-3′.

PCR reactions were optimized for the number of cycles to ensure product intensity within the logarithmic phase of amplification.

### 3. Northern-Blot Analysis

Human multiple-tissue blots (BD Biosciences Clontech) were hybridized with a 32P-labeled PCR product of CDKN3. Prehybridization, hybridization, and washing were performed according to the supplier's recommendations. The blots were autoradiographed with intensifying screens at −80 degree Centigrade for 7 days.

### 4. Western-Blot Analysis

Cells were lysed with RIPA buffer [50 mM Tris-HCl (pH8.0), 150 mM NaCl, 1% NP-40, 0.5% deoxychorate-Na, 0.1% SDS] containing protease inhibitor (Protease Inhibitor Cocktail Set III; CALBIOCHEM). Protein samples were separated by SDS-polyacrylamide gels and electroblotted onto Hybond-ECL nitrocellulose membranes (GE Healthcare Bio-sciences). Blots were incubated with a mouse monoclonal anti-CDKN3 (KAP) antibody (BD Bioscience Pharmingen), a rabbit polyclonal anti-EF-1delta antibody (NOVUS Biologicals), a mouse monoclonal anti-EF-1alpha antibody (Upstate), a rabbit polyclonal anti-Akt antibody (Cell Signaling Technology, Inc.), rabbit polyclonal anti-phospho-Akt (Ser473) antibody (Santa Cruz Biotechnology, Inc.), a mouse monoclonal anti-beta-actin antibody (SIGMA), a mouse monoclonal anti-Flag antibody (SIGMA), rabbit polyclonal anti-c-Myc antibody (Santa Cruz Biotechnology, Inc.) or a rat monoclonal anti-HA antibody (Roche Diagnostics Corporation). Antigen-antibody complexes were detected using secondary antibodies conjugated to horseradish peroxidase (GE Healthcare Bio-sciences). Protein bands were visualized by ECL Western Blotting Detection Reagents (GE Healthcare Bio-sciences), as previously described (Kato et al., 2005; Suzuki et al., 2005). A mouse monoclonal anti-CDKN3 (KAP) antibody (BD Bioscience Pharmingen) and a rabbit polyclonal anti-EF-1delta antibody (NOVUS Biologicals) were individually proved to be specific to human CDKN3 and EF-1delta by western-blot analysis using lysates of NSCLC cells that expressed either of the endogenous proteins or not (see FIG. 19A).

### 5. Immunohistochemistry and Tissue Microarray

To investigate the presence of CDKN3 or EF-1delta protein in clinical samples, the sections were stained by ENVISION+ Kit/horseradish peroxidase (HRP) (DakoCytomation). Briefly, anti-CDKN3 antibody (BD Bioscience Pharmingen) or anti-EF-1delta antibody (NOVUS Biologicals) was added after blocking endogenous peroxidase and proteins, and the sections were incubated with HRP-labeled anti-mouse or rabbit IgG as the secondary antibody. Substrate-chromogen was added and the specimens were counterstained with hematoxylin.

The tumor tissue microarrays were constructed as published previously (Chin, S. F., et al., Mol. Pathol. 56: 275-279 (2003); Callagy, G., et al., Mol. Pathol. 12: 27-34 (2003); Callagy, G., et al., J. Pathol. 205: 388-396 (2005)). The tissue area for sampling was selected based on a visual alignment with the corresponding HE-stained section on a slide. Three, four, or five tissue cores (diameter 0.6 mm; height 3-4 mm) taken from the donor tumor blocks were placed into a recipient paraffin block using a tissue microarrayer (Beecher Instruments). A core of normal tissue was punched from each case. 5-micro m sections of the resulting microarray block were used for immunohistochemical analysis. Positivity of CDKN3 or EF-1delta protein was assessed according to staining intensity as absent or positive by three independent investigators without prior knowledge of the clinical follow-up data. Cases were accepted only as positive if reviewers independently defined them as such.

### 6. Statistical Analysis.

Using contingency tables, a correlation between clinicopathological variables such as age, gender, tumor size (pT), and lymph-node metastasis (pN) with the positivity of CDKN3 and/or EF-1delta was determined by tissue-microarray analysis. Tumor-specific survival curves were calculated from the date of surgery to the time of death related to NSCLC, or to the last follow-up observation. Kaplan-Meier curves were calculated for each relevant variable and for CDKN3 and/or EF-1delta expression; differences in survival times among patient subgroups were analyzed using the log-rank test. Risk factors associated with the prognosis were evaluated using Cox's proportional-hazard regression model with a step-down procedure. Proportional-hazard assumptions were checked and satisfied; only those variables with statistically significant results in univariate analysis were included in a multivariate analysis. The criterion for removing a variable was the likelihood ratio statistic, which was based on the maximum partial likelihood estimate (default P value of 0.05 for removal from the model).

### 7. RNA Interference Assay.

As noted above, a vector-based RNA interference (RNAi) system, psiH1BX3.0, to direct the synthesis of siRNAs in mammalian cells has been previously established (Suzuki, C., Cancer Res. 63: 7038-7041 (2003)). 10 micro g of siRNA-expression vector was transfected into NSCLC cell lines with 30 micro L of Lipofectamine 2000 (Invitrogen). The transfected cells were cultured for five days in the presence of appropriate concentrations of geneticin (G418), after which cell numbers and viability were measured by Giemsa staining and triplicate MTT assays. The target sequences of the synthetic oligonucleotides for RNAi were as follows:

control 1 (EGFP: gene, a mutant of Aequorea
victoria GFP),
(SEQ ID NO: 23)
5′-GAAGCAGCACGACTTCTTC-3′;
control 2 (Luciferase: Photinus pyralis
luciferase gene),
(SEQ ID NO: 15)
5′-CGTACGCGGAATACTTCGA-3′;
control 3 (Scramble: chloroplast Euglena
gracilis gene coding for 5S and 16S rRNAs),
(SEQ ID NO: 16)
5′-GCGCGCTTTGTAGGATTCG-3′;
siRNA-CDKN3-A (si-A),
(SEQ ID NO: 49)
5′-TATAGAGTCCCAAACCTTC-3′;
siRNA-CDKN3-B (si-B),
(SEQ ID NO: 50)
5′-TACACTGCTATGGAGGACT-3′;
siRNA-EF-1delta-1 (si-1),
(SEQ ID NO: 51)
5′-GTGGAGAACCAGAGTCTGC-3′;
siRNA-EF-1delta-2 (si-2),
(SEQ ID NO: 52)
5′-CATCCAGAAATCCCTGGCT-3′.

To validate the instant RNAi system, individual control siRNAs (EGFP, Luciferase and Scramble) were initially confirmed using semiquantitative RT-PCR to decrease expression of the corresponding target genes that had been transiently transfected into COS-7 cells. Down-regulation of CDKN3 and EF-1delta expression by si-CDKN3s and si-EF-1deltas, but not by controls, was confirmed with semiquantitative RT-PCR in the cell lines used for this assay.

### 8. Immunohistochemistry and Tissue Microarray

To investigate the presence of CDKN3 or EF-1delta protein in clinical samples, the sections were stained using ENVISION+ Kit/horseradish peroxidase (HRP) (DakoCytomation). Briefly, anti-CDKN3 antibody (BD Bioscience Pharmingen) or anti-EF-1delta antibody (NOVUS Biologicals) was added after blocking endogenous peroxidase and proteins, and the sections were incubated with HRP-labeled anti-mouse or rabbit IgG as the secondary antibody. Substrate-chromogen was added and the specimens were counterstained with hematoxylin.

The tumor tissue microarrays were constructed as published previously (Chin et al., 2003; Callagy et al., 2003, 2005). The tissue area for sampling was selected based on a visual alignment with the corresponding HE-stained section on a slide. Three, four, or five tissue cores (diameter 0.6 mm; height 3-4 mm) taken from the donor tumor blocks were placed into a recipient paraffin block using a tissue microarrayer (Beecher Instruments). A core of normal tissue was punched from each case. 5-micro m sections of the resulting microarray block were used for immunohistochemical analysis. Positivity of CDKN3 or EF-1delta protein was assessed according to staining intensity as absent or positive by three independent investigators without prior knowledge of the clinical follow-up data. The intensity of CDKN3 or EF-1delta staining was evaluated using following criteria: strong positive (2+), dark brown staining in more than 50% of tumor cells completely obscuring cytoplasm; weak positive (1+), any lesser degree of brown staining appreciable in tumor cell cytoplasm; absent (scored as 0), no appreciable staining in tumor cells. Cases were accepted only as positive if reviewers independently defined them as such.

### 9. Statistical Analysis

Using contingency tables, attempts were made to correlate clinicopathological variables such as age, gender, tumor size (pT), and lymph-node metastasis (pN) with the positivity of CDKN3 and/or EF-1delta determined by tissue-microarray analysis. Tumor-specific survival curves were calculated from the date of surgery to the time of death related to NSCLC, or to the last follow-up observation. Kaplan-Meier curves were calculated for each relevant variable and for CDKN3 and/or EF-1delta expression; differences in survival times among patient subgroups were analyzed using the log-rank test. Risk factors associated with the prognosis were evaluated using Cox's proportional-hazard regression model with a step-down procedure. Proportional-hazard assumptions were checked and satisfied; only those variables with statistically significant results in univariate analysis were included in a multivariate analysis. The criterion for removing a variable was the likelihood ratio statistic, which was based on the maximum partial likelihood estimate (default P value of 0.05 for removal from the model).

### 10. Matrigel Invasion Assay

Using FuGENE 6 Transfection Reagent (Roche Diagnostics) according to the manufacturer's instructions, NIH-3T3 cells were transfected with plasmids expressing CDKN3 or mock plasmids. Transfected cells were harvested and suspended in DMEM without FCS. Before the cell suspension was prepared, the dried layer of Matrigel matrix (Becton Dickinson Labware) was rehydrated with DMEM for 2 hours at room temperature. Then, DMEM containing 10% FCS was added to each lower chamber of 24-well Matrigel invasion chambers and cell suspension was added to each insert of the upper chamber. The plates of inserts were incubated for 22 hours at 37 degree Centigrade. After incubation, cells invading through the Matrigel-coated inserts were fixed and stained by Giemsa.

### 11. Synthesized Cell-Permeable Peptide

19-amino acid peptide sequence corresponding to a part of EF-1delta protein that contained possible binding sites of CDKN3 were covalently linked at its NH₂-terminus to a membrane transducing 11 poly-arginine sequence (11R; refs. Futaki et al., Hayama et al., 2006, 2007). Five cell permeable peptides were synthesized: 11R-EF-1delta₇₃₋₉₁, RRRRRRRRRRR-GGG-TSGDHGELVVRIASLEVEN; 11R-EF-1delta₉₀₋₁₀₈, RRRRRRRRRRR-GGG-ENQSLRGVVQELQQAISKL; 11R-EF-1delta₁₀₈₋₁₂₆, RRRRRRRRRRR-GGG-LEARLNVLEKSSPGHRATA; 11R-EF-1delta₁₂₅₋₁₄₃, RRRRRRRRRRR-GGG-TAPQTQHVSPMRQVEPPAK; 11R-EF-1delta₁₄₂₋₁₆₀, RRRRRRRRRRR-GGG-AKKPATPAEDDEDDDIDLF. Peptides were purified by preparative reverse-phase high-pressure liquid chromatography. LC319 cells were incubated with the 11R peptides at the concentration of 2.5, 5.0 and 7.5 micro M for 5 days. The medium was changed at every 48 hours at the appropriate concentrations of each peptide and the viability of cells was evaluated by MTT assay at 5 days after the treatment.
12. Immunoprecipitation and MALDI-TOF-MS mapping of CDKN3-Associated Proteins.

Cell extracts from lung-cancer cell line LC319 were pre-cleared by incubation at 4 degree Centigrade for 1 hour with 100 micro L of protein G-agarose beads in a final volume of 2 ml of immunoprecipitation buffer [0.5% NP-40, 50 mM Tris-HCl, 150 mM NaCl] in the presence of protease inhibitor. After centrifugation at 1000 rpm for 5 min at 4 degree Centigrade, the supernatant was incubated at 4 degree Centigrade with anti-CDKN3 monoclonal antibody or normal mouse IgG, for 2 hours. The beads were then collected by centrifugation at 5000 rpm for 2 min and washed six times with 1 ml of each immunoprecipitation buffer. The washed beads were resuspended in 50 micro L of Laemmli sample buffer and boiled for 5 min, and the proteins were separated by 5-10% SDS PAGE gels (BIO RAD). After electrophoresis, the gels were stained with silver. Protein bands specifically found in extracts immunoprecipitated with anti-CDKN3 monoclonal antibody were excised and served for matrix-assisted laser desorption/ionization-time of flight mass spectrometry (MALDI-TOF-MS) analysis (AXIMA-CFR plus, SHIMADZU BIOTECH).

### 13. Phosphatase Assay.

For phosphatase treatment, cell extract was incubated with λ-phosphatase (New England Biolabs) in phosphatase buffer or buffer alone for 1 hour at 37 degree Centigrade, and subsequently used for immunoblotting.

### Example 23

### CDKN3 Expression in Lung Tumors and Normal Tissues

To search for novel target molecules for development of therapeutic agents and/or diagnostic biomarkers, genes were first screened for showing 3-fold higher expression in more than 50% of 101 lung cancers analyzed by cDNA microarray. Among 27,648 genes screened, it was identified that the gene encoding cyclin-dependent kinase inhibitor 3 (CDKN3) was overexpressed frequently in lung cancers and increase of CDKN3 expression was confirmed in 12 of 14 additional NSCLC cases (6 of 7 adenocarcinomas (ADCs) and in 6 of 7 squamous-cell carcinomas (SCCs) (FIG. 16A). Interestingly, much higher expression patterns of CDKN3 was observed in brain metastasis as well as advanced primary lung tumors (adenocarcinomas, ADCs), compared to those in earlier-stage primary lung tumors (FIG. 16B). Northern blotting with CDKN3 cDNA as a probe identified a strong signal corresponding to 0.9-kb transcript in testis and a very weak signal in thymus, colon, stomach, and bone marrow among the 23 normal human tissues examined (FIG. 16C). The expression of CDKN3 protein was also examined with anti-CDKN3 antibody on six normal tissues (heart, liver, kidney, lung, colon, and testis), and found that CDKN3 expressed abundantly in testis (mainly in cytoplasm of primary spermatocytes) and lung cancers, but its expression was hardly detectable in the remaining five normal tissues (FIG. 17A).

### Example 24

### Association of CDKN3 Overexpression with Poor Prognosis

To verify the biological and clinicopathological significance of CDKN3, CDKN3 protein expression in clinical NSCLCs was examined by means of tissue microarrays containing NSCLC tissues from 385 patients as well as SCLC tissues from 15 patients. Positive staining for CDKN3 (the cytoplasm and nucleus) was observed in 65.7% of surgically-resected NSCLCs (253/385) and in 80.0% of SCLCs (12/15), while no staining was observed in any of normal lung tissues examined (FIG. 17B). A correlation between positive staining and various clinicopathological parameters was then examined in 385 NSCLC patients. The sample size of SCLCs was too small to be evaluated further. Gender (higher in male; P=0.0054 by Fisher's exact test), histological classification (higher in non-ADCs; P<0.0001 Fisher's exact test), and pN stage (higher in N1, N2; P=0.0057 by Fisher's exact test) were significantly associated with the CDKN3 positivity (Table 4A). NSCLC patients whose tumors showed positive staining of CDKN3 revealed shorter tumor-specific survival periods compared to those with absent CDKN3 expression (P<0.0001 by the Log-rank test) (FIG. 17C). By univariate analysis, elderly (>=65), male gender, non-ADC histological classification, advanced pT stage, advanced pN stage, and CDKN3 positivity were all significantly related to poor tumor-specific survival among NSCLC patients (Table 4B). In multivariate analysis of the prognostic factors, elderly, advanced pT stage, advanced pN stage, and CDKN3 positivity were indicated to be independent prognostic factors (Table 4B).

TABLE 4A
Association between CDKN3-positivity in NSCLC
tissues and patients' characteristics

| | | CDKN3 | CDKN3 | P-value |
|---|---|---|---|---|
| | Total | positive | negative | strong/weak |
| | n = 385 | n = 253 | n = 132 | vs negative |
| Gender | | | | |
| Male | 264 | 186 | 78 | 0.0054* |
| Female | 121 | 67 | 54 | |
| Age (years) | | | | |
| <65 | 188 | 129 | 59 | 0.2828 |
| >=65 | 197 | 124 | 73 | |
| Histological type | | | | |
| ADC | 243 | 140 | 103 | <0.0001*, ** |
| SCC | 102 | 80 | 22 | |
| Others | 40 | 33 | 7 | |
| pT factor | | | | |
| T1 + T2 | 274 | 176 | 98 | 0.3463 |
| T3 + T4 | 111 | 77 | 34 | |
| pN factor | | | | |
| N0 | 237 | 143 | 94 | 0.0057* |
| N1 + N2 | 148 | 110 | 38 | |

ADC, adenocarcinoma;
SCC, squamous-cell carcinoma
Others, large-cell carcinoma plus adenosquamous-cell carcinoma
*P < 0.05 (Fisher's exact test)
**ADC versus non-ADC histology

TABLE 4B
Cox's proportional hazards model analysis of prognostic
factors in NSCLCs

| | Hazards | | Unfavorable/ | |
|---|---|---|---|---|
| Variables | ratio | 95% CI | Favorable | P-value |
| Univariate analysis | | | | |
| CDKN3 | 2.121 | 1.488-3.025 | Strong(+) or | <0.0001* |
| | | | Weak(+)/(−) | |
| Age (years) | 1.425 | 1.060-1.918 | 65<=/<65 | 0.0192* |
| Gender | 1.626 | 1.164-2.273 | Male/Female | 0.0044* |
| Histological type | 1.438 | 1.072-1.929 | non-ADC/ADC¹ | 0.0153* |
| pT factor | 1.913 | 1.413-2.590 | T3 + T4/T1 + T2 | <0.0001* |
| pN factor | 2.420 | 1.805-3.243 | N1 + N2/N0 | <0.0001* |
| Multivariate analysis | | | | |
| CDKN3 | 1.897 | 1.313-2.742 | Strong(+) or | 0.0007* |
| | | | Weak(+)/(−) | |
| Age (years) | 1.797 | 1.327-2.433 | 65<=/<65 | 0.0002* |
| Gender | 1.357 | 0.938-1.963 | Male/Female | 0.1053 |
| Histological type | 0.993 | 0.713-1.383 | non-ADC/ADC¹ | 0.9680 |
| pT factor | 1.895 | 1.389-2.584 | T3 + T4/T1 + T2 | <0.0001* |
| pN factor | 2.284 | 1.690-3.086 | N1 + N2/N0 | <0.0001* |

¹ADC, adenocarcinoma
*P < 0.05

### Example 25

### Identification of EF-1Beta-Gamma-Delta/ValRS as the Novel Molecules Interacting with CDKN3

To elucidate the function of CDKN3 in carcinogenesis, proteins that would interact with CDKN3 in lung cancer cells were sought. Cell extracts from LC319 cells were immunoprecipitated with anti-CDKN3 monoclonal antibody or mouse IgG (negative control). Following separation by SDS-PAGE, protein complexes were silver-stained. Protein bands of 140-, 50-, 31-, and 25-kDa, which were seen in immunoprecipitates by anti-CDKN3 antibody, but not in those by mouse IgG, were excised, trypsin-digested, and subjected to mass spectrometry analysis. Peptides from 140-, 50-, 31-, and 25-kDa bands matched sequences in valyl-tRNA synthetase (valyi-tRNA synthetase, ValRS; 140-kDa), eukaryotic translation elongation factor 1gamma (EF-1gamma; 50-kDa), eukaryotic translation elongation factor 1 delta (EF-1delta; 31-kDa), eukaryotic translation elongation factor 1 beta (EF-1beta; 25-kDa), respectively (FIG. 18A).

These four proteins include the guanine-nucleotide exchange complex of elongation factor-1 that is responsible for protein synthesis. To investigate the expression pattern of components of the guanine-nucleotide exchange complex of elongation factor-1 and its related molecules in NSCLC cells, mRNA expressions of ValRS, EF-1delta, EF-1alpha1, EF-1beta, EF-1gamma, and CDK1 were analyzed by semiquantitative RT-PCR experiments. The expression patterns of CDKN3 in lung cancers were very similar to those of EF-1delta (FIG. 18B). It was further confirmed that CDKN3 and EF-1delta proteins were co-activated in lung-cancer cell lines examined by western-blot analysis (FIG. 19A). A previous report demonstrated the oncogenic potential of EF-1delta in mammalian cells (Joseph, P., et al., J Biol Chem. 277: 6131-6136 (2002)).

From these findings, the functional relevance of CDKN3 to EF-1delta in cancer cells was investigated. The cognate interaction between endogenous CDKN3 and EF-1delta was examined by immunoprecipitation experiment in LC319 cells, in which these two genes were expressed abundantly (FIG. 20A). Their subcellular localization was investigated in LC319 cells synchronized with aphidicolin by immunocytochemical analysis using mouse monoclonal anti-CDKN3 and rabbit polyclonal anti-EF-1delta antibodies. Co-localization of the proteins was detected mainly in the cytoplasm and nucleus through the cell cycle (representative images are shown in FIG. 20B).

### Example 26

### Effect of EF-1Delta on Growth and Progression of NSCLCs

To clarify the clinicopathological significance of EF-1delta, EF-1delta protein expression was examined in clinical NSCLCs with tissue microarrays containing lung-cancer tissues from 385 patients. Positive staining for EF-1delta (the cytoplasm and nucleus) was observed in 67.5% of surgically-resected NSCLCs (260/385) (FIG. 19B). Staining for EF-1delta was hardly observed in any of normal lung tissues examined. The expression of CDKN3 protein was significantly concordant with EF-1delta expression in these tumors (P<0.0001 by Fisher's exact test). Positive staining of EF-1delta in NSCLCs was significantly associated with gender (higher in male; P=0.0004 by Fisher's exact test), histological type (higher in non-ADC; P<0.0001 by Fisher's exact test), and advanced pN stage (higher in N1, N2; P=0.0141 by Fisher's exact test), and 5 year-survival (P=0.0006 by the Log-rank test) (FIG. 19C; Table 5A). In multivariate analysis of the prognostic factors, age, pT stage, pN stage, and EF-1delta positivity were indicated to be an independent prognostic factor (Table 5B).

To further assess whether EF-1delta is biologically essential for growth or survival of lung-cancer cells, we designed and constructed plasmids to express siRNA against EF-1delta (si-EF-1delta-1 and -2), and three different control plasmids (siRNAs for EGFP, LUC, or SCR), and transfected them into lung cancer cells to suppress expression of endogenous EF-1delta. The amount of EF-1delta transcript in the cells transfected with si-1 was significantly decreased in comparison with cells transfected with any of the three control siRNAs (FIG. 22B); si-2 showed almost no suppressive effect on CDKN3 expression. Transfection of si-1 also resulted in significant decreases in cell viability and colony numbers measured by MTT and colony-formation assays (FIG. 22B). These results suggested that CDKN3 could promote the growth and/or progression of NSCLCs through interaction with and/or activating EF-1delta.

TABLE 5A
Association between EF-1delta-positivity in NSCLC
tissues and patients' characteristics

| | | EF-1delta | EF-1delta | P-value |
|---|---|---|---|---|
| | Total | positive | negative | positive vs |
| | n = 385 | n = 260 | n = 125 | negative |
| Gender | | | | |
| Male | 264 | 194 | 70 | 0.0004* |
| Female | 121 | 66 | 55 | |
| Age (years) | | | | |
| <65 | 188 | 134 | 54 | 0.1292 |
| >=65 | 197 | 126 | 71 | |
| Histological | | | | |
| type | | | | |
| ADC | 243 | 145 | 98 | <0.0001*, ** |
| SCC | 102 | 79 | 23 | |
| Others | 40 | 36 | 4 | |
| pT factor | | | | |
| T1 + T2 | 260 | 179 | 81 | 0.1519 |
| T3 + T4 | 125 | 95 | 30 | |
| pN factor | | | | |
| N0 | 237 | 149 | 88 | 0.0141* |
| N1 + N2 | 148 | 111 | 37 | |

ADC, adenocarcinoma;
SCC, squamous-cell carcinoma
Others, large-cell carcinoma plus adenosquamous-cell carcinoma
*P < 0.05 (Fisher's exact test)
**ADC versus non-ADC histology

TABLE 5B
Cox's proportional hazards model analysis of
prognostic factors in NSCLCs

| | Hazards | | Unfavorable/ | |
|---|---|---|---|---|
| Variables | ratio | 95% CI | Favorable | P-value |
| Univariate analysis | | | | |
| EF-1delta | 1.813 | 1.282-2.562 | Strong(+) or | 0.0008* |
| | | | Weak(+)/(−) | |
| Age (years) | 1.425 | 1.060-1.918 | 65<=/<65 | 0.0192* |
| Gender | 1.626 | 1.164-2.273 | Male/Female | 0.0044* |
| Histological type | 1.438 | 1.072-1.929 | non-ADC/ADC¹ | 0.0153* |
| pT factor | 1.913 | 1.413-2.590 | T3 + T4/T1 + T2 | <0.0001* |
| pN factor | 2.420 | 1.805-3.243 | N1 + N2/N0 | <0.0001* |
| Multivariate | | | | |
| analysis | | | | |
| EF-1delta | 1.589 | 1.102-2.290 | Strong(+) or | 0.0131* |
| | | | Weak(+)/(−) | |
| Age (years) | 1.839 | 1.354-2.498 | 65<=/<65 | <0.0001* |
| Gender | 1.340 | 0.925-1.942 | Male/Female | 0.1222 |
| Histological type | 1.021 | 0.731-1.426 | non-ADC/ADC¹ | 0.9023 |
| pT factor | 1.838 | 1.348-2.505 | T3 + T4/T1 + T2 | 0.0001* |
| pN factor | 2.345 | 1.733-3.172 | N1 + N2/N0 | <0.0001* |

¹ADC, adenocarcinoma
*P < 0.05

### Example 27

### CDKN3 Mediated-Dephosphorylation of EF-1Delta

Western-blot analysis detected two different sizes of EF-1delta protein in lung cancer cells (FIG. 20A), whereas EF-1delta was reportedly phosphorylated at its serine and threonine residues in vitro (Minella O, et al., Biosci Rep. 3:119-27 (1998)). To examine a possibility of the EF-1delta phosphorylation in vivo, we incubated extracts from COS-7 cells that overexpressed Flag-HA-tagged EF-1delta in the presence or absence of protein phosphatase, and analyzed the molecular weight of EF-1delta protein by western-blot analysis. The measured weight of the majority of EF-1delta protein in the extracts treated with phosphatase was smaller than that in the untreated cells (FIG. 20C, left panel). On the other hand, the molecular weight of Flag-HA-tagged EF-1beta and Flag-HA-tagged EF-1gamma proteins was not changed after treatment with phosphatase (FIG. 20C, middle right panels).

Furthermore, we confirmed that phosphorylated form of EF-1delta was present in lung-cancer LC319 cells (FIG. 20D, left panel). Since CDKN3 encodes dual-specificity protein phosphatase, we then examined CDKN3-induced dephosphorylation of EF-1delta in lung cancer cells. We transfected into LC319 cells the Flag-HA-tagged CDKN3-expression vector. Western-blot analyses using anti-EF-1delta antibody indicated that endogenous EF-1delta was dephosphorylated in a CDKN3-dose-dependent manner (FIG. 20D, right panel).

To confirm specific dephosphorylation of EF-1delta by CDKN3, the Flag-HA-tagged CDKN3-expression vector and Flag-HA-tagged EF-1delta-expression vector were transfected to COS-7 cells, and detected the reduction of phosphorylated EF-1delta protein by overexpression of CDKN3 (FIG. 21A, left panel). Immunoprecipitation of EF-1delta and CDKN3 with anti-Flag antibody followed by immunoblotting with pan-phospho-specific antibodies (phospho-serine, -threonine, or -tyrosine) indicated dephosphorylation of EF-1delta at its serine residues (FIG. 21A, right panel). No effects on threonine and tyrosine residues were observed by overexpression of CDKN3 (data not shown).

### Example 28

### Identification of the CDKN3-Binding Region in EF-1Delta

The biological importance of the association of these two proteins and its potential as a therapeutic target for lung cancer was subsequently investigated. To determine the domain in EF-1delta that is required for interaction with CDKN3, each construct of EF-1delta with FLAG-HA-sequence at its N- and C-terminals were transfected into LC319 cells (EF-1delta72-160, EF-1delta161-281, EF-1delta1-160, EF-1delta72-281, and full-length EF-1delta1-281; FIG. 21B). Immunoprecipitation with monoclonal anti-Flag antibody indicated that EF-1delta72-160, EF-1delta1-160, EF-1delta72-281, and EF-1delta1-281 were able to interact with CDKN3, but EF-1delta161-281 was not (FIG. 21B). These experiments suggested that the 89 amino-acid polypeptide (codons 72-160; SEQ ID NO: 48) containing leucine zipper motif in EF-1delta should play an important role in the interaction with CDKN3.

### Example 29

### Growth-Suppression of NSCLC Cells by siRNA Against CDKN3 and EF-1Delta

To assess whether CDKN3 is essential for growth or survival of lung-cancer cells, plasmids were designed and constructed to express siRNA against CDKN3 (si-A and -B), and three different control plasmids (siRNAs for EGFP, Luciferase (LUC), or Scramble (SCR)), and transfected them into LC319 cells (FIG. 22A) and A549 cells (data not shown). The amount of CDKN3 transcript in the cells transfected with si-A was most significantly decreased in comparison with cells transfected with any of the three control siRNAs, while si-B showed almost no suppressive effect on CDKN3 expression (FIG. 22A: upper left panel). In accord with its suppressive effect on gene expression, transfected si-A caused decreases in cell viability and colony numbers measured by MTT and colony-formation assays, but no such effects were observed by three controls or si-B (FIG. 22A: right upper and lower panels).

To further assess whether EF-1delta is biologically essential for growth or survival of lung-cancer cells, plasmids were designed and constructed to express siRNA against EF-1delta (si-EF-1delta-1 and -2), and three different control plasmids (siRNAs for EGFP, LUC, or SCR), and transfected them into LC319 cells to suppress expression of endogenous EF-1delta. The amount of EF-1delta transcript in the cells transfected with si-1 was significantly decreased in comparison with cells transfected with any of the three control siRNAs (FIG. 22B upper left panel); si-2 showed almost no suppressive effect on EF-1delta expression. Transfection of si-1 also resulted in significant decreases in cell viability and colony numbers measured by MTT and colony-formation assays (FIG. 22B right upper and lower panels). These results suggested that CDKN3 may promote the growth and/or progression of NSCLCs through interaction with and/or activating EF-1delta.

### Example 30

### Overexpression of CDKN3 Increases Cellular Invasion and is Sufficient to Activate Akt

As the immunohistochemical analysis on tissue microarray had indicated that lung-cancer patients with CDKN3 strong-positive tumors showed shorter cancer-specific survival period than patients whose tumors were negative for CDKN3 (FIGS. 19B and 19C), Matrigel invasion assays were performed to determine whether CDKN3 might play a role in cellular invasive ability. Invasion of in NIH-3T3 cells transfected with CDKN3-expression vector through Matrigel was significantly enhanced (FIG. 19C), compared to the control cells transfected with mock-vector, suggesting that CDKN3 could also contribute to the highly malignant phenotype of lung-cancer cells. On the other hand, EF-1alpha, which was known to associate with the EF-1 beta, gamma, delta, and ValRS, appears to be implicated in multiple functions.

So, to investigate the expression pattern of CDKN3 and EF-1alpha (EF-1alpha1 and EF-1alpha2) in NSCLC cells, mRNA expression of CDKN3, EF-1alpha1 and EF-1alpha2 was analyzed by semiquantitative RT-PCR experiments. The expression patterns of EF-1alpha2 in lung cancers were similar to those of EF-1delta (FIG. 23). EF-1alpha2 is likely to be an important human oncogene, expression of EF-1alpha2 transforms rodent fibroblasts and increases their tumorigenicity in nude mice, and (Lee, 2003; Anand et al., 2002). On the other hand, EF-1alpha is likely to be regulated by the EF-1beta-gamma-delta/ValRS, but little is known on how EF-1alpha is regulated as a multiple functional protein (Minella et al., 1998). Recent report also indicated that EF-1alpha2 is an activator of Akt and enhances cellular invasion and migration in an Akt- and PI3K-dependent manner. To determine whether CDKN3 might be involved in Akt activation, CDKN3 were transiently overexpressed in LC319 cells and used by western-blot analysis to determine the phosphorylation status of Akt. The phosphorylation of Ser473 serve as a surrogate marker of Akt activation was then investigated. As shown in FIG. 19D, LC319 cells that transiently overexpressed CDKN3 increased the level of phosphorylation of Ser473 relative to control cells transfected mock-vector. To determine whether PI3K activity is required for CDKN3-dependent increase in cellular invasion, we performed invasion assays in the presence of LY294002. These assays showed that PI3K inhibition reduced the extent of invasion significantly in CDKN3-overexpressing cells in a LY294002-dose dependent manner (FIG. 23, top panel). On the other hands, LY29400 had little inhibitory effect on invasion in the control cells transfected with mock-vector (FIG. 23, bottom panel).

### Example 31

### Growth Inhibition of NSCLC Cells by Dominant-Negative Peptides of CDKN3

Then, to investigate the functional significance of interaction between CDKN3 and EF-1delta for growth or survival of lung-cancer cells, bioactive cell-permeable peptides expected to inhibit the binding of these two proteins were developed. Next 5 different peptides of 19 amino acid sequence that included in codons 73-160 of EF-1delta were synthesized (FIG. 24A). These peptides were covalently linked at NH₂-terminalus to a membrane transducing 11 arginine-residues (11R). The effect on growth by addition of the five 11R-EF-1delta peptides into culture medium of LC319 cells was evaluated, wherein the treatment with the 11R-EF-1delta₉₀₋₁₀₈ peptide resulted in significant decreases in cell viability as measured by MTT assay (FIG. 24B, upper panel). Addition of the 11R-EF-1delta₉₀₋₁₀₈ into the culture medium of LC319 cells reduced complex formation between endogenous CDKN3 and EF-1delta by an immunoprecipitation experiment (FIG. 24B, lower panel). These data indicated that 11R-EF-1delta₉₀₋₁₀₈ could specifically inhibit the interaction CDKN3 and EF-1delta.

### Discussion:

Recent acceleration in identification and characterization of novel molecular targets for cancer therapy has focused considerable interest on the development of new types of anticancer agents (Kelly, K., et al., J. Clin. Oncol. 19: 3210-3218 (2001)). So far, numerous targeted therapies are being investigated for lung cancers, but the ranges of tumor types that respond as well as the effectiveness of the treatments are still very limited. Molecular-targeted drugs are expected to be highly specific to malignant cells, with minimal adverse effects due to their well-defined mechanisms of action. As an approach to that goal, a promising strategy is to use the power of genome-wide expression analysis to effectively identify genes that are overexpressed in cancer cells. In addition, tissue microarrays were used to analyze hundreds of archived clinical samples for validation of the potential target proteins with combination of high-throughput screening of loss-of-function effects by means of the RNAi technique. Using this approach it is shown herein that CDKN3 is frequently overexpressed in clinical lung-cancer samples, and cell lines, and that the gene product plays indispensable roles in the growth and progression of lung-cancer cells.

CDKN3 (also named as KAP) belongs to a family of dual specificity protein phosphatases and was initially identified as a protein interacting with cdk2 or cdc2, indicating that CDKN3 may play a role in cell cycle regulation (Gyuris et al., 1993; Hannon et al., 1994; Brown et al., 1999). Overexpression of CDKN3 has been reported in in situ and invasive ductal carcinoma (Lee, S. W., et al., Mol Cell Biol. 20: 1723-1732 (2000)), however its oncogenic function remains unclear.

EF-1delta, a subunit of the elongation factor-1 complex, which is known to function as guanine nucleotide exchange factor and is responsible for the enzymatic delivery of aminoacyl tRNAs to the ribosome was discovered as a novel intracellular target molecule of CDKN3. Aminoacyl-tRNA is the donor of amino acid in ribosomal protein synthesis. The tRNA molecule is aminoacylated with the corresponding amino acid by an aminoacyl-tRNA synthetase. The aminoacyl-tRNA is converted to a ternary complex with elongation factor-1alpha (EF-1alpha), to give the immediate precursor of amino acid for protein synthesis. The elongation factor-1 (EF-1) is composed of the guanine-nucleotide exchange complex of four different subunits (EF-1beta, EF-1gamma, EF-1delta, and ValRS) and EF-1alpha, a G-protein, responsible for the binding of aminoacyl t-RNA to the ribosome (Brandsma et al., 1995; Riis et al., 1990; Nygard et al., 1990; Motorin et al., 1988; Motorin et al., 1991). The EF-1beta-gamma-delta/ValRS is phosphorylated by protein kinase C(PKC), casein kinase II (CK2) and cyclin dependent kinase 1 (CDK1). EF-1delta as a component of the EF-1 complex is known to be phosphorylated by PKC at least in mammals (Venema, R. C., et al, J Biol Chem. 266, 11993-11998. (1991); Venema R. C., et al, J Biol Chem. 266, 12574-12580. (1991)). On the other hand, overexpression of EF-1delta could transform NIH3T3 cells and make them tumorigenic in nude mice, and blocking EF-1delta with its antisense mRNA, furthermore, resulted in a significant reversal of its oncogenic potential (Joseph, P., et al., J Biol Chem. 277: 6131-6136 (2002); Lei, Y. X., et al., Teratog Carcinog Mutagen. 22: 377-383 (2002)).

On the other hand, EF-1alpha is involved in multiple cellular functions and is regulated by the EF-1beta-gamma-delta/ValRS (Minella O, et al., Biosci Rep. 3:119-27 (1998)). Recent reports indicated that EF-1alpha2, one of the two isoforms of EF-1alpha, could stimulate cell migration and invasion in breast cancer cells (Amiri A, et al., Oncogene 26: 3027-40 (2007)), whereas, it was overexpressed in metastatic rat mammary adenocarcinoma cell lines relative to non-metastatic controls (Pencil S D, Breast Cancer Res Treat. 25: 165-74 (1993); Edmonds B T, et al., J Cell Sci. 109: 2705-14 (1996)).

A previous report has shown that the increase in EF-1beta-gamma-delta/ValRS activity would be related to phosphorylation of EF-1gamma by CDK1, in parallel, phosphorylation of EF-1delta would lead to inhibition of ValRS and therefore to specific inhibition of poly(valine) synthesis (Monnier et al., 2001). On the other hand, overexpression of EF-1delta could transform NIH3T3 cells and make them tumorigenic in nude mice. Blocking EF-1delta with its antisense mRNA, furthermore, resulted in a significant reversal of its oncogenic potential (Joseph et al., 2002; Lei et al., 2002). In addition, EF-1 alpha is involved in multiple cellular functions and is regulated by the EF-1beta-gamma-delta/ValRS (Minella et al., 1998). Recent reports have shown that EF-1alpha2, one of the two isoforms of EF-1 alpha, stimulates cell migration and invasion in breast cancer cells (Amiri et al., 2006). Furthermore, EF-1 alpha2 may have a role in metastatic development and it is over-expressed in metastatic rat mammary adenocarcinoma cell lines relative to non-metastatic controls (Pencil et al., 1993; Edmonds et al., 1996).

The treatment of NSCLC cells herein, with specific siRNA to reduce expression of CDKN3 or EF-1delta, resulted in growth suppression. It was confirmed that EF-1delta was co-expressed with CDKN3 in lung cancer cells, and is a physiological substrate of CDKN3 phosphatase in vivo, suggesting that CDKN3 could have a growth-promoting function in lung tumors through dephosphorylation of EF-1delta. Clinicopathologic evidence obtained through our tissue microarray experiments showed that NSCLC patients with strongly CDKN3 and/or EF-1delta positive tumors showed shorter survival periods than those with negative or weak staining for CDKN3 and EF-1delta, raising the possibility that overexpressed CDKN3 and/or EF-1delta could prompt a highly malignant phenotype of lung cancer cells. Furthermore, it was shown that transducible 11R-EF-1delta₉₀₋₁₀₈ peptides could inhibit a functional complex formation of CDKN3 and EF-1delta and resulted in the suppression of cancer cell growth.

The specific siRNA to reduce expression of CDKN3 or EF-1delta resulted in growth suppression of NSCLC cells. It was confirmed that EF-1delta was co-expressed with CDKN3 in lung cancer cells, and is likely to be a physiological substrate of CDKN3 phosphatase in vivo suggesting that CDKN3 could have a growth-promoting function in lung tumors through dephosphorylation of EF-1delta. Furthermore, clinicopathologic evidence obtained through present tissue microarray experiments showed that NSCLC patients with CDKN3 and/or EF-1delta positive tumors showed shorter survival periods than those with negative staining for CDKN3 and EF-1delta, raising the possibility that overexpressed CDKN3 and/or EF-1delta could prompt a highly malignant phenotype of lung cancer cells. A combination of present data suggests that association of CDKN3 with EF-1beta-gamma-delta/ValRS might lead to the dephosphorylation of EF-1delta and activates cellular function of tumor cells, thus resulting in the tumor growth and/or progression.

In summary, dual specificity protein phosphatase, CDKN3 is likely to play an essential role for growth-promoting pathway of lung cancers through dephosphorylation of its newly revealed interacting-molecule, EF-1delta. CDKN3 and EF-1delta could be useful as prognostic biomarkers in clinic, and targeting the enzymatic activity of CDKN3 or interaction of CDKN3 with EF-1delta should be a promising therapeutic approach to develop new types of anti-cancer drugs.

### INDUSTRIAL APPLICABILITY

As demonstrated herein, cell growth is suppressed by double-stranded molecules that specifically target the EBI3, CDKN3 and/or EF-1delta gene. Thus, these novel double-stranded molecules are useful candidates for the development of anti-cancer pharmaceuticals. For example, agents that block the expression of EBI3, DLX5, NPTX1, CDKN3 and/or EF-1delta protein and/or prevent its activity may find therapeutic utility as anti-cancer agents, particularly anti-cancer agents for the treatment of lung cancer, more particularly for the treatment of NSCLC and SCLC.

The expression of human genes EBI3, DLX5, NPTX1, CDKN3 and EF-1delta are markedly elevated in lung cancer, as compared to normal organs. Accordingly, these genes can be conveniently used as diagnostic markers of lung cancer and the proteins encoded thereby find utility in diagnostic assays of lung cancer.

Furthermore, the methods described herein are also useful in diagnosis of lung cancer, including small-cell lung carcinomas (SCLCs) and non-small cell lung cancers (NSCLCs), as well as the prediction of the poor prognosis of the patients with these diseases. Moreover, the present invention provides a likely candidate for development of therapeutic approaches for cancer including lung cancers.

In one aspect, the present invention relates to the discovery that EBI3 levels are elevated in the sera of lung-cancer patients as compared to that of normal controls. Accordingly, the EBI3 protein has utility as a diagnostic marker, particularly a serological marker for lung cancer. Using the serum level of EBI3 as an index, the present invention provides methods for diagnosing, as well as monitoring the progress of cancer treatment, in cancer patients. The prior art fails to provide a suitable serological marker for lung cancer. Novel serological marker EBI3 of the present invention may improve the sensitivity for detection of lung cancer. In addition, the combination of EBI3 and CEA or pro-GRP contributes to increase the sensitivity for detecting pancreatic cancer.

Furthermore, EBI3, DLX5, CDKN3, NPTX1 or EF-1delta polypeptide is a useful target for the development of anti-cancer pharmaceuticals. For example, agents that bind EBI3, DLX5, NPTX1, CDKN3 or EF-1delta or block the expression of EBI3, DLX5, NPTX1, CDKN3 or EF-1delta or prevent its activity, or inhibit the binding between CDKN3 and EF-1delta may find therapeutic utility as anti-cancer agents, particularly anti-cancer agents for the treatment of lung cancer.

All publications, databases, sequences, patents, and patent applications cited herein are hereby incorporated by reference.

While the invention has been described in detail and with reference to specific embodiments thereof, it will be apparent to one skilled in the art that various changes and modifications can be made therein without departing from the spirit and scope of the invention, the metes and bounds of which are set by the appended claims.

1. An isolated double-stranded molecule that, when introduced into a cell, inhibits in vivo expression of EBI3, CDKN3, EF-1delta or NPTXR as well as cell proliferation, said molecule comprising a sense strand and an antisense strand complementary thereto, said strands hybridized to each other to form the double-stranded molecule.

2. The double-stranded molecule of claim 1, wherein the sense strand comprises the sequence corresponding to a target sequence selected from the group consisting of SEQ ID NOs: 18, 20, 49, 51, 84, and 85.

3. The double-stranded molecule of claim 2, wherein the double stranded molecule is an oligonucleotide of between about 19 and about 25 nucleotides in length.

4. The double-stranded molecule of claim 1, which consists of a single polynucleotide comprising both the sense and antisense strands linked by an intervening single-strand.

5. The double-stranded molecule of claim 4, which has the general formula 5′-[A]-[B]-[A′]-3′, wherein [A] is the sense strand comprising a sequence corresponding to a target sequence selected from the group consisting of SEQ ID NOs: 18, 20, 49, 51, 84, and 85, [B] is the intervening single-strand consisting of 3 to 23 nucleotides, and [A′] is the antisense strand comprising a complementary sequence to [A].

6. A vector expressing the double-stranded molecule of claim 1.

7. A method for treating a cancer expressing at least one gene selected from the group consisting of EBI3, CDKN3, EF-1delta or NPTXR gene, wherein the method comprises the step of administering at least one isolated double-stranded molecule or vector expressing the double-stranded molecule of claim 1.

8. The method of claim 7, wherein the cancer to be treated is lung cancer.

9. A composition for treating a cancer expressing at least one gene selected from the group consisting of EBI3, CDKN3, EF-1delta and NPTXR gene, wherein the composition comprises at least one isolated double-stranded molecule or vector expressing the double-stranded molecule of claim 1.

10. The composition of claim 9, wherein the cancer to be treated is lung cancer.

11. A method for diagnosing lung cancer, said method comprising the steps of:
(a) determining the expression level of the gene in a subject-derived biological sample by any one of the method selected from the group consisting of:
(i) detecting the mRNA selected from the group of EBI3, DLX5 and CDKN3,
(ii) detecting the protein selected from the group of EBI3, DLX5 and CDKN3, and
(iii) detecting the biological activity of the protein selected from the group of EBI3, DLX5 and CDKN3; and
(b) relating an increase in the expression level determined in step (a) as compared to a normal control level of the gene to the presence of lung cancer.

12. The method of claim 11, wherein the expression level determined in step (a) is at least 10% greater than the normal control level.

13. The method of claim 11, wherein the expression level determined in step (a) is determined by detecting the binding of an antibody against the protein selected from the group consisting of EBI3, DLX5 and CDKN3.

14. The method of claim 11, wherein the subject-derived biological sample comprises biopsy, sputum, blood, pleural effusion or urine.

15. A method for assessing or determining the prognosis of a patient with lung cancer, which method comprises the steps of
(a) detecting the expression level of a gene in a patient-derived biological sample;
(b) comparing the detected expression level to a control level; and
(c) determining the prognosis of the patient based on the comparison of (b)
and wherein the gene is selected from the group consisting of EBI3, DLX5, CDKN3 and EF-1delta.

16. The method of claim 15, wherein the control level is determined as good prognosis and an increase of the expression level compared to the control level is determined as poor prognosis.

17. The method of claim 15, wherein the increase is at least 10% greater than the control level.

18. The method of claim 15, wherein the expression level is determined by any one method selected from the group consisting of:
(a) detecting mRNA of EBI3, DLX5, CDKN3 or EF-1delta;
(b) detecting the EBI3, DLX5, CDKN3 or EF-1delta protein; and
(c) detecting the biological activity of the EBI3, DLX5, CDKN3 or EF-1delta protein.

19. The method of claim 15, wherein the patient derived biological sample comprises biopsy, sputum or blood, pleural effusion or urine.

20. A kit for diagnosing lung cancer or assessing or determining the prognosis of a patient with lung cancer, which comprises a reagent selected from the group consisting of:
(a) a reagent for detecting mRNA of a gene;
(b) a reagent for detecting the protein encoded by the gene; and
(c) a reagent for detecting the biological activity of the protein
and wherein the gene is selected from the group consisting of EBI3, DLX5, CDKN3 and EF-1delta.

21. The kit of claim 20, wherein the reagent is a probe to a gene transcript of the gene.

22. The kit of claim 20, wherein the reagent is an antibody against the protein encoded by the gene.

23. A method for diagnosing lung cancer in a subject, comprising the steps of:
(a) providing a blood sample from a subject to be diagnosed;
(b) determining a level of EBI3 protein in the blood sample;
(c) comparing the EBI3 level determined in step (b) with that of a normal control, wherein a high EBI3 level in the blood sample, compared to the normal control, indicates that the subject suffers from a lung cancer.

24. The method of claim 23, wherein the blood sample is selected from the group consisting of whole blood, serum, and plasma.

25. The method of claim 23, wherein the EBI3 protein is detected by immunoassay.

26. The method of claim 25, wherein the immunoassay is an ELISA.

27. The method of claim 23, further comprising the steps of:
(d) determining a level of CEA in the blood sample;
(e) comparing the CEA level determined in step (d) with that of a normal control, wherein either or both of high EBI3 and high CEA levels in the blood sample, compared to the normal control, indicate that the subject suffers from a lung cancer.

28. The method of claim 27, wherein the lung cancer is NSCLC.

29. The method of claim 23, further comprising the steps of:
(d) determining a level of CYFRA in the blood sample;
(e) comparing the CYFRA level determined in step (d) with that of a normal control, wherein either or both of high EBI3 and high CYFRA levels in the blood sample, compared to the normal control, indicate that the subject suffers from a lung cancer.

30. The method of claim 29, wherein the lung cancer is SCC.

31. The method of claim 23, further comprising the steps of:
(d) determining a level of pro-GRP in the blood sample;
(e) comparing the pro-GRP level determined in step (d) with that of a normal control, wherein either or both of high EBI3 and high pro-GRP levels in the blood sample, compared to the normal control, indicate that the subject suffers from a lung cancer.

32. The method of claim 31, wherein the lung cancer is SCLC.

33. A kit for detecting a cancer expressing EBI3, wherein the kit comprises:
(i) an immunoassay reagent for determining a level of EBI3 in a blood sample; and
(ii) a positive control sample for EBI3.

34. The kit of claim 33, which further comprises:
(iii) an immunoassay reagent for determining a level of CEA, CYFRA and/or pro-GRP in a blood sample; and
(iv) a positive control sample for CEA, CYFRA and/or pro-GRP.

35. The kit of claim 34, wherein the positive control sample is positive for EBI3, CEA, CYFRA and/or pro-GRP.

36.-39. (canceled)

40. A method of screening for a candidate compound for treating or preventing lung cancer, or inhibiting lung cancer cell growth, said method comprising the steps of:
(a) contacting a test compound with a polypeptide encoded by a polynucleotide of EBI3, DLX5, or CDKN3;
(b) detecting the binding activity between the polypeptide and the test compound; and
(c) selecting a compound that binds to the polypeptide.

41. A method of screening for a candidate compound for treating or preventing lung cancer, or inhibiting lung cancer cell growth, said method comprising the steps of:
(a) contacting a test compound with a polypeptide encoded by a polynucleotide of EBI3, DLX5 or CDKN3;
(b) detecting the biological activity of the polypeptide of step (a); and
(c) selecting the candidate compound that suppresses the biological activity of the polypeptide encoded by the polynucleotide of EBI3, DLX5 or CDKN3 as compared to the biological activity of said polypeptide detected in the absence of the test compound.

42. The method of claim 41, wherein the biological activity is selected from the group of the facilitation consisting of the cell proliferation, cell invasion, extracellular secretion, phosphatase activity and Akt phosphorylation.

43. The method of claim 42, wherein the phosphatase activity was detected with EF-1delta.

44. A method of screening for a candidate compound for treating or preventing lung cancer or inhibiting lung cancer cell growth, said method comprising the steps of
(a) contacting a test compound with a cell expressing EBI3, DLX5 or CDKN3 and
(b) selecting the candidate compound that reduces the expression level of EBI3, DLX5 or CDKN3 in comparison with the expression level detected in the absence of the test compound.

45. A method of screening for a candidate compound for treating or preventing lung cancer or inhibiting lung cancer cell growth, said method comprising the steps of:
(a) contacting a test compound with a cell into which a vector, comprising the transcriptional regulatory region of EBI3, DLX5 or CDKN3 and a reporter gene that is expressed under the control of the transcriptional regulatory region, has been introduced;
(b) measuring the expression or activity of said reporter gene; and
(c) selecting a candidate compound that reduces the expression or activity level of said reporter gene as compared to a control.

46. A method of screening for a candidate compound for treating or preventing lung cancer or inhibiting lung cancer cell growth, said method comprising the steps of:
(a) contacting a CDKN3 polypeptide or functional equivalent thereof with an interaction partner selected from group consisting of VRS polypeptide, EF-1alpha polypeptide, EF-1beta polypeptide, EF-1gamma polypeptide, EF-1delta polypeptide and functional equivalent thereof, in the presence of a test compound;
(b) detecting the binding between the polypeptides; and
(c) selecting the candidate compound that inhibits the binding between these polypeptides.

47. The method of claim 46, wherein the functional equivalent of EF-1delta polypeptide comprises the polypeptide consisting of SEQ ID NO: 48.

48. The method of claim 46, wherein the functional equivalent of CDKN3 polypeptide comprises an amino acid sequence of VRS polypeptide, EF-1alpha polypeptide, EF-1beta polypeptide, EF-1gamma polypeptide or EF-1delta binding domain.

49. A method of screening for a compound for treating or preventing lung cancer, said method comprising the steps of:
(a) contacting a test compound with cells which over-expressing CDKN3;
(b) measuring the phosphorylation of Akt Ser473; and
(c) selecting a candidate compound that reduces the phosphorylation as compared to a control.

50.-69. (canceled)


SEQUENCE LISTING (91 sequences)

SEQ ID NO: 1 - DNA, 1149 bp, Homo sapiens
ccgcagccatgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccagctacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtagcaagggcttcccgctgcctccagacagcacctgggtcctcgccaccctaagccccgggacacctgttggagggcggatgggatctgcctagcctgggctggagtccttgctttgctgctgctgagctgccgggcaacctcagatgaccgacttttccctttgagcctcagtttctctagctgagaaatggagatgtactactctctcctttacctttacctttaccacagtgcagggctgactgaactgtcactgtgagatattttttattgtttaattagaaaagaattgttgttgggctgggcgcagtggatcgcacctgtaatcccagtcactgggaagccgacgtgggagggtagcttgaggccaggagctcgaaaccagtccgggccacacagcaagaccccatctctaaaaaattaatataaatataaaataaaaaaaaaaaaaa

SEQ ID NO: 2 - protein, 229 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 3 - DNA, 1419 bp, Homo sapiens
cggagacagagacttcacgactcccagtctcctcctcgccgcggccgccgcctcctccttctctcctcctcctcttcctcctcctccctcgctcccacagccatgtctgcttagaccagagcagccccacagccaactagggcagctgccgccgccacaacagcaaggacagccgctgccgccgcccgtgagcgatgacaggagtgtttgacagaagggtccccagcatccgatccggcgacttccaagctccgttccagacgtccgcagctatgcaccatccgtctcaggaatcgccaactttgcccgagtcttcagctaccgattctgactactacagccctacggggggagccccgcacggctactgctctcctacctcggcttcctatggcaaagctctcaacccctaccagtatcagtatcacggcgtgaacggctccgccgggagctacccagccaaagcttatgccgactatagctacgctagctcctaccaccagtacggcggcgcctacaaccgcgtcccaagcgccaccaaccagccagagaaagaagtgaccgagcccgaggtgagaatggtgaatggcaaaccaaagaaagttcgtaaacccaggactatttattccagctttcagctggccgcattacagagaaggtttcagaagactcagtacctcgccttgccggaacgcgccgagctggccgcctcgctgggattgacacaaacacaggtgaaaatctggtttcagaacaaaagatccaagatcaagaagatcatgaaaaacggggagatgcccccggagcacagtcccagctccagcgacccaatggcgtgtaactcgccgcagtctccagcggtgtgggagccccagggctcgtcccgctcgctcagccaccaccctcatgcccaccctccgacctccaaccagtccccagcgtccagctacctggagaactctgcatcctggtacacaagtgcagccagctcaatcaattcccacctgccgccgccgggctccttacagcacccgctggcgctggcctccgggacactctattagatgggctgctctctcttactctcttttttgggactactgtgttttgctgttctagaaaatcataaagaaaggaattcatatggggaagttcggaaaactgaaaaagattcatgtgtaaagcttttttttgcatgtaagttattgcatttcaaaagaccccccctttttttacagaggactttttttgcgcaactgtggacactttcaatggtgccttgaaatctatgacctcaacttttcaaaagacttttttcaatgttattttagccatgtaaataagtgtagatagaggaattaaactgtatattctggataaataaaattatttcgaccatgaaaaaaaaaaaaaaaaaaa

SEQ ID NO: 4 - protein, 289 aa, Homo sapiens
MTGVFDRRVPSIRSGDFQAPFQTSAAMHHPSQESPTLPESSATDSDYYSPTGGAPHGYCSPTSASYGKALNPYQYQYHGVNGSAGSYPAKAYADYSYASSYHQYGGAYNRVPSATNQPEKEVTEPEVRMVNGKPKKVRKPRTIYSSFQLAALQRRFQKTQYLALPERAELAASLGLTQTQVKIWFQNKRSKIKKIMKNGEMPPEHSPSSSDPMACNSPQSPAVWEPQGSSRSLSHHPHAHPPTSNQSPASSYLENSASWYTSAASSINSHLPPPGSLQHPLALASGTLY

SEQ ID NO: 5 - DNA, 844 bp, Homo sapiens
gcacgagctgcagagggaggcggcactggtctcgacgtggggcggccagcgatgaagccgcccagttcaatacaaacaagtgagtttgactcatcagatgaagagcctattgaagatgaacagactccaattcatatatcatggctatctttgtcacgagtgaattgttctcagtttctcggtttatgtgctcttccaggttgtaaatttaaagatgttagaagaaatgtccaaaaagatacagaagaactaaagagctgtggtatacaagacatatttgttttctgcaccagaggggaactgtcaaaatatagagtcccaaaccttctggatctctaccagcaatgtggaattatcacccatcatcatccaatcgcagatggagggactcctgacatagccagctgctgtgaaataatggaagagcttacaacctgccttaaaaattaccgaaaaaccttaatacactgctatggaggacttgggagatcttgtcttgtagctgcttgtctcctactatacctgtctgacacaatatcaccagagcaagccatagacagcctgcgagacctaagaggatccggggcaatacagaccatcaagcaatacaattatcttcatgagtttcgggacaaattagctgcacatctatcatcaagagattcacaatcaagatctgtatcaagataaaggaattcaaatagcatatatatgaccatgtctgaaatgtcagttctctagcataatttgtattgaaatgaaaccaccagtgttatcaacttgaatgtaaatgtacatgtgcagatattcctaaagttttattgacaaaaaaaaaaaaaaaaaa

SEQ ID NO: 6 - protein, 212 aa, Homo sapiens
MKPPSSIQTSEFDSSDEEPIEDEQTPIHISWLSLSRVNCSQFLGLCALPGCKFKDVRRNVQKDTEELKSCGIQDIFVFCTRGELSKYRVPNLLDLYQQCGIITHHHPIADGGTPDIASCCEIMEELTTCLKNYRKTLIHCYGGLGRSCLVAACLLLYLSDTISPEQAIDSLRDLRGSGAIQTIKQYNYLHEFRDKLAAHLSSRDSQSRSVSR

SEQ ID NO: 7 - DNA, 1031 bp, Homo sapiens
cccgcgtccgccgattcctcctccttggtcgccgcgtccttggctggcgtcagaaaaatggctacaaacttcctagcacatgagaagatctggttcgacaagttcaaatatgacgacgcagaaaggagattctacgagcagatgaacgggcctgtggcaggtgcctcccgccaggagaacggcgccagcgtgatcctccgtgacattgcgagagccagagagaacatccagaaatccctggctggaagctcaggccccggggcctccagcggcaccagcggagaccacggtgagctcgtcgtccggattgccagtctggaagtggagaaccagagtctgcgtggcgtggtacaggagctgcagcaggccatctccaagctggaggcccggctgaacgtgctggagaagagctcgcctggccaccgggccacggccccacagacccagcacgtatctcccatgcgccaagtggagcccccagccaagaagccagccacaccagcagaggatgacgaggatgatgacattgacctgtttggcagtgacaatgaggaggaggacaaggaggcggcacagctgcgggaggagcggctacggcagtacgcggagaagaaggccaagaagcctgcactggtggccaagtcctccatcctgctggatgtcaagccttgggatgatgagacggacatggctcagctggaggcctgtgtgcgctctatccagctggacgggctggtctggggggcttccaagctggtgcccgtgggctacggtatccggaagctacagattcagtgtgtggtggaggacgacaaggtggggacagacttgctggaggaggagatcaccaagtttgaggagcacgtgcagagtgtcgatatcgcagctttcaacaagatctgaagcctgagtgtgtgtacgtgcgcgcgtgcgtgaggccctgccacgattaaagactgagaccggcaaaaaaaaaaaaaaaaaaaaaaaaaaaaaaaaaaaaaaaaaaaaaaaaaaaaaaaaaaaaaaaa

SEQ ID NO: 8 - protein, 281 aa, Homo sapiens
MATNFLAHEKIWFDKFKYDDAERRFYEQMNGPVAGASRQENGASVILRDIARARENIQKSLAGSSGPGASSGTSGDHGELVVRIASLEVENQSLRGVVQELQQAISKLEARLNVLEKSSPGHRATAPQTQHVSPMRQVEPPAKKPATPAEDDEDDDIDLFGSDNEEEDKEAAQLREERLRQYAEKKAKKPALVAKSSILLDVKPWDDETDMAQLEACVRSIQLDGLVWGASKLVPVGYGIRKLQIQCVVEDDKVGTDLLEEEITKFEEHVQSVDIAAFNKI

SEQ ID NO: 9 - DNA, 20 bp, Artificial
  An artificially synthesized primer for PCR
tgttctccatggctccctac

SEQ ID NO: 10 - DNA, 20 bp, Artificial
  An artificially synthesized primer for PCR
agctccctgacgcttgtaac

SEQ ID NO: 11 - DNA, 21 bp, Artificial
  An artificially synthesized primer for PCR
gaggtgatagcattgctttcg

SEQ ID NO: 12 - DNA, 21 bp, Artificial
  An artificially synthesized primer for PCR
caagtcagtgtacaggtaagc

SEQ ID NO: 13 - DNA, 20 bp, Artificial
  An artificially synthesized primer for northern blot probe
tgttctccatggctccctac

SEQ ID NO: 14 - DNA, 20 bp, Artificial
  An artificially synthesized primer for northern blot probe
ctacttgcccaggctcattg

SEQ ID NO: 15 - DNA, 18 bp, Artificial
  An artificially synthesized target sequence for siRNA
cgtacgcggaatacttcg

SEQ ID NO: 16 - DNA, 19 bp, Artificial
  An artificially synthesized target sequence for siRNA
gcgcgctttgtaggattcg

SEQ ID NO: 17 - DNA, 21 bp, Artificial
  An artificially synthesized target sequence for siRNA
uacuugcccaggcucauuguu

SEQ ID NO: 18 - DNA, 19 bp, Artificial
  An artificially synthesized target sequence for siRNA
caatgagcctgggcaagta

SEQ ID NO: 19 - DNA, 21 bp, Artificial
  An artificially synthesized target sequence for siRNA
aacagcuggacauccgugauu

SEQ ID NO: 20 - DNA, 19 bp, Artificial
  An artificially synthesized target sequence for siRNA
tcacggatgtccagctgtt

SEQ ID NO: 21 - DNA, 22 bp, Artificial
  An artificially synthesized primer for PCR
ctcgctcagccaccaccctcat

SEQ ID NO: 22 - DNA, 26 bp, Artificial
  An artificially synthesized primer for PCR
agttgaggtcatagatttcaaggcac

SEQ ID NO: 23 - DNA, 19 bp, Artificial
  An artificially synthesized target sequence for siRNA
gaagcagcacgacttcttc

SEQ ID NO: 24 - DNA, 19 bp, Artificial
  An artificially synthesized target sequence for siRNA
ccagccagagaaagaagtg

SEQ ID NO: 25 - DNA, 19 bp, Artificial
  An artificially synthesized target sequence for siRNA
gtgcagccagctcaatcaa

SEQ ID NO: 26 - DNA, 4308 bp, Homo sapiens
gcgcctgacgggagcgtcgtgctcaggggtgtcctctcgtcctgcgtccgcgcccagcgccccgcgccccgcgctgttcctcgtgagaccggcgggcggcgagccgcgcggcccccggggcagtgtcggacacggcgggcgcgcactcgcaggcggggcacggccgcccccgccaggacccgcaggcccggaaacgctccctgtcacaaaggggggaacacgtgggcgccggctgccggggcggcgatcttagggaactagggtcacctggagagccgcccaccgtctctgcccgctcgactcctccgcccgggccgctcggccggtccagccgcggccggcgcctggctgtgaggtggattcccggcccagtctgaccatctccctccagtttttccacttcgttcggaccttctcataactatgtccaccctctacgtctcccctcacccagatgccttccccagcctccgagccctcatagccgctcgctatggggaggctggggagggtcccggatggggaggagcccacccccgcatctgtctccagccacccccgactagcaggactccctttcccccaccccgcctgccggccctggagcaggggcccggtgggctctgggtgtggggggccacggctgtggcccagctgctgtggccagcaggcctggggggcccagggggcagccgggcggctgtccttgtccaacagtgggtcagttacgccgacacggagttaataccagctgcctgtggagcaacgctgccggccctgggactccgaagctcggcccaggacccccaggctgtgctgggggccctgggcagggccctgagccccttggaggagtggcttcggctgcacacctacttggccggggaggcccccactctggctgacctggcggctgtcacagccttgctgctgcctttccgatacgtcctagacccacctgcccgccggatctggaataatgtgactcgctggtttgtcacgtgtgtccggcagccagaattccgagccgtgctaggagaagtggttctatactcaggagccaggcctctctctcatcagccaggccccgaggctcctgccctcccaaagacagctgctcagctcaagaaagaggcaaagaaacgggagaagctagagaaattccaacagaagcagaagatccaacagcagcagccacctccaggggagaagaaaccaaaaccagagaagagggagaaacgggatcctggggtcattacctatgacctcccaaccccacccggggaaaagaaagatgtcagtggccccatgcccgactcctacagccctcggtatgtggaggctgcctggtacccttggtgggagcagcagggcttcttcaagccagagtatgggcgtcctaatgtgtcagcagcaaatccccgaggtgtcttcatgatgtgcatcccaccccccaatgtgacaggctccctgcacctgggccatgcactcaccaacgccatccaggactccctgactcgatggcaccgcatgcgtggggagaccaccctgtggaaccctggctgtgaccatgcaggtattgccacccaggtggtggtggagaagaagctatggcgtgagcagggactgagccggcaccagctgggccgcgaggcctttctacaggaagtctggaagtggaaggaggagaaaggtgaccggatttaccaccagttgaagaagcttggcagctccttggactgggatcgagcctgtttcaccatggaccctaaactctcagcagctgtgacagaggcctttgtccggcttcacgaggaaggcatcatctatcgcagtacccgccttgttaactggtcctgcaccctcaactccgccatctctgacattgaggtggataagaaggagctgacaggtcgcaccctgctctccgtgcctggctacaaggagaaggtggagttcggggtcctcgtgtcctttgcctataaggtccaaggctcagatagcgacgaggaggtggtggtggcaacaactcggatcgagacaatgctgggagatgtggctgtagctgtgcaccccaaagataccagataccagcacctgaaggggaagaacgtgatccacccattcctgtctcggagccttcccattgtcttcgatgaatttgtggacatggactttggcacaggtgctgtgaagatcacccccgcacatgaccaaaatgactatgaagttgggcagcggcacgggctggaggccatcagcatcatggactcccggggggccctcatcaatgtgcctccgcctttcctgggcctgcccaggtttgaggccaggaaagcggtgctggtggcgctgaaggagcggggactgttccgtggcattgaggacaaccccatggtggtgccactttgcaaccggtcgaaggacgtggtagagcctctgctgcggccgcagtggtacgttcgctgcggggagatggcccaggctgccagcgccgctgtgactcggggtgacctccgcatcctgcctgaggcccatcagcgcacatggcatgcctggatggacaacatccgggagtggtgcatttccaggcagctgtggtggggccatcgcatcccagcctactttgtcactgtcagtgacccagcggtgccccctggggaggaccctgatgggcggtactgggtgagtggacgcaatgaggcggaggcccgggagaaggcagccaaggagttcggagtgtcccctgacaagatcagtctccagcaagatgaggatgtattggatacctggttctcctctggcctcttccccttatccattttgggctggcccaaccagtcagaagacctgagtgtgttctaccccgggacactgctggagaccggtcatgacatcctcttcttctgggtggcccggatggtcatgctgggcctgaagctcacgggcaggctgccctttagagaggtctacctccatgccatcgtgcgagatgctcacggccggaagatgagcaagtctctaggcaatgtcatcgatcccctggacgtcatctatggaatctccctgcagggcctccacaaccagctgctgaacagcaacctggatcccagcgaggtggagaaggccaaagaagggcagaaagctgacttcccagcggggattcctgaatgtggcaccgatgctctccggtttggattatgtgcctacatgtcccagggtcgtgacatcaacctggatgtgaaccggatactgggttaccgccacttctgcaacaagctctggaatgccaccaagtttgcccttcgtggccttgggaagggttttgtgccctcacccacctcccagcccggaggccatgagagcctggtggaccgctggatccgcagccgcctgacagaggctgtgaggctcagcaatcaaggcttccaggcctacgacttcccggccgtcaccactgcccagtacagcttctggctctatgagctctgtgatgtctacttggagtgcctgaaacctgtactgaatggggtggaccaggtggcagctgagtgtgcccgccagaccctgtacacttgcctggacgttggcctgcggctgctctcacccttcatgcccttcgtgacggaggagctgttccagaggctgccccggaggatgccgcaagctccccctagcctctgtgttaccccctacccggagccctcagagtgctcctggaaggaccccgaggcagaagccgcccttgagctggcgctaagcatcacgcgagccgtgcgctccctgcgggccgactacaacctcacccggatccggcctgactgtttcctggaagtggcggatgaggccacgggcgccctggcatcggcggtgtcgggctacgtgcaggccctggccagcgcaggtgtggtggctgttctggccctgggggctcccgccccccagggttgcgctgtggctctggcttctgatcgctgctccatccacctgcagcttcaggggctggtggaccctgcacgggagctgggcaagctgcaagccaagcgagttgaggcccagcggcaggcccagcgtctgcgggaacgccgtgctgcctcgggctatcctgtcaaggtgccgctcgaagtccaggaggcagatgaagccaagctccaacagacagaagcagagctcaggaaggtggatgaggccatcgccctattccagaagatgctgtgatccaccacccagcttcacccctcacccccagcggctcaccatggggatggcagcaataaaatattttcccacaaaaaaaaaaaaaaaaaa

SEQ ID NO: 27 - protein, 1264 aa, Homo sapiens
MSTLYVSPHPDAFPSLRALIAARYGEAGEGPGWGGAHPRICLQPPPTSRTPFPPPRLPALEQGPGGLWVWGATAVAQLLWPAGLGGPGGSRAAVLVQQWVSYADTELIPAACGATLPALGLRSSAQDPQAVLGALGRALSPLEEWLRLHTYLAGEAPTLADLAAVTALLLPFRYVLDPPARRIWNNVTRWFVTCVRQPEFRAVLGEVVLYSGARPLSHQPGPEAPALPKTAAQLKKEAKKREKLEKFQQKQKIQQQQPPPGEKKPKPEKREKRDPGVITYDLPTPPGEKKDVSGPMPDSYSPRYVEAAWYPWWEQQGFFKPEYGRPNVSAANPRGVFMMCIPPPNVTGSLHLGHALTNAIQDSLTRWHRMRGETTLWNPGCDHAGIATQVVVEKKLWREQGLSRHQLGREAFLQEVWKWKEEKGDRIYHQLKKLGSSLDWDRACFTMDPKLSAAVTEAFVRLHEEGIIYRSTRLVNWSCTLNSAISDIEVDKKELTGRTLLSVPGYKEKVEFGVLVSFAYKVQGSDSDEEVVVATTRIETMLGDVAVAVHPKDTRYQHLKGKNVIHPFLSRSLPIVFDEFVDMDFGTGAVKITPAHDQNDYEVGQRHGLEAISIMDSRGALINVPPPFLGLPRFEARKAVLVALKERGLFRGIEDNPMVVPLCNRSKDVVEPLLRPQWYVRCGEMAQAASAAVTRGDLRILPEAHQRTWHAWMDNIREWCISRQLWWGHRIPAYFVTVSDPAVPPGEDPDGRYWVSGRNEAEAREKAAKEFGVSPDKISLQQDEDVLDTWFSSGLFPLSILGWPNQSEDLSVFYPGTLLETGHDILFFWVARMVMLGLKLTGRLPFREVYLHAIVRDAHGRKMSKSLGNVIDPLDVIYGISLQGLHNQLLNSNLDPSEVEKAKEGQKADFPAGIPECGTDALRFGLCAYMSQGRDINLDVNRILGYRHFCNKLWNATKFALRGLGKGFVPSPTSQPGGHESLVDRWIRSRLTEAVRLSNQGFQAYDFPAVTTAQYSFWLYELCDVYLECLKPVLNGVDQVAAECARQTLYTCLDVGLRLLSPFMPFVTEELFQRLPRRMPQAPPSLCVTPYPEPSECSWKDPEAEAALELALSITRAVRSLRADYNLTRIRPDCFLEVADEATGALASAVSGYVQALASAGVVAVLALGAPAPQGCAVALASDRCSIHLQLQGLVDPARELGKLQAKRVEAQRQAQRLRERRAASGYPVKVPLEVQEADEAKLQQTEAELRKVDEAIALFQKML

SEQ ID NO: 28 - DNA, 4078 bp, Homo sapiens
ggctgccggggcggcgatcttagggaactagggtcacctggagagccgcccaccgtctctgcccgctcgactcctccgcccgggccgctcggccggtccagccgcggccggcgcctggctgtgaggtggattcccggcccagtctgaccatctccctccagtttttccacttcgttcggaccttctcataactatgtccaccctctacgtctcccctcacccagatgccttccccagcctccgagccctcatagccgctcgctatggggaggctggggagggtcccggatggggaggagcccacccccgcatctgtctccagccacccccgactagcaggactagctttcccccaccccgcctgccggccctggagcaggggcccggtgggctctgggtgtggggggccacggctgtggcccagctgctgtggccagcaggcctggggggcccagggggcagccgggcggctgtccttgtccaacagtgggtcagttacgccgacacggagttaataccagctgcctgtggagcaacgctgccggccctgggactccgaagctcggcccaggacccccaggctgtgctgggggccctgggcagggccctgagccccttggaggagtggcttcggctgcacacctacttggccggggaggcccccactctggctgacctggcggctgtcacagccttgctgctgcctttccgatacgtcctagacccacctgcccgccggatctggaataatgtgactcgctggtttgtcacgtgtgtccggcagccagaattccgagccgtgctaggagaagtggttctatactcaggagccaggcctctctctcatcagccaggccccgaggctcctgccctcccaaagacagctgctcagctcaagaaagaggcaaagaaacgggagaagctagagaaattccaacagaagcagaagatccaacagcagcagccacctccaggggagaagaaaccaaaaccagagaagagggagaaacgggatcctggggtcattacctatgacctcccaaccccacccggggaaaagaaagatgtcagtggccccatgcccgactcctacagccctcggtatgtggaggctgcctggtacccttggtgggagcagcagggcttcttcaagccagagtatgggcgtcctaatgtgtcagcagcaaatccccgaggtgtcttcatgatgtgcatcccaccccccaatgtgacaggctccctgcacctgggccatgcactcaccaacgccatccaggactccctgactcgatggcaccgcatgcgtggggagaccaccctgtggaaccctggctgtgaccatgcaggtattgccacccaggtggtggtggagaagaagctatggcgtgagcagggactgagccggcaccagctgggccgcgaggcctttctacaggaagtctggaagtggaaggaggagaaaggtgaccggatttaccaccagttgaagaagcttggcagctccttggactgggatcgagcctgttttaccatggaccctaaactctcagcagctgtgacagaggcctttgtccggcttcacgaggaaggcatcatctatcgcagtacccgccttgttaactggtcctgcaccctcaactccgccatctctgacattgaggtggataagaaggagctgacaggtcgcaccctgctctccgtgcctggctacaaggagaaggtggagttcggggtcctcgtgtcctttgcctataaggtccaaggctcagatagcgacgaggaggtggtggtggcaacaactcggatcgagacaatgctgggagatgtggctgtagctgtgcaccccaaagataccagataccagcacctgaaggggaagaacgtgatccacccattcctgtctcggagccttcccattgtcttcgatgaatttgtggacatggactttggcacaggtgctgtgaagatcacccccgcacatgaccaaaatgactatgaagttgggcagcggcacgggctggaggccatcagcatcatggactcccggggggccctcatcaatgtgcctccgcctttcctgggcctgcccaggtttgaggccaggaaagcggtgctggtggcgctgaaggagcggggactgttccgtggcattgaggacaaccccatggtggtgccactttgcaaccggtcgaaggacgtggtagagcctctgctgcggccgcagtggtacgttcgctgcggggagatggcccaggctgccagcgccgctgtgactcggggtgacctccgcatcctgcctgaggcccatcagcgcacatggcatgcctggatggacaacatccgggagtggtgcatttccaggcagctgtggtggggccatcgcatcccagcctactttgtcactgtcagtgacccagcggtgccccctggggaggaccctgatgggcggtactgggtgagtggacgcaatgaggcggaggcccgggagaaggcagccaaggagttcggagtgtcccctgacaagatcagtctccagcaagatgaggatgtattggatacctggttctcctctggcctcttccccttatccattttgggctggcccaaccagtcagaagacctgagtgtgttctaccccgggacactgctggagaccggtcatgacatcctcttcttctgggtggcccggatggtcatgctgggcctgaagctcacgggcaggctgccctttagagaggtctacctccatgccatcgtgcgagatgctcacggccggaagatgagcaagtctctaggcaatgtcatcgatcccctggacgtcatctatggaatctccctgcagggcctccacaaccagctgctgaacagcaacctggatcccagcgaggtggagaaggccaaagaagggcagaaagctgacttcccagcggggattcctgaatgtggcaccgatgctctccggtttggattatgtgcctacatgtcccagggtcgtgacatcaacctggatgtgaaccggatactgggttaccgccacttctgcaacaagctctggaatgccaccaagtttgcccttcgtggccttgggaagggttttgtgccctcacccacctcccagcccggaggccatgagagcctggtggaccgctggatccgcagccgcctgacagaggctgtgaggctcagcaatcaaggcttccaggcctacgacttcccggccgtcaccactgcccagtacagcttctggctctatgagctctgtgatgtctacttggagtgcctgaaacctgtactgaatggggtggaccaggtggcagctgagtgtgcccgccagaccctgtacacttgcctggacgttggcctgcggctgctctcacccttcatgcccttcgtgacggaggagctgttccagaggctgccccggaggatgccgcaagctccccctagcctctgtgttaccccctacccggagccctcagagtgctcctggaaggaccccgaggcagaagccgcccttgagctggcgctaagcatcacgcgagccgtgcgctccctgcgggccgactacaacctcacccggatccggcctgactgtttcctggaagtggcggatgaggccacgggcgccctggcatcggcggtgtcgggctacgtgcaggccctggccagcgcaggtgtggtggctgttctggccctgggggctcccgccccccagggttgcgctgtggctctggcttctgatcgctgctccatccacctgcagcttcaggggctggtggaccctgcacgggagctgggcaagctgcaagccaagcgagttgaggcccagcggcaggcccagcgtctgcgggaacgccgtgctgcctcgggctatcctgtcaaggtgccgctcgaagtccaggaggcagatgaagccaagctccaacagacagaagcagagctcaggaaggtggatgaggccatcgccctattccagaagatgctgtgatccaccacccagcttcacccctcacccccagcggctcaccatggggatggcagcaataaaatattttcccacaaaaaaaaaaaaaaaaaa

SEQ ID NO: 29 - protein, 1264 aa, Homo sapiens [sequence omitted]

SEQ ID NO: 30 - DNA, 1128 bp, Homo sapiens
gccggaagtggccccagcctcgaggccgggcgtcttcggtcatctccggcgcttctagggctggttcccgtcatcttcgggagccgtggaggtacgaacttaagacatgcctattttattaatttacttccaaacgcaacgaaaggtccatggacaatttgtgggccatttaattcagggcccccaattcgtacgtggagaagtgggaatgcaaaagtactttgacctttaaccttcggtccggcgcggtggagggaaacgcctccgtctctatataaggaattttccggtctcttcgggtcctttttcctctcttcagcgtggggcgcccacaatttgcgcgctctctttctgctgctccccagctctcggatacagccgacaccatgggtttcggagacctgaaaagccctgccggcctccaggtgctcaacgattacctggcggacaagagctacatcgaggggtatgtgccatcacaagcagatgtggcagtatttgaagccgtgtccagcccaccgcctgccgacttgtgtcatgccctacgttggtataatcacatcaagtcttacgaaaaggaaaaggccagcctgccaggagtgaagaaagctttgggcaaatatggtcctgccgatgtggaagacactacaggaagtggagctacagatagtaaagatgatgatgacattgacctctttggatctgatgatgaggaggaaagtgaagaagcaaagaggctaagggaagaacgtcttgcacaatatgaatcaaagaaagccaaaaaacctgcacttgttgccaagtcttccatcttactagatgtgaaaccttgggatgatgagacagatatggcgaaattagaggagtgcgtcagaagcattcaagcagacggcttagtctggggctcatctaaactagttccagtgggatacggaattaagaaacttcaaatacagtgtgtagttgaagatgataaagttggaacagatatgctggaggagcagatcactgcttttgaggactatgtgcagtccatggatgtggctgctttcaacaagatctaaaatccatcctggatcatggcatttaaataaaagattgaaagattacaaaaaaaaaaaaaaaaaa

SEQ ID NO: 31 - protein, 225 aa, Homo sapiens
MGFGDLKSPAGLQVLNDYLADKSYIEGYVPSQADVAVFEAVSSPPPADLCHALRWYNHIKSYEKEKASLPGVKKALGKYGPADVEDTTGSGATDSKDDDDIDLFGSDDEEESEEAKRLREERLAQYESKKAKKPALVAKSSILLDVKPWDDETDMAKLEECVRSIQADGLVWGSSKLVPVGYGIKKLQIQCVVEDDKVGTDMLEEQITAFEDYVQSMDVAAFNKI

SEQ ID NO: 32 - DNA, 1435 bp, Homo sapiens
atcaccatggcggctgggaccctgtacacgtatcctgaaaactggagggccttcaaggctctcatcgctgctcagtacagcggggctcaggtccgcgtgctctccgcaccaccccacttccattttggccaaaccaaccgcacccctgaatttctccgcaaatttcctgccggcaaggtcccagcatttgagggtgatgatggattctgtgtgtttgagagcaacgccattgcctactatgtgagcaatgaggagctgcggggaagtactccagaggcagcagcccaggtggtgcagtgggtgagctttgctgattccgatatagtgcccccagccagtacctgggtgttccccaccttgggcatcatgcaccacaacaaacaggccactgagaatgcaaaggaggaagtgaggcgaattctggggctgctggatgcttacttgaagacgaggacttttctggtgggcgaacgagtgacattggctgacatcacagttgtctgcaccctgttgtggctctataagcaggttctagagccttctttccgccaggcctttcccaataccaaccgctggttcctcacctgcattaaccagccccagttccgggctgtcttgggggaagtgaaactgtgtgagaagatggcccagtttgatgctaaaaagtttgcagagacccaacctaaaaaggacacaccacggaaagagaagggttcacgggaagagaagcagaagccccaggctgagcggaaggaggagaaaaaggcggctgcccctgctcctgaggaggagatggatgaatgtgagcaggcgctggctgctgagcccaaggccaaggaccccttcgctcacctgcccaagagtacctttgtgttggatgaatttaagcgcaagtactccaatgaggacacactctctgtggcactgccatatttctgggagcactttgataaggacggctggtccctgtggtactcagagtatcgcttccctgaagaactcactcagaccttcatgagctgcaatctcatcactggaatgttccagcgactggacaagctgaggaagaatgccttcgccagtgtcatcctttttggaaccaacaatagcagctccatttctggagtctgggtcttccgaggccaggagcttgcctttccgctgagtccagattggcaggtggactacgagtcatacacatggcggaaactggatcctggcagcgaggagacccagacgctggttcgagagtacttttcctgggagggggccttccagcatgtgggcaaagccttcaatcagggcaagatcttcaagtgaacatctcttgccatcacctagctgcctgcacctgcccttcagggagatgggggtcattaaaggaaactgaacattgaaaaaaaaaaaaaaaaaaaaaaaaaaaaaaaaaaaaaaa

SEQ ID NO: 33 - protein, 437 aa, Homo sapiens
MAAGTLYTYPENWRAFKALIAAQYSGAQVRVLSAPPHFHFGQTNRTPEFLRKFPAGKVPAFEGDDGFCVFESNAIAYYVSNEELRGSTPEAAAQVVQWVSFADSDIVPPASTWVFPTLGIMHHNKQATENAKEEVRRILGLLDAYLKTRTFLVGERVTLADITVVCTLLWLYKQVLEPSFRQAFPNTNRWFLTCINQPQFRAVLGEVKLCEKMAQFDAKKFAETQPKKDTPRKEKGSREEKQKPQAERKEEKKAAAPAPEEEMDECEQALAAEPKAKDPFAHLPKSTFVLDEFKRKYSNEDTLSVALPYFWEHFDKDGWSLWYSEYRFPEELTQTFMSCNLITGMFQRLDKLRKNAFASVILFGTNNSSSISGVWVFRGQELAFPLSPDWQVDYESYTWRKLDPGSEETQTLVREYFSWEGAFQHVGKAFNQGKIFK

SEQ ID NO: 34 - DNA, 23 bp, Artificial
  An artificially synthesized primer for PCR
gtgaattgttctcagtttctcgg

SEQ ID NO: 35 - DNA, 23 bp, Artificial
  An artificially synthesized primer for PCR
tctcttgatgatagatgtgcagc

SEQ ID NO: 36 - DNA, 23 bp, Artificial
  An artificially synthesized primer for PCR
tggctacaaacttcctagcacat

SEQ ID NO: 37 - DNA, 23 bp, Artificial
  An artificially synthesized primer for PCR
ctccaccacacactgaatctgta

SEQ ID NO: 38 - DNA, 20 bp, Artificial
  An artificially synthesized primer for PCR
taagcatcacgcgagccgtg

SEQ ID NO: 39 - DNA, 23 bp, Artificial
  An artificially synthesized primer for PCR
ggatggagcagcagcgatcagaa

SEQ ID NO: 40 - DNA, 23 bp, Artificial
  An artificially synthesized primer for PCR
agactggttaatgataacaatgc

SEQ ID NO: 41 - DNA, 23 bp, Artificial
  An artificially synthesized primer for PCR
ggtctcaaaattctgtgacaaat

SEQ ID NO: 42 - DNA, 20 bp, Artificial
  An artificially synthesized primer for PCR
cagaagcattcaagcagacg

SEQ ID NO: 43 - DNA, 20 bp, Artificial
  An artificially synthesized primer for PCR
atgccatgatccaggatgga

SEQ ID NO: 44 - DNA, 23 bp, Artificial
  An artificially synthesized primer for PCR
ggtggactacgagtcatacacat

SEQ ID NO: 45 - DNA, 21 bp, Artificial
  An artificially synthesized primer for PCR
cagtttcctttaatgaccccc

SEQ ID NO: 46 - DNA, 20 bp, Artificial
  An artificially synthesized primer for PCR
agcctagcatcccatgtcaa

SEQ ID NO: 47 - DNA, 21 bp, Artificial
  An artificially synthesized primer for PCR
gaagacgaagtacagctgaag

SEQ ID NO: 48 - protein, 89 aa, Artificial
  CDKN3-binding sequence in EF-1delta
GTSGDHGELVVRIASLEVENQSLRGVVQELQQAISKLEARLNVLEKSSPGHRATAPQTQHVSPMRQVEPPAKKPATPAEDDEDDDIDLF

SEQ ID NO: 49 - DNA, 19 bp, Artificial
  An artificially synthesized target sequence for siRNA
tatagagtcccaaaccttc

SEQ ID NO: 50 - DNA, 19 bp, Artificial
  An artificially synthesized target sequence for siRNA
tacactgctatggaggact

SEQ ID NO: 51 - DNA, 19 bp, Artificial
  An artificially synthesized target sequence for siRNA
gtggagaaccagagtctgc

SEQ ID NO: 52 - DNA, 19 bp, Artificial
  An artificially synthesized target sequence for siRNA
catccagaaatccctggct

SEQ ID NO: 53 - DNA, 19 bp, Artificial
  An artificially synthesized target sequence for siRNA
ugguuuacaugucgacuaa

SEQ ID NO: 54 - DNA, 19 bp, Artificial
  An artificially synthesized target sequence for siRNA
ugguuuacauguuuucuga

SEQ ID NO: 55 - DNA, 19 bp, Artificial
  An artificially synthesized target sequence for siRNA
ugguuuacauguuuuccua

SEQ ID NO: 56 - DNA, 19 bp, Artificial
  An artificially synthesized target sequence for siRNA
ugguuuacauguuguguga

SEQ ID NO: 57 - DNA, 3528 bp, Homo sapiens
ctttttcgcaacgggtttgccgccagaacacaggtgtcgtgaaaactacccctaaaagccaaaatgggaaaggaaaagactcatatcaacattgtcgtcattggacacgtagattcgggcaagtccaccactactggccatctgatctataaatgcggtggcatcgacaaaagaaccattgaaaaatttgagaaggaggctgctgagatgggaaagggctccttcaagtatgcctgggtcttggataaactgaaagctgagcgtgaacgtggtatcaccattgatatctccttgtggaaatttgagaccagcaagtactatgtgactatcattgatgccccaggacacagagactttatcaaaaacatgattacagggacatctcaggctgactgtgctgtcctgattgttgctgctggtgttggtgaatttgaagctggtatctccaagaatgggcagacccgagagcatgcccttctggcttacacactgggtgtgaaacaactaattgtcggtgttaacaaaatggattccactgagccaccctacagccagaagagatatgaggaaattgttaaggaagtcagcacttacattaagaaaattggctacaaccccgacacagtagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagctttgagtgaagctcttcctggggacaatgtgggcttcaatgtcaagaatgtgtctgtcaaggatgttcgtcgtggcaacgttgctggtgacagcaaaaatgacccaccaatggaagcagctggcttcactgctcaggtgattatcctgaaccatccaggccaaataagcgccggctatgcccctgtattggattgccacacggctcacattgcatgcaagtttgctgagctgaaggaaaagattgatcgccgttctggtaaaaagctggaagatggccctaaattcttgaagtctggtgatgctgccattgttgatatggttcctggcaagcccatgtgtgttgagagcttctcagactatccacctttgggtcgctttgctgttcgtgatatgagacagacagttgcggtgggtgtcatcaaagcagtggacaagaaggctgctggagctggcaaggtcaccaagtctgcccagaaagctcagaaggctaaatgaatattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgtgttgcatagaaagcttcatgttgctaaaccaatgttaagtgaatctttggaaacaaaatgtttccaaattactgggatgtgcatgttgaaacgtgggttaaaatgactgggcagtgaaagttgactatttgccatgacataagaaataagtgtagtggctagtgtacaccctatgagtggaagggtccattttgaagtcagtggagtaagctttatgccagtttgatggtttcacaagttctattgagtgctattcagaataggaacaaggttctaatagaaaaagatggcaatttgaagtagctataaaattagactaatctacattgcttttctcctgcagagtctaataccttttatgctttgataattagcagtttgtctacttggtcactaggaatgaaactacatggtaataggcttaacaggtgtaatagcccacttactcctgaatctttaagcatttgtgcatttgaaaaatgcttttcgcgatcttcctgctgggattacaggcatgagccactgtgcctgacctcccatatgtaaaagtgtctaaaggtttttttttggttataaaaggaaaatttttgcttaagtttgaaggataggtaaaattaaaggacatgctttctgtttgtgtgatggtttttaaaaattttttttaagatggagttcttgttgcccaggctagaatgcaatggcaaaatctcactgcaatctcctcctcctgggttcaagcaattctcctacttcagcctcccaagtagctgggattacaggcatgtgctaatttggtgtttttaatagagatgaggtttttccatgttggtcaggctggtctcaaactcctgaccttaggtgatcgcctcggcctcctaaagtgctggaattacaggcatgagccaccatgcctggccaggacatgtgttcttaaggacatgctaagcaggagttaaagcagcccaagagataaggcctcttaaagtgactggcaatgtgtattgctcaagattcaaaggtacttgaattggccatagacaagtctgtaatgaagtgttatcgttttccctcatctgagtctgaattagataaaatgccttcccatcagccagtgctctgaggtatcaagtctaaattgaactagagatttttgtccttagtttctttgctatctaatgtttacacaagtaaatagtctaagatttgctggatgacagaaaaaacaggtaaggcctttaatagatggccaatagatgccctgataatgaaagttgacacctgtaagatttaccagtagagaattcttgacatgcaaggaagcaagatttaactgaaaaattgttcccactggaagcaggaatgagtcagtttacttgcatatactgagattgagattaacttcctgtgaaacccagtgtcttagacaactgtggcttgagcaccacctgctggtattcattacaaacttgctcactacaataaatgaattttaagctttaaaaaaaaaaaaaaaaaa

SEQ ID NO: 58 - protein, 462 aa, Homo sapiens
MGKEKTHINIVVIGHVDSGKSTTTGHLIYKCGGIDKRTIEKFEKEAAEMGKGSFKYAWVLDKLKAERERGITIDISLWKFETSKYYVTIIDAPGHRDFIKNMITGTSQADCAVLIVAAGVGEFEAGISKNGQTREHALLAYTLGVKQLIVGVNKMDSTEPPYSQKRYEEIVKEVSTYIKKIGYNPDTVAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEALSEALPGDNVGFNVKNVSVKDVRRGNVAGDSKNDPPMEAAGFTAQVIILNHPGQISAGYAPVLDCHTAHIACKFAELKEKIDRRSGKKLEDGPKFLKSGDAAIVDMVPGKPMCVESFSDYPPLGRFAVRDMRQTVAVGVIKAVDKKAAGAGKVTKSAQKAQKAK

SEQ ID NO: 59 - DNA, 2794 bp, Homo sapiens
cggcaggaccgagcgcggcaggcggctggcccagcgcagccagcgcggcccgaaggacgggagcaggcggccgagcaccgagcgctgggcaccgggcaccgagcggcggcggcacgcgaggcccggccccgagcagcgcccccgcccgccgcggcctccagcccggccccgcccagcgccggcccgcggggatgcggagcggcgggcgccggaggccgcggcccggctaggcccgcgctcgcgcccggacgcggcggcccgaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 60 - protein, 480 aa, Homo sapiens [sequence omitted]

SEQ ID NO: 61 - protein, 19 aa, Artificial
  An artificially synthesised dominant negative peptide
ENQSLRGVVQELQQAISKL

SEQ ID NO: 62 - protein, 33 aa, Artificial
  An artificially synthesised dominant negative peptide
RRRRRRRRRRRGGGENQSLRGVVQELQQAISKL

SEQ ID NO: 63 - protein, 9 aa, Artificial
  An artificially synthesised Tat sequence
RKKRRQRRR

SEQ ID NO: 64 - protein, 16 aa, Artificial
  An artificially synthesised Penetratin sequence
RQIKIWFQNRRMKWKK

SEQ ID NO: 65 - protein, 21 aa, Artificial
  An artificially synthesised Buforin II sequence
TRSSRAGLQFPVGRVHRLLRK

SEQ ID NO: 66 - protein, 27 aa, Homo sapiens
GWTLNSAGYLLGKINLKALAALAKKIL

SEQ ID NO: 67 - protein, 18 aa, Artificial
  An artificially synthesised MAP (model amphipathic peptide) sequence
KLALKLALKALKAALKLA

SEQ ID NO: 68 - protein, 16 aa, Artificial
  An artificially synthesised K-FGF sequence
AAVALLPAVLLALLAP

SEQ ID NO: 69 - protein, 5 aa, Artificial
  An artificially synthesised Ku70 sequence
VPMLK

SEQ ID NO: 70 - protein, 5 aa, Artificial
  An artificially synthesised Ku70-PMLKE sequence
PMLKE

SEQ ID NO: 71 - protein, 28 aa, Artificial
  An artificially synthesised Prion sequence
MANLGYWLLALFVTMWTDVGLCKKRPKP

SEQ ID NO: 72 - protein, 18 aa, Artificial
  An artificially synthesised pVEC sequence
LLIILRRRIRKQAHAHSK

SEQ ID NO: 73 - protein, 21 aa, Artificial
  An artificially synthesised Pep-1 sequence
KETWWETWWTEWSQPKKKRKV

SEQ ID NO: 74 - protein, 18 aa, Artificial
  An artificially synthesised SynB1 sequence
RGGRLSYSRRRFSTSTGR

SEQ ID NO: 75 - protein, 15 aa, Artificial
  An artificially synthesised Pep-7 sequence
SDLWEMMMVSLACQY

SEQ ID NO: 76 - protein, 12 aa, Artificial
  An artificially synthesised HN-1 sequence
TSPLNIHNGQKL

SEQ ID NO: 77 - protein, 11 aa, Artificial
  An artificially synthesised poly-arginine-11 sequence
RRRRRRRRRRR

SEQ ID NO: 78 - DNA, 5441 bp, Homo sapiens
agtgggggcctgatagcgcggcggtgtggaccgcgcggccgaagagcgcggcgcccagagcgcgggccgctcgcggagccacagcccgagccgggtcccagccggagccgagccccagccgagccgagccgggcccggagcgcccggtgcccgcagccatgccggccggccgcgccgcgcgcacctgtgcgctgctcgccctctgcctcctgggcgccggggcccaggatttcgggccgacgcgcttcatctgcacctcggtgcccgtggacgccgacatgtgcgccgcgtccgtggccgccggcggcgccgaggagctccggagcagcgtgctgcagctccgcgagacggtgctgcagcagaaggagaccatcctgagccagaaggagaccatccgcgagctgaccgccaagctgggccgctgcgagagccagagcacgctggaccccggagccggcgaggcccgggcgggcggcggccgcaagcagcccggctcgggcaagaacaccatgggcgacctgtcccggacaccggccgccgagacgctcagccaactcgggcaaactttgcaatcgctcaaaacccgcctggagaacctcgagcagtacagccgcctcaattcctccagccagaccaacagcctcaaggatctgctgcagagcaagatcgatgagctggagaggcaggtgctgtcccgggtgaacaccctggaggagggcaaggggggccccaggaacgacaccgaggagagggtcaagatcgagaccgccctgacctccctgcaccagcggatcagcgagctcgagaaaggtcagaaagacaaccgccctggagacaagttccagctcacattcccactgcggaccaactatatgtatgccaaggtgaagaagagcctgccagagatgtacgccttcactgtctgcatgtggctcaagtccagcgccacgccaggtgtgggcacgcccttctcctacgctgtgcccggccaggccaacgagctggtcctcattgagtggggcaacaaccccatggagatcctcatcaatgacaaggtggccaagttgccttttgtcatcaatgatggcaagtggcaccacatctgtgtcacctggaccacccgggacggggtctgggaggcctaccaggatggcacgcagggtggcagtggcgagaacttggcgccctatcaccccatcaagccccagggcgtgctggtgctgggccaggagcaggacactctgggtggtgggtttgatgccacccaggcatttgtgggtgagctggcccacttcaacatctgggaccgcaagctgacccccggggaggtgtacaacctggccacctgcagcaccaaggctctgtccggcaatgtcatcgcctgggctgaatcccacatcgagatctacggaggggccaccaagtggaccttcgaggcctgtcgccagatcaactgagcacggcaggccaggctgagcccgcccgccctcgccccctgcttgtgcggcgatgatctgttttgtgcgtctcttctctcccttttccccaggaatgaaccgaggccgtcgcccctgcacacgcacacgcacacagcctggttttgtcctcatgcacacgaagcagcccctgctcccatctgtccctgaggaagccccacttctctgtaggagcccggactctctcaggcatgccccattcacagctgaagtgggtgctgcaacgtcttgaacaaggcagaagttggtgagaggatctgtgtgtgcgtgtctacatgtgtgtgtctacgtgtgtgcgtgcgtggctgggggaggccttttctttgaggacgtacctcatttccttctttcttctggctttggaaaaatctcatgatgaaaattcatatttgccaactttgttagctgcgtgcgtgctttggggttggtgcaacctcagtacacgcatttgtctttgtttgcaaacctttctcagagcgacatatctttatattgatgtaataaatgtcttttagtggtttgtcaaaggccgggggcgggggctctctacagagaatttttattttgtaatagaagtgaactgtctctgaagggtgaaggcaggccgtcctgggatggtaccctgtgctctcccgtggaggagaggggatggctgaggacactggcccttaccccagggccagacagcatccatccctgctgtttgcatctgagagcagcatggggcctgggaggtcggcctgtgtgcccagctcagctagctctgccccaggacggccctgccctcgaccttcccacctcctcagatcctgcaaggctggggtctgcccctcccttctcacctctggagctgtgctgcactgcttcagcccagagggccctgagagaggagcgtgccacccacagcccgggaagccgggccccagcacccctctcctttggcctccggcagtgcagaccagaggggaccttttaaggaaagaagccgtgtttcgatgaagacctggccacatggggccactgggacttcaacccagcccatcggtgggaaggtcctttttgggggcctttgacagccatatccctcccagcacaccaggcgccaggtgagctggttcagacccctccaggggtactccagagacctcacgtgtggagccaggcctggccagggcaggggcctgaaacccactcctccatctcatggggctcacggcctacagcagcccacaagctgccactggccggcgacactgacacctgagcagtgtccagaacctttttgcctttttttgttccccgtgaaaagcaacatggacatttccttctagtccttccaaggaggggagagaagtgtatgtgcatttgtgtgtgtgtgtgtgtgttgtgtgtgtgtgtgcgctaagtgagaaagagagcaggctcgggaggccctgcccagggtaggaggagcttcctgctttgcaccatctggtggtcgcacgccctgagggcaccccgactctgtctccaggagtctcatcagcaaaccgctgacaagtctttctagaaattctactgcactgcctggctcagctgcagctgcagacatttctgcaggaggagcaggtgtttctgtcttctgttccttctagggccacctgtccccttaaacacaggtccacgttgtgtcaagaacctagtgcatctgtgtgtgtctgtcagtgtctctgtgtcagtgttcttgtgggtgtctgcacggtacccggccgccgttctgcaatgcatcactcccgcagaggggggtgcagatcaggcgccgtgctgcgcgttgttgttcaacagtggctttttcttagataatcgtgcttcctcagcgcccgtcgggttgtggcatccttggatctgcagggatcttctccgtttgcatgttcctcggggtggcgtgttccttgctccctgggtccgacatgtgttcccgcacctgcatggactgccccggttctgtgttgtgtgccgagtgccgcccagtgttctgtgaccacccgtgtagctactgaaaatggctgggtaagcaagtcaagggtgttggaggaggtcaagagagagctcagtttccctctccccctccccaaacacaccaagaagcatttttaacgtgtaggttgagaacaagcctaaaggattcccacagctgggagccagcaagagagcttggagtcgcctctctagaccagatctagccccaccctcactccagccatctcggagcccttgtgtaggcaacgcccggtgcgggctgtgtggggtgctcccctgccagcacctccggccagccccgcccctgccgatctactggaccgcagaccaccttctgcccccgtgggccaggtgggagctgtccgttcaggaccatgagccatcctctgccctgactagcgaggggcagagcacaccccagtgcttacgcctccacccctgcagcctcctggcccgctcaccttcctcacccctcctctgacccacccatggtgccagggccgaagctgacctttagctccctcctgccccttgctagggtctgagccaagcccctcgactccctcactgtgttgacacttggcactttgctggccccgagaaaggtcgatgacacagccgcaaatctaatccacgtagttcccatttactccttaatctgattgatgttccctcttgcactgaataatacatgcctctctcaggtaagccattttataaaacaagaagataaaaagcactgttgaggcagtgtttgcttttgccgagctggtgtccgacagctccctgggtgtccggggtgggagagctgttgacagaagctctccgggccctcaggggcttagatcccacttgagtcgtaagccttcttgcttttgataacacagtattatttctcttactgtagaagaaaaagtttattaccaaacaagagtatttttatgaaagaaaaggacaaacctataaattaactcaacctatatctcccttgaaaatactttcaggctccaccaaaacgtagaactgaaagcatgtattttggaagaaagagatacattttgtatgctttcttttccttttgtagattcccagtttattttctaagactgcaaagatcactttgtcaccagccctgggacctgagaccaagggggtgtcttgtgggcagtgagggggtgaggagaggctggcatgaggttcagtcattccagtgagctccaaagaggggccacctgttctcaaaagcatgttggggaccaggaggtaaaactggccatttatggtgaacctgtgtcttggagctgacttactaagtggaatgagccgaggatttgaatatcagttctaaccttgatagaagaaccttgggttacatgtggttcacattaagaggatagaatcctttggaatcttatggcaaccaaatgtggcttgacgaagtcgtggtttcatctcttaaacacagtgtgtaaatttattcaactaacgatgggaaatgtattacttctgtacacagtggactgaagtgcaatttgttgaaagggaacaagtcattgaagagaaaaaaaaaaagcccaatacttagagtcccaattttgtctcatttgccaaaaaaaaaaaaaaaaaaaaaaagcaaaccccctatggttgatattgttataatgtatatactgtataatatgaaagagaatcgatgtatctcactttttcattatttgctaaccaaagctgtacatttttcatatgatctgcagccttttgggtatcaaatgggtcaaaaccatgggacctgccacctcccatcagcaattctggaaatgcactatttctactggtattcttgctttttttttttttttcattttcttgctgaaatgacatgaattgttgagtttatttttacccagtaaagagtggagaaagact

SEQ ID NO: 79 - protein, 432 aa, Homo sapiens
MPAGRAARTCALLALCLLGAGAQDFGPTRFICTSVPVDADMCAASVAAGGAEELRSSVLQLRETVLQQKETILSQKETIRELTAKLGRCESQSTLDPGAGEARAGGGRKQPGSGKNTMGDLSRTPAAETLSQLGQTLQSLKTRLENLEQYSRLNSSSQTNSLKDLLQSKIDELERQVLSRVNTLEEGKGGPRNDTEERVKIETALTSLHQRISELEKGQKDNRPGDKFQLTFPLRTNYMYAKVKKSLPEMYAFTVCMWLKSSATPGVGTPFSYAVPGQANELVLIEWGNNPMEILINDKVAKLPFVINDGKWHHICVTWTTRDGVWEAYQDGTQGGSGENLAPYHPIKPQGVLVLGQEQDTLGGGFDATQAFVGELAHFNIWDRKLTPGEVYNLATCSTKALSGNVIAWAESHIEIYGGATKWTFEACRQIN

SEQ ID NO: 80 - DNA, 19 bp, Artificial
  An artificially synthesized primer for PCR
gttggggaccggaggtaaa

SEQ ID NO: 81 - DNA, 20 bp, Artificial
  An artificially synthesized primer for PCR
aaaccacgacttcgtcaagc

SEQ ID NO: 82 - DNA, 19 bp, Artificial
  An artificially synthesized target sequence for siRNA
ctcgggcaaactttgcaat

SEQ ID NO: 83 - DNA, 19 bp, Artificial
  An artificially synthesized target sequence for siRNA
ggtgaagaagagcctgcca

SEQ ID NO: 84 - DNA, 19 bp, Artificial
  An artificially synthesized target sequence for siRNA
gacaatggctggcaccaca

SEQ ID NO: 85 - DNA, 19 bp, Artificial
  An artificially synthesized target sequence for siRNA
catcaagcctcatgggatc

SEQ ID NO: 86 - DNA, 5814 bp, Homo sapiens
cggccgcggcgacagctccagctccggctccggctccggctccggctccggctcccgcgcctgccccgctcggcccagcgcgcccgggctccgcgccccgaccccgccgccgcgcctgccgggggcctcgggcgcccccgccgcccgcctcacgctgaagttcctggccgtgctgctggccgcgggcatgctggcgttcctcggtgccgtcatctgcatcatcgccagcgtgcccctggcggccagcccggcgcgggcgctgcccggcggcgccgacaatgcttcggtcgcctcgggcgccgccgcgtccccgggcccgcagcggagcctgagcgcgctgcacggcgcgggcggttcagccgggccccccgcgctgcccggggcacccgcggccagcgcgcacccgctgccgcccgggcccctgttcagccgcttcctgtgcacgccgctggctgctgcctgcccgtcgggggcccagcagggggacgcggcgggcgctgcgccgggcgagcgcgaagagctgctgctgctgcagagcacggccgagcagctgcgccagacggcgctgcagcaggaggcgcgcatccgcgccgaccaggacaccatccgtgagctcaccggcaagctgggccgctgcgagagcggcctgccgcgcggcctccagggcgccgggccccgccgcgacaccatggccgacgggccctgggactcgcctgcgctcattctggagctggaggacgccgtgcgcgccctgcgggaccgcatcgaccgcctggagcaggagcttccagcccgtgtgaacctctcagctgccccagccccagtctctgctgtgcccaccggcctacactccaagatggaccagctggaggggcagctgctggcccaggtgctggcactggagaaggagcgtgtggccctcagccacagcagccgccggcagaggcaggaagtggaaaaggagttggacgtcctgcagggtcgtgtggctgagctggagcacgggtcctcagcctacagtcctccagatgccttcaagatcagcatccccatccgtaacaactacatgtacgcccgcgtgcggaaggctctgcccgagctctacgcattcaccgcctgcatgtggctgcggtccaggtccagcggcaccggccagggcacccccttctcctactcagtgcccgggcaggccaacgagattgtactgctagaggcgggccatgagcccatggagctgctgatcaacgacaaggtggcccagctgcccctgagcctgaaggacaatggctggcaccacatctgcatcgcctggaccacaagggatggcctatggtctgcctaccaggacggggagctgcagggctccggtgagaacctggctgcctggcaccccatcaagcctcatgggatccttatcttgggccaggagcaggataccctgggtggccggtttgatgccacccaggcctttgtcggtgacattgcccagtttaacctgtgggaccacgccctgacaccagcccaggtcctgggcattgccaactgcactgcgccactgctgggcaacgtccttccctgggaagacaagttggtggaggcctttgggggtgcaacaaaggctgccttcgatgtctgcaaggggagggccaaggcatgaggggccacctcatccagggcccctcccttgcctgccactttggggacttgaggggggtcatattccctcctcagcctgcccacgcactggccttccctcctgccccactcctggctgtgcctcccatttcccctcacctgtacccacacctccagaatgccctgccctgcgagtgtgtcccctgtccccacctgagtggggaggagcgtctcaagtgaacagtgggagcctgcccacctggcactgcactggagttgtctcttaccccaccctccctgcccatcaactgtatctgatttcactaattttgacagcacccccagtagggtaggattgtgtatgagggggaccccactatctcagtggtgggggtggccgcccgcccccttgtcccccatgcaacaggcccagtggcttccccttcagggccacaacaggctgtagaaggggatgacgaggacatcagaggttagacttaccctcctccctctttccaccagctgccagtcaagggcagtgggatctcgatggagcctccccccccccccacccatgcctccctcttcctcctctttcctcctctctttgtgtgtagcggtttgaatgttggttccatgcctggcccagccccacctcagtctccaggacattcctttcccagctccagcctggagggaaggggacaaagaccccaggaggccaaagggctgcagtcaccccttgtgctcacccatagtgatggccactggtatagtcatcgctctccctccatgccaaggacaggacttggaccgcttcagcctgggctgggagcagccctaaggtagaggcctcatggcccaggagaccccacctctggcagagccacattacctaccctgtgcatggtcctggggcagcaaggaagaagctcagagggtggggagaagcatgaagcagtgagcagagcactgggtgagagggagaagaccttggttcctagccagccctgctaatgtgctgtgtggccttctgtaagtccctgccctctctgggcctggccttcctcattcgtgagctgaggccctcgctttggtcatttgctctccagattgggtgtgagcttctctgtgattccaggtggatatgtggggaaagctctggtgaccctgggcttcgcaggggtagatcccaggactcggcagtggatgggatgcagccagtcatgggttagggtcagcagagactcagagtccagggcaaggttcaaggcagactaacctcatgcatggattgtaaaaaaccagctccctttggatcaacccagcctggcacccttgcctgtctgagagtgtctcaaagggctgatggcttcctggtccccttgagtcatcaccagcttccccaagagagtgtcagaatcttaagagctgagaggccgggcacggtggctcacgcctgtaatcccagcactttgggaggctgagacaggcagatcacttgaggtcaggagttcgaagtcagcctggccaacgtggtgaaaccccatcttcactaaaaatacaaaacttagctggttaggtggtgcatgcctgtagtcccagctactcgggaggccgaggcagaagaatctcttgaactgaggaggtggaggttgcagtgagccgagatcacgccattgcactccagcctgggcaacagagcaagactccatctcaaaaaaataataataatcttaaagatgagaaaagccaccccatctggcaccacagctgcatcttgcttgtgagaaatggggaagagttcagggaggacacgtgacctgcacaggatcacagagcatggggcagagccaggactagagctcagggcatctgactccctcttcagtgttcttccccctccatgttgcctgcccctgaagacctttgagttcagtctacacctaagcaggtagacatccgcgaggtcagatgctttccaacatgacacctgaacatcttcctttatgcaacacccaaacatcttggcatccccaccccaggaagtgcggggaggaggttatgatccctgggcgcttcggcagaatggagagctgaggtgtccctcccctgctagtcacctaccaggtgtctgagcagctgcatgctccctggctcaagtgggcactgtaccttttgcctgcctttttgttccctatctccactccctgaggccacttagcctgagacatgatgcaagagctgcaggccggggggctcagtgccatggaagctactccaagttgcattgcctcccgcgcccagatcctgctttccatttcgagaacataaatagattgcccagcccctccagtacaatcccactggaagaaaaggcaatggcgggcttcagccagacctgctgagacctaggttgccacggtaacagccaaagacatcaacccaagtgctgggtcaagtgtctcatcatactggcactgttgctggggtgacggcagaattcagaacttcaatttcagtgacgccaagcttgatgtgtttctgttattgttttgaagaaggtagctcttgtggaggacttgggagaaggatggggtcttaggaaggaggtgacagcacttgcatggtcacttgagcccacacacacgctcaaccccaagtcctttatgctttgtcacagtgaagatgagacctctgacgtccaagccttgttcctgtgctgcatcacccactcagccttccaaagggaacaggaacaaatttccccagcaccactgtttgggtcccgcttttcctatcttctgctgcccctgagcacatccaagcagacagggaaagaggagtcagacatggcccagtcacatcctgagctgctcctggctgataaccacgatggagcccgtgtttgtcctgccatctggcactgcactgagtgtggcacaggcaccgtcctgttgatctcacaacacagttctaagttaggacgttcttggctccgttagacaggtgaggaaactggggcacagagaggtgatgtcatctgcctggtgtcaatcagctagcaagtgatggagcccagatttcaaaccaaagggggttacgtccaggggctgagttcccactcacctgtgtagagtgccatctgggcaccattgctccagacgtgttccgacccctttcccagcccacagggcttgaagtgaaggaacagaggcagggggtgggccagccccagggccagggtccccttggtgaagccgtgccagggggctcagctgcttcagggaatgtgtccctcccaccatgggccagagcttcagcccttctttagctcagctagagttcacaggagagccaaaaaagaaaaggaagctgagcatctcccgagtcctgggcagggaaggggagggaaattgctgcttctccaactcttgcttggggccaagccctgcaccagttgcttcccagctgttatctgccagatcttcccatcttgtggcatgtggtgcccccaccaacatcccaaggggaccaatccccttgccaccactttgcatcacctgggaccacagatttggacaggaagggctctgagaagaggccaaagccctcattttacagatgaggaagctgaagcccggggaggggagcgaccctcaaggccacccagctggacacgggagacttgagcccagccttctgactgcattcagccctctctaggacgcagcagcctctccccagcactgagtcccccctcctttgtgtgtcccagcacccttggcctgagtaaacttggaaaggggctccctcccagagaagggactactctcttcacccctttattccagctgcctgccaccccagacccccacctcccaccctgacccccgacccctgggtggggaaggggctcacatgggcccaggctgagtgtgagtgagcatgtcaagttgtctgacactgtgacattagtgcaccctactgacaacccctccccagccttgcccctttctcctctccctgttttgtacataaattgacatgagctgcaacatgtgtgcgtgtgtgtgcgtgtgtgtgtgtgtgtatgtgtgtgtgatctgtgtcatggttttgttacctttttgtttttgtaaacttgaatgttcaaaataaacatgctgtttactctga

SEQ ID NO: 87 - protein, 500 aa, Homo sapiens
MKFLAVLLAAGMLAFLGAVICIIASVPLAASPARALPGGADNASVASGAAASPGPQRSLSALHGAGGSAGPPALPGAPAASAHPLPPGPLFSRFLCTPLAAACPSGAQQGDAAGAAPGEREELLLLQSTAEQLRQTALQQEARIRADQDTIRELTGKLGRCESGLPRGLQGAGPRRDTMADGPWDSPALILELEDAVRALRDRIDRLEQELPARVNLSAAPAPVSAVPTGLHSKMDQLEGQLLAQVLALEKERVALSHSSRRQRQEVEKELDVLQGRVAELEHGSSAYSPPDAFKISIPIRNNYMYARVRKALPELYAFTACMWLRSRSSGTGQGTPFSYSVPGQANEIVLLEAGHEPMELLINDKVAQLPLSLKDNGWHHICIAWTTRDGLWSAYQDGELQGSGENLAAWHPIKPHGILILGQEQDTLGGRFDATQAFVGDIAQFNLWDHALTPAQVLGIANCTAPLLGNVLPWEDKLVEAFGGATKAAFDVCKGRAKA

SEQ ID NO: 88 - protein, 126 aa, Artificial
  An artificially synthesized epitope peptide sequence
QDFGPTRFICTSVPVDADMCAASVAAGGAEELRSSNVLQLRETVLQQKETILSQKETIRELTAKLGRCESQSTLDPGAGEARAGGGRKQPGSGKNTMGDLSRTPAAETLSQLGQTLQSLKTRLENL

SEQ ID NO: 89 - protein, 134 aa, Artificial
  An artificially synthesized epitope peptide sequence
KVAKLPFVINDGKWHHICVTWTTRDGVWEAYQDGTQGGSGENLAPYHPIKPQGVLVLGQEQDTLGGGFDATQAFVGELAHFNIWDRKLTPGEVYNLATCSTKALSGNVIAWAESHIEIYGGATKWTFEACRQIN

SEQ ID NO: 90 - DNA, 1841 bp, Homo sapiens
actgcgccgccaccgtcaataggtggaccccctcccggagataaaaccgccggcgccggcgccgccagtccctctggctgagacctcggctccggaatcactgcagcccccctcgccctgagccagagcaccccgggtcccgccagcccctcacactcccagcaaaatgggcaaggagaagacccacatcaacatcgtggtcatcggccacgtggactccggaaagtccaccaccacgggccacctcatctacaaatgcggaggtattgacaaaaggaccattgagaagttcgagaaggaggcggctgagatggggaagggatccttcaagtatgcctgggtgctggacaagctgaaggcggagcgtgagcgcggcatcaccatcgacatctccctctggaagttcgagaccaccaagtactacatcaccatcatcgatgcccccggccaccgcgacttcatcaagaacatgatcacgggtacatcccaggcggactgcgcagtgctgatcgtggcggcgggcgtgggcgagttcgaggcgggcatctccaagaatgggcagacgcgggagcatgccctgctggcctacacgctgggtgtgaagcagctcatcgtgggcgtgaacaaaatggactccacagagccggcctacagcgagaagcgctacgacgagatcgtcaaggaagtcagcgcctacatcaagaagatcggctacaacccggccaccgtgccctttgtgcccatctccggctggcacggtgacaacatgctggagccctcccccaacatgccgtggttcaagggctggaaggtggagcgtaaggagggcaacgcaagcggcgtgtccctgctggaggccctggacaccatcctgccccccacgcgccccacggacaagcccctgcgcctgccgctgcaggacgtgtacaagattggcggcattggcacggtgcccgtgggccgggtggagaccggcatcctgcggccgggcatggtggtgacctttgcgccagtgaacatcaccactgaggtgaagtcagtggagatgcaccacgaggctctgagcgaagctctgcccggcgacaacgtcggcttcaatgtgaagaacgtgtcggtgaaggacatccggcggggcaacgtgtgtggggacagcaagtctgacccgccgcaggaggctgctcagttcacctcccaggtcatcatcctgaaccacccggggcagattagcgccggctactccccggtcatcgactgccacacagcccacatcgcctgcaagtttgcggagctgaaggagaagattgaccggcgctctggcaagaagctggaggacaaccccaagtccctgaagtctggagacgcggccatcgtggagatggtgccgggaaagcccatgtgtgtggagagcttctcccagtacccgcctctcggccgcttcgccgtgcgcgacatgaggcagacggtggccgtaggcgtcatcaagaacgtggagaagaagagcggcggcgccggcaaggtcaccaagtcggcgcagaaggcgcagaaggcgggcaagtgaagcgcgggcgcccgcggcgcgaccctccccggcggcgccgcgctccgaaccccggcccggcccccgccccgcccccgccccgcgcgccgctccggcgccccgcacccccgccaggcgcatgtctgcacctccgcttgccagaggccctcggtcagcgactggatgctcgccatcaaggtccagtggaagttcttcaagaggaaaggcgcccccgccccaggcttccgcgcccagcgctcgccacgctcagtgcccgttttaccaataaactgagcgaccccag

SEQ ID NO: 91 - protein, 463 aa, Homo sapiens
MGKEKTHINIVVIGHVDSGKSTTTGHLIYKCGGIDKRTIEKFEKEAAEMGKGSFKYAWVLDKLKAERERGITIDISLWKFETTKYYITIIDAPGHRDFIKNMITGTSQADCAVLIVAAGVGEFEAGISKNGQTREHALLAYTLGVKQLIVGVNKMDSTEPAYSEKRYDEIVKEVSAYIKKIGYNPATVPFVPISGWHGDNMLEPSPNMPWFKGWKVERKEGNASGVSLLEALDTILPPTRPTDKPLRLPLQDVYKIGGIGTVPVGRVETGILRPGMVVTFAPVNITTEVKSVEMHHEALSEALPGDNVGFNVKNVSVKDIRRGNVCGDSKSDPPQEAAQFTSQVIILNHPGQISAGYSPVIDCHTAHIACKFAELKEKIDRRSGKKLEDNPKSLKSGDAAIVEMVPGKPMCVESFSQYPPLGRFAVRDMRQTVAVGVIKNVEKKSGGAGKVTKSAQKAQKAGK